(19) United States
(12) Patent Application Publication
(10) Pub. No.: US 2025/0297246 A1
(43) Pub. Date: Sep. 25, 2025

(54) MODIFIED PRIME EDITING GUIDE RNAS

(71) Applicant: Prime Medicine, Inc., Cambridge, MA (US)

(72) Inventors: Andrew V. Anzalone, Cambridge, MA (US); John Stiller, Allston, MA (US); David Wiley, Dedham, MA (US)

(21) Appl. No.: 18/712,922

(22) PCT Filed: Nov. 23, 2022
(86) PCT No.: PCT/US22/50874

§ 371 Date: May 23, 2024

Related U.S. Application Data
(60) Provisional application No. 63/283,076, filed on Nov. 24, 2021.
(60) Provisional application No. 63/417,857, filed on Oct. 20, 2022.

(51) Int. Cl. C12N 15/11; C12N 9/12; C12N 9/22; C12N 15/90

(52) U.S. Cl. CPC C12N 15/11; C12N 9/1276; C12N 9/226; C12N 15/907; C12Y 207/07049; C12N 2310/20

(57) ABSTRACT

Provided herein are compositions and methods related to modified prime editing guide RNAs.

### CROSS REFERENCE TO RELATED APPLICATIONS

This application is a § 371 national-stage application based on PCT/US2022/050874, filed Nov. 23, 2022, which claims the benefit of U.S. Provisional Application No. 63/283,076, filed Nov. 24, 2021, and U.S. Provisional Application No. 63/417,857, filed Oct. 20, 2022, the entire contents of each are hereby incorporated by reference.

### REFERENCE TO SEQUENCE LISTING

The instant application contains a Sequence Listing which has been submitted electronically in XML format via EFS-Web, and is hereby incorporated by reference in its entirety. Said XML copy, created on Mar. 9, 2023, is named PMB_00101_SeqList_ST26 and is 3,944,448 bytes in size is hereby incorporated by reference in its entirety.

### BACKGROUND

Prime editing is a gene editing technology that allows researchers to make nucleotide substitutions, insertions, deletions, or combinations thereof in the DNA of cells. Prime editing can be used to correct disease associated gene mutations, and can be used for treating disease with a genetic component. There is a need for improved prime PEgRNAs that have desirable properties, such as the ability to facilitate prime editing with improved efficiency.

### SUMMARY

Provided herein are prime editing guide RNAs (PEgRNAs) useful in prime editing, as well as methods of using and making such PEgRNAs.

In some aspects, provided herein are prime editing guide RNA (PEgRNA) s comprising: (a) a spacer that comprises a region of complementarity to a search target sequence in a target strand of a double stranded target DNA; (b) a guide RNA (gRNA) core capable of binding to a Cas protein; (c) an extension arm comprising: (i) an editing template that comprises an intended edit compared to the double stranded target DNA, and (ii) a primer binding site (PBS) that comprises a region of complementarity to a region upstream of a nick site in a non-target strand of the double stranded target DNA; and (d) a 3′ nucleic acid motif selected from the group consisting of SEQ ID NOs 1-15.

In some aspects, also provided herein are prime editing guide RNA (PEgRNA) s comprising: (a) a spacer that comprises a region of complementarity to a search target sequence in target strand of a double stranded target DNA; (b) a guide RNA (gRNA) core capable of binding to a Cas protein; (c) an extension arm comprising: (i) an editing template that comprises an intended edit compared to the double stranded target DNA, and (ii) a primer binding site (PBS) that comprises a region of complementarity to a region upstream of a nick site in a non-target strand of the double stranded target DNA; and (d) a 3′ nucleic acid motif, wherein the 3′ nucleic acid motif comprises a sequence selected from the group consisting of: (A) a G-quadruplex or a C-quadruplex derived from a VEGF gene promoter, (B) a pseudoknot derived from a potato roll leaf virus (PLRV), (C) a MS2 protein binding sequence, (D) a Moloney Murine leukemia virus (MMLV) reverse transcriptase recruitment sequence, or a Moloney Murine leukemia virus (MMLV) replication recognition sequence.

In some embodiments, 3′ nucleic acid motif is the G-quadruplex or the C-quadruplex derived from a VEGF gene promoter (e.g., wherein the G-quadruplex comprises SEQ ID NO: 10 and/or the C-quadruplex comprises SEQ ID NO: 11). In some embodiments, the 3′ nucleic acid motif is the pseudoknot derived from a potato roll leaf virus (PLRV) (e.g., wherein the pseudoknot comprises SEQ ID NO: 4). In some embodiments, the 3′ nucleic acid motif comprises the MS2 protein binding sequence (e.g., wherein the MS2 protein binding sequence if SEQ ID NO: 9). In some embodiments, the 3′ nucleic acid motif comprises the MMLV reverse transcriptase recruitment sequence (e.g., the MMLV reverse transcriptase recruitment sequence comprises SEQ ID NO: 8). In some embodiments, the 3′ nucleic acid motif comprises MMLV replication recognition sequence.

In some embodiments, the MMLV replication recognition sequence comprises a sequence selected from the group consisting of SEQ ID NO:s 12-15. In some embodiments, the 3′ nucleic acid motif comprises SEQ ID NO: 1, 2, 3, 5, 6, or 7.

As provided herein, the PEgRNA comprises, in 5′ to 3′ order, the spacer, the gRNA core, the editing template, the PBS, and the 3′ nucleic acid motif. In some embodiments, the PEgRNA further comprises a linker immediately 5′ of the 3′ nucleic acid motif. The linker may be 2 to 12 nucleotides in length, such as 8 nucleotides in length. In some embodiments, the linker does not form a secondary structure. In some embodiments, the linker does not have perfect complementarity with the PBS sequence, editing template, the scaffold, and/or the extension arm. In some embodiments, the linker has no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, or no more than 15% complementarity to the extension arm.

In some aspects, provided herein are prime editing guide RNA (PEgRNA) comprising: (a) a spacer that comprises a region of complementarity to a search target sequence in target strand of a double stranded target DNA; (b) an extension arm comprising: (i) an editing template that comprises an intended edit compared to the double stranded target DNA, and (ii) a primer binding site (PBS) that comprises a region of complementarity to a region upstream of a nick site in a non-target strand of the double stranded target DNA; and (c) a guide RNA (gRNA) core comprising at least 80% identity to SEQ ID NO: 16 and containing one or more modifications relative to SEQ ID NO: 16, the one or more modifications comprising: (A) a first insertion between nucleotides 12 and 13 and a second insertion between nucleotides 16 and 17, wherein the first insertion is the reverse complement of the second insertion; (B) a first insertion between nucleotides 52 and 53 and a second insertion between nucleotides 56 and 57, wherein the first insertion is the reverse complement of the second insertion; (C) complementary substitutions of nucleotides 2 and 29, 3 and 28, 4 and 27, 51 and 58, or combinations thereof; (D) replacement of nucleotides 11-12 with a replacement sequence 1 and replacement of nucleotides 17-18 with a replacement sequence 2, wherein the replacement sequences 1 is at least 3 nucleotides in length and wherein the replacement sequence 2 is the reverse compliment of replacement sequence 1; (E) a T to G or T to C substitution at nucleotide 5 and a complementary substitution at nucleotide 26; or (F) any combination thereof.

In some embodiments, the gRNA core comprises the first insertion between nucleotides 12 and 13 and the second insertion between nucleotides 16 and 17. The insertion may be 1 to 6 nucleotides in length. In some embodiments, the first insertion comprises the sequence UGCUG. In some embodiments, the second insertion comprises the sequence CAGCA.

In some embodiments, the one or more modification comprises a replacement of nucleotides 49-52 with a replacement sequence 1 and replacement of nucleotides 57-60 with replacement sequence 2, wherein the replacement sequence 2 is the reverse complement of the replacement sequence 1; optionally wherein the replacement sequence 1 is 7-11 nucleotides in length; optionally wherein the replacement sequence 1 is 7-9 nucleotides in length; optionally wherein the replacement sequence 1 comprises GCGUCUC, GCGUCCC, GCGUCCA, GCGUGUGA, GCGUAGCC, GCGUGCAGA, GCGUACCCU, or GCGUUGUCG.

In some embodiments, the first insertion is 1 to 3 nucleotides in length, for example, first insertion may comprise a sequence selected from the group consisting of C, CC, CA, CG, A, AC, AA, AG, CCC, CCAC, CCAAC, and CCACAC.

In some embodiments, the gRNA core comprises the first insertion between nucleotides 52 and 53 and the second insertion between nucleotides 56 and 57. The first insertion may be 1 to 8 nucleotides in length. In some embodiments, the gRNA core comprises the complementary substitutions of nucleotides 2 and 29, 3 and 28, 4 and 27, 11 and 18, 12 and 17, 51 and 58, or combinations thereof. In some embodiments, the gRNA core comprises a U to A substitution at nucleotide 2. In some embodiments, the gRNA core comprises a U to A substitution at nucleotide 3. In some embodiments, the gRNA core comprises a U to A substitution at nucleotide 4. In some embodiments, the gRNA core comprises a U to G substitution at nucleotide 51 and optionally an A to C substitution at nucleotide 58.

In some embodiments, the gRNA core comprises the replacement of nucleotides 11-12 with the replacement sequence 1 and the replacement of nucleotides 17-18 with the replacement sequence 2.

In some embodiments, the replacement sequence 1 is 3 to 5 nucleotides in length, for example, the replacement sequence 1 may comprise a sequence selected from the group consisting of CAGC, CCGC, GGAC, UGC, UCC, GAGGC, AGC, GGC, CGCA, GCACA, GGUC, and GGG. In some embodiments, the gRNA core further comprises a U to A substitution at nucleotide 5 and an A to U substitution at nucleotide 26. In some embodiments, the gRNA core comprises complementary substitutions at nucleotides 52 and 57. In some embodiments, the gRNA core comprises a U to G substitution at nucleotide 52 and an A to C substitution at nucleotide 57. In some embodiments, the gRNA core comprises a U to C substitution at nucleotide 52 and an A to G substitution at nucleotide 57. In some embodiments, the gRNA core comprises complementary substitutions at nucleotides 49 and 60. In some embodiments, the gRNA core comprises an A to G substitution at nucleotide 49 and a U to C substitution at nucleotide 60.

In some aspects, provided herein are prime editing guide RNA (PEgRNA) comprising: (a) a spacer that comprises a region of complementarity to a search target sequence in target strand of a double stranded target DNA; (b) an extension arm comprising: (i) an editing template that comprises an intended edit compared to the double stranded target DNA, and (ii) a primer binding site (PBS) that comprises a region of complementarity to a region upstream of a nick site in a non-target strand of the double stranded target DNA; and (c) a guide RNA (gRNA) core comprising at least 80% identity to SEQ ID NO: 16 and containing one or more modifications relative to SEQ ID NO: 16, the one or more modifications comprising: (A) a U to A substitution at nucleotide 5 and an A to U substitution at nucleotide 26, and (B) a modification selected from the group consisting of: a. a first insertion between nucleotides 12 and 13 having the sequence of UGCUG and a second insertion between nucleotides 16 and 17 having the sequence of CAGCA; b. a first insertion between nucleotides 12 and 13 having the sequence of UGCUG and a second insertion between nucleotides 16 and 17 having the sequence of CAGCA, an A to G substitution at nucleotide 49, a U to C substitution at nucleotide 60, a U to G substitution at nucleotide 51, and an A to C substitution at nucleotide 58; c. a replacement of nucleotides 11-12 with replacement sequence 1 and a replacement of nucleotides 17-18 with replacement sequence 2, wherein the replacement sequence 1 comprises GGG and wherein the replacement sequence 2 comprises UCC; d. a replacement of nucleotides 11-12 with replacement sequence 1 and a replacement of nucleotides 17-18 with replacement sequence 2, wherein the replacement sequence 1 comprises GGG and wherein the replacement sequence 2 comprises UCC, an A to G substitution at nucleotide 49, a U to C substitution at nucleotide 60, a U to G substitution at nucleotide 51, and an A to C substitution at nucleotide 58; or e. a replacement of nucleotides 11-12 with replacement sequence 1 and a replacement of nucleotides 17-18 with replacement sequence 2, wherein the replacement sequence 1 and replacement sequence 2 comprises sequences CAGC and GCUG, CCGC and GCGG, GGAC and GUCC, GC and GC, CC and GG, GAGGC and GUCUC, AGC and GCU, GGC and GCC, CGCA and UGCG, GCACA and UGUGC, or GGUC and GGCC.

In some embodiments, the gRNA core comprises nucleotides 62-76 of SEQ ID NO: 16.

In some embodiments, the one or more modifications comprises a replacement of nucleotides 49-52 with a replacement sequence 1 and replacement of nucleotides 57-60 with replacement sequence 2, wherein the replacement sequence 2 is the reverse complement of the replacement sequence 1; optionally wherein the replacement sequence 1 is 7-11 nucleotides in length; optionally wherein the replacement sequence 1 is 7-9 nucleotides in length; optionally wherein the replacement sequence 1 comprises GCGUCUC, GCGUCCC, GCGUCCA, GCGUGUGA, GCGUAGCC, GCGUGCAGA, GCGUACCCU, or GCGUUGUCG.

In some aspects, provided herein are prime editing guide RNAs (PEgRNAs) comprising: a spacer that comprises a region of complementarity to a search target sequence in target strand of a double stranded target DNA; an extension arm comprising: an editing template that comprises an intended edit compared to the double stranded target DNA, and a primer binding site (PBS) that comprises a region of complementarity to a region upstream of a nick site in a non-target strand of the double stranded target DNA; and a guide RNA (gRNA) core comprising a sequence selected from the group consisting of SEQ ID NO:s 17-61, 3860-4253, 4255-4349, 4351-4359, and 4452.

In some embodiments, the gRNA core comprises a sequence selected from the group consisting of SEQ ID NOs: 4294, 4319, 4322, 4286, 4290, 4346, 4271, 4264, 4317, 4330, 4312, 4356, 4280, and 4452. In some embodiments, the gRNA core comprises SEQ ID NO: 4354.

In some embodiments, the PEgRNA comprises a 3′ nucleic acid motif selected from the group consisting of SEQ ID NOs 1-15, such as SEQ ID NO: 1, 2, 3, 5, 6, or 7. In some embodiments, the PEgRNA comprises a 3′ nucleic acid motif, wherein the 3′ nucleic acid motif comprises a sequence selected from the group consisting of: a G-quadruplex or a C-quadruplex derived from a VEGF gene promoter, a pseudoknot derived from a potato roll leaf virus (PLRV), a MS2 protein binding sequence, a Moloney Murine leukemia virus (MMLV) reverse transcriptase recruitment sequence, or a Moloney Murine leukemia virus (MMLV) replication recognition sequence.

In some embodiments, the selected 3′ nucleic acid motif is the G-quadruplex or the C-quadruplex derived from a VEGF gene promoter. The G-quadruplex may comprise SEQ ID NO: 10. The C-quadruplex may comprise SEQ ID NO: 11. In some embodiments, the selected 3′ nucleic acid motif is the pseudoknot derived from a potato roll leaf virus (PLRV). The pseudoknot may comprise SEQ ID NO: 4. In some embodiments, the 3′ nucleic acid motif comprises the MS2 protein binding sequence, such as SEQ ID NO: 9. In some embodiments, the 3′ nucleic acid motif comprises the MMLV reverse transcriptase recruitment sequence, such as the MMLV reverse transcriptase recruitment sequence comprises SEQ ID NO: 8. In some embodiments, the selected 3′ nucleic acid motif comprises MMLV replication recognition sequence, such as a sequence selected from the group consisting of SEQ ID NO:s 12-15.

In some embodiments, the PEgRNA comprises, in 5′ to 3′ order, the spacer, the gRNA core, the editing template, the PBS, and the 3′ nucleic acid motif. In some embodiments, the PEgRNA further comprises a linker immediately 5′ of the 3′ nucleic acid motif. The linker is 2 to 12 nucleotides in length, such 8 nucleotides in length. In some embodiments, the linker does not form a secondary structure. In some embodiments, the linker does not have perfect complementarity with the PBS sequence, the editing template, the scaffold, and/or the extension arm.

The linker may comprise no more than 90%, no more than 85%, no more than 80%, no more than 75%, no more than 70%, no more than 65%, no more than 60%, no more than 55%, no more than 50%, no more than 45%, no more than 40%, no more than 35%, no more than 30%, no more than 25%, no more than 20%, or no more than 15% complementarity to the extension arm.

In some aspects, provided herein are prime editing guide RNAs (PEgRNAs) comprising: (a) a spacer that comprises a region of complementarity to a search target sequence in target strand of a double stranded target DNA; (b) a guide RNA (gRNA) core capable of binding to a Cas protein; (c) an extension arm comprising: (i) an editing template that comprises an intended edit compared to the double stranded target DNA; and (ii) a primer binding site (PBS) that comprises a region of complementarity to a region upstream of a nick site in a non-target strand of the double stranded target DNA, and (d) a tag sequence that is the reverse complement of a sequence within the editing template.

In any embodiment disclosed herein the tag sequence may be from 4 nucleotides to 22 nucleotides in length, such as from 4 nucleotides to 10 nucleotides in length, from 4 nucleotides to 9 nucleotides in length, from 6 nucleotides to 8 nucleotides in length, 6 nucleotides in length, or 8 nucleotides in length.

In some embodiments, the tag sequence does not have perfect complementarity with the PBS, the gRNA core, and/or the spacer. In some embodiments, the PEgRNA comprises, in 5′ to 3′ order, the spacer, the gRNA core, the editing template, the PBS, and the tag sequence. In some embodiments, the PEgRNA comprises, in 5′ to 3′ order, the editing template, the PBS, the tag sequence, the spacer, and the gRNA core.

In some embodiments, the PEgRNA comprises a linker between the PBS and the tag sequence. The linker may be from 2 to 12 nucleotides in length, such as from 4 nucleotides to 8 nucleotides in length, 4 to 6 nucleotides in length, 8 nucleotides in length, 6 nucleotides in length, or 4 nucleotides in length.

In some embodiments, the linker does not have perfect complementarity with the PBS, the gRNA core, and/or the spacer. In some embodiments, the linker does not form a secondary structure.

In some embodiments, the gRNA core comprises a sequence selected from the group consisting of SEQ ID NOs: 16-60, 3860-4359, and 4452.

Also provided herein are prime editing system comprising: (a) a PEgRNA disclosed herein or one or more polynucleotides encoding the PEgRNAs disclosed herein; and (b) a prime editor comprising a Cas protein and a DNA polymerase or one or more polynucleotides encoding the prime editor. In some embodiments, Cas protein has a nickase activity. The Cas protein is a Cas9 may comprise a mutation in an HNH domain.

In some embodiments, the Cas9 comprises at least 80%, 85%, 90%, 95%, 96%, 97%, 98%, 99%, or 100% identity to compared to SEQ ID NO: 4442. The Cas protein may be a Cas12a, Cas12b, Cas12c, Cas12d, Cas12e, Cas14a, Cas14b, Cas14c, Cas14d, Cas14c, Cas14f, Cas14g, Cas14h, Cas14u, or Casφ.

In some embodiments, the DNA polymerase is a reverse transcriptase, such as a retrovirus reverse transcriptase. In some embodiments, the reverse transcriptase comprises at least 80%, 85%, 90%, 95%, 96%, 97%, 98%, 99%, or 100% identity to SEQ ID NO: 4444. In some embodiments, the Cas protein and the DNA polymerase are fused or linked in a fusion protein. In some embodiments, the fusion protein comprises the sequence of SEQ ID NO: 4440.

In some embodiments, the one or more polynucleotides comprise (a) a first sequence encoding an N-terminal portion of the Cas protein and an intein-N and (b) a second sequence encoding an intein-C, a C-terminal portion of the Cas protein and the DNA polymerase.

In some embodiments, the prime editing system comprises one or more vectors that comprise the one or more polynucleotide encoding the PEgRNA and the one or more polynucleotides encoding the prime editor. The one or more vectors may be, for example, AAV vectors. The one or more polynucleotides may be mRNA.

Also provided herein are lipid nanoparticle (LNP) or ribonucleoprotein (RNP) comprising the prime editing system disclosed herein.

In some aspects, provided herein are methods for editing a double stranded target DNA, the method comprising contacting the target DNA with (a) a PEgRNA disclosed herein and a prime editor comprising a Cas9 nickase and a reverse transcriptase, (b) a prime editing system of disclosed herein, or (c) the LNP or RNP disclosed herein. Target DNA disclosed herein may be in a cell.

In some embodiments, the editing efficiency for editing a double stranded target DNA is at least 1.1-fold, 1.2-fold, 1.3-fold, 1.4-fold, 1.5-fold, 1.6-fold, 1.7-fold, 1.8-fold, 1.9-fold, 2-fold, 2.1-fold, 2.2-fold, 2.3-fold, 2.4-fold, or 2.5-fold higher compared to the editing efficiency with a control PEgRNA having the same spacer and extension arm, wherein the control PEgRNA contains a gRNA core having the sequence of SEQ ID NO: 16 and does not contain a 3′ nucleic acid motif or a tag.

In some embodiments, the gRNA core comprises a sequence selected from the group consisting of SEQ ID NOs: 4352, 3860, 3862, 3865, 3908, 3915, 3982, 3991, 4035, 4261, 4262, 4263, 4264, 4265, 4266, 4268, 4277, 4278, 4280, 4283, 4284, 4285, 4286, 4269, 4287, 4288, 4289, 4290, 4291, 4270, 4271, 4272, 4274, 4275, 4276, 4292, 4301, 4302, 4304, 4305, 4306, 4309, 4293, 4311, 4312, 4313, 4315, 4316, 4317, 4319, 4320, 4294, 4321, 4322, 4323, 4295, 4296, 4297, 4299, 4324, 4333, 4334, 4338, 4339, 4341, 4342, 4343, 4345, 4346, 4348, 4349, 4328, 4329, 4330, and 4332.

In certain aspects, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, wherein the PEgRNA comprises one or more nucleic acid moieties at its 3′ end. In some embodiments, the PEgRNAs comprise, in 5′ to 3′ order, the spacer, the gRNA core, the editing template, and the PBS.

In some embodiments, the one or more (e.g., two or more, three or more, four or more, or five or more) nucleic acid moieties comprise a hairpin (e.g., hairpin comprising a region of self-complementarity, optionally wherein the region of self-complementary comprises 2, 3, 4, 5, 6, 7, 8, 9 or 10 contiguous complementary basepairs), a quadruplex (e.g., a G-quadruplex or a C-quadruplex, optionally wherein the G-quadruplex or the C-quadruplex is derived from a VEGF gene promoter), a tRNA sequence (e.g., a tRNA sequence, optionally wherein the tRNA sequence is a tRNA (Proline) sequence), an aptamer (e.g., an aptamer derived from a viral protein-binding sequence, optionally wherein the aptamer comprises a viral reverse transcriptase recruitment sequence, optionally wherein the aptamer comprises a MS2 protein binding sequence or a Moloney Murine leukemia (MMLV) reverse transcriptase recruitment sequence), and/or a pseudoknot (e.g., pseudoknot is derived form a potato roll leaf virus (PLRV), or any combination thereof. In some embodiments, the one or more nucleic acid moieties comprise a structure derived form a replication recognition sequence of a retrovirus, optionally wherein the retrovirus is a Moloney Murine leukemia (MMLV). For example, the one or more nucleic acid moieties may comprise a configuration as set forth in Table 4. In some embodiments, the one or more nucleic acid moieties comprise a nucleic acid sequence selected from SEQ ID NOs 1-15.

In certain embodiments, the PEgRNAs provided herein comprise a linker immediately 5′ of the one or more nucleic acid moieties. In some embodiments, the linker is 1, 2, 3, 4, 5, 6, 7, 8, 9, 10, 11, 12, 13, 14, 15, 16 nucleotides in length. In some embodiments, the linker is 2 to 13 nucleotides in length. In some embodiments, the linker is 8 nucleotides long. In some embodiments, the linker does not form a secondary structure. In some embodiments, the linker does not have a region of complementarity to the PBS sequence. In some embodiments, the linker does not have a region of complementarity to the editing template.

In certain embodiments, the gRNA core of the PEgRNAs provided herein comprises one or more sequence modifications compared to SEQ ID NO. 16. In some embodiments, the one or more (e.g., two or more, three or more, four or more, or five or more) sequence modifications comprises a gRNA core difference set forth in Table 1. In some embodiments, the gRNA core of a PEgRNA comprises a gRNA core sequence as set forth in Table 1 or Table 2. In some embodiments, the one or more sequence modifications comprises a sequence modification in the direct repeat. For example, the direct repeat may comprise at least one flip of an A/U basepair in the lower stem of the direct repeat, optionally wherein the lower stem does not contain 2, 3, 4, or more contiguous A-U basepairs; and/or at least one flip of an A/U basepair in the direct repeat comprises a flip of the fourth A/U basepair in the lower stem of the direct repeat. An exemplary PEgRNA gRNA core structure with one flip of an A-U basepair in the lower stem of the direct repeat is shown in FIG. 12.

In some embodiments, the sequence modification in the direct repeat comprises an extension in the upper stem of the direct repeat. The extension in the upper stem of the direct repeat may be 1, 2, 3, 4, 5, 6, 7, 8, 9, 10, 11, 12, 13, 14, 15, or 16 basepairs in length. In some embodiments, the direct repeat comprises a sequence selected from SEQ ID NOs: 26-37. In some embodiments, the one or more sequence modifications comprises a modification in the second stem loop. In some embodiments, the modification in the second stem loop comprises a flip of a G/C basepair in the second stem loop. In some embodiments, the gRNA core comprises a nucleic acid sequence selected from SEQ ID NOs: 21, 22 or 25. In some embodiments, the gRNA core comprises a sequence selected from SEQ ID NOs: 16-61.

In certain aspects, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, wherein the gRNA core comprises one or more sequence modifications compared to SEQ ID NO. 16.

In some embodiments, the PEgRNAs comprise, in 5′ to 3′ order, the spacer, the gRNA core, the editing template, and the PBS. In some embodiments, the one or more (e.g., two or more, three or more, four or more, or five or more) sequence modifications comprises a gRNA core difference set forth in Table 1. In some embodiments, the gRNA core of a PEgRNA comprises a gRNA core sequence set forth in Table 1 or Table 2. In some embodiments, the one or more sequence modifications comprises a sequence modification in the direct repeat. For example, the direct repeat may comprise at least one flip of an A-U basepair in a lower stem of the direct repeat, optionally wherein the lower stem does not contain 2, 3, 4, or more contiguous A-U basepairs; and/or at least one flip of an A/U basepair in the direct repeat comprises a flip of the fourth A/U basepair in the lower stem of the direct repeat. In some embodiments, the sequence modification in the direct repeat comprises an extension in the upper stem of the direct repeat. The extension in the upper stem of the direct repeat may be from 1, 2, 3, 4, 5, 6, 7, 8, 9, 10, 11, 12, 13, 14, 15, or 16 basepairs. In some embodiments, the direct repeat comprises a sequence selected from SEQ ID NOs: 26-37. In some embodiments, the one or more sequence modifications comprises a modification in the second stem loop. In some embodiments, the modification in the second stem loop comprises a flip of a G/C basepair in the second stem loop. In some embodiments, the gRNA core comprises a nucleic acid sequence selected from SEQ ID NOs: 21, 22 or 25. In some embodiments, the gRNA core comprises a sequence selected from SEQ ID NOs: 16-61.

In certain embodiments, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, and v) a tag sequence that comprises a region of complementarity to the PBS and/or the editing template. In some embodiments, the PEgRNAs comprise, in 5′ to 3′ order, the spacer, the gRNA core, the editing template, and the PBS. In some embodiments, the PEgRNAs comprise, in 5′ to 3′ order, the editing template, the spacer, the tag sequence, the spacer, and the gRNA core.

In some embodiments, the gRNA core comprises a first gRNA core sequence comprising a 5′ half of the gRNA core and a second gRNA core sequence comprising a 3′ half of the gRNA core, and wherein the PEgRNA comprises, in 5′ to 3′ order: the spacer, the first gRNA core sequence, the editing template, the PBS, the tag sequence, and the second gRNA core sequence. In some embodiments, the spacer comprises a first spacer sequence comprising the 5′ half of the spacer and a second spacer sequence comprising the 3′ half of the spacer, wherein the tag sequence is between the first spacer sequence and the second spacer sequence. In some embodiments, the tag sequence comprises a region of complementarity to the editing template. In some embodiments, the tag sequence comprises a region of complementarity to the PBS. In some embodiments, the tag sequence comprises a region of complementarity to the editing template and does not have substantial complementarity to the PBS. In some embodiments, the tag sequence comprises a region of complementarity to the editing template and does not have complementarity to the PBS. In some embodiments, the tag sequence and the editing template each comprises a region of complementarity to each other, wherein the 3′ half of the region of complementarity in the editing template is at a position between 1, 2, 3, 4, 5, 6, 7, 8, 9, 10, 11, 12, 13, 14, 15, 16 bases 5′ of the 3′ half of the editing template, wherein region of complementarity in the tag sequence is at a 5′ portion of the tag sequence. In some embodiments, the tag sequence does not have substantial complementarity to the spacer. In some embodiments, the tag does not have complementarity to the spacer. In some embodiments, the tag sequence is 1, 2, 3, 4, 5, 6, 7, 8, 9, 10, 11, 12, 13, 14, 15, 16 nucleotides in length. In some embodiments, the tag sequence is at least 4, at least 6, at least 8 nucleotides in length. In some embodiments, the tag sequence comprises a nucleic acid sequence selected from SEQ REF NOs 62-1960. In some embodiments, the PEgRNA comprises one or more nucleic acid moieties at its 3′ half. In some embodiments, the PEgRNA comprise, in 5′ to 3′ order, the spacer, the gRNA core, the editing template, and the PBS.

In some embodiments, the one or more nucleic acid moieties comprise a hairpin (e.g., hairpin comprising a region of self-complementarity, optionally wherein the region of self-complementary comprises 2, 3, 4, 5, 6, 7, 8, 9, or 10 contiguous complementary basepairs), a quadruplex (e.g., a G-quadruplex or a C-quadruplex, optionally wherein the G-quadruplex or the C-quadruplex is derived from a VEGF gene promoter), a tRNA sequence (e.g., a tRNA sequence, optionally wherein the tRNA sequence is a tRNA (Proline) sequence), an aptamer (e.g., an aptamer derived from a viral protein-binding sequence, optionally wherein the aptamer comprises a viral reverse transcriptase recruitment sequence, optionally wherein the aptamer comprises a MS2 protein binding sequence or a Moloney Murine leukemia (MMLV) reverse transcriptase recruitment sequence), and/or a pseudoknot (e.g. pseudoknot is derived form a potato roll leaf virus (PLRV)), or any combination thereof. In some embodiments, the one or more nucleic acid moieties comprise a structure derived form a replication recognition sequence of a retrovirus, optionally wherein the retrovirus is a Moloney Murine leukemia (MMLV). For example, the one or more nucleic acid moieties may comprise a configuration as set forth in Table 3. In some embodiments, the one or more nucleic acid moieties comprise a nucleic acid sequence selected from SEQ ID NOs 1-15.

In some embodiments, the PEgRNA comprises a linker. In some embodiments, the linker is: i) immediately 5′ of the one or more nucleic acid moieties, ii) immediately 5′ of the tag sequence, iii) immediately 3′ of the tag sequence, iv) immediately 3′ of the spacer, v) immediately 5′ of the spacer, vi) immediately 3′ of the gRNA core, and/or vii) immediately 5′ of the gRNA core. In some embodiments, the linker is 1, 2, 3, 4, 5, 6, 7, 8, 9, 10, 11, 12, 13, 14, 15, 16 nucleotides in length. In some embodiments, the linker is 2 to 12 nucleotides in length. In some embodiments, the linker is 8 nucleotides long. In some embodiments, the linker does not form a secondary structure. In some embodiments, the linker does not have a region of complementarity to the PBS sequence. In some embodiments, the linker does not have a region of complementarity to the editing template. In some embodiments, the linker comprises a nucleic acid sequence selected from SEQ REF NOs 1961-3859.

In certain embodiments, the gRNA core of the PEgRNAs provided herein comprises one or more sequence modifications compared to SEQ ID NO. 16. In some embodiments, the one or more (e.g., two or more, three or more, four or more, or five or more) sequence modifications comprises a gRNA core difference set forth in Table 1. In some embodiments, the gRNA core of a PEgRNA comprises a gRNA core sequence set forth in Table 1 or Table 2. In some embodiments, the one or more sequence modifications comprises a sequence modification in the direct repeat. For example, the direct repeat may comprise at least one flip of an A-U basepair in a lower stem of the direct repeat, optionally wherein the lower stem does not contain 2, 3, 4, or more contiguous A-U basepairs; and/or at least one flip of an A/U basepair in the direct repeat comprises a flip of the fourth A/U basepair in the lower stem of the direct repeat. In some embodiments, the sequence modification in the direct repeat comprises an extension in the upper stem of the direct repeat. The extension in the upper stem of the direct repeat may be from 1, 2, 3, 4, 5, 6, 7, 8, 9, 10, 11, 12, 13, 14, 15, or 16 basepairs. In some embodiments, the direct repeat comprises a sequence selected from SEQ ID NOs: 26-37. In some embodiments, the one or more sequence modifications comprises a modification in the second stem loop. In some embodiments, the modification in the second stem loop comprises a flip of a G/C basepair in the second stem loop. In some embodiments, the gRNA core comprises a nucleic acid sequence selected from SEQ ID NOs: 21, 22 or 25. In some embodiments, the gRNA core comprises a sequence selected from SEQ ID NOs: 16-61.

In some aspects, PEgRNAs provided herein comprise in 5′ to 3′ order: i) a spacer that comprises a region of complementarity to a search target sequence in target strand of a double stranded target DNA; ii) 5′ part of a guide RNA (gRNA) core comprising a direct repeat and a first stem loop; iii) an editing template that comprises an intended edit compared to the double stranded target DNA; iv) a primer binding site (PBS) that comprises a region of complementarity to a region upstream of a nick site in a non-target strand of the double stranded target DNA; and v) a 3′ part of a gRNA core comprising a second stem loop. In some embodiments, the PEgRNA further comprises a tag sequence that comprises a region of complementarity to the PBS and/or the editing template. In some embodiments, the tag sequence is positioned 3′ of the 3′ part of a gRNA core.

In some embodiments, the tag sequence comprises a region of complementarity to the editing template. In some embodiments, the tag sequence comprises a region of complementarity to the PBS. In some embodiments, the tag sequence comprises a region of complementarity to the editing template and does not have substantial complementarity to the PBS. In some embodiments, the tag sequence comprises a region of complementarity to the editing template and does not have complementarity to the PBS. In some embodiments, the 5′ end of the tag sequence comprises a region of complementarity to a position between 1, 2, 3, 4, 5, 6, 7, 8, 9, 10, 11, 12, 13, 14, 15, 16 bases 5′ of the 3′ end of the editing template. In some embodiments, the tag sequence does not have substantial complementarity to the spacer. In some embodiments, the tag does not have complementarity to the spacer. In some embodiments, the tag sequence is 1, 2, 3, 4, 5, 6, 7, 8, 9, 10, 11, 12, 13, 14, 15, 16 nucleotides in length. The tag sequence may be at least 4, at least 6, or at least 8 nucleotides in length. In some embodiments, the tag sequence comprises a nucleic acid sequence selected from SEQ REF NOs 62-1960. In some embodiments, the PEgRNA comprises one or more nucleic acid moieties at its 3′ end. In some embodiments, the PEgRNA comprise, in 5′ to 3′ order, the spacer, the gRNA core, the editing template, and the PBS.

In some embodiments, the one or more nucleic acid moieties comprise a hairpin (e.g., hairpin comprising a region of self-complementarity, optionally wherein the region of self-complementary comprises 2, 3, 4, 5, 6, 7, 8, 9, or 10 contiguous complementary basepairs), a quadruplex (e.g., a G-quadruplex or a C-quadruplex, optionally wherein the G-quadruplex or the C-quadruplex is derived from a VEGF gene promoter), a tRNA sequence (e.g., a tRNA sequence, optionally wherein the tRNA sequence is a tRNA (Proline) sequence), an aptamer (e.g., an aptamer derived from a viral protein-binding sequence, optionally wherein the aptamer comprises a viral reverse transcriptase recruitment sequence, optionally wherein the aptamer comprises a MS2 protein binding sequence or a Moloney Murine leukemia (MMLV) reverse transcriptase recruitment sequence), and/or a pseudoknot (e.g., pseudoknot is derived form a potato roll leaf virus (PLRV)), or any combination thereof. In some embodiments, the one or more nucleic acid moieties comprise a structure derived form a replication recognition sequence of a retrovirus, optionally wherein the retrovirus is a Moloney Murine leukemia (MMLV). For example, the one or more nucleic acid moieties may comprise a configuration as set forth in Table 2. In some embodiments, the one or more nucleic acid moieties (e.g., a nucleic acid moiety at the 3′ end of the PEgRNA) comprise a nucleic acid sequence selected from SEQ ID NOs 1-15.

In some embodiments, the PEgRNA comprises a linker. In some embodiments, the linker is: i) immediately 5′ of the one or more nucleic acid moieties, ii) immediately 5′ of the tag sequence, iii) immediately 3′ of the tag sequence, iv) immediately 3′ of the spacer, v) immediately 5′ of the spacer, vi) immediately 3′ of the gRNA core, and/or vii) immediately 5′ of the gRNA core. In some embodiments, the linker is 1, 2, 3, 4, 5, 6, 7, 8, 9, 10, 11, 12, 13, 14, 15, 16 nucleotides in length. The linker may be 2-12 nucleotides in length. The linker may be 8 nucleotides in length. In some embodiments, the linker does not form a secondary structure. In some embodiments, the linker does not have a region of complementarity to the PBS sequence. In some embodiments, the linker does not have a region of complementarity to the editing template.

In some embodiments, the 5′ part of a gRNA core and the 3′ part of a guide RNA (gRNA) core comprises one or more sequence modifications compared to SEQ ID NO. 16. In some embodiments, the one or more sequence modifications comprises a gRNA core difference set forth in Table 1 or Table 2.

In some embodiments, the one or more sequence modifications comprises a sequence modification in the direct repeat. For example, the direct repeat may comprise at least one flip of an A-U basepair in a lower stem of the direct repeat, optionally wherein the lower stem does not contain 2, 3, 4, or more contiguous A-U basepairs; and/or at least one flip of an A/U basepair in the direct repeat comprises a flip of the fourth A/U basepair in the lower stem of the direct repeat. In some embodiments, the sequence modification in the direct repeat comprises an extension in the upper stem of the direct repeat. The extension in the upper stem of the direct repeat may be from 1, 2, 3, 4, 5, 6, 7, 8, 9, 10, 11, 12, 13, 14, 15, or 16 basepairs. In some embodiments, the direct repeat comprises a sequence selected from SEQ ID NOs: 26-37. In some embodiments, the one or more sequence modifications comprises a modification in the second stem loop. In some embodiments, the modification in the second stem loop comprises a flip of a G/C basepair in the second stem loop. In some embodiments, the gRNA core comprises a nucleic acid sequence selected from SEQ ID NOs: 21, 22 or 25. In some embodiments, the gRNA core comprises a sequence selected from SEQ ID NOs: 16-61. In some embodiments, the linker comprises a nucleic acid sequence selected from SEQ REF NOs 1961-3859.

In some aspects, PEgRNAs provided herein comprise: i) a first sequence comprising a spacer that comprises a region of complementarity to a search target sequence in target strand of a double stranded target DNA, and a first half of a gRNA core; and ii) a second sequence comprising a second half of the gRNA core, an editing template that comprises an intended edit compared to the double stranded target DNA; a primer binding site (PBS) that comprises a region of complementarity to a region upstream of a nick site in a non-target strand of the double stranded target DNA; and, wherein the gRNA core comprises a direct repeat, a first stem loop, and a second stem loop.

In certain aspects, PEgRNAs provided herein comprise i) a first sequence comprising an editing template that comprises an intended edit compared to the double stranded target DNA; a primer binding site (PBS) that comprises a region of complementarity to a region upstream of a nick site in a non-target strand of the double stranded target DNA; a spacer that comprises a region of complementarity to a search target sequence in target strand of a double stranded target DNA; and a first half of a gRNA core; and ii) a second sequence comprising a second half of a gRNA core, wherein the gRNA core comprises a direct repeat, a first stem loop, and a second stem loop.

In some embodiments, the first sequence is on a first RNA molecule and the second sequence is on a second RNA molecule. In some embodiments, the spacer and the first sequence and the second sequence are on the same RNA molecule. In some embodiments, the first half of the gRNA core and the second half of the gRNA core are selected from the paired first half gRNA core sequences and second half gRNA sequences provided in Table 2.

In certain embodiments, the PEgRNAs further comprise a tag sequence that comprises a region of complementarity to the PBS and/or the editing template. In some embodiments, the PEgRNAs comprise, in 5′ to 3′ order, the spacer, the first half of the gRNA core, the second half of the gRNA core, the editing template, the PBS, and the tag sequence. In some embodiments, the PEgRNAs comprise, in 5′ to 3′ order: the editing template, the spacer, the tag sequence, the spacer, the first half of the gRNA core, and the second half of the gRNA core.

In some embodiments, the tag sequence comprises a region of complementarity to the editing template and does not have substantial complementarity to the PBS. In some embodiments, the tag sequence comprises a region of complementarity to the editing template and does not have complementarity to the PBS. In some embodiments, the 5′ end of the tag sequence comprises a region of complementarity to a position between 1, 2, 3, 4, 5, 6, 7, 8, 9, 10, 11, 12, 13, 14, 15, 16 bases 5′ of the 3′ end of the editing template. In some embodiments, the tag sequence does not have substantial complementarity to the spacer. In some embodiments, the tag does not have complementarity to the spacer. In some embodiments, the tag sequence is 1, 2, 3, 4, 5, 6, 7, 8, 9, 10, 11, 12, 13, 14, 15, 16 nucleotides in length. The tag sequence may be at least 4 nucleotides in length, at least 6 nucleotides in length, or at least 8 nucleotides in length. The tag sequence may be 2-12 nucleotides in length. In some embodiments, the tag sequence comprises a nucleic acid sequence selected from SEQ REF NOs 62-1960. In some embodiments, the PEgRNA comprises one or more nucleic acid moieties at its 3′ half. In some embodiments, the PEgRNA comprise, in 5′ to 3′ order, the spacer, the gRNA core, the editing template, and the PBS.

In some embodiments, the one or more nucleic acid moieties comprise a hairpin (e.g., hairpin comprising a region of self-complementarity, optionally wherein the region of self-complementary comprises 2, 3, 4, 5, 6, 7, 8, 9, or 10 contiguous complementary basepairs), a quadruplex (e.g., a G-quadruplex or a C-quadruplex, optionally wherein the G-quadruplex or the C-quadruplex is derived from a VEGF gene promoter), a tRNA sequence (e.g., a tRNA sequence, optionally wherein the tRNA sequence is a tRNA (Proline) sequence), an aptamer (e.g., an aptamer derived from a viral protein-binding sequence, optionally wherein the aptamer comprises a viral reverse transcriptase recruitment sequence, optionally wherein the aptamer comprises a MS2 protein binding sequence or a Moloney Murine leukemia (MMLV) reverse transcriptase recruitment sequence), and/or a pseudoknot (e.g., pseudoknot is derived form a potato roll leaf virus (PLRV)), or any combination thereof. In some embodiments, the one or more nucleic acid moieties comprise a structure derived form a replication recognition sequence of a retrovirus, optionally wherein the retrovirus is a Moloney Murine leukemia (MMLV). For example, the one or more nucleic acid moieties may comprise a configuration as set forth in Table 2. In some embodiments, the one or more nucleic acid moieties comprise a nucleic acid sequence selected from SEQ ID NOs 1-15.

In some embodiments, the PEgRNA comprises a linker. In some embodiments, the linker is: i) immediately 5′ of the one or more nucleic acid moieties, ii) immediately 5′ of the tag sequence, iii) immediately 3′ of the tag sequence, iv) immediately 3′ of the spacer, v) immediately 5′ of the spacer, vi) immediately 3′ of the gRNA core, and/or vii) immediately 5′ of the gRNA core. In some embodiments, the linker is 1, 2, 3, 4, 5, 6, 7, 8, 9, 10, 11, 12, 13, 14, 15, 16 nucleotides in length. In some embodiments, the linker does not form a secondary structure. In some embodiments, the linker does not have a region of complementarity to the PBS sequence. In some embodiments, the linker does not have a region of complementarity to the editing template.

In some embodiments, the 5′ part of a gRNA core and the 3′ part of a guide RNA (gRNA) core comprises one or more sequence modifications compared to SEQ ID NO. 16. In some embodiments, the one or more sequence modifications comprises a gRNA core difference set forth in Table 1 or Table 2. In some embodiments, the linker comprises a nucleic acid sequence selected from SEQ REF NOs 1961-3859.

In some embodiments, the one or more sequence modifications comprises a sequence modification in the direct repeat. For example, the direct repeat may comprise at least one flip of an A-U basepair in a lower stem of the direct repeat, optionally wherein the lower stem does not contain 2, 3, 4, or more contiguous A-U basepairs; and/or at least one flip of an A/U basepair in the direct repeat comprises a flip of the fourth A/U basepair in the lower stem of the direct repeat. In some embodiments, the sequence modification in the direct repeat comprises an extension in the upper stem of the direct repeat. The extension in the upper stem of the direct repeat may be from 1, 2, 3, 4, 5, 6, 7, 8, 9, 10, 11, 12, 13, 14, 15, or 16 basepairs. In some embodiments, the direct repeat comprises a sequence selected from SEQ ID NOs: 26-37. In some embodiments, the one or more sequence modifications comprises a modification in the second stem loop. In some embodiments, the modification in the second stem loop comprises a flip of a G/C basepair in the second stem loop. In some embodiments, the gRNA core comprises a nucleic acid sequence selected from SEQ ID NOs: 21, 22 or 25. In some embodiments, the gRNA core comprises a sequence selected from SEQ ID NOs: 16-61.

In certain aspects, provided herein are methods of making a PEgRNA as described herein, the method comprising ligating the first sequence to the second sequence.

In certain aspects, provided herein are methods of making a PEgRNA, the method comprising synthesizing a polynucleotide comprising a sequence encoding a PEgRNA as described herein.

In certain aspects, provided herein are PEgRNA systems comprising a PEgRNA as described herein.

In certain aspects, provided herein are prime editing complexes comprising: (i) the PEgRNA of this disclosure or the PEgRNA system of this disclosure; and (ii) a prime editor comprising a DNA binding domain and a DNA polymerase domain. In some embodiments, the DNA binding domain is a CRISPR associated (Cas) protein domain. In some embodiments, the Cas protein domain has nickase activity. In some embodiments, the Cas protein domain is a Cas9. In some embodiments, the Cas9 comprises a mutation in an HNH domain. In some embodiments, the Cas9 comprises a H840A mutation in the HNH domain. In some embodiments, the Cas protein domain is a Cas12b. In some embodiments, the Cas protein domain is a Cas12a, Cas12b, Cas12c, Cas12d, Cas12e, Cas14a, Cas14b, Cas14c, Cas14d, Cas14e, Cas14f, Cas14g, Cas14h, Cas14u, or a Casφ. In some embodiments, the DNA polymerase domain is a reverse transcriptase. Many reverse transcriptase enzymes have DNA-dependent DNA synthesis abilities in addition to RNA-dependent DNA synthesis abilities, i.e., reverse transcription). In some embodiments, the reverse transcriptase is a retrovirus reverse transcriptase. In some embodiments, the reverse transcriptase is a Moloney murine leukemia virus (M-MLV) reverse transcriptase. In some embodiments, the DNA polymerase and the programmable DNA binding domain are fused or linked to form a fusion protein. In some embodiments, the fusion protein comprises the sequence of SEQ ID NO: 4440.

In certain aspects, provided herein are lipid nanoparticles (LNPs) or ribonucleoproteins (RNPs) comprising a prime editing complex or a component thereof. One embodiment provides a polynucleotide encoding the PEgRNA of this disclosure, the PEgRNA system of this disclosure, or the fusion protein of this disclosure. In some embodiments, the polynucleotide is an mRNA. In some embodiments, the polynucleotide is operably linked to a regulatory element. In some embodiments, the regulatory element is an inducible regulatory element.

In certain aspects, provided herein are vectors comprising the polynucleotide of above embodiments. In some embodiments, the vector is an AAV vector.

In certain aspects, provided herein are isolated cells comprising a PEgRNA of this disclosure, the PEgRNA system of this disclosure, a prime editing complex of this disclosure, a LNP or RNP of any one of the above embodiments, a polynucleotide of any one of the above embodiments, and/or a vector of any one the above embodiments. In some embodiments, the cell is a human cell.

In some aspects, provided herein are pharmaceutical compositions comprising at least one of (i) the PEgRNAs of this disclosure, the PEgRNA systems of this disclosure, the prime editing complex of this disclosure, an LNP or RNP of an embodiment described herein, the polynucleotide of any one of the above embodiments, the vector of any one of the above embodiments, and/or a cell of any one of the above embodiments; and at least one (ii) a pharmaceutically acceptable carrier.

In certain aspects, provided herein are methods for editing a gene, the method comprising contacting the gene with any one of (i) the PEgRNAs of this disclosure or any one of the PEgRNA systems of this disclosure and (ii) a prime editor comprising a DNA binding domain and a DNA polymerase domain, wherein the PEgRNA directs the prime editor to incorporate the intended nucleotide edit in the gene, thereby editing the gene.

In certain aspects, also provided herein are methods for editing a gene, the method comprising contacting the gene with the prime editing complex disclosed herein, wherein the PEgRNA directs the prime editor to incorporate the intended nucleotide edit in the gene, thereby editing the gene.

In some embodiments, the prime editor synthesizes a single stranded DNA encoded by the editing template, wherein the single stranded DNA replaces the editing target sequence and results in incorporation of the intended nucleotide edit into a region corresponding to the editing target in the gene. In some embodiments, the gene is in a cell. In some embodiments, the cell is a mammalian cell. In some embodiments, the cell is a human cell. In some embodiments, the cell is a primary cell. In some embodiments, the cell is in a subject. In some embodiments, the subject is a human. In some embodiments, the method further comprises administering the cell to the subject after incorporation of the intended nucleotide edit.

In certain embodiments, provided herein are cells generated by any one of above methods. In certain embodiments, provided herein are a population of cells generated by any one of the methods provided herein.

### BRIEF DESCRIPTION OF THE DRAWINGS

The novel features of the methods and compositions provided herein are set forth with particularity in the appended claims. A better understanding of the features and advantages of the methods and compositions provided herein will be obtained by reference to the following detailed description that sets forth illustrative embodiments, in which the principles of the methods and compositions provided herein are utilized, and the accompanying drawings of which:

FIG. 1 show exemplary nucleic acid moieties (e.g., for inclusion on the 3′ end of a PEgRNA disclosed herein). FIG. 1 discloses SEQ ID NOS 1, 7, 3, 6, 5 and 4, respectively, in order of appearance.

FIG. 2 are three graphs showing nucleic acid moieties increase editing efficiency. Each violin plot represents combination 48 unique PEgRNAs (1 spacer, 3 unique edits, 4 PBS lengths, 4 RTT lengths).

FIG. 3 shows an exemplary PEgRNA with spacer and PBS.

FIG. 4 shows an exemplary PEgRNAs with spacer, RTT, PBS, linker, and tag.

FIG. 5 shows an exemplary PEgRNA an exemplary PEgRNAs with spacer, RTT, PBS, linker, and tag with arrow pointing to the “0” position used in to FIGS. 6 and 7.

FIG. 6 are graphs showing the effect of the tag start position on prime editing efficiency.

FIG. 7 are graphs showing the effect of the tag start position on prime editing efficiency.

FIG. 8 shows an exemplary PEgRNA gRNA core (SEQ ID NO: 16). The dashed line and arrows represent two potential positions where the PEgRNA can be split into two RNA molecules for separate synthesis (e.g., prior to ligation).

FIG. 9 are graphs showing editing efficiencies by LegRNAs.

FIG. 10 shows a schematic illustrating exemplary oligonucleotide library design. FIG. 10 discloses SEQ ID NOS 7063-7065, respectively, in order of appearance.

FIG. 11 shows a schematic illustrated exemplary amplified oligonucleotide library. FIG. 11 discloses SEQ ID NOS 7066, 7054, 7067-7069, respectively, in order of appearance.

FIG. 12 shows an exemplary structure of a spacer and gRNA core of a PEgRNA, with one flip of an A-U basepair in the lower stem of the direct repeat and a 5-nucleotide extension in the upper stem of the direct repeat (SEQ ID NO: 7070). The rest of the PEgRNA, e.g., editing template and PBS, are not shown.

FIG. 13A-C are graphs showing the effect of tag (Comp Tag) binding position on prime editing efficiency. FIG. 13A shows binding position on prime editing efficiency for tags that are 4 nucleotides in length. FIG. 13B shows binding position on prime editing efficiency for tags that are 6 nucleotides in length. FIG. 13C shows binding position on prime editing efficiency for tags that are 8 nucleotides in length.

FIG. 14 is a graph showing the effect of tag (CompTag) length on prime editing efficiency for the pool of tags that bind entirely within the edit template of the PEgRNA.

### DETAILED DESCRIPTION

Provided herein, in some embodiments, are compositions and methods related to modified prime editing guide RNAs (PEgRNAs) useful, for example, in prime editing applications. In certain embodiments, provided herein are compositions and methods for introducing intended nucleotide edits in target DNA, e.g., correction of mutations in a gene, including a gene associated with a disease. Compositions provided herein can comprise PEgRNAs that can guide prime editors (PEs) to specific DNA targets and introduce nucleotide edits on the target gene.

The following description and examples illustrate embodiments of the present disclosure in detail. It is to be understood that this disclosure is not limited to the particular embodiments described herein and as such can vary. Those of skill in the art will recognize that there are numerous variations and modifications of this disclosure, which are encompassed within its scope. Although various features of the present disclosure can be described in the context of a single embodiment, the features can also be provided separately or in any suitable combination. Conversely, although the present disclosure can be described herein in the context of separate embodiments for clarity, the present disclosure can also be implemented in a single embodiment.

### Definitions

Unless defined otherwise, all technical and scientific terms used herein have the same meaning as is commonly understood by one of ordinary skill in the art.

The terminology used herein is for the purpose of describing particular embodiments only and is not intended to be limiting. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof as used herein mean “comprising”.

Unless otherwise specified, the words “comprising”, “comprise”, “comprises”, “having”, “have”, “has”, “including”, “includes”, “include”, “containing”, “contains” and “contain” are inclusive or open-ended and do not exclude additional, unrecited elements or method steps.

Reference to “some embodiments”, “an embodiment”, “one embodiment”, or “other embodiments” means that a particular feature or characteristic described in connection with the embodiments is included in at least one or more embodiments, but not necessarily all embodiments, of the present disclosure.

The term “about” or “approximately” means within 10% of a given value, rounded up where only integers are applicable. For example, about 100 means from 90 to 110 and about 7 means from 6 to 8. Where particular values are described in the application and claims, unless otherwise stated, the value should be assumed to be modified by the term “about”.

As used herein, a “cell” can generally refer to a biological cell. A cell can be the basic structural, functional and/or biological unit of a living organism. A cell can originate from any organism having one or more cells. Some non-limiting examples include: a prokaryotic cell, eukaryotic cell, a bacterial cell, an archacal cell, a cell of a single-cell eukaryotic organism, a protozoa cell, a cell from a plant, an animal cell, a cell from an invertebrate animal (e.g. fruit fly, cnidarian, echinoderm, nematode, etc.), a cell from a vertebrate animal (e.g., fish, amphibian, reptile, bird, mammal), a cell from a mammal (e.g., a pig, a cow, a goat, a sheep, a rodent, a rat, a mouse, a non-human primate, a human, etc.), et cetera. Sometimes a cell may not originate from a natural organism (e.g., a cell can be synthetically made, sometimes termed an artificial cell).

In some embodiments, the cell is a human cell. A cell may be of or derived from different tissues, organs, and/or cell types. In some embodiments, the cell is a primary cell. In some embodiments, the term primary cell means a cell isolated from an organism, e.g., a mammal, which is grown in tissue culture (i.e., in vitro) for the first time before subdivision and transfer to a subculture. In some non-limiting examples, mammalian primary cells can be modified through introduction of one or more polynucleotides, polypeptides, and/or prime editing compositions (e.g., through transfection, transduction, electroporation and the like) and further passaged. Such modified mammalian primary cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a fibroblast. In some embodiments, the cell is a stem cell. In some embodiments, the cell is a pluripotent stem cell. In some embodiments, the cell is an induced pluripotent stem cell (iPSC). In some embodiments, the cell is a stem cell. In some embodiments, the cell is an embryonic stem cell (ESC). In some embodiments, the cell is a human stem cell. In some embodiments, the cell is a human pluripotent stem cell. In some embodiments, the cell is a human fibroblast. In some embodiments, the cell is an induced human pluripotent stem cell (iPSC). In some embodiments, the cell is a human stem cell. In some embodiments, the cell is a human embryonic stem cell.

In some embodiments, a cell is not isolated from an organism but forms part of a tissue or organ of an organism, e.g., a mammal. In some non-limiting examples, mammalian cells include muscle cells (e.g., cardiac muscle cells, smooth muscle cells, myosatellite cells), epithelial cells (e.g., mammary epithelial cells, intestinal epithelial cells, hepatocytes), endothelial cells, glial cells, neural cells, formed elements of the blood (e.g., lymphocytes, bone marrow cells), precursors of any of these somatic cell types, and stem cells. In some embodiments, the cell is a primary muscle cell. In some embodiments, the cell is a myosatellite cell (a satellite cell). In some embodiments, the cell is a human myosatellite cell (a satellite cell). In some embodiments, the cell is a stem cell. In some embodiments, the cell is a human stem cell.

In some embodiments, the cell is a differentiated cell. In some embodiments, cell is a fibroblast. In some embodiments, the cell is a differentiated muscle cell, a myosatellite cell, a differentiated epithelial cell, or a differentiated neuron cell. In some embodiments, the cell is a skeletal muscle cell. In some embodiments, the skeletal muscle cell is differentiated from an iPSC, ESC or myosatellite cell. In some embodiments, the cell is a differentiated human cell. In some embodiments, cell is a human fibroblast. In some embodiments, the cell is a differentiated human muscle cell. In some embodiments, cell is a human myosatellite cell. In some embodiments, the cell is a human skeletal muscle cell. In some embodiments, the human skeletal muscle cell is differentiated from a human iPSC, human ESC or human myosatellite cell. In some embodiments, the cell is differentiated from a human iPSC or human ESC.

In some embodiments, the cell comprises a prime editor, a PEgRNA, a ngRNA, a prime editing system, or a prime editing complex. In some embodiments, the cell is from a human subject. In some embodiments, the human subject has a disease or condition associated with a mutation to be corrected by prime editing. In some embodiments, the cell is from a human subject, and comprises a prime editor, a PEgRNA, a ngRNA, a prime editing system, or a prime editing complex for correction of the mutation. In some embodiments, the cell is from the human subject and the mutation has been edited or corrected by prime editing. In some embodiments, the cell is in a human subject, and comprises a prime editor, a PEgRNA, a ngRNA, a prime editing system, or a prime editing complex for correction of the mutation. In some embodiments, the cell is from the human subject and the mutation has been edited or corrected by prime editing.

The term “substantially” as used herein may refer to a value approaching 100% of a given value. In some embodiments, the term may refer to an amount that may be at least about 70%, 80%, 90%, 91%, 92%, 93%, 94%, 95%, 96%, 97%, 98%, 99%, 99.9%, or 99.99% of a total amount. In some embodiments, the term may refer to an amount that may be about 100% of a total amount.

The terms “protein” and “polypeptide” can be used interchangeably to refer to a polymer of two or more amino acids joined by covalent bonds (e.g., an amide bond) that can adopt a three-dimensional conformation. In some embodiments, a protein or polypeptide comprises at least 10 amino acids, 15 amino acids, 20 amino acids, 30 amino acids or 50 amino acids joined by covalent bonds (e.g., amide bonds). In some embodiments, a protein comprises at least two amide bonds. In some embodiments, a protein comprises multiple amide bonds. In some embodiments, a protein comprises an enzyme, enzyme precursor proteins, regulatory protein, structural protein, receptor, nucleic acid binding protein, a biomarker, a member of a specific binding pair (e.g., a ligand or aptamer), or an antibody. In some embodiments, a protein may be a full-length protein (e.g., a fully processed protein having certain biological function). In some embodiments, a protein may be a variant or a fragment of a full-length protein. For example, in some embodiments, a Cas9 protein domain comprises an H840A amino acid substitution compared to a naturally occurring S. pyogenes Cas9 protein. A variant of a protein or enzyme, for example a variant reverse transcriptase, comprises a polypeptide having an amino acid sequence that is about 60% identical, about 70% identical, about 80% identical, about 90% identical, about 95% identical, about 96% identical, about 97% identical, about 98% identical, about 99% identical, about 99.5% identical, or about 99.9% identical to the amino acid sequence of a reference protein.

In some embodiments, a protein comprises one or more protein domains or subdomains. As used herein, the term “polypeptide domain”, “protein domain”, or “domain” when used in the context of a protein or polypeptide, refers to a polypeptide chain that has one or more biological functions, e.g., a catalytic function, a protein-protein binding function, or a protein-DNA function. In some embodiments, a protein comprises multiple protein domains. In some embodiments, a protein comprises multiple protein domains that are naturally occurring. In some embodiments, a protein comprises multiple protein domains from different naturally occurring proteins. For example, in some embodiments, a prime editor may be a fusion protein comprising a Cas9 protein domain of S. pyogenes and a reverse transcriptase protein domain of Moloney murine leukemia virus. A protein that comprises amino acid sequences from different origins or naturally occurring proteins may be referred to as a fusion, or chimeric protein.

In some embodiments, a protein comprises a functional variant or functional fragment of a full-length wild type protein. A “functional fragment” or “functional portion”, as used herein, refers to any portion of a reference protein (e.g., a wild type protein) that encompasses less than the entire amino acid sequence of the reference protein while retaining one or more of the functions, e.g., catalytic or binding functions. For example, a functional fragment of a reverse transcriptase may encompass less than the entire amino acid sequence of a wild type reverse transcriptase, but retains the ability under at least one set of conditions to catalyze the polymerization of a polynucleotide. When the reference protein is a fusion of multiple functional domains, a functional fragment thereof may retain one or more of the functions of at least one of the functional domains. For example, a functional fragment of a Cas9 may encompass less than the entire amino acid sequence of a wild type Cas9, but retains its DNA binding ability and lacks its nuclease activity partially or completely.

A “functional variant” or “functional mutant”, as used herein, refers to any variant or mutant of a reference protein (e.g., a wild type protein) that encompasses one or more alterations to the amino acid sequence of the reference protein while retaining one or more of the functions, e.g., catalytic or binding functions. In some embodiments, the one or more alterations to the amino acid sequence comprises amino acid substitutions, insertions or deletions, or any combination thereof. In some embodiments, the one or more alterations to the amino acid sequence comprises amino acid substitutions. For example, a functional variant of a reverse transcriptase may comprise one or more amino acid substitutions compared to the amino acid sequence of a wild type reverse transcriptase, but retains the ability under at least one set of conditions to catalyze the polymerization of a polynucleotide. When the reference protein is a fusion of multiple functional domains, a functional variant thereof may retain one or more of the functions of at least one of the functional domains. For example, in some embodiments, a functional fragment of a Cas9 may comprise one or more amino acid substitutions in a nuclease domain, e.g., an H840A amino acid substitution, compared to the amino acid sequence of a wild type Cas9, but retains the DNA binding ability and lacks the nuclease activity partially or completely.

The term “function” and its grammatical equivalents as used herein may refer to a capability of operating, having, or serving an intended purpose. Functional may comprise any percent from baseline to 100% of an intended purpose. For example, functional may comprise or comprise about 5%, 10%, 15%, 20%, 25%, 30%, 35%, 40%, 45%, 50%, 55%, 60%, 65%, 70%, 75%, 80%, 85%, 90%, 95%, or up to about 100% of an intended purpose. In some embodiments, the term functional may mean over or over about 100% of normal function, for example, 125%, 150%, 175%, 200%, 250%, 300%, 400%, 500%, 600%, 700% or up to about 1000% of an intended purpose. In some embodiments, a protein or polypeptides includes naturally occurring amino acids (e.g., one of the twenty amino acids commonly found in peptides synthesized in nature, and known by the one letter abbreviations A, R, N, C, D, Q, E, G, H, I, L, K, M, F, P, S, T, W, Y and V). In some embodiments, a protein or polypeptides includes non-naturally occurring amino acids (e.g., amino acids which is not one of the twenty amino acids commonly found in peptides synthesized in nature, including synthetic amino acids, amino acid analogs, and amino acid mimetics). In some embodiments, a protein or polypeptide is modified.

In some embodiments, a protein comprises an isolated polypeptide. The term “isolated” means free or removed to varying degrees from components which normally accompany it as found in the natural state or environment. For example, a polypeptide naturally present in a living animal is not isolated, and the same polypeptide partially or completely separated from the coexisting materials of its natural state is isolated.

In some embodiments, a protein is present within a cell, a tissue, an organ, or a virus particle. In some embodiments, a protein is present within a cell or a part of a cell (e.g., a bacteria cell, a plant cell, or an animal cell). In some embodiments, the cell is in a tissue, in a subject, or in a cell culture. In some embodiments, the cell is a microorganism (e.g., a bacterium, fungus, protozoan, or virus). In some embodiments, a protein is present in a mixture of analytes (e.g., a lysate). In some embodiments, the protein is present in a lysate from a plurality of cells or from a lysate of a single cell.

The terms “homologous,” “homology,” or “percent homology” as used herein refer to the degree of sequence identity between an amino acid or polynucleotide sequence and a corresponding reference sequence. “Homology” can refer to polymeric sequences, e.g., polypeptide or DNA sequences that are similar. Homology can mean, for example, nucleic acid sequences with at least about: 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or 100% identity. In other embodiments, a “homologous sequence” of nucleic acid sequences may exhibit 93%, 95% or 98% sequence identity to the reference nucleic acid sequence. For example, a “region of homology to a genomic region” can be a region of DNA that has a similar sequence to a given genomic region in the genome. A region of homology can be of any length that is sufficient to promote binding of a spacer, primer binding site or protospacer sequence to the genomic region. For example, the region of homology can comprise at least 5, 10, 11, 12, 13, 14, 15, 16, 17, 18, 19, 20, 21, 22, 23, 24, 25, 30, 35, 40, 45, 50, 55, 60, 65, 70, 75, 80, 85, 90, 95, 100, 200, 300, 400, 500, 600, 700, 800, 900, 1000, 1100, 1200, 1300, 1400, 1500, 1600, 1700, 1800, 1900, 2000, 2100, 2200, 2300, 2400, 2500, 2600, 2700, 2800, 2900, 3000, 3100 or more bases in length such that the region of homology has sufficient homology to undergo binding with the corresponding genomic region.

When a percentage of sequence homology or identity is specified, in the context of two nucleic acid sequences or two polypeptide sequences, the percentage of homology or identity generally refers to the alignment of two or more sequences across a portion of their length when compared and aligned for maximum correspondence. When a position in the compared sequence can be occupied by the same base or amino acid, then the molecules can be homologous at that position. Unless stated otherwise, sequence homology or identity is assessed over the specified length of the nucleic acid, polypeptide or portion thereof. In some embodiments, the homology or identity is assessed over a functional portion or specified portion of the length.

Alignment of sequences for assessment of sequence homology can be conducted by algorithms known in the art, such as the Basic Local Alignment Search Tool (BLAST) algorithm, which is described in Altschul et al, J. Mol. Biol. 215:403-410, 1990. A publicly available, internet interface, for performing BLAST analyses is accessible through the National Center for Biotechnology Information. Additional known algorithms include those published in: Smith & Waterman, “Comparison of Biosequences”, Adv. Appl. Math. 2:482, 1981; Needleman & Wunsch, “A general method applicable to the search for similarities in the amino acid sequence of two proteins” J. Mol. Biol. 48:443, 1970; Pearson & Lipman “Improved tools for biological sequence comparison”, Proc. Natl. Acad. Sci. USA 85:2444, 1988; or by automated implementation of these or similar algorithms. Global alignment programs may also be used to align similar sequences of roughly equal size. Examples of global alignment programs include NEEDLE (available at www.ebi.ac.uk/Tools/psa/emboss_needle/) which is part of the EMBOSS package (Rice P et al., Trends Genet., 2000; 16:276-277), and the GGSEARCH program https://fasta.bioch.virginia.edu/fasta_www2/, which is part of the FASTA package (Pearson W and Lipman D, 1988, Proc. Natl. Acad. Sci. USA, 85:2444-2448). Both of these programs are based on the Needleman-Wunsch algorithm which is used to find the optimum alignment (including gaps) of two sequences along their entire length. A detailed discussion of sequence analysis can also be found in Unit 19.3 of Ausubel et al (“Current Protocols in Molecular Biology” John Wiley & Sons Inc, 1994-1998, Chapter 15, 1998). Unless otherwise stated, percent identity should be determined based on an alignment between a query sequence and a reference sequence performed with Needleman-Wunsch alignment with Gap Costs set to Existence: 11 Extension: 1 where percent identity is calculated by dividing the number of identities by the length of the alignment.

A skilled person understands that amino acid (or nucleotide) positions may be determined in homologous sequences based on alignment, for example, “H840” in a reference Cas9 sequence may correspond to H839, or another position in a Cas9 homolog.

The term “polynucleotide” or “nucleic acid molecule” can be any polymeric form of nucleotides, including DNA, RNA, a hybridization thereof, or RNA-DNA chimeric molecules. In some embodiments, a polynucleotide comprises cDNA, genomic DNA, mRNA, tRNA, rRNA, or microRNA. In some embodiments, a polynucleotide is double stranded, e.g., a double-stranded DNA in a gene. In some embodiments, a polynucleotide is single-stranded or substantially single-stranded, e.g., single-stranded DNA or an mRNA. In some embodiments, a polynucleotide is a cell-free nucleic acid molecule. In some embodiments, a polynucleotide circulates in blood. In some embodiments, a polynucleotide is a cellular nucleic acid molecule. In some embodiments, a polynucleotide is a cellular nucleic acid molecule in a cell circulating in blood.

Polynucleotides can have any three-dimensional structure. The following are nonlimiting examples of polynucleotides: a gene or gene fragment (for example, a probe, primer, EST or SAGE tag), an exon, an intron, intergenic DNA (including, without limitation, heterochromatic DNA), messenger RNA (mRNA), transfer RNA (tRNA), ribosomal RNA (rRNA), a ribozyme, cDNA, a recombinant polynucleotide, a branched polynucleotide, a plasmid, a vector, isolated DNA, isolated RNA, sgRNA, guide RNA, a nucleic acid probe, a primer, an snRNA, a long non-coding RNA, a snoRNA, a siRNA, a miRNA, a tRNA-derived small RNA (tsRNA), an antisense RNA, an shRNA, or a small rDNA-derived RNA (srRNA).

In some embodiments, a polynucleotide comprises deoxyribonucleotides, ribonucleotides or analogs thereof. In some embodiments, a polynucleotide comprises modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component.

In some embodiments, a polynucleotide is composed of a specific sequence of four nucleotide bases: adenine (A); cytosine (C); guanine (G); thymine (T); and uracil (U) for thymine when the polynucleotide is RNA. In some embodiments, the polynucleotide may comprise one or more other nucleotide bases, such as inosine (I), which is read by the translation machinery as guanine (G). Consistent with the ST.26 standard, RNA sequences provided herein, e.g., PEgRNA sequences, may contain “T”s instead of “U”s.

In some embodiments, a polynucleotide may be modified. As used herein, the terms “modified” or “modification” refers to chemical modification with respect to the A, C, G, T and U nucleotides. In some embodiments, modifications may be on the nucleoside base and/or sugar portion of the nucleosides that comprise the polynucleotide. In some embodiments, the modification may be on the internucleoside linkage (e.g., phosphate backbone). In some embodiments, multiple modifications are included in the modified nucleic acid molecule. In some embodiments, a single modification is included in the modified nucleic acid molecule.

The term “complement”, “complementary”, or “complementarity” as used herein, refers to the ability of two polynucleotide molecules to basepair with each other. Complementary polynucleotides may basepair via hydrogen bonding, which may be Watson Crick, Hoogsteen or reversed Hoogsteen hydrogen bonding. For example, an adenine on one polynucleotide molecule will basepair to a thymine or uracil on a second polynucleotide molecule, a cytosine on one polynucleotide molecule will basepair to a guanine on a second polynucleotide molecule, a guanine on one polynucleotide molecule will basepair to a cytosine or a uracil on a second polynucleotide molecule, and a thymine or uracil on one polynucleotide molecule will basepair to an adenine or a guanine on a second polynucleotide molecule. Two polynucleotide molecules are complementary to each other when a first polynucleotide molecule comprising a first nucleotide sequence can basepair with a second polynucleotide molecule comprising a second nucleotide sequence. For instance, the two DNA molecules 5′-ATGC-3′ and 5′-GCAT-3′ are complementary, and the complement of the DNA molecule 5′-ATGC-3′ is 5′-GCAT-3′. A percentage of complementarity indicates the percentage of nucleotides in a polynucleotide molecule which can basepair with a second polynucleotide molecule (e.g., 5, 6, 7, 8, 9, 10 out of 10 being 50%, 60%, 70%, 80%, 90%, and 100% complementary, respectively). “Perfectly complementary” means that all the contiguous nucleotides of a polynucleotide molecule will basepair with the same number of contiguous nucleotides in a second polynucleotide molecule. “Substantially complementary” as used herein refers to a degree of complementarity that can be 70%, 75%, 80%, 85%, 90%, 95%, 97%, 98%, or 99% over all or a portion of two polynucleotide molecules. In some embodiments, the portion of complementarity may be a region of 10, 15, 20, 25, 30, 35, 40, 45, 50, or more nucleotides. “Substantial complementary” can also refer to a 100% complementarity over a portion of two polynucleotide molecules. In some embodiments, the portion of complementarity between the two polynucleotide molecules is at least 60%, 65%, 70%, 75%, 80%, 85%, 90%, 95%, 97%, 98%, or 99% of the length of at least one of the two polynucleotide molecules or a functional or defined portion thereof.

As used herein, “expression” refers to the process by which polynucleotides are transcribed into mRNA and/or the process by which polynucleotides, e.g., the transcribed mRNA, translated into peptides, polypeptides, or proteins. If the polynucleotide is derived from genomic DNA, expression may include splicing of the mRNA in a eukaryotic cell. In some embodiments, expression of a polynucleotide, e.g., a gene or a DNA encoding a protein, is determined by the amount of the protein encoded by the gene after transcription and translation of the gene. In some embodiments, expression of a polynucleotide, e.g., a gene or a DNA encoding a protein, is determined by the amount of a functional form of the protein encoded by the gene after transcription and translation of the gene. In some embodiments, expression of a gene is determined by the amount of the mRNA, or transcript, that is encoded by the gene after transcription the gene. In some embodiments, expression of a polynucleotide, e.g., an mRNA, is determined by the amount of the protein encoded by the mRNA after translation of the mRNA. In some embodiments, expression of a polynucleotide, e.g., an mRNA or coding RNA, is determined by the amount of a functional form of the protein encoded by the polypeptide after translation of the polynucleotide.

The term “sequencing” as used herein, may comprise capillary sequencing, bisulfite-free sequencing, bisulfite sequencing, TET-assisted bisulfite (TAB) sequencing, ACE-sequencing, high-throughput sequencing, Maxam-Gilbert sequencing, massively parallel signature sequencing, Polony sequencing, 454 pyrosequencing, Sanger sequencing, Illumina sequencing, SOLID sequencing, Ion Torrent semiconductor sequencing, DNA nanoball sequencing, Heliscope single molecule sequencing, single molecule real time (SMRT) sequencing, nanopore sequencing, shot gun sequencing, RNA sequencing, or any combination thereof.

The terms “equivalent” or “biological equivalent” are used interchangeably when referring to a particular molecule, or biological or cellular material, and means a molecule having minimal homology to another molecule while still maintaining a desired structure or functionality.

The term “encode” as it is applied to polynucleotides refers to a polynucleotide which is said to “encode” another polynucleotide, a polypeptide, or an amino acid if, in its native state or when manipulated by methods well known to those skilled in the art, it can be used as polynucleotide synthesis template, e.g., transcribed into an RNA, reverse transcribed into a DNA or cDNA, and/or translated to produce an amino acid, or a polypeptide or fragment thereof. In some embodiments, a polynucleotide comprising three contiguous nucleotides form a codon that encodes a specific amino acid. In some embodiments, a polynucleotide comprises one or more codons that encode a polypeptide. In some embodiments, a polynucleotide comprising one or more codons comprises a mutation in a codon compared to a wild-type reference polynucleotide. In some embodiments, the mutation in the codon encodes an amino acid substitution in a polypeptide encoded by the polynucleotide as compared to a wild-type reference polypeptide.

The term “mutation” as used herein refers to a change and/or alteration in an amino acid sequence of a protein or nucleic acid sequence of a polynucleotide. Such changes and/or alterations may comprise the substitution, insertion, deletion and/or truncation of one or more amino acids, in the case of an amino acid sequence, and/or nucleotides, in the case of nucleic acid sequence, compared to a reference amino acid or nucleic acid sequence. In some embodiments, the reference sequence is a wild-type sequence. In some embodiments, a mutation in a nucleic acid sequence of a polynucleotide encodes a mutation in the amino acid sequence of a polypeptide. In some embodiments, the mutation in the amino acid sequence of the polypeptide or the mutation in the nucleic acid sequence of the polynucleotide is a mutation associated with a disease state.

The term “subject” and its grammatical equivalents as used herein may refer to a human or a non-human. A subject may be a mammal. A human subject may be male or female. A human subject may be of any age. A subject may be a human embryo. A human subject may be a newborn, an infant, a child, an adolescent, or an adult. A human subject may be up to about 100 years of age. A human subject may be in need of treatment for a genetic disease or disorder.

The terms “treatment” or “treating” and their grammatical equivalents may refer to the medical management of a subject with an intent to cure, ameliorate, or ameliorate a symptom of, a disease, condition, or disorder. Treatment may include active treatment, that is, treatment directed specifically toward the improvement of a disease, condition, or disorder. Treatment may include causal treatment, that is, treatment directed toward removal of the cause of the associated disease, condition, or disorder. In addition, this treatment may include palliative treatment, that is, treatment designed for the relief of symptoms rather than the curing of the disease, condition, or disorder. Treatment may include supportive treatment, that is, treatment employed to supplement another specific therapy directed toward the improvement of the disease, condition, or disorder. In some embodiments, a condition may be pathological. In some embodiments, a treatment may not completely cure or prevent a disease, condition, or disorder. In some embodiments, a treatment ameliorates, but does not completely cure or prevent a disease, condition, or disorder. In some embodiments, a subject may be treated for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of the subject.

The term “ameliorate” and its grammatical equivalents means to decrease, suppress, attenuate, diminish, arrest, or stabilize the development or progression of a disease.

The terms “prevent” or “preventing” means delaying, forestalling, or avoiding the onset or development of a disease, condition, or disorder for a period of time. Prevent also means reducing risk of developing a disease, disorder, or condition. Prevention includes minimizing or partially or completely inhibiting the development of a disease, condition, or disorder. In some embodiments, a composition, e.g., a pharmaceutical composition, prevents a disorder by delaying the onset of the disorder for 12 hours, 24 hours, 2 days, 3 days, 4 days, 5 days, 6 days, 7 days, 2 weeks, 3 weeks, 4 weeks, 2 months, 3 months, 4 months, 5 months, 6 months, 1 year, 2 years, 3 years, 4 years, 5 years, 6 years, indefinitely, or life of a subject.

The term “effective amount” or “therapeutically effective amount” may refer to a quantity of a composition, for example a composition comprising a construct, that can be sufficient to result in a desired activity upon introduction into a subject as disclosed herein. An effective amount of the prime editing compositions can be provided to the target gene or cell, whether the cell is ex vivo or in vivo.

An effective amount can be the amount to induce, for example, at least about a 2-fold change (increase or decrease) or more in the amount of target nucleic acid modulation (e.g., expression of a gene to produce functional a protein) observed relative to a negative control. An effective amount or dose can induce, for example, about 2-fold increase, about 3-fold increase, about 4-fold increase, about 5-fold increase, about 6-fold increase, about 7-fold increase, about 8-fold increase, about 9-fold increase, about 10-fold increase, about 25-fold increase, about 50-fold increase, about 100-fold increase, about 200-fold increase, about 500-fold increase, about 700-fold increase, about 1000-fold increase, about 5000-fold increase, or about 10,000-fold increase in target gene modulation (e.g., expression of a target gene to produce a functional protein).

The amount of target gene modulation may be measured by any suitable method known in the art. In some embodiments, the “effective amount” or “therapeutically effective amount” is the amount of a composition that is required to ameliorate the symptoms of a disease relative to an untreated patient. In some embodiments, an effective amount is the amount of a composition sufficient to introduce an alteration in a gene of interest in a cell (e.g., a cell in vitro or in vivo).

### Prime Editing

The term “prime editing” refers to programmable editing of a target DNA using a prime editor complexed with a PEgRNA to incorporate an intended nucleotide edit into the target DNA through target-primed DNA synthesis. A target polynucleotide, e.g., a target gene of prime editing may comprise a double stranded DNA molecule having two complementary strands: a first strand that may be referred to as a “target strand” or a “non-edit strand”, and a second strand that may be referred to as a “non-target strand,” or an “edit strand.” In some embodiments, in a prime editing guide RNA (PEgRNA), a spacer sequence is complementary or substantially complementary to a specific sequence on the target strand, which may be referred to as a “search target sequence”. In some embodiments, the spacer sequence anneals with the target strand at the search target sequence. The target strand may also be referred to as the “non-Protospacer Adjacent Motif (non-PAM strand).” In some embodiments, the non-target strand may also be referred to as the “PAM strand”. In some embodiments, the PAM strand comprises a protospacer sequence and optionally a protospacer adjacent motif (PAM) sequence. In prime editing using a Cas-protein-based prime editor, a PAM sequence refers to a short DNA sequence immediately adjacent to the protospacer sequence on the PAM strand of the target gene. A PAM sequence may be specifically recognized by a programmable DNA binding protein, e.g., a Cas nickase or a Cas nuclease In some embodiments, a specific PAM is characteristic of a specific programmable DNA binding protein, e.g., a Cas nickase or a Cas nuclease. A protospacer sequence refers to a specific sequence in the PAM strand of the target gene that is complementary to the search target sequence. In a PEgRNA, a spacer sequence may have a substantially identical sequence as the protospacer sequence on the edit strand of a target gene, except that the spacer sequence may comprise Uracil (U) and the protospacer sequence may comprise Thymine (T).

In some embodiments, the double stranded target DNA comprises a nick site on the PAM strand (or non-target strand). As used herein, a “nick site” refers to a specific position in between two nucleotides or two basepairs of the double stranded target DNA. In some embodiments, the position of a nick site is determined relative to the position of a specific PAM sequence. In some embodiments, the nick site is the particular position where a nick will occur when the double stranded target DNA is contacted with a nickase, for example, a Cas nickase, that recognizes a specific PAM sequence. In some embodiments, the nick site is upstream of a specific PAM sequence on the PAM strand of the double stranded target DNA. In some embodiments, the nick site is downstream of a specific PAM sequence on the PAM strand of the double stranded target DNA. In some embodiments, the nick site is 3 basepairs upstream of the PAM sequence, and the PAM sequence is recognized by a Streptococcus pyogenes Cas9 nickase, a P. lavamentivorans Cas9 nickase, a C. diphtheriae Cas9 nickase, a N. cinerea Cas9, a S. aureus Cas9, or a N. lari Cas9 nickase. In some embodiments, the nick site is 3 basepairs upstream of the PAM sequence, and the PAM sequence is recognized by a Cas9 nickase, wherein the Cas9 nickase comprises a nuclease active HNH domain and a nuclease inactive RuvC domain. In some embodiments, the nick site is 2 basepairs upstream of the PAM sequence, and the PAM sequence is recognized by a S. thermophilus Cas9 nickase.

In some embodiments, a PEgRNA complexes with and directs a prime editor to bind to the search target sequence of the target gene. In some embodiments, the bound prime editor generates a nick on the edit strand (PAM strand) of the target gene at the nick site. In some embodiments, a primer binding site (PBS) of the PEgRNA anneals with a free 3′ end formed at the nick site, and the prime editor initiates DNA synthesis from the nick site, using the free 3′ end as a primer. Subsequently, a single-stranded DNA encoded by the editing template of the PEgRNA is synthesized. In some embodiments, the newly synthesized single-stranded DNA comprises one or more intended nucleotide edits compared to the endogenous target gene sequence. In some embodiments, the editing template of a PEgRNA is complementary to a sequence in the edit strand except for one or more mismatches at the intended nucleotide edit positions in the editing template partially complementary to the editing template may be referred to as an “editing target sequence”. Accordingly, in some embodiments, the newly synthesized single stranded DNA has identity or substantial identity to a sequence in the editing target sequence, except for one or more insertions, deletions, or substitutions at the intended nucleotide edit positions.

In some embodiments, the newly synthesized single-stranded DNA equilibrates with the editing target on the edit strand of the target gene for pairing with the target strand of the target gene. In some embodiments, the editing target sequence of the target gene is excised by a flap endonuclease (FEN), for example, FEN1. In some embodiments, the FEN is an endogenous FEN, for example, in a cell comprising the target gene. In some embodiments, the FEN is provided as part of the prime editor, either linked to other components of the prime editor or provided in trans. In some embodiments, the newly synthesized single stranded DNA, which comprises the intended nucleotide edit, replaces the endogenous single stranded editing target sequence on the edit strand of the target gene. In some embodiments, the newly synthesized single stranded DNA and the endogenous DNA on the target strand form a heteroduplex DNA structure at the region corresponding to the editing target sequence of the target gene. In some embodiments, the newly synthesized single-stranded DNA comprising the nucleotide edit is paired in the heteroduplex with the target strand of the target DNA that does not comprise the nucleotide edit, thereby creating a mismatch between the two otherwise complementary strands. In some embodiments, the mismatch is recognized by DNA repair machinery, e.g., an endogenous DNA repair machinery. In some embodiments, through DNA repair, the intended nucleotide edit is incorporated into the target gene.

### Modified PEgRNAs

In certain aspects, provided herein are modified PEgRNAs. The term “prime editing guide RNA”, or “PEgRNA”, refers to a guide polynucleotide that comprises one or more intended nucleotide edits for incorporation into the target DNA. In some embodiments, the PEgRNA associates with and directs a prime editor to incorporate the one or more (e.g., two or more, three or more, four or more, or five or more) intended nucleotide edits into the target gene via prime editing. “Nucleotide edit” or “intended nucleotide edit” refers to a specified deletion of one or more nucleotides at one specific position, insertion of one or more nucleotides at one specific position, substitution of a single nucleotide, or other alterations at one specific position to be incorporated into the sequence of the target gene. Intended nucleotide edit may refer to the edit on the editing template as compared to the sequence on the target strand of the target gene, or may refer to the edit encoded by the editing template on the newly synthesized single stranded DNA that replaces the editing target sequence, as compared to the editing target sequence. In some embodiments, a PEgRNA comprises a spacer sequence that is complementary or substantially complementary to a search target sequence on a target strand of the target gene. In some embodiments, the PEgRNA comprises a gRNA core that associates with a DNA binding domain, e.g., a CRISPR-Cas protein domain, of a prime editor. In some embodiments, the PEgRNA further comprises an extended nucleotide sequence comprising one or more intended nucleotide edits compared to the endogenous sequence of the target gene, wherein the extended nucleotide sequence may be referred to as an extension arm. In certain embodiments, the PEgRNA comprises a primer binding site sequence (PBS) that can initiate target-primed DNA synthesis. In some embodiments, the PEgRNA comprises an editing template that comprises one or more intended nucleotide edits to be incorporated in the target gene by prime editing. In some embodiments, the extension arm comprises a PBS. In some embodiments, the extension arm comprises an editing template that comprises one or more intended nucleotide edits to be incorporated in the target gene by prime editing.

A “primer binding site” (PBS or primer binding site sequence) is a single-stranded portion of the PEgRNA that comprises a region of complementarity to the PAM strand (i.e. the non-target strand or the edit strand). The PBS is complementary or substantially complementary to a sequence on the PAM strand of the double stranded target DNA that is immediately upstream of the nick site. In some embodiments, in the process of prime editing, the PEgRNA complexes with and directs a prime editor to bind the search target sequence on the target strand of the double stranded target DNA, and generates a nick at the nick site on the non-target strand of the double stranded target DNA. In some embodiments, the PBS is complementary to or substantially complementary to, and can anneal to, a free 3′ end on the non-target strand of the double stranded target DNA at the nick site. In some embodiments, the PBS annealed to the free 3′ end on the non-target strand can initiate target-primed DNA synthesis.

An “editing template” of a PEgRNA is a single-stranded portion of the PEgRNA that is 5′ of the PBS and comprises a region of complementarity to the PAM strand (i.e. the non-target strand or the edit strand), and comprises one or more intended nucleotide edits compared to the endogenous sequence of the double stranded target DNA. In some embodiments, the editing template and the PBS are immediately adjacent to each other. Accordingly, in some embodiments, a PEgRNA in prime editing comprises a single-stranded portion that comprises the PBS and the editing template immediately adjacent to each other. In some embodiments, the single stranded portion of the PEgRNA comprising both the PBS and the editing template is complementary or substantially complementary to an endogenous sequence on the PAM strand (i.e. the non-target strand or the edit strand) of the double stranded target DNA except for one or more non-complementary nucleotides at the intended nucleotide edit positions. As used herein, regardless of relative 5′-3′ positioning in other context, the relative positions as between the PBS and the editing template, and the relative positions as among elements of a PEgRNA, are determined by the 5′ to 3′ order of the PEgRNA as a single molecule regardless of the position of sequences in the double stranded target DNA that may have complementarity or identity to elements of the PEgRNA. In some embodiments, the editing template is complementary or substantially complementary to a sequence on the PAM strand that is immediately downstream of the nick site, except for one or more non-complementary nucleotides at the intended nucleotide edit positions. The endogenous, e.g., genomic, sequence that is complementary or substantially complementary to the editing template, except for the one or more non-complementary nucleotides at the position corresponding to the intended nucleotide edit, may be referred to as an “editing target sequence”. In some embodiments, the editing template has identity or substantial identity to a sequence on the target strand that is complementary to, or having the same position in the genome as, the editing target sequence, except for one or more insertions, deletions, or substitutions at the intended nucleotide edit positions. In some embodiments, the editing template encodes a single stranded DNA, wherein the single stranded DNA has identity or substantial identity to the editing target sequence except for one or more insertions, deletions, or substitutions at the positions of the one or more intended nucleotide edits.

### Spacers

A spacer may guide a prime editing complex to a genomic locus with identical or substantially identical sequence during prime editing. In some embodiments, the PEgRNA comprises a spacer. In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

In some embodiments, a spacer sequence comprises a region that has substantial complementarity to a search target sequence on the target strand of a double stranded target DNA. In some embodiments, the spacer sequence of a PEgRNA is identical or substantially identical to a protospacer sequence on the edit strand of the target gene (except that the protospacer sequence comprises thymine and the spacer sequence may comprise uracil). In some embodiments, the spacer sequence is at least about 70%, 75%, 80%, 85%, 90%, 95%, or 100% complementary to a search target sequence in the target gene. In some embodiments, the spacer comprises is substantially complementary to the search target sequence.

In some embodiments, the length of the spacer varies from at least 10 nucleotides to 100 nucleotides. For examples, a spacer may be at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, at least 50 nucleotides, at least 60 nucleotides, at least 70 nucleotides, at least 80 nucleotides, at least 90 nucleotides, at least 100 nucleotides. In some embodiments, the spacer is 16 nucleotides, 17 nucleotides, 18 nucleotides, 19 nucleotides, 20 nucleotides, 21 nucleotides, 22 nucleotides, 23 nucleotides, 24 nucleotides, or 25 nucleotides in length. In some embodiments, the spacer is from 15 nucleotides to 30 nucleotides in length, 15 to 25 nucleotides in length, 18 to 22 nucleotides in length, 10 to 20 nucleotides in length, 20 to 30 nucleotides in length, 30 to 40 nucleotides in length, 40 to 50 nucleotides in length, 50 to 60 nucleotides in length, 60 to 70 nucleotides in length, 70 to 80 nucleotides in length, or 90 nucleotides to 100 nucleotides in length. In some embodiments, the spacer is 20 nucleotides in length. In some embodiments, the spacer is 17 to 18 nucleotides in length.

As used herein in a PEgRNA or a nick guide RNA sequence, or fragments thereof such as a spacer, PBS, or RTT sequence, unless indicated otherwise, it should be appreciated that the letter “T” or “thymine” indicates a nucleobase in a DNA sequence that encodes the PEgRNA or guide RNA sequence, and is intended to refer to a uracil (U) nucleobase of the PEgRNA or guide RNA or any chemically modified uracil nucleobase known in the art, such as 5-methoxyuracil.

### Primer Binding Site (PBS)

A PEgRNA may comprise a primer binding site (PBS) and an editing template (e.g., an RTT). The extension arm of a PEgRNA may comprise a PBS and an editing template. In some embodiments, a PBS may be partially complementary to the spacer. In some embodiments, the editing template (e.g., RTT) is partially complementary to the spacer. In some embodiments, the editing template (e.g., RTT) and the primer binding site (PBS) are each partially complementary to the spacer.

An extension arm of a PEgRNA may comprise a primer binding site sequence (PBS, or PBS sequence) that hybridizes with a free 3′ end of a single stranded DNA in the target gene generated by nicking with a prime editor. The length of the PBS sequence may vary depending on, e.g., the prime editor components, the search target sequence and other components of the PEgRNA. In some embodiments, the length of the primer binding site (PBS) varies from at least 2 nucleotides to 50 nucleotides. For examples, a primer binding site (PBS) may be at least 2 nucleotides, at least 3 nucleotides, at least 4 nucleotides, at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 30 nucleotides, at least 40 nucleotides, or at least 50 nucleotides in length. In some embodiments, the PBS is at least 6 nucleotides in length. In some embodiments, the PBS is about 4 to 16 nucleotides, about 6 to 16 nucleotides, about 6 to 18 nucleotides, about 6 to 20 nucleotides, about 8 to 20 nucleotides, about 10 to 20 nucleotides, about 12 to 20 nucleotides, about 14 to 20 nucleotides, about 16 to 20 nucleotides, or about 18 to 20 nucleotides in length. In some embodiments, the PBS is about 7 to 15 nucleotides in length. In some embodiments, the PBS is 6, 7, 8, 9, 10, 11, 12, 13, 14, 15, 16, 17, 18, 19, or 20 nucleotides in length. In some embodiments, the PBS is 8, 9, 10, 11, 12, 13, or 14 nucleotides in length.

The PBS may be complementary or substantially complementary to a DNA sequence in the edit strand of the target gene. By annealing with the edit strand at a free hydroxy group, e.g., a free 3′ end generated by prime editor nicking, the PBS may initiate synthesis of a new single stranded DNA encoded by the editing template at the nick site. In some embodiments, the PBS is at least about 70%, 75%, 80%, 85%, 90%, 95%, or 100% complementary to a region of the edit strand of the target gene. In some embodiments, the PBS is perfectly complementary, or 100% complementary, to a region of the edit strand of the target gene.

An extension arm of a PEgRNA may comprise an editing template that serves as a DNA synthesis template for the DNA polymerase in a prime editor during prime editing.

The length of an editing template may vary depending on, e.g., the prime editor components, the search target sequence and other components of the PEgRNA. In some embodiments, the editing template serves as a DNA synthesis template for a reverse transcriptase, and the editing template is referred to as a reverse transcription editing template (RTT).

The editing template (e.g., RTT), in some embodiments, is 2, 3, 4, 5, 6, 7, 8, 9, 10, 11, 12, 13, 14, 15, 16, 17, 18, 19, 20, 21, 22, 23, 24, or 25 nucleotides in length. In some embodiments, the RTT is 12, 13, 14, 15, 16, 17, 18, 19, 20, 21, 22, 23, 24, or 25 nucleotides in length. In some embodiments, the RTT is 16, 17, 18, 19, 20, 21, 22, 23, 24, 25, 26, 27, 28, 29, 30, 31, 32, 33, 34, 35, 36, 37, 38, 39, or 40 nucleotides in length.

In some embodiments, the editing template (e.g., RTT) sequence is about 70%, 75%, 80%, 85%, 90%, 95%, or 99% complementary to the editing target sequence on the edit strand of the target gene. In some embodiments, the editing template sequence (e.g., RTT) is substantially complementary to the editing target sequence. In some embodiments, the editing template sequence (e.g., RTT) is complementary to the editing target sequence except at positions of the intended nucleotide edits to be incorporated into the target gene. In some embodiments, the editing template comprises a nucleotide sequence comprising about 85% to about 95% complementarity to an editing target sequence in the edit strand in the target gene. In some embodiments, the editing template comprises about 86%, about 87%, about 88%, about 89%, about 90%, about 91%, about 92%, about 93%, about 94%, about 95%, about 96%, about 97%, about 98%, or about 99% complementarity to an editing target sequence in the edit strand of the target gene.

In some embodiments, a PEgRNA includes only RNA nucleotides and forms an RNA polynucleotide. In some embodiments, a PEgRNA is a chimeric polynucleotide that includes both RNA and DNA nucleotides. For example, a PEgRNA can include DNA in the spacer sequence, the gRNA core, or the extension arm. In some embodiments, a PEgRNA comprises DNA in the spacer sequence. In some embodiments, the entire spacer sequence of a PEgRNA is a DNA sequence. In some embodiments, the PEgRNA comprises DNA in the gRNA core, for example, in a stem region of the gRNA core. In some embodiments, the PEgRNA comprises DNA in the extension arm, for example, in the editing template. An editing template that comprises a DNA sequence may serve as a DNA synthesis template for a DNA polymerase in a prime editor, for example, a DNA-dependent DNA polymerase. Accordingly, the PEgRNA may be a chimeric polynucleotide that comprises RNA in the spacer, gRNA core, and/or the PBS sequences and DNA in the editing template.

Components of a PEgRNA may be arranged in a modular fashion. In some embodiments, the spacer and the extension arm comprising a primer binding site sequence (PBS) and an editing template, e.g., a reverse transcriptase template (RTT), can be interchangeably located in the 5′ portion of the PEgRNA, the 3′ portion of the PEgRNA, or in the middle of the gRNA core. For example, in some embodiments, a PEgRNA comprises, from 5′ to 3′: a spacer, a gRNA core, an editing template, and a PBS. In some embodiments, a PEgRNA comprises, from 5′ to 3′: an editing template, a PBS, a spacer, and a gRNA core. In some embodiments, the PBS and/or the editing template is positioned within the gRNA core, i.e., flanked by a first half of the gRNA core and a second half of the gRNA core.

In certain embodiments, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, wherein the PEgRNA further comprises one or more nucleic acid moieties at its 3′ end.

In some embodiments, the PEgRNA comprises, in 5′ to 3′ order, the spacer, the gRNA core, the editing template, and the PBS.

In certain embodiments, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, wherein the gRNA core comprises one or more sequence modifications compared to SEQ ID NO. 16.

In some embodiments, the PEgRNA comprises, in 5′ to 3′ order, the spacer, the gRNA core, the editing template, and the PBS.

In certain embodiments, PEgRNAs provided herein comprise i) a spacer that comprises a region of complementarity to a search target sequence in target strand of a double stranded target DNA; ii) a guide RNA (gRNA) core comprising a direct repeat, a first stem loop, and a second stem loop; iii) an editing template that comprises an intended edit compared to the double stranded target DNA; and iv) a primer binding site (PBS) that comprises a region of complementarity to a region upstream of a nick site in a non-target strand of the double stranded target DNA, and v) a tag sequence that comprises a region of complementarity to the PBS and/or the editing template.

In some embodiments, the PEgRNA comprises, in 5′ to 3′ order, the spacer, the gRNA core, the editing template, the PBS, and the tag sequence.

In some embodiments, the PEgRNA comprises, in 5′ to 3′ order, the editing template, the PBS, the tag sequence, the spacer, and the gRNA core.

In certain embodiments, PEgRNAs provided herein comprise in 5′ to 3′ order: i) a spacer that comprises a region of complementarity to a search target sequence in target strand of a double stranded target DNA; ii) 5′ part of a guide RNA (gRNA) core; iii) an editing template that comprises an intended edit compared to the double stranded target DNA; iv) a primer binding site (PBS) that comprises a region of complementarity to a region upstream of a nick site in a non-target strand of the double stranded target DNA; and v) a 3′ part of a gRNA core. In some embodiments, the 5′ part of the gRNA core and the 3′ part of the gRNA core form a complete functional gRNA core that can associate with a programmable DNA binding protein of a prime editor, e.g., a Cas9 nickase. In some embodiments, the 5′ part of the gRNA core comprises a direct repeat, a first stem loop, and a 5′ half of a second stem loop. In some embodiments, the 3′ part of the gRNA core comprises a 3′ half of a second stem loop and a third stem loop. In some embodiments, the PEgRNA further comprises a tag sequence that comprises a region of complementarity to the PBS and/or the editing template.

In certain embodiments, PEgRNAs provided herein comprise: i) a first sequence comprising a spacer that comprises a region of complementarity to a search target sequence in target strand of a double stranded target DNA, and a first half of a gRNA core; and ii) a second sequence comprising a second half of the gRNA core, an editing template that comprises an intended edit compared to the double stranded target DNA; a primer binding site (PBS) that comprises a region of complementarity to a region upstream of a nick site in a non-target strand of the double stranded target DNA; and, wherein the gRNA core comprises a direct repeat, a first stem loop, and a second stem loop. In certain embodiments, PEgRNAs provided herein comprise i) a first sequence comprising an editing template that comprises an intended edit compared to the double stranded target DNA; a primer binding site (PBS) that comprises a region of complementarity to a region upstream of a nick site in a non-target strand of the double stranded target DNA; a spacer that comprises a region of complementarity to a search target sequence in target strand of a double stranded target DNA; and a first half of a gRNA core; and ii) a second sequence comprising a second half of a gRNA core, wherein the gRNA core comprises a direct repeat, a first stem loop, and a second stem loop. In some embodiments, the first half of the gRNA core comprises a direct repeat, a first stem loop, and a 5′ half of a second stem loop. In some embodiments, the second part of the gRNA core comprises a 3′ half of a second stem loop and a third stem loop. In some embodiments, the first half of the gRNA core comprises a first half of a direct repeat. In some embodiments, the second half of the gRNA core comprises a second half of a direct repeat, a first stem loop, a second stem loop, and a third stem loop.

In some embodiments, the first sequence is on a first molecule and the second sequence is on a second molecule.

In some embodiments, the first sequence and the second sequence are on the same molecule.

In some embodiments, the first half of the gRNA core and the second half of the gRNA core are selected from the paired first half gRNA core sequences and second half gRNA sequences provided in Table 2.

Provided herein in some embodiments are example sequences for PEgRNA spacers, PBS, RTT, and ngRNA spacers for a prime editing system comprising a nuclease that recognizes the PAM sequence “NGG.” In some embodiments, a PAM motif on the edit strand comprises an “NGG” motif, wherein N is any nucleotide. In some embodiments, a PEgRNA of this disclosure is part of a prime editing system that recognizes the PAM motif CGG. In some embodiments, a PEgRNA of this disclosure is part of a prime editing system that recognizes the PAM motif AGG.

Modified gRNA Cores

In some embodiments, a gRNA core of a PEgRNA associates with a programmable DNA binding domain in a prime editor. In some embodiments, the gRNA core comprises a direct repeat, a first stem loop, and a second stem loop. In some embodiments, the gRNA core further comprises a third stem loop. A guide RNA core (also referred to herein as the gRNA core, gRNA scaffold, or gRNA backbone sequence) of a PEgRNA may contain a polynucleotide sequence that binds to a DNA binding domain (e.g., Cas9) of a prime editor. The gRNA core may interact with a prime editor as described herein, for example, by association with a DNA binding domain, such as a DNA nickase of the prime editor.

One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

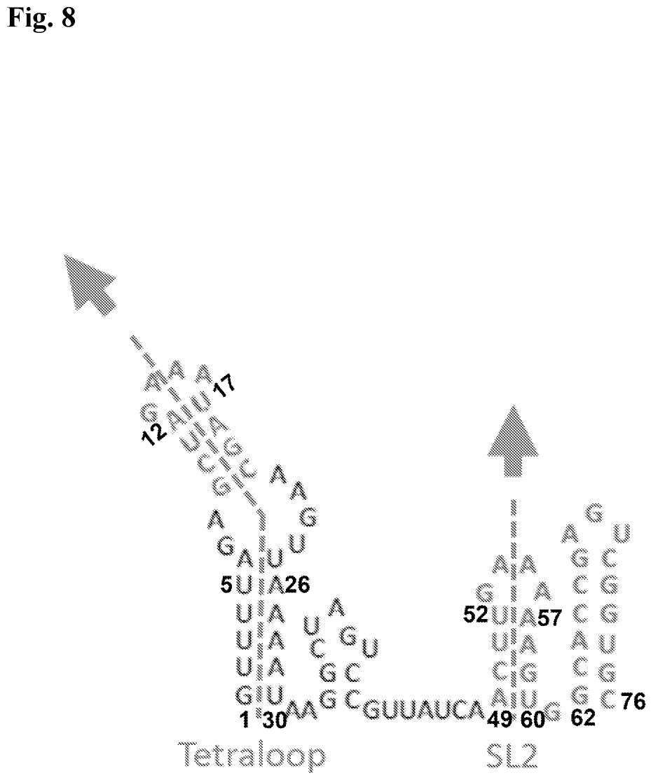

In some embodiments, the gRNA core comprises regions and secondary structures involved in binding with specific CRISPR Cas proteins. For example, in a Cas9 based prime editing system, the gRNA core of a PEgRNA may comprise one or more regions of a basepaired regions. In some embodiments, a gRNA core capable of binding to a Cas9 comprises, from 5′ to 3′: a repeat sequence, a loop structure, an antirepeat sequence, a first stem loop, a second stem loop, and a third stem loop. An exemplary structure of the gRNA core is shown in FIG. 8; the sequence in FIG. 8 is the canonical SpCas9 sgRNA scaffold. As used herein, a repeat sequence and an antirepeat sequence refer to the nucleic acid secondary structure formed by the direct repeat region, formed by basepairing between sequences equivalent to the crRNA and tracrRNA of a Cas9 guide RNA. The repeat sequence and the antirepeat sequence may be connected by a loop structure, and the secondary structure formed by basepairing between the repeat and antirepeat sequence may be referred to as the direct repeat region (alternatively, the repeat, antirepeat, and the connecting loop structure may be referred to as the tetraloop). In some embodiments, the direct repeat region of the gRNA core comprises one or more basepaired regions: a basepaired “lower stem” (G1 to A6 and U25 to U30 in FIG. 8) adjacent to the spacer sequence and a basepaired “upper stem” (G9 to A12 and U17 to C20 in FIG. 8) following the lower stem, where the lower stem and upper stem may be connected by a “bulge” comprising unpaired RNAs. As used herein, positions of alterations to the gRNA core may be referred to in the context of the secondary structure of the gRNA core. For example, a “first basepair in the direct repeat (or lower stem)” refers to the basepair between the 5′ most nucleotide in the repeat sequence and the complementary nucleotide that is the 3′ most nucleotide in the antirepeat sequence (G1 and A30 in FIG. 8), and a “second basepair in the direct repeat (or lower stem)” refers to the basepair between the second 5′ most nucleotide in the repeat sequence and the complementary nucleotide in the antirepeat sequence (U2 and A29 in FIG. 8). Similarly, the “start” or “beginning” basepair of a second stem loop refers to the basepair formed between the 5′ most nucleotide in the second stem loop and the complementary nucleotide in the complementary portion of the second stem loop (A49 and U60 in FIG. 8). The “end” or “last” basepair of a second stem loop refers to, wherein the second stem loop is formed by basepairing of a 5′ portion of the stem and a 3′ portion of the stem connected by a loop, the basepair formed between the 3′ most nucleotide in the 5′ portion of the stem and the complementary nucleotide in the complementary 3′ portion of the stem (U52 and A57 in FIG. 8).

The gRNA core may further comprise, 3′ to the direct repeat, a first stem loop, a second stem loop, and a third stem loop. In some embodiments, the gRNA core may comprise a direct repeat, and at least one, at least two, or at least three stem loops. As used herein, a stem loop (or a hairpin loop) is basepairing pattern that can occur in single-stranded nucleic acids. In some embodiments, a stem loop may be formed when two regions of the same nucleic acid strand are at least partially complementary in nucleotide sequence when read in opposite directions, therefore, the base-pairs can form a double helix that comprises an unpaired loop. Stem loops within a gRNA core described herein may be numbered starting from the 5′ to the 3′ end of the gRNA core. For example, the “first stem loop” would be the first stem loop (not including any direct repeats) at the 5′ end proximal to the direct repeat of the gRNA core sequence. A “second stem loop” would be the second stem loop (not including any direct repeats) following the first stem loop in a 5′ to 3′ direction, and so on.

In some embodiments, the gRNA core comprises nucleotide alterations as compared to a wild type gRNA core, e.g., a canonical SpCas9 gRNA scaffold as set forth in SEQ ID NO: 16. For example, in some embodiments, one or more nucleotides in the gRNA core is deleted, inserted, and/or substituted as compared to a canonical SpCas9 gRNA scaffold as set forth in SEQ ID NO: 16. In some embodiments, the gRNA core of a PEgRNA is capable of binding to a Cas9 (e.g. nCas9) in a prime editor, and comprise one or more nucleotide alterations or modifications as compared to a wild type CRISPR-Cas9 guide RNA scaffold, e.g., a canonical SpCas9 gRNA scaffold as set forth in SEQ ID NO: 16. In some embodiments, the gRNA core comprises one or more nucleotide insertions, deletions, and/or substitutions in the direct repeat as compared to a canonical SpCas9 gRNA scaffold as set forth in SEQ ID NO: 16. Potential advantages associated with such modified gRNA cores may include improved prime editing efficiency and/or improved manufacturing via a split synthesis scheme.

In some embodiments, the gRNA core comprises one or more nucleotide insertions, deletions, and/or substitutions in the lower stem or upper stem of the direct repeat. In some embodiments, the gRNA core comprises one or more nucleotide substitutions in the lower stem of the direct repeat. In some embodiments, the gRNA core comprises one or more nucleotide insertions in the upper stem of the direct repeat. In some embodiments, the gRNA core comprises one or more nucleotide insertions, deletions, and/or substitutions in the first stem loop as compared to a canonical SpCas9 gRNA scaffold as set forth in SEQ ID NO: 16. In some embodiments, the gRNA core comprises one or more nucleotide insertions, deletions, and/or substitutions in the second stem loop as compared to a canonical SpCas9 gRNA scaffold as set forth in SEQ ID NO: 16. In some embodiments, the gRNA core comprises one or more nucleotide insertions in the second stem loop. In some embodiments, the gRNA core comprises one or more nucleotide insertions, deletions, and/or substitutions in the third stem loop as compared to a canonical SpCas9 gRNA scaffold as set forth in SEQ ID NO: 16. In some embodiments, the gRNA core comprises one or more nucleotide insertions, deletions, and/or substitutions as compared to a wild type CRISPR-Cas9 guide RNA scaffold, e.g., a canonical SpCas9 gRNA scaffold as set forth in SEQ ID NO: 16, and comprises a third stem loop that has the same sequence as the third stem loop of the wild type CRISPR-Cas9 guide RNA scaffold.

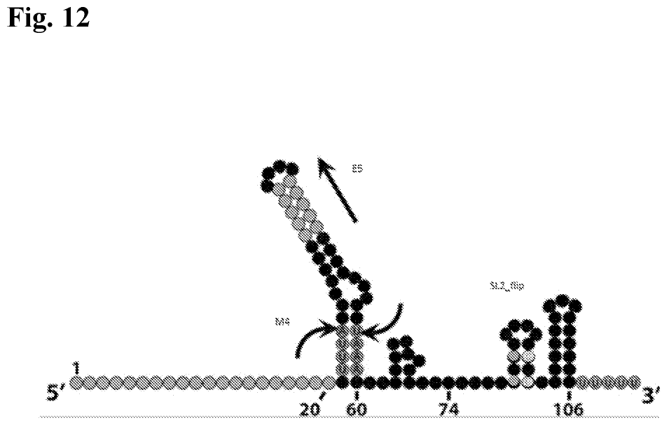

In some embodiments, RNA nucleotides in the lower stem, upper stem, an/or the stem loop regions may be replaced with one or more DNA sequences. In some embodiments, the gRNA core comprises unmodified or wild type RNA sequences in the nexus and/or the bulge regions. In some embodiments, the gRNA core does not include long stretches of A-U pairs, for example, a GUUUU-AAAAC pairing element. Exemplary gRNA core structures are shown in FIG. 8 and FIG. 12.

In some embodiments, the PEgRNA comprises a guide RNA (gRNA) core that associates with a DNA binding domain, e.g., a CRISPR-Cas protein domain, of a prime editor. In some embodiments, the PEgRNA comprises a guide RNA (gRNA) core that associates with a DNA binding domain, e.g., a Cas9 domain, of a prime editor. In certain aspects, the gRNA core of the PEgRNAs provided herein comprises one or more sequence modifications compared to SEQ ID NO. 16. In some embodiments, the one or more (e.g., two or more, three or more, four or more, or five or more) sequence modifications comprises a gRNA core difference set forth in Table 1 or Table 2. In some embodiments, the gRNA core comprises a sequence selected from SEQ ID NOs: 16-61. In some embodiments, the gRNA core comprises a first gRNA core sequence comprising a 5′ half of the gRNA core and a second gRNA core sequence comprising a 3′ half of the gRNA core, and wherein the PEgRNA comprises, in 5′ to 3′ order: the spacer, the first gRNA core sequence, the editing template, the PBS, the tag sequence, and the second gRNA core sequence. The 5′half and the 3′half can form a functional gRNA core for association/binding with a programmable DNA binding protein, e.g., a Cas protein. One of skill in the art will recognize that different prime editors having different DNA binding domains from different DNA binding proteins may require different gRNA core sequences specific to the DNA binding protein. In some embodiments, the gRNA core is capable of binding to a Cas9-based prime editor. In some embodiments, the gRNA core is capable of binding to a Cpf1-based prime editor. In some embodiments, the gRNA core is capable of binding to a Cas12b-based prime editor.

In some embodiments, the gRNA core of the PEgRNAs provided herein comprises one or more sequence modifications compared to SEQ ID NO. 16. In some embodiments, the one or more sequence modifications comprises a gRNA core alteration compared to SEQ ID No.: 16 set forth in Table 1. In some embodiments, the gRNA core comprises a gRNA core sequence set forth in Table 1 or Table 2.

In some embodiments, the one or more sequence modifications comprises a sequence modification in the direct repeat. In some embodiments, sequence modification in the gRNA core of a PEgRNA comprises one or more nucleotide flips. As used herein, the term “flip” refers to the modification of a sequence such that nucleotide bases that that base-pair with each other in the stem of a loop or hairpin structure are exchanged for each other. For example, an original unmodified stem structure may comprise an A/U basepair, with A in a first strand (or region) and U in the complementary strand (or region) of the stem structure. An A/U to U/A basepair flip substitutes the Adenosine in the first strand (or region) with a Uracil and substitutes the Uracil in the complementary strand (or region) with an Adenosine, thereby “flipping” the A/U basepair to an U/A basepair. In some embodiments, a flip of nucleotides can be used, for example, to break-up sequences containing repeats of the same base (for example sequences of at least 3, 4, 5, 6, or 7 consecutive A nucleotides, U nucleotides, C nucleotides, or G nucleotides) present in a nucleic acid molecule without disrupting its secondary structure. An example of an A/U flip that breaks-up a series of 4 consecutive A nucleotides and U nucleotides at the fourth position in the lower stem of a direct repeat without disrupting the gRNA core's secondary structure is illustrated in FIG. 12. In some embodiments, instead of a flip, the original basepair is replaced with an alternative basepair (e.g., an A/U basepair is replaced with a C/G or G/C basepair).

In some embodiments, the direct repeat of the gRNA core may comprise at least one flip of an A-U basepair in a lower stem of the direct repeat, optionally wherein the lower stem does not contain 2, 3, 4, or more contiguous A-U basepairs; and/or at least one flip of an A/U basepair in the direct repeat comprises a flip of the fourth A/U basepair in the lower stem of the direct repeat.

In some embodiments, the sequence modification in the direct repeat comprises insertion of one or more nucleotides in the upper stem of the direct repeat of the gRNA core, thereby resulting in an extension of the upper stem as compared to a wild type gRNA core, e.g., as set forth in SEQ ID NO: 16. The extension in the upper stem may be from 1, 2, 3, 4, 5, 6, 7, 8, 9, 10, 11, 12, 13, 14, 15, or 16 basepairs. In some embodiments, the gRNA core comprises a sequence selected from SEQ ID NOs: 26-37.

In some embodiments, the one or more sequence modifications comprises a sequence modification in the second stem loop.

In some embodiments, the modification in the second stem loop comprises a flip of a G/C basepair. In some embodiments, the modification in the second stem loop comprises a flip of an A/U basepair in the second stem loop. In some embodiments, the modification in the second stem loop comprises substitution of a A/U basepair with a G/C basepair. In some embodiments, the modification in the second stem loop comprises substitution of a U/A basepair with a G/C basepair. In some embodiments, the modification in the second stem loop comprises substitution of a A/U basepair with a G/C basepair, and further comprises a substitution of a U/A basepair with a G/C basepair. In some embodiments, the gRNA core comprises a nucleic acid sequence selected from SEQ ID NOs: 21, 22 or 25.

Exemplary gRNA core sequences and sequence modifications are shown in Table 1 and Table 2. In some embodiments, the gRNA core comprises a sequence selected from SEQ ID NOs: 16-61, 3860-4359, and 4452.

In some embodiments, the one or more sequence modifications comprises a modification in a third stem loop of the gRNA core. In some embodiments, the modification in the third stem loop comprises a flip of a G/C basepair. In some embodiments, the modification in the third stem loop comprises a flip of an A/U basepair.

The gRNA core may comprise any one of modifications described in Table 1 or Table 2, or any combination thereof.

In some embodiments, the gRNA core has a flipped 1st A-U basepair in the direct repeat. In some embodiments, the gRNA core has a flipped 2nd A-U base in the direct repeat. In some embodiments, the gRNA core has a flipped 3rd A-U basepair in the direct repeat. In some embodiments, the gRNA core has a flipped 4th A-U basepair in the direct repeat.

In some embodiments, the gRNA core comprises a substitution of an A-U basepair (bp) with a G-C Bp at the fourth basepair of the second stem loop. In some embodiments, the gRNA core comprises a substitution of an A-U Bp with a C-G Bp at the fourth basepair of second stem loop.

In some embodiments, the gRNA core comprises a five basepair extension of the upper stem of the direct repeat (tgctg and cagca). In some embodiments, the gRNA has a “flip and extension” (M4 and E5), as described in Nelson, J. W., Randolph, P. B., Shen, S. P. et al. Engineered pegRNAs improve prime editing efficiency. Nat Biotechnol (2021). The M4 modification is flipping the 4th A-U basepair in the direct repeat of gRNA core. The E5 modification is extending the end of the upper stem of the direct repeat with a five bp sequence (tgctg and cagca).

In some embodiments, a gRNA core comprises a M4 modification. In some embodiments, a gRNA core comprises a E5 modification. In some embodiments, a gRNA core comprises a M4 modification and a E5 modification.

In some embodiments, a gRNA core comprises a substitution of a A/U basepair with a G/C basepair in the second stem loop. In some embodiments, the gRNA core comprises a substitution of a A/U basepair with a G/C basepair at the first basepair of the second stem loop.

In some embodiments, the gRNA core has a 1 basepair extension in the upper stem of the direct repeat sequence (c and g). In some embodiments, the gRNA core has a 2 basepair extension in the upper stem of the direct repeat sequence (cc and gg). In some embodiments, the gRNA core has a 2 basepair extension in the upper stem of the direct repeat sequence (ca and tg). In some embodiments, the gRNA core has a 2 basepair extension in the upper stem of the direct repeat sequence (cg and tg). In some embodiments, the gRNA core has a 1 basepair extension in the upper stem of the direct repeat sequence (a and t). In some embodiments, the gRNA core has a 2 basepair extension in the upper stem of the direct repeat sequence (ac and gt). In some embodiments, the gRNA core has a 2 basepair extension in the upper stem of the direct repeat sequence (aa and tt). In some embodiments, the gRNA core has a 2 basepair extension in the upper stem of the direct repeat sequence (ag and tt). In some embodiments, the gRNA core has a 3 basepair extension in the upper stem of the direct repeat sequence (ccc and ggg). In some embodiments, the gRNA core has a 4 basepair extension in the upper stem of the direct repeat sequence (ccac and gtgg). In some embodiments, the gRNA core has a 5 basepair extension in the upper stem of the direct repeat sequence (ccaac and gttgg). In some embodiments, the gRNA core has a 6 basepair extension in the upper stem of the direct repeat sequence (ccacac and gtgtgg).

In some embodiments, the gRNA core has a 1 basepair extension in the second stem loop sequence (c and g). In some embodiments, the gRNA core has a 2 basepair extension in the second stem loop sequence (cc and gg). In some embodiments, the gRNA core has a 2 basepair extension in the second stem loop sequence (ca and tg). In some embodiments, the gRNA core has a 2 basepair extension in the second stem loop sequence (cg and tg). In some embodiments, the gRNA core has a 1 basepair extension in the second stem loop sequence (a and t). In some embodiments, the gRNA core has a 2 basepair extension in the second stem loop sequence (ac and gt). In some embodiments, the gRNA core has a 2 basepair extension in the second stem loop sequence (aa and tt). In some embodiments, the gRNA core has a 2 basepair extension in the second stem loop sequence (ag and tt). In some embodiments, the gRNA core has a 3 basepair extension in the second stem loop sequence (ccc and ggg). In some embodiments, the gRNA core has a 4 basepair extension in the second stem loop sequence (ccac and gtgg). In some embodiments, the gRNA core has a 5 basepair extension in the second stem loop sequence (ccaac and gttgg). In some embodiments, the gRNA core has a 6 basepair extension in the second stem loop sequence (ccacac and gtgtgg).

In some embodiments, the gRNA core has a 1 basepair extension in the third stem loop sequence (c and g). In some embodiments, the gRNA core has a 2 basepair extension in the third stem loop sequence (cc and gg). In some embodiments, the gRNA core has a 2 basepair extension in the third stem loop sequence (ca and tg). In some embodiments, the gRNA core has a 2 basepair extension in the third stem loop sequence (cg and tg). In some embodiments, the gRNA core has a 1 basepair extension in the third stem loop sequence (a and t). In some embodiments, the gRNA core has a 2 basepair extension in the third stem loop sequence (ac and gt). In some embodiments, the gRNA core has a 2 basepair extension in the third stem loop sequence (aa and tt). In some embodiments, the gRNA core has a 2 basepair extension in the third stem loop sequence (ag and tt). In some embodiments, the gRNA core has a 3 basepair extension in the third stem loop sequence (ccc and ggg). In some embodiments, the gRNA core has a 4 basepair extension in the third stem loop sequence (ccac and gtgg). In some embodiments, the gRNA core has a 5 basepair extension in the third stem loop sequence (ccaac and gttgg). In some embodiments, the gRNA core has a 6 basepair extension in the third stem loop sequence (ccacac and gtgtgg).

In some embodiments, as compared to editing efficiency with a control PEgRNA having a gRNA core without modifications, a gRNA core modification increase efficiency of editing by at least 10%, at least 20%, at least 30%, at least 40%, at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%. Exemplary nucleotide sequence modifications in the gRNA core of a PEgRNA are provided in Table 1. Modifications compared to a canonical SpCas9 gRNA scaffold sequence are indicated in the third column (“Modification description”). Although gRNA core sequences provided in Table 1 are RNA sequences. “T” is used instead of “U” in the sequences for consistency with the ST.26 standard.

TABLE 1
Exemplary gRNA Core Sequences

| SEQ | | | |
|---|---|---|---|
| ID | gRNA Core | | |
| NO. | name | Modification description | gRNA core sequence |
| 16 | Canonical | none | GTTTTAGAGCTAGAAATAGCAA |
| | SpCas9 gRNA | | GTTAAAATAAGGCTAGTCCGTT |
| | core | | ATCAACTTGAAAAAGTGGCACC |
| | | | GAGTCGGTGC |
| 17 | M1 | Flipping the 1st A-U Basepair | GATTTAGAGCTAGAAATAGCAA |
| | | at the beginning of tetraloop | GTTAAATTAAGGCTAGTCCGTT |
| | | (U to A substitution at | ATCAACTTGAAAAAGTGGCACC |
| | | nucleotide 2; A to U | GAGTCGGTGC |
| | | substitution at nucleoitde 29) | |
| 18 | M2 | Flipping the 2nd A-U Basepair | GTATTAGAGCTAGAAATAGCAA |
| | | at the beginning of tetraloop | GTTAATATAAGGCTAGTCCGTT |
| | | (U to A substitution at | ATCAACTTGAAAAAGTGGCACC |
| | | nucleotide 3; A to U | GAGTCGGTGC |
| | | substitution at nucleoitde 28) | |
| 19 | M3 | Flipping the 3rd A-U Basepair | GTTATAGAGCTAGAAATAGCAA |
| | | at the beginning of tetraloop | GTTATAATAAGGCTAGTCCGTT |
| | | (U to A substitution at | ATCAACTTGAAAAAGTGGCACC |
| | | nucleotide 4; A to U | GAGTCGGTGC |
| | | substitution at nucleoitde 27) | |
| 20 | M4 | Flipping the 4th A-T Basepair | GTTTAAGAGCTAGAAATAGCAA |
| | | at the beginning of tetraloop | GTTTAAATAAGGCTAGTCCGTT |
| | | (U to A substitution at | ATCAACTTGAAAAAGTGGCACC |
| | | nucleotide 5; A to U | GAGTCGGTGC |
| | | substitution at nucleoitde 26) | |
| 21 | sl2 gc | Converting an A-U basepair to | GTTTTAGAGCTAGAAATAGCAA |
| | | a G-C basepair at the Fourth | GTTAAAATAAGGCTAGTCCGTT |
| | | Basepair of StemLoop2 (U to | ATCAACTGGAAACAGTGGCACC |
| | | G substitution at nucleotide | GAGTCGGTGC |
| | | 52; A to C substitution at | |
| | | nucleotide 57) | |
| 22 | sl2 cg | converting an A-U basepair to | GTTTTAGAGCTAGAAATAGCAA |
| | | a C-G basepair at the Fourth | GTTAAAATAAGGCTAGTCCGTT |
| | | Basepair of StemLoop2 (U to | ATCAACTCGAAAGAGTGGCACC |
| | | C substitution at nucleotide | GAGTCGGTGC |
| | | 52; A to G substitution at | |
| | | nucleotide 57) | |
| 23 | E5 | 5 basepair insertion in | GTTTTAGAGCTATGCTGGAAAC |
| | | tetraloop upper stem (UGCUG | AGCATAGCAAGTTAAAATAAG |
| | | between nucleotides 12 and | GCTAGTCCGTTATCAACTTGAA |
| | | 13; CAGCA between | AAAGTGGCACCGAGTCGGTGC |
| | | nucleotides 16 and 17) | |
| 24 | F + E | M4 and E5 | GTTTAAGAGCTATGCTGGAAAC |
| | | | AGCATAGCAAGTTTAAATAAGG |
| | | | CTAGTCCGTTATCAACTTGAAA |
| | | | AAGTGGCACCGAGTCGGTGC |
| 25 | sl2_flip | M4 and conversion of an A-U | GTTTAAGAGCTAGAAATAGCAA |
| | | basepair to a G-C pair at the | GTTTAAATAAGGCTAGTCCGTT |
| | | first basepair of StemLoop2 | ATCAGCGTGAAAACGCGGCAC |
| | | (A to G substitution at | CGAGTCGGTGC |
| | | nucleotide 49; U to G | |
| | | substitution at nucleotide 51; | |
| | | A to C substitution at | |
| | | nucleotide 58, U to C | |
| | | substitution at nucleotide 60) | |
| 26 | TetraLoop_L0 | M4 and extension at the start | GTTTAAGAGCTACGAAAGTAGC |
| | | of tetraloop with 1 Basepair | AAGTTTAAATAAGGCTAGTCCG |
| | | (insertion of C between | TTATCAACTTGAAAAAGTGGCA |
| | | nucleotides 12 and 13, | CCGAGTCGGTGC |
| | | insertion of G between | |
| | | nucleotides 16 and 17) | |
| 27 | TetraLoop_L1 | M4 and extension at the start | GTTTAAGAGCTACCGAAAGGTA |
| | | of tetraloop with 2 Basepair | GCAAGTTTAAATAAGGCTAGTC |
| | | (insertion of CC between | CGTTATCAACTTGAAAAAGTGG |
| | | nucleotides 12 and 13, | CACCGAGTCGGTGC |
| | | insertion of GG between | |
| | | nucleotides 16 and 17) | |
| 28 | TetraLoop_L2 | M4 and extension at the start | GTTTAAGAGCTACAGAAATGTA |
| | | of tetraloop with 2 Basepair | GCAAGTTTAAATAAGGCTAGTC |
| | | (insertion of CA between | CGTTATCAACTTGAAAAAGTGG |
| | | nucleotides 12 and 13, | CACCGAGTCGGTGC |
| | | insertion of UG between | |
| | | nucleotides 16 and 17) | |
| 29 | TetraLoop_L3 | M4 and extension at the start | GTTTAAGAGCTACGGAAATGTA |
| | | of tetraloop with 2 Basepair | GCAAGTTTAAATAAGGCTAGTC |
| | | (insertion of CG between | CGTTATCAACTTGAAAAAGTGG |
| | | nucleotides 12 and 13, | CACCGAGTCGGTGC |
| | | insertion of UG between | |
| | | nucleotides 16 and 17) | |
| 30 | TetraLoop_L4 | M4 and extension at the start | GTTTAAGAGCTAAGAAATTAGC |
| | | of tetraloop with 1 Basepair | AAGTTTAAATAAGGCTAGTCCG |
| | | (insertion of A between | TTATCAACTTGAAAAAGTGGCA |
| | | nucleotides 12 and 13, | CCGAGTCGGTGC |
| | | insertion of U between | |
| | | nucleotides 16 and 17) | |
| 31 | TetraLoop_L5 | M4 and extension at the start | GTTTAAGAGCTAACGAAAGTTA |
| | | of tetraloop with 2 Basepair | GCAAGTTTAAATAAGGCTAGTC |
| | | (insertion of AC between | CGTTATCAACTTGAAAAAGTGG |
| | | nucleotides 12 and 13, | CACCGAGTCGGTGC |
| | | insertion of GU between | |
| | | nucleotides 16 and 17) | |
| 32 | TetraLoop_L6 | M4 and extension at the start | GTTTAAGAGCTAAAGAAATTTA |
| | | of tetraloop with 2 Basepair | GCAAGTTTAAATAAGGCTAGTC |
| | | (insertion of AA between | CGTTATCAACTTGAAAAAGTGG |
| | | nucleotides 12 and 13, | CACCGAGTCGGTGC |
| | | insertion of UU between | |
| | | nucleotides 16 and 17) | |
| 33 | TetraLoop_L7 | M4 and extension at the start | GTTTAAGAGCTAAGGAAATTTA |
| | | of tetraloop with 2 Basepair | GCAAGTTTAAATAAGGCTAGTC |
| | | (insertion of AG between | CGTTATCAACTTGAAAAAGTGG |
| | | nucleotides 12 and 13, | CACCGAGTCGGTGC |
| | | insertion of UU between | |
| | | nucleotides 16 and 17) | |
| 34 | TetraLoop_L8 | M4 and extension at the start | GTTTAAGAGCTACCCGAAAGG |
| | | of tetraloop with 3 Basepair | GTAGCAAGTTTAAATAAGGCTA |
| | | (insertion of CCC between | GTCCGTTATCAACTTGAAAAAG |
| | | nucleotides 12 and 13, | TGGCACCGAGTCGGTGC |
| | | insertion of GGG between | |
| | | nucleotides 16 and 17) | |
| 35 | TetraLoop_L9 | M4 and extension at the start | GTTTAAGAGCTACCACGAAAGT |
| | | of tetraloop with 4 Basepair | GGTAGCAAGTTTAAATAAGGCT |
| | | (insertion of CCAC between | AGTCCGTTATCAACTTGAAAAA |
| | | nucleotides 12 and 13, | GTGGCACCGAGTCGGTGC |
| | | insertion of GUGG between | |
| | | nucleotides 16 and 17) | |
| 36 | TetraLoop_L10 | M4 and extension at the start | GTTTAAGAGCTACCAACGAAAG |
| | | of tetraloop with 5 Basepair | TTGGTAGCAAGTTTAAATAAGG |
| | | (insertion of CCAAC between | CTAGTCCGTTATCAACTTGAAA |
| | | nucleotides 12 and 13, | AAGTGGCACCGAGTCGGTGC |
| | | insertion of GUUGG between | |
| | | nucleotides 16 and 17) | |
| 37 | TetraLoop_L11 | M4 and extension at the start | GTTTAAGAGCTACCACACGAAA |
| | | of tetraloop with 6 Basepair | GTGTGGTAGCAAGTTTAAATAA |
| | | (insertion of CCACAC | GGCTAGTCCGTTATCAACTTGA |
| | | between nucleotides 12 and | AAAAGTGGCACCGAGTCGGTG |
| | | 13, insertion of GUGUGG | C |
| | | between nucleotides 16 and | |
| | | 17) | |
| 38 | Loop2_L0 | M4 and extension at the start | GTTTAAGAGCTAGAAATAGCAA |
| | | of stem loop 2 with 1 basepair | GTTTAAATAAGGCTAGTCCGTT |
| | | (insertion of C between | ATCAACTTCGAAAGAAGTGGCA |
| | | nucleotides 52 and 53; | CCGAGTCGGTGC |
| | | insertion of G between | |
| | | nucletoides 56 and 57) | |
| 39 | Loop2_L1 | M4 and extension at the start | GTTTAAGAGCTAGAAATAGCAA |
| | | of stem loop 2 with 2 basepair | GTTTAAATAAGGCTAGTCCGTT |
| | | (insertion of CC between | ATCAACTTCCGAAAGGAAGTGG |
| | | nucleotides 52 and 53; | CACCGAGTCGGTGC |
| | | insertion of GG between | |
| | | nucletoides 56 and 57) | |
| 40 | Loop2_L2 | M4 and extension at the start | GTTTAAGAGCTAGAAATAGCAA |
| | | of stem loop 2 with 2 basepair | GTTTAAATAAGGCTAGTCCGTT |
| | | (insertion of CA between | ATCAACTTCAGAAATGAAGTGG |
| | | nucleotides 52 and 53; | CACCGAGTCGGTGC |
| | | insertion of UG between | |
| | | nucletoides 56 and 57) | |
| 41 | Loop2_L3 | M4 and extension at the start | GTTTAAGAGCTAGAAATAGCAA |
| | | of stem loop 2 with 2 basepair | GTTTAAATAAGGCTAGTCCGTT |
| | | (insertion of CG between | |
| | | nucleotides 52 and 53; | |
| | | insertion of UG between | ATCAACTTCGGAAATGAAGTGG |
| | | nucletoides 56 and 57) | CACCGAGTCGGTGC |
| 42 | Loop2_L4 | M4 and extension at the start | GTTTAAGAGCTAGAAATAGCAA |
| | | of stem loop 2 with 1 basepair | GTTTAAATAAGGCTAGTCCGTT |
| | | (insertion of A between | ATCAACTTAGAAATAAGTGGCA |
| | | nucleotides 52 and 53; | CCGAGTCGGTGC |
| | | insertion of U between | |
| | | nucletoides 56 and 57) | |
| 43 | Loop2_L5 | M4 and extension at the start | GTTTAAGAGCTAGAAATAGCAA |
| | | of stem loop 2 with 2 basepair | GTTTAAATAAGGCTAGTCCGTT |
| | | (insertion of AC between | ATCAACTTACGAAAGTAAGTGG |
| | | nucleotides 52 and 53; | CACCGAGTCGGTGC |
| | | insertion of GU between | |
| | | nucletoides 56 and 57) | |
| 44 | Loop2_L6 | M4 and extension at the start | GTTTAAGAGCTAGAAATAGCAA |
| | | of stem loop 2 with 2 basepair | GTTTAAATAAGGCTAGTCCGTT |
| | | (insertion of AA between | ATCAACTTAAGAAATTAAGTGG |
| | | nucleotides 52 and 53; | CACCGAGTCGGTGC |
| | | insertion of UU between | |
| | | nucletoides 56 and 57) | |
| 45 | Loop2_L7 | M4 and extension at the start | GTTTAAGAGCTAGAAATAGCAA |
| | | of stem loop 2 with 2 basepair | GTTTAAATAAGGCTAGTCCGTT |
| | | (insertion of AG between | ATCAACTTAGGAAATTAAGTGG |
| | | nucleotides 52 and 53; | CACCGAGTCGGTGC |
| | | insertion of UU between | |
| | | nucletoides 56 and 57) | |
| 46 | Loop2_L8 | M4 and extension at the start | GTTTAAGAGCTAGAAATAGCAA |
| | | of stem loop 2 with 3 basepair | GTTTAAATAAGGCTAGTCCGTT |
| | | (insertion of CCC between | ATCAACTTCCCGAAAGGGAAGT |
| | | nucleotides 52 and 53; | GGCACCGAGTCGGTGC |
| | | insertion of GGG between | |
| | | nucletoides 56 and 57) | |
| 47 | Loop2_L9 | M4 and extension at the start | GTTTAAGAGCTAGAAATAGCAA |
| | | of stem loop 2 with 4 basepair | GTTTAAATAAGGCTAGTCCGTT |
| | | (insertion of CCAC between | ATCAACTTCCACGAAAGTGGAA |
| | | nucleotides 52 and 53; | GTGGCACCGAGTCGGTGC |
| | | insertion of GUGG between | |
| | | nucletoides 56 and 57) | |
| 48 | Loop2_L10 | M4 and extension at the start | GTTTAAGAGCTAGAAATAGCAA |
| | | of stem loop 2 with 5 basepair | GTTTAAATAAGGCTAGTCCGTT |
| | | (insertion of CCAAC between | ATCAACTTCCAACGAAAGTTGG |
| | | nucleotides 52 and 53; | AAGTGGCACCGAGTCGGTGC |
| | | insertion of GUUGG between | |
| | | nucletoides 56 and 57) | |
| 49 | Loop2_L11 | M4 and extension at the start | GTTTAAGAGCTAGAAATAGCAA |
| | | of stem loop 2 with 6 basepair | GTTTAAATAAGGCTAGTCCGTT |
| | | (insertion of CCACAC | ATCAACTTCCACACGAAAGTGT |
| | | between nucleotides 52 and | GGAAGTGGCACCGAGTCGGTG |
| | | 53; insertion of GUGUGG | C |
| | | between nucletoides 56 and | |
| | | 57) | |
| 50 | Loop3_L0 | M4 and Extension at the start | GTTTAAGAGCTAGAAATAGCAA |
| | | of Stem Loop3 with a 1 | GTTTAAATAAGGCTAGTCCGTT |
| | | Basepair sequence (c and g) | |
| | | | ATCAACTTGAAAAAGTGGCACC |
| | | | GCAGTGCGGTGC |
| 51 | Loop3_L1 | M4 and Extension at the start | GTTTAAGAGCTAGAAATAGCAA |
| | | of stem loop3 with a 2 | GTTTAAATAAGGCTAGTCCGTT |
| | | Basepair sequence (cc and gg) | ATCAACTTGAAAAAGTGGCACC |
| | | | GCCAGTGGCGGTGC |
| 52 | Loop3_L2 | M4 and Extension at the start | GTTTAAGAGCTAGAAATAGCAA |
| | | of stem loop3 with a 2 | GTTTAAATAAGGCTAGTCCGTT |
| | | basepair sequence (ca and tg) | ATCAACTTGAAAAAGTGGCACC |
| | | | GCAAGTTGCGGTGC |
| 53 | Loop3_L3 | M4 and Extension at the start | GTTTAAGAGCTAGAAATAGCAA |
| | | of stem loop3 with a 2 | GTTTAAATAAGGCTAGTCCGTT |
| | | basepair sequence (cg and tg) | ATCAACTTGAAAAAGTGGCACC |
| | | | GCGAGTTGCGGTGC |
| 54 | Loop3_L4 | M4 and Extension at the start | GTTTAAGAGCTAGAAATAGCAA |
| | | of stem loop3 with a 1 | GTTTAAATAAGGCTAGTCCGTT |
| | | basepair sequence (a and t) | ATCAACTTGAAAAAGTGGCACC |
| | | | GAAGTTCGGTGC |
| 55 | Loop3_L5 | M4 and Extension at the start | GTTTAAGAGCTAGAAATAGCAA |
| | | of stem loop3 with a 2 | GTTTAAATAAGGCTAGTCCGTT |
| | | basepair sequence (ac and gt) | ATCAACTTGAAAAAGTGGCACC |
| | | | GACAGTGTCGGTGC |
| 56 | Loop3_L6 | M4 and Extension at the start | GTTTAAGAGCTAGAAATAGCAA |
| | | of stem loop3 with a 2 | GTTTAAATAAGGCTAGTCCGTT |
| | | basepair sequence (aa and tt) | ATCAACTTGAAAAAGTGGCACC |
| | | | GAAAGTTTCGGTGC |
| 57 | Loop3_L7 | M4 and Extension at the start | GTTTAAGAGCTAGAAATAGCAA |
| | | of stem loop3 with a 2 | GTTTAAATAAGGCTAGTCCGTT |
| | | basepair sequence (ag and tt) | ATCAACTTGAAAAAGTGGCACC |
| | | | GAGAGTTTCGGTGC |
| 58 | Loop3_L8 | M4 and Extension at the start | GTTTAAGAGCTAGAAATAGCAA |
| | | of stem loop3 with a 3 | GTTTAAATAAGGCTAGTCCGTT |
| | | basepair sequence (ccc and | ATCAACTTGAAAAAGTGGCACC |
| | | ggg) | GCCCAGTGGGCGGTGC |
| 59 | Loop3_L9 | M4 and Extension at the start | GTTTAAGAGCTAGAAATAGCAA |
| | | of stem loop3 with a 4 | GTTTAAATAAGGCTAGTCCGTT |
| | | Basepair sequence (ccac and | ATCAACTTGAAAAAGTGGCACC |
| | | gtgg) | GCCACAGTGTGGCGGTGC |
| 60 | Loop3_L10 | M4 and Extension at the start | GTTTAAGAGCTAGAAATAGCAA |
| | | of stem soop3 with a 5 | GTTTAAATAAGGCTAGTCCGTT |
| | | Basepair sequence (ccaac and | ATCAACTTGAAAAAGTGGCACC |
| | | gttgg) | GCCAACAGTGTTGGCGGTGC |
| 61 | Loop3_L11 | M4 and Extension at the start | GTTTAAGAGCTAGAAATAGCAA |
| | | of stem loop3 with a 6 | GTTTAAATAAGGCTAGTCCGTT |
| | | Basepair sequence (ccacac and | ATCAACTTGAAAAAGTGGCACC |
| | | gtgtgg) | GCCACACAGTGTGTGGCGGTG |
| | | | C |
| 4280 | T_e1b_13-2 | M4; replacement of | GTTTAAGAGCGGGGAAATCCG |
| | | nucleotides 11-12 with GGG; | CAAGTTTAAATAAGGCTAGTCC |
| | | replacement of nucleotides 17- | GTTATCAACTTGAAAAAGTGGC |
| | | 18 with UCC | ACCGAGTCGGTGC |
| 4452 | T_e1b_13_SL2 | M4; replacement of | GTTTAAGAGCGGGGAAATCCG |
| | | nucleotides 11-12 with GGG; | CAAGTTTAAATAAGGCTAGTCC |
| | | replacement of nucleotides 17- | GTTATCAGCGTGAAAACGCGGC |
| | | 18 with UCC; A to G | ACCGAGTCGGTGC |
| | | substitution at nucleotide 49; | |
| | | U to G substitution at | |
| | | nucleotide 51; A to C | |
| | | substitution at nucleotide 58, | |
| | | U to C substitution at | |
| | | nucleotide 60) | |

### Nucleic Acid Moieties

In some embodiments, the PEgRNA comprises one or more nucleic acid moieties (e.g., hairpin, pseudoknot, quadruplex, tRNA sequence, aptamer) in addition to the spacer, gRNA core, primer binding site, and editing template. In some embodiments such nucleic acid moieties are positioned on the 3′ end of the PEgRNA.

In some embodiments, the nucleic acid moiety comprise a hairpin. In some embodiments, a hairpin is a nucleic acid secondary structure formed by intramolecular basepairing between a two regions of the same strand, which are typically complementary in nucleotide sequence when read in opposite directions. The two regions base-pair to form a double helix that ends in an unpaired loop. As described herein, the hairpin may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the hairpin is 14 nucleotides in length. In some embodiments, the hairpin is 18 nucleotides in length. In some embodiments, the hairpin is 22 nucleotides in length. In some embodiments, the hairpin comprises 2, 3, 4, 5, 6, 7, 8, 9, 10, 11, 12, 13, 14, 15 or more contiguous complementary basepairs. In some embodiments, the hairpin comprises 4, 5, 6, 7, 8, 9, or 10 contiguous complementary basepairs. In some embodiments, the hairpin comprises 4-8 contiguous complementary basepairs. In some embodiments, the hairpin comprises 5 contiguous complementary basepairs. In some embodiments, the hairpin comprises 7 contiguous complementary basepairs.

In some embodiments, the nucleic acid moiety comprises a pseudoknot. As used herein, a pseudoknot, includes, but is not limited to a nucleic acid secondary structure containing at least two stem-loop structures in which half of one stem is intercalated between the two halves of another stem. Several distinct folding topologies of pseudoknots exist, including, for example, the H type. In the H-type fold, the bases in the loop of a hairpin form intramolecular pairs with bases outside of the stem. This causes the formation of a second stem and loop, resulting in a pseudoknot with two stems and two loops. As described herein, the pseudoknot may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the pseudoknot is 22 nucleotides in length.

In some embodiments, the nucleic acid moiety comprises a quadruplex. In some embodiments, quadruplexes are noncanonical four-stranded, nucleic acid secondary structures that can be formed, in some contexts, in guanine-rich or cysteine-rich DNA and RNA sequences. As described herein, the quadruplexes may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the quadruplex is 18 nucleotides in length. In some embodiments, the quadruplex is rich in Guanine (a G-quadruplex). In some embodiments, the quadruplex is rich in Cytosine (a C-quadruplex).

In some embodiments, the nucleic acid moiety comprises an aptamer. In some embodiments, an aptamer comprises a short, single-stranded nucleic acid oligomer that can bind to a specific target molecule. Aptamers may assume a variety of shapes due to their tendency to form helices and single-stranded loops. As described herein, the aptamer may be between 5 and 50 nucleotides in length, between 10 and 40 nucleotides in length, or at least 15 and 30 nucleotides in length. The hairpin may be at least 10 nucleotides in length, at least 15 nucleotides in length, at least 20 nucleotides in length, at least 25 nucleotides in length, or at least 30 nucleotides in length. In some embodiments, the aptamer is 19 nucleotides in length. In some embodiments, the aptamer is 33 nucleotides in length.

In some embodiments, the nucleic acid moiety comprises a tRNA sequence. A tRNA sequence may be long (e.g., at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, or at least 75 nucleotides) In some embodiments, a tRNA sequence may be short (less than 25 nucleotides, less than 20 nucleotides, less than 15 nucleotides, or less than 10 nucleotides). As described herein, the tRNA sequences may be between 5 and 80 nucleotides in length, between 10 and 70 nucleotides in length, or at least 15 and 60 nucleotides in length. The hairpin may be at least 10 nucleotides in length, at least 15 nucleotides in length, at least 20 nucleotides in length, at least 25 nucleotides in length, at least 30 nucleotides in length, at least 40 nucleotides in length, at least 50 nucleotides in length, at least 60 nucleotides in length, or at least 70 nucleotides in length. In some embodiments, the aptamer is 18 nucleotides in length. In some embodiments, the aptamer is 61 nucleotides in length.

Exemplary moieties can be found in Table 4. A person of skill in the art would appreciate that the present disclosure is not limited by the sequences and structures in Table 4 as the configurations in Table 4 are examples of a broader class of moieties included in the present disclosure.

In some embodiments, the one or more nucleic acid moieties comprise a hairpin (e.g., hairpin comprising a region of self-complementarity, optionally wherein the region of self-complementary comprises 2, 3, 4, 5, 6, 7, 8, 9, 10 or more contiguous complementary basepairs), a quadruplex (e.g., a G-quadruplex or a C-quadruplex, optionally wherein the G-quadruplex or the C-quadruplex is derived from a VEGF gene promoter), a tRNA sequence (e.g., a tRNA sequence, optionally wherein the tRNA sequence is a tRNA (Proline) sequence), an aptamer (e.g., an aptamer derived from a viral protein-binding sequence, optionally wherein the aptamer comprises a viral reverse transcriptase recruitment sequence, optionally wherein the aptamer comprises a MS2 protein binding sequence or a Moloney Murine leukemia (MMLV) reverse transcriptase recruitment sequence), and/or a pseudoknot (e.g. pseudoknot is derived form a potato roll leaf virus (PLRV)), or any combination thereof.

In some embodiments, the one or more nucleic acid moieties comprise a structure derived form a replication recognition sequence of a retrovirus. In some embodiments, the nucleic acid moiety comprises a sequence derived from a replication recognition sequence of a Moloney Murine leukemia virus (MMLV). In some embodiments, the one or more nucleic acid moieties comprise a nucleic acid sequence selected from SEQ ID NOs 12-15.

In some embodiments, the one or more nucleic acid moieties comprises a hairpin. In some embodiments, the hairpin comprises a sequence of any one of SEQ ID Nos: 1-3 or 5-7.

In some embodiments, the one or more nucleic acid moieties comprises a pseudoknot. In some embodiments, the pseudoknot is derived from potato roll-leaf virus. In some embodiments, the pseudoknot comprises the sequence of SEQ ID NO: 4. In some embodiments, the one or more nucleic acid moieties comprises a MS2 hairpin. In some embodiments, the nucleotide sequence of the MS2 hairpin (or also referred to as the “MS2 aptamer”) is: GCCAACATGAGGATCACCCATGTCTGCAGGGCC (SEQ ID NO: 4446). In some embodiments, the nucleotide sequence of the MS2 aptamer comprises the sequence of SEQ ID NO: 9. In some embodiments, a MS2 coat protein (MCP) recognizes the MS2 hairpin. In some embodiments, the amino acid sequence of the MCP is:

(SEQ ID NO: 4447)
GSASNFTQFVLVDNGGTGDVTVAPSNFANGVAEWISSNSRSQAYKVTCSV
RQSSAQNRKYTIKVEVPKVATQTVGGEELPVAGWRSYLNMELTIPIFATN
SDCELIVKAMQGLLKDGNPIPSAIAANSGIY.

In some embodiments, the one or more nucleic acid moieties comprises a G-quadruplex or a C-quadruplex. In some embodiments, the one or more nucleic acid moieties comprises a quadruplex from a VEGF gene promoter. In some embodiments, the quadruplex comprises the sequence of SEQ ID NO: 10 or 11.

In some embodiments, the PEgRNA comprises one or more nucleic acid moieties at its 3′ end. In some embodiments, the PEgRNA comprises one or more nucleic acid moieties at its 5′ end.

TABLE 3
Exemplary Nucleic Acid Motif Sequences

| SEQ | | | | |
|---|---|---|---|---|
| ID | | | | |
| NO. | Name | Name description | Motif Sequence | Motif length |
| 1 | hp_1 | hairpin 1 | CGGGTCTCTACGTGGGG | 22 |
| | | | GCCCG | |
| 2 | hp_1 | hairpin 1 | CGCGTCTCTACGTGGGG | 22 |
| | | | GCGCG | |
| 3 | hp_3 | hairpin 3 | GGCGCGAAAGCGCC | 14 |
| 4 | PLRV_22 | potato roll leaf | GCGGCACCGTCCGCCCA | 22 |
| | | virus pseudoknot | AACGG | |
| 5 | hp_5 | hairpin 5 | GCCCGGCGAAAGCCGGG | 18 |
| | | | C | |
| 6 | hp_4 | hairpin 4 | GCCCGGCTTCGGCCGGG | 18 |
| | | | C | |
| 7 | hp_2 | hairpin 2 | GGCGCTTCGGCGCC | 14 |
| 8 | MMLV-RT | MML Vaptamer | TTACCACGCGCTCTTAA | 33 |
| | aptamer | sequence that can | CTGCTAGCGCCATGGC | |
| | | recruit MMLV RT | | |
| 9 | MS2 | MS2 protein | ACATGAGGATCACCCAT | 19 |
| | | binding sequence. | GT | |
| 10 | G quad/ | G-quadruplex in | GGGCGGGCCGGGGGCG | 18 |
| | G4_VEGF | VEGF promoter | GG | |
| 11 | C quad/ | C-quadruplex in | CCCCGCCCCGGCCGCCC | 18 |
| | iM_VEGF | VEGF promoter | C | |
| 12 | tRNA_PBS_ | MMLV endogenous | GCTCCTCTGATTGACTA | 61 |
| | long | binding for | CCCGTCAGCGGGGGTCT | |
| | | replication | TTTGGGGGCTCGTCCGG | |
| | | | GATCGGGAGT | |
| 13 | tRNA_PBS_ | MMLV endogenous | ACTCCCGATCCCGGACG | 61 |
| | long_RC | binding for | AGCCCCCAAAAGACCCC | |
| | | replication | CGCTGACGGGTAGTCAA | |
| | | (reverse | TCAGAGGAGC | |
| | | complement) | | |
| 14 | tRNA_PBS_ | MMLV endogenous | TGGGGGCTCGTCCGGGA | 18 |
| | short | binding for | T | |
| | | replication | | |
| 15 | tRNA_PBS_ | MMLV endogenous | ATCCCGGACGAGCCCCC | 18 |
| | short_RC | binding for | A | |
| | | replication | | |
| | | (reverse | | |
| | | complement) | | |
| 4453 | evopreQ1 | Prequeosine1-1 | CGCGGTTCTATCTAGTT | 37 |
| | | riboswitch aptamer | ACGCGTTAAACCAACTA | |
| | | | GAA | |

TABLE 4
Exemplary Nucleic Acid Motif Structural Configurations

| Moiety Type | Structural Configuration |
|---|---|
| Hairpin (hp_1) (SEQ ID NO: 1) | 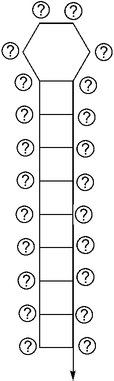 |
| Pseudoknot (PLRV_22) (SEQ ID NO: 4) | 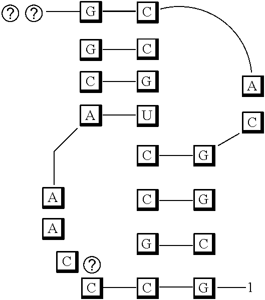 |
| tRNA sequence (short) (SEQ ID NO: 14) | 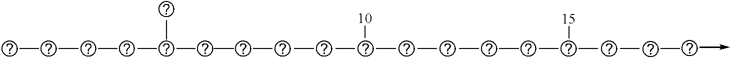 |
| rTNA sequence (long) (SEQ ID NO: 12) | 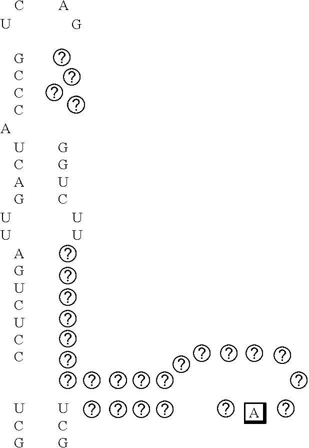 |
| Aptamer (MMLV-RT) (SEQ ID NO: 8) | 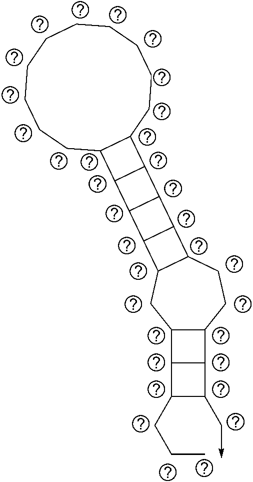 |
| Aptamer (MS2) (SEQ ID NO: 4) | 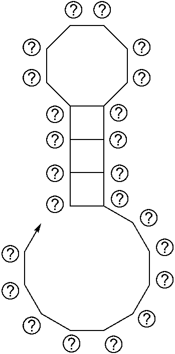 |
| Quadruplex (G quad/ G4_VEGF) (SEQ ID NO: 7071) | [symbol] |
| Quadruplex (C quad/ iM_VEGF) | [symbol] |

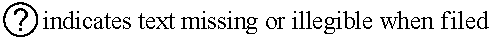

### Tag Sequences

In some embodiments, the PEgRNA comprises a tag sequence in addition to the spacer, gRNA core, primer binding site, and editing template. In some embodiments, the tag sequence comprises a region of complementarity to the editing template. In some embodiments, the tag sequence comprises a region of complementarity to the PBS. In some embodiments, the tag sequence comprises a region of complementarity to the editing template and/or the PBS. In some embodiments, the tag sequence comprises a region of complementarity to the editing template and does not have substantial complementarity to the PBS. In some embodiments, the tag sequence comprises a region of complementarity to the editing template and does not have complementarity to the PBS. In some embodiments, the tag sequence and the editing template each comprises a region of complementarity to each other, wherein the 3′ end of the region of complementarity in the editing template is at a position 1, 2, 3, 4, 5, 6, 7, 8, 9, 10, 11, 12, 13, 14, 15, 16 or more bases 5′ of the 3′ half of the editing template. In some embodiments, the region of complementarity in the tag sequence is at a 5′ portion of the tag sequence. In some embodiments, the tag sequence does not have substantial complementarity to the spacer. In some embodiments, the tag does not have complementarity to the spacer. In some embodiments, the tag sequence is 1, 2, 3, 4, 5, 6, 7, 8, 9, 10, 11, 12, 13, 14, 15, 16 nucleotides in length. In some embodiments, the tag sequence is at least 4, at least 6, at least 8 nucleotides in length. In some embodiments, the tag sequence comprises a nucleic acid sequence selected from SEQ REF NOs 62-1960. Exemplary Tag sequences can be found in Table 5.

Lengthy table referenced here
US20250297246A1-20250925-T00001
Please refer to the end of the specification for access instructions.

### Linkers

In some embodiments, the PEgRNA comprises a linker. In some embodiments, the linker is: i) immediately 5′ of the one or more nucleic acid moieties, ii) immediately 5′ of the tag sequence, iii) immediately 3′ of the tag sequence, iv) immediately 3′ of the spacer, v) immediately 5′ of the spacer, vi) immediately 3′ of the gRNA core, or vii) immediately 5′ of the gRNA core. In some embodiments, the linker is 1, 2, 3, 4, 5, 6, 7, 8, 9, 10, 11, 12, 13, 14, 15, 16 nucleotides in length. In some embodiments, the linker is 2 to 12 nucleotides in length. In some embodiments, the linker is 5 to 20 nucleotides in length. In some embodiments, the linker is 3 to 10, 3 to 15, 3 to 20, 3 to 25, 3 to 30, 3 to 35, 3 to 40, or 3 to 50 nucleotides in length. In some embodiments, the linker is 8 nucleotides in length. In some embodiments, the linker does not form a secondary structure. In some embodiments, the linker does not have a region of complementarity to the PBS sequence. In some embodiments, the linker does not have a region of complementarity to the editing template. In some embodiments, the linker comprises a sequence selected from SEQ REF NOs 1961-3859. As used herein, a linker can be any chemical group or molecule linking two molecules/moieties, e.g., the components of the PEgRNA.

### LegRNAs

Also provided herein are legRNAs. In some embodiments, the PEgRNA is a legRNA. As used herein, a “legRNA” is a PEgRNA comprising a spacer, a gRNA core, a PBS, and an editing template (e.g., an RTT sequence), wherein the PBS and the editing template is positioned within the gRNA core. A legRNA disclosed herein may comprise any 3′ moiety or other modification disclosed herein.

In certain embodiments, the legRNAs comprise in 5′ to 3′ order: i) a spacer that comprises a region of complementarity to a search target sequence in target strand of a double stranded target DNA; ii) a 5′ part of a guide RNA (gRNA) core; iii) an editing template that comprises an intended edit compared to the double stranded target DNA; iv) a primer binding site (PBS) that comprises a region of complementarity to a region upstream of a nick site in a non-target strand of the double stranded target DNA; and v) a 3′ part of a gRNA core. In some embodiments, the 5′ part of the gRNA core comprises a direct repeat, a first stem loop, and a 5′ half of a second stem loop. In some embodiments, the 3′ part of the gRNA core comprises a 3′ half of a second stem loop and a third stem loop. In some embodiments, the 5′ part of the gRNA core and the 3′ part of the gRNA core are “split” between the 30^{th} and the 31^{st}, the 31^{st} and the 32^{nd}, the 32^{nd} and the 33^{rd}, the 33^{rd} and the 34^{th}, the 34^{th} and the 35^{th}, the 35^{th} and the 36^{th}, the 36^{th} and the 37^{th}, the 37^{th} and the 38^{th}, the 38^{th} and the 39^{th}, or the 39^{th} and 40^{th} nucleotides of the full gRNA core sequence, wherein the position numbering of the nucleotides is as set forth in SEQ ID NO: 16. In some embodiments, the 5′ part of the gRNA core and the 3′ part of the gRNA core are “split” at between the 50^{th} and the 51^{st}, the 51^{st} and the 52^{nd}, the 52^{nd} and the 55^{rd}, the 55^{rd} and the 54^{th}, the 54^{th} and the 55^{th}, the 55^{th} and the 56^{th}, the 56^{th} and the 57^{th}, the 57^{th} and the 58^{th}, the 58^{th} and the 59^{th}, or the 59^{th} and 60^{th} nucleotides of the full gRNA core sequence, wherein the position numbering of the nucleotides is as set forth in SEQ ID NO: 16. In some embodiments, the 5′ part of the gRNA core and the 3′ part of the gRNA core are split between the 54^{th} and the 55^{th} nucleotides of the full gRNA core sequence, wherein the position numbering of the nucleotides is as set forth in SEQ ID NO: 16. In some embodiments, the 5′ part of the gRNA core comprises the sequence GTTTAAGAGCTAGAAATAGCAAGTTTAAATAAGGCTAGTCCGTTATCAGCGTGA (SEQ ID NO: 6376). In some embodiments, the 3′ part of the gRNA core comprises the sequence AAACGCGGCACCGAGTCGGTGC (SEQ ID NO: 6377).

Exemplary legRNA are found in Table 6 below.

In some embodiments, the PEgRNA further comprises a tag sequence that comprises a region of complementarity to the PBS and/or the editing template.

The legRNA may comprise a tag sequence, an aptamer, a hairpin, a quadruplex, a tRNA, a pseudoknot, a linker, or any nucleic acid moieties as described herein. In some embodiments, the legRNA comprises a linker. In some embodiments, the linker is: i) immediately 5′ of the one or more nucleic acid moieties, ii) immediately 5′ of the tag sequence, iii) immediately 3′ of the tag sequence, iv) immediately 3′ of the spacer, v) immediately 5′ of the spacer, vi) immediately 3′ of the gRNA core, and/or vii) immediately 5′ of the gRNA core. In some embodiments, the linker is 1, 2, 3, 4, 5, 6, 7, 8, 9, 10, 11, 12, 13, 14, 15, 16 nucleotides in length. In some embodiments, the linker does not form a secondary structure. In some embodiments, the linker does not have a region of complementarity to the PBS sequence. In some embodiments, the linker does not have a region of complementarity to the editing template. In some embodiments, the linker comprises a nucleic acid sequence selected from SEQ REF NOs 1961-3859. As used herein, a linker can be any chemical group or a molecule linking two molecules or moieties, e.g., the components of the legRNA.

TABLE 6
Exemplary LegRNA sequences

| SEQ ID | | | |
|---|---|---|---|
| NO. | Edit | Variant | PEgRNA |
| 4360 | ATP7B_H1069Q_ | LegRNA_ATP7B_H1069Q_ | GTTTGGTGACTGCCACGCCCAGTTTAAGAGCTAGAAATAGCAAGT |
| | noPAM_82 | noPAM_82_Linker0 | TTAAATAAGGCTAGTCCGTTATCAGCGTGAAACACCCCTTGGGCG |
| | | | TGGCAAACGCGGCACCGAGTCGGTGC |
| 4361 | ATP7B_H1069Q_ | LegRNA_ATP7B_H1069Q_ | GTTTGGTGACTGCCACGCCCAGTTTAAGAGCTAGAAATAGCAAGT |
| | noPAM_82 | noPAM_82_Linker2 | TTAAATAAGGCTAGTCCGTTATCAGCGTGAAACACCCCTTGGGCG |
| | | | TGGCCAAAACGCGGCACCGAGTCGGTGC |
| 4362 | ATP7B_H1069Q_ | LegRNA_ATP7B_H1069Q_ | GTTTGGTGACTGCCACGCCCAGTTTAAGAGCTAGAAATAGCAAGT |
| | noPAM_82 | noPAM_82_Linker4 | TTAAATAAGGCTAGTCCGTTATCAGCGTGAAACACCCCTTGGGCG |
| | | | TGGCCCTAAAACGCGGCACCGAGTCGGTGC |
| 4363 | ATP7B_H1069Q_ | LegRNA_ATP7B_H1069Q_ | GTTTGGTGACTGCCACGCCCAGTTTAAGAGCTAGAAATAGCAAGT |
| | noPAM_82 | noPAM_82_Linker6 | TTAAATAAGGCTAGTCCGTTATCAGCGTGAAACACCCCTTGGGCG |
| | | | TGGCGGCCTAAAACGCGGCACCGAGTCGGTGC |
| 4364 | ATP7B_H1069Q_ | LegRNA_ATP7B_H1069Q_ | GTTTGGTGACTGCCACGCCCAGTTTAAGAGCTAGAAATAGCAAGT |
| | noPAM_82 | noPAM_82_Linker8 | TTAAATAAGGCTAGTCCGTTATCAGCGTGAAACACCCCTTGGGCG |
| | | | TGGCGGCCTGGGAAACGCGGCACCGAGTCGGTGC |
| 4365 | ATP7B_H1069Q_ | LegRNA_ATP7B_H1069Q_ | GTTTGGTGACTGCCACGCCCAGTTTAAGAGCTAGAAATAGCAAGT |
| | noPAM_88 | noPAM_88_Linker0 | TTAAATAAGGCTAGTCCGTTATCAGCGTGAAACACCCCTTGGGCG |
| | | | TGGCAGTCACAAACGCGGCACCGAGTCGGTGC |
| 4366 | ATP7B_H1069Q_ | LegRNA_ATP7B_H1069Q_ | GTTTGGTGACTGCCACGCCCAGTTTAAGAGCTAGAAATAGCAAGT |
| | noPAM_88 | noPAM_88_Linker2 | TTAAATAAGGCTAGTCCGTTATCAGCGTGAAACACCCCTTGGGCG |
| | | | TGGCAGTCACAGAAACGCGGCACCGAGTCGGTGC |
| 4367 | ATP7B_H1069Q_ | LegRNA_ATP7B_H1069Q_ | GTTTGGTGACTGCCACGCCCAGTTTAAGAGCTAGAAATAGCAAGT |
| | noPAM_88 | noPAM_88_Linker4 | TTAAATAAGGCTAGTCCGTTATCAGCGTGAAACACCCCTTGGGCG |
| | | | TGGCAGTCACAGGAAAACGCGGCACCGAGTCGGTGC |
| 4368 | ATP7B_H1069Q_ | LegRNA_ATP7B_H1069Q_ | GTTTGGTGACTGCCACGCCCAGTTTAAGAGCTAGAAATAGCAAGT |
| | noPAM_88 | noPAM_88_Linker6 | TTAAATAAGGCTAGTCCGTTATCAGCGTGAAACACCCCTTGGGCG |
| | | | TGGCAGTCACACAGAAAAACGCGGCACCGAGTCGGTGC |
| 4369 | ATP7B_H1069Q_ | LegRNA_ATP7B_H1069Q_ | GTTTGGTGACTGCCACGCCCAGTTTAAGAGCTAGAAATAGCAAGT |
| | noPAM_88 | noPAM_88_Linker8 | TTAAATAAGGCTAGTCCGTTATCAGCGTGAAACACCCCTTGGGCG |
| | | | TGGCAGTCACCAGGTTCAAAACGCGGCACCGAGTCGGTGC |
| 4370 | ATP7B_R778L_ | LegRNA_ATP7B_R778L_ | GTTGCCAAGTGTTCCAGCCACGTTTAAGAGCTAGAAATAGCAAGT |
| | noPAM_132 | noPAM_132_Linker0 | TTAAATAAGGCTAGTCCGTTATCAGCGTGATTGCCCTGGGCCGGT |
| | | | GGCTGGAACACTAAACGCGGCACCGAGTCGGTGC |
| 4371 | ATP7B_R778L_ | LegRNA_ATP7B_R778L_ | GTTGCCAAGTGTTCCAGCCACGTTTAAGAGCTAGAAATAGCAAGT |
| | noPAM_132 | noPAM_132_Linker2 | TTAAATAAGGCTAGTCCGTTATCAGCGTGATTGCCCTGGGCCGGT |
| | | | GGCTGGAACACTCCAAACGCGGCACCGAGTCGGTGC |
| 4372 | ATP7B_R778L_ | LegRNA_ATP7B_R778L_ | GTTGCCAAGTGTTCCAGCCACGTTTAAGAGCTAGAAATAGCAAGT |
| | noPAM_132 | noPAM_132_Linker4 | TTAAATAAGGCTAGTCCGTTATCAGCGTGATTGCCCTGGGCCGGT |
| | | | GGCTGGAACACTCACCAAACGCGGCACCGAGTCGGTGC |
| 4373 | ATP7B_R778L_ | LegRNA_ATP7B_R778L_ | GTTGCCAAGTGTTCCAGCCACGTTTAAGAGCTAGAAATAGCAAGT |
| | noPAM_132 | noPAM_132_Linker6 | TTAAATAAGGCTAGTCCGTTATCAGCGTGATTGCCCTGGGCCGGT |
| | | | GGCTGGAACACTCCTCATAAACGCGGCACCGAGTCGGTGC |
| 4374 | ATP7B_R778L_ | LegRNA_ATP7B_R778L_ | GTTGCCAAGTGTTCCAGCCACGTTTAAGAGCTAGAAATAGCAAGT |
| | noPAM_132 | noPAM_132_Linker8 | TTAAATAAGGCTAGTCCGTTATCAGCGTGATTGCCCTGGGCCGGT |
| | | | GGCTGGAACACTCCTCACTCAAACGCGGCACCGAGTCGGTGC |
| 4375 | ATP7B_R778L_ | LegRNA_ATP7B_R778L_ | GTTGCCAAGTGTTCCAGCCACGTTTAAGAGCTAGAAATAGCAAGT |
| | noPAM_117 | noPAM_117_Linker0 | TTAAATAAGGCTAGTCCGTTATCAGCGTGAGCCCTGGGCCGGTGG |
| | | | CTGGAACACTTAAACGCGGCACCGAGTCGGTGC |
| 4376 | ATP7B_R778L_ | LegRNA_ATP7B_R778L_ | GTTGCCAAGTGTTCCAGCCACGTTTAAGAGCTAGAAATAGCAAGT |
| | noPAM_117 | noPAM_117_Linker2 | TTAAATAAGGCTAGTCCGTTATCAGCGTGAGCCCTGGGCCGGTGG |
| | | | CTGGAACACTTAAAAACGCGGCACCGAGTCGGTGC |
| 4377 | ATP7B_R778L_ | LegRNA_ATP7B_R778L_ | GTTGCCAAGTGTTCCAGCCACGTTTAAGAGCTAGAAATAGCAAGT |
| | noPAM_117 | noPAM_117_Linker4 | TTAAATAAGGCTAGTCCGTTATCAGCGTGAGCCCTGGGCCGGTGG |
| | | | CTGGAACACTTTCCTAAACGCGGCACCGAGTCGGTGC |
| 4378 | ATP7B_R778L_ | LegRNA_ATP7B_R778L_ | GTTGCCAAGTGTTCCAGCCACGTTTAAGAGCTAGAAATAGCAAGT |
| | noPAM_117 | noPAM_117_Linker6 | TTAAATAAGGCTAGTCCGTTATCAGCGTGAGCCCTGGGCCGGTGG |
| | | | CTGGAACACTTTCAGAGAAACGCGGCACCGAGTCGGTGC |
| 4379 | ATP7B_R778L_ | LegRNA_ATP7B_R778L_ | GTTGCCAAGTGTTCCAGCCACGTTTAAGAGCTAGAAATAGCAAGT |
| | noPAM_117 | noPAM_117_Linker8 | TTAAATAAGGCTAGTCCGTTATCAGCGTGAGCCCTGGGCCGGTGG |
| | | | CTGGAACACTTTAAAATTTAAACGCGGCACCGAGTCGGTGC |
| 4380 | NCF1_delGT_16 | LegRNA_NCF1_delGT_ | GTCACCAGGAACATGTACCTGGTTTAAGAGCTAGAAATAGCAAG |
| | | 16_Linker0 | TTTAAATAAGGCTAGTCCGTTATCAGCGTGATTCCCCCAGGTGTA |
| | | | CATGTTCCTGAAACGCGGCACCGAGTCGGTGC |
| 4381 | NCF1_delGT_16 | LegRNA_NCF1_delGT_ | GTCACCAGGAACATGTACCTGGTTTAAGAGCTAGAAATAGCAAG |
| | | 16_Linker2 | TTTAAATAAGGCTAGTCCGTTATCAGCGTGATTCCCCCAGGTGTA |
| | | | CATGTTCCTGCTAAACGCGGCACCGAGTCGGTGC |
| 4382 | NCF1_delGT_16 | LegRNA_NCF1_delGT_ | GTCACCAGGAACATGTACCTGGTTTAAGAGCTAGAAATAGCAAG |
| | | 16_Linker4 | TTTAAATAAGGCTAGTCCGTTATCAGCGTGATTCCCCCAGGTGTA |
| | | | CATGTTCCTGCAAGAAACGCGGCACCGAGTCGGTGC |
| 4383 | NCF1_delGT_16 | LegRNA_NCF1_delGT_ | GTCACCAGGAACATGTACCTGGTTTAAGAGCTAGAAATAGCAAG |
| | | 16_Linker6 | TTTAAATAAGGCTAGTCCGTTATCAGCGTGATTCCCCCAGGTGTA |
| | | | CATGTTCCTGTGCTCTAAACGCGGCACCGAGTCGGTGC |
| 4384 | NCF1_delGT_16 | LegRNA_NCF1_delGT_ | GTCACCAGGAACATGTACCTGGTTTAAGAGCTAGAAATAGCAAG |
| | | 16_Linker8 | TTTAAATAAGGCTAGTCCGTTATCAGCGTGATTCCCCCAGGTGTA |
| | | | CATGTTCCTGCTTCTTCGAAACGCGGCACCGAGTCGGTGC |
| 4385 | NCF1_delGT_10 | LegRNA_NCF1_delGT_ | GTCACCAGGAACATGTACCTGGTTTAAGAGCTAGAAATAGCAAG |
| | | 10_Linker0 | TTTAAATAAGGCTAGTCCGTTATCAGCGTGATTCCCCCAGGTGTA |
| | | | CATGAAACGCGGCACCGAGTCGGTGC |
| 4386 | NCF1_delGT_10 | LegRNA_NCF1_delGT_ | GTCACCAGGAACATGTACCTGGTTTAAGAGCTAGAAATAGCAAG |
| | | 10_Linker2 | TTTAAATAAGGCTAGTCCGTTATCAGCGTGATTCCCCCAGGTGTA |
| | | | CATGATAAACGCGGCACCGAGTCGGTGC |
| 4387 | NCF1_delGT_10 | LegRNA_NCF1_delGT_ | GTCACCAGGAACATGTACCTGGTTTAAGAGCTAGAAATAGCAAG |
| | | 10_Linker4 | TTTAAATAAGGCTAGTCCGTTATCAGCGTGATTCCCCCAGGTGTA |
| | | | CATGAATAAAACGCGGCACCGAGTCGGTGC |
| 4388 | NCF1_delGT_10 | LegRNA_NCF1_delGT_ | GTCACCAGGAACATGTACCTGGTTTAAGAGCTAGAAATAGCAAG |
| | | 10_Linker6 | TTTAAATAAGGCTAGTCCGTTATCAGCGTGATTCCCCCAGGTGTA |
| | | | CATGACTACTAAACGCGGCACCGAGTCGGTGC |
| 4389 | NCF1_delGT_10 | LegRNA_NCF1_delGT_ | GTCACCAGGAACATGTACCTGGTTTAAGAGCTAGAAATAGCAAG |
| | | 10_Linker8 | TTTAAATAAGGCTAGTCCGTTATCAGCGTGATTCCCCCAGGTGTA |
| | | | CATGACTAAAACAAACGCGGCACCGAGTCGGTGC |
| 4390 | HEK3_6G_C_18 | LegRNA_HEK3_6G_C_ | GGCCCAGACTGAGCACGTGAGTTTAAGAGCTAGAAATAGCAAGT |
| | | 18_Linker0 | TTAAATAAGGCTAGTCCGTTATCAGCGTGATTCCTCTGGCATCAC |
| | | | GTGCTCAGAAACGCGGCACCGAGTCGGTGC |
| 4391 | HEK3_6G_C_18 | LegRNA_HEK3_6G_C_ | GGCCCAGACTGAGCACGTGAGTTTAAGAGCTAGAAATAGCAAGT |
| | | 18_Linker2 | TTAAATAAGGCTAGTCCGTTATCAGCGTGATTCCTCTGGCATCAC |
| | | | GTGCTCAGAGAAACGCGGCACCGAGTCGGTGC |
| 4392 | HEK3_6G_C_18 | LegRNA_HEK3_6G_C_ | GGCCCAGACTGAGCACGTGAGTTTAAGAGCTAGAAATAGCAAGT |
| | | 18_Linker4 | TTAAATAAGGCTAGTCCGTTATCAGCGTGATTCCTCTGGCATCAC |
| | | | GTGCTCAGAAAGAAACGCGGCACCGAGTCGGTGC |
| 4393 | HEK3_6G_C_18 | LegRNA_HEK3_6G_C_ | GGCCCAGACTGAGCACGTGAGTTTAAGAGCTAGAAATAGCAAGT |
| | | 81_Linker6 | TTAAATAAGGCTAGTCCGTTATCAGCGTGATTCCTCTGGCATCAC |
| | | | GTGCTCAGATTCCAAAACGCGGCACCGAGTCGGTGC |
| 4394 | HEK3_6G_C_18 | LegRNA_HEK3_6G_C_ | GGCCCAGACTGAGCACGTGAGTTTAAGAGCTAGAAATAGCAAGT |
| | | 18_Linker8 | TTAAATAAGGCTAGTCCGTTATCAGCGTGATTCCTCTGGCATCAC |
| | | | GTGCTCAGGCACAAAGAAACGCGGCACCGAGTCGGTGC |
| 4395 | HEK3_CTTins_ | LegRNA_HEK3_CTTins | GGCCCAGACTGAGCACGTGAGTTTAAGAGCTAGAAATAGCAAGT |
| | 36 | 36_Linker0 | TTAAATAAGGCTAGTCCGTTATCAGCGTGATTCCTCTGCCATCAA |
| | | | AGCGTGCTCAGTCAAACGCGGCACCGAGTCGGTGC |
| 4396 | HEK3_CTTins_ | LegRNA_HEK3_CTTins_ | GGCCCAGACTGAGCACGTGAGTTTAAGAGCTAGAAATAGCAAGT |
| | 36 | 36_Linker2 | TTAAATAAGGCTAGTCCGTTATCAGCGTGATTCCTCTGCCATCAA |
| | | | AGCGTGCTCAGTCCAAAACGCGGCACCGAGTCGGTGC |
| 4397 | HEK3_CTTins_ | LegRNA_HEK3_CTTins_ | GGCCCAGACTGAGCACGTGAGTTTAAGAGCTAGAAATAGCAAGT |
| | 36 | 36_Linker4 | TTAAATAAGGCTAGTCCGTTATCAGCGTGATTCCTCTGCCATCAA |
| | | | AGCGTGCTCAGTCCACAAAACGCGGCACCGAGTCGGTGC |
| 4398 | HEK3_CTTins_ | LegRNA_HEK3_CTTins_ | GGCCCAGACTGAGCACGTGAGTTTAAGAGCTAGAAATAGCAAGT |
| | 36 | 36_Linker6 | TTAAATAAGGCTAGTCCGTTATCAGCGTGATTCCTCTGCCATCAA |
| | | | AGCGTGCTCAGTCCCATTCAAACGCGGCACCGAGTCGGTGC |
| 4399 | HEK3_CTTins_ | LegRNA_HEK3_CTTins_ | GGCCCAGACTGAGCACGTGAGTTTAAGAGCTAGAAATAGCAAGT |
| | 36 | 36_Linker8 | TTAAATAAGGCTAGTCCGTTATCAGCGTGATTCCTCTGCCATCAA |
| | | | AGCGTGCTCAGTCCAACTGATAAACGCGGCACCGAGTCGGTGC |
| 4400 | EMX1_1G_C_ | LegRNA_EMX1_1G_C_ | GAGTCCGAGCAGAAGAAGAAGTTTAAGAGCTAGAAATAGCAAGT |
| | 30 | 30_Linker0 | TTAAATAAGGCTAGTCCGTTATCAGCGTGAGATGGGAGCCCTTGT |
| | | | TCTTCTGCTCGGAAACGCGGCACCGAGTCGGTGC |
| 4401 | EMX1_1G_C_ | LegRNA_EMX1_1G_C_ | GAGTCCGAGCAGAAGAAGAAGTTTAAGAGCTAGAAATAGCAAGT |
| | 30 | 30_Linker2 | TTAAATAAGGCTAGTCCGTTATCAGCGTGAGATGGGAGCCCTTGT |
| | | | TCTTCTGCTCGGTGAAACGCGGCACCGAGTCGGTGC |
| 4402 | EMX1_1G_C_ | LegRNA_EMX1_1G_C_ | GAGTCCGAGCAGAAGAAGAAGTTTAAGAGCTAGAAATAGCAAGT |
| | 30 | 30_Linker4 | TTAAATAAGGCTAGTCCGTTATCAGCGTGAGATGGGAGCCCTTGT |
| | | | TCTTCTGCTCGGGTTGAAACGCGGCACCGAGTCGGTGC |
| 4403 | EMX1_1G_C_ | LegRNA_EMX1_1G_C_ | GAGTCCGAGCAGAAGAAGAAGTTTAAGAGCTAGAAATAGCAAGT |
| | 03 | 30_Linker6 | TTAAATAAGGCTAGTCCGTTATCAGCGTGAGATGGGAGCCCTTGT |
| | | | TCTTCTGCTCGGTGAAAAAAACGCGGCACCGAGTCGGTGC |
| 4404 | EMX1_1G_C_ | LegRNA_EMX1_1G_C_ | GAGTCCGAGCAGAAGAAGAAGTTTAAGAGCTAGAAATAGCAAGT |
| | 30 | 30_Linker8 | TTAAATAAGGCTAGTCCGTTATCAGCGTGAGATGGGAGCCCTTGT |
| | | | TCTTCTGCTCGGTAAACAGTAAACGCGGCACCGAGTCGGTGC |
| 4405 | EMX1_TGCins_ | LegRNA_EMX1_TGCins_ | GAGTCCGAGCAGAAGAAGAAGTTTAAGAGCTAGAAATAGCAAGT |
| | 12 | 12_Linker0 | TTAAATAAGGCTAGTCCGTTATCAGCGTGAGAGCCCTTCGCATTC |
| | | | TTCTGCTCAAACGCGGCACCGAGTCGGTGC |
| 4406 | EMX1_TGCins_ | LegRNA_EMX1_TGCins_ | GAGTCCGAGCAGAAGAAGAAGTTTAAGAGCTAGAAATAGCAAGT |
| | 12 | 12_Linker2 | TTAAATAAGGCTAGTCCGTTATCAGCGTGAGAGCCCTTCGCATTC |
| | | | TTCTGCTCCCAAACGCGGCACCGAGTCGGTGC |
| 4407 | EMX1_TGCins_ | LegRNA_EMX1_TGCins_ | GAGTCCGAGCAGAAGAAGAAGTTTAAGAGCTAGAAATAGCAAGT |
| | 12 | 12_Linker4 | TTAAATAAGGCTAGTCCGTTATCAGCGTGAGAGCCCTTCGCATTC |
| | | | TTCTGCTCCCCCAAACGCGGCACCGAGTCGGTGC |
| 4408 | EMX1_TGCins_ | LegRNA_EMX1_TGCins_ | GAGTCCGAGCAGAAGAAGAAGTTTAAGAGCTAGAAATAGCAAGT |
| | 12 | 12_Linker6 | TTAAATAAGGCTAGTCCGTTATCAGCGTGAGAGCCCTTCGCATTC |
| | | | TTCTGCTCCTAAAGAAACGCGGCACCGAGTCGGTGC |
| 4409 | EMX1_TGCins_ | LegRNA_EMX1_TGCins_ | GAGTCCGAGCAGAAGAAGAAGTTTAAGAGCTAGAAATAGCAAGT |
| | 12 | 12_Linker8 | TTAAATAAGGCTAGTCCGTTATCAGCGTGAGAGCCCTTCGCATTC |
| | | | TTCTGCTCCAGTCCCCAAACGCGGCACCGAGTCGGTGC |
| 4410 | FANCF_delACC_ | LegRNA_FANCF_delACC_ | GGAATCCCTTCTGCAGCACCGTTTAAGAGCTAGAAATAGCAAGTT |
| | 4 | 4_Linker0 | TAAATAAGGCTAGTCCGTTATCAGCGTGAAAGCGATCCAGCTGCA |
| | | | GAAGGAAACGCGGCACCGAGTCGGTGC |
| 4411 | FANCF_delACC_ | LegRNA_FANCF_delACC_ | GGAATCCCTTCTGCAGCACCGTTTAAGAGCTAGAAATAGCAAGTT |
| | 4 | 4_Linker2 | TAAATAAGGCTAGTCCGTTATCAGCGTGAAAGCGATCCAGCTGCA |
| | | | GAAGGCTAAACGCGGCACCGAGTCGGTGC |
| 4412 | FANCF_delACC_ | LegRNA_FANCF_delACC_ | GGAATCCCTTCTGCAGCACCGTTTAAGAGCTAGAAATAGCAAGTT |
| | 4 | 4_Linker4 | TAAATAAGGCTAGTCCGTTATCAGCGTGAAAGCGATCCAGCTGCA |
| | | | GAAGGCTAAAAACGCGGCACCGAGTCGGTGC |
| 4413 | FANCF_delACC_ | LegRNA_FANCF_delACC_ | GGAATCCCTTCTGCAGCACCGTTTAAGAGCTAGAAATAGCAAGTT |
| | 4 | 4_Linker6 | TAAATAAGGCTAGTCCGTTATCAGCGTGAAAGCGATCCAGCTGCA |
| | | | GAAGGCCCTACAAACGCGGCACCGAGTCGGTGC |
| 4414 | FANCF_delACC_ | LegRNA_FANCF_delACC_ | GGAATCCCTTCTGCAGCACCGTTTAAGAGCTAGAAATAGCAAGTT |
| | 4 | 4_Linker8 | TAAATAAGGCTAGTCCGTTATCAGCGTGAAAGCGATCCAGCTGCA |
| | | | GAAGGCTAATAGTAAACGCGGCACCGAGTCGGTGC |
| 4415 | FANCF_5G_T_ | LegRNA_FANCF_5G_T_ | GGAATCCCTTCTGCAGCACCGTTTAAGAGCTAGAAATAGCAAGTT |
| | 9 | 9_Linker0 | TAAATAAGGCTAGTCCGTTATCAGCGTGAAAGCGATCAAGGTGCT |
| | | | GCAGAAAACGCGGCACCGAGTCGGTGC |
| 4416 | FANCF_5G_T_ | LegRNA_FANCF_5G_T_ | GGAATCCCTTCTGCAGCACCGTTTAAGAGCTAGAAATAGCAAGTT |
| | 9 | 9_Linker2 | TAAATAAGGCTAGTCCGTTATCAGCGTGAAAGCGATCAAGGTGCT |
| | | | GCAGACAAAACGCGGCACCGAGTCGGTGC |
| 4417 | FANCF_5G_T_ | LegRNA_FANCF_5G_T | GGAATCCCTTCTGCAGCACCGTTTAAGAGCTAGAAATAGCAAGTT |
| | 9 | 9_Linker4 | TAAATAAGGCTAGTCCGTTATCAGCGTGAAAGCGATCAAGGTGCT |
| | | | GCAGACATCAAACGCGGCACCGAGTCGGTGC |
| 4418 | FANCF_5G_T_ | LegRNA_FANCF_5G_T_ | GGAATCCCTTCTGCAGCACCGTTTAAGAGCTAGAAATAGCAAGTT |
| | 9 | 9_Linker6 | TAAATAAGGCTAGTCCGTTATCAGCGTGAAAGCGATCAAGGTGCT |
| | | | GCAGACCTGTCAAACGCGGCACCGAGTCGGTGC |
| 4419 | FANCF_5G_T_ | LegRNA_FANCF_5G_T_ | GGAATCCCTTCTGCAGCACCGTTTAAGAGCTAGAAATAGCAAGTT |
| | 9 | 9_Linker8 | TAAATAAGGCTAGTCCGTTATCAGCGTGAAAGCGATCAAGGTGCT |
| | | | GCAGACAGACTTCAAACGCGGCACCGAGTCGGTGC |
| 4420 | RHO_P23H_ | LegRNA_RHO_P23H_ | GGCTCAGCCAGGTAGTACTGGTTTAAGAGCTAGAAATAGCAAGT |
| | noPAM_229 | PAM_229_Linker0 | TTAAATAAGGCTAGTCCGTTATCAGCGTGAACGCAGCCCCTTCGA |
| | | no | GTACCCACAGTACTACCTGGAAACGCGGCACCGAGTCGGTGC |
| 4421 | RHO_P23H_ | LegRNA_RHO_P23H_ | GGCTCAGCCAGGTAGTACTGGTTTAAGAGCTAGAAATAGCAAGT |
| | noPAM_229 | noPAM_229_Linker2 | TTAAATAAGGCTAGTCCGTTATCAGCGTGAACGCAGCCCCTTCGA |
| | | | GTACCCACAGTACTACCTGGACAAACGCGGCACCGAGTCGGTGC |
| 4422 | RHO_P23H_ | LegRNA_RHO_P23H_ | GGCTCAGCCAGGTAGTACTGGTTTAAGAGCTAGAAATAGCAAGT |
| | noPAM_229 | noPAM_229_Linker4 | TTAAATAAGGCTAGTCCGTTATCAGCGTGAACGCAGCCCCTTCGA |
| | | | GTACCCACAGTACTACCTGGAAAAAAACGCGGCACCGAGTCGGT |
| | | | GC |
| 4423 | RHO_P23H_ | LegRNA_RHO_P23H_ | GGCTCAGCCAGGTAGTACTGGTTTAAGAGCTAGAAATAGCAAGT |
| | noPAM_229 | noPAM_229_Linker6 | TTAAATAAGGCTAGTCCGTTATCAGCGTGAACGCAGCCCCTTCGA |
| | | | GTACCCACAGTACTACCTGGGACCAAAAACGCGGCACCGAGTCG |
| | | | GTGC |
| 4424 | RHO_P23H_ | LegRNA_RHO_P23H_ | GGCTCAGCCAGGTAGTACTGGTTTAAGAGCTAGAAATAGCAAGT |
| | noPAM_229 | noPAM_229_Linker8 | TTAAATAAGGCTAGTCCGTTATCAGCGTGAACGCAGCCCCTTCGA |
| | | | GTACCCACAGTACTACCTGGATAATATGAAACGCGGCACCGAGT |
| | | | CGGTGC |
| 4425 | RHO_P23H_ | LegRNA_RHO_P23H_ | GAGTACTGTGGGTACTCGAAGGTTTAAGAGCTAGAAATAGCAAG |
| | noPAM_160 | noPAM_160_Linker0 | TTTAAATAAGGCTAGTCCGTTATCAGCGTGAGTGTGGTACGCAGC |
| | | | CCCTTCGAGTACCCACAGAAACGCGGCACCGAGTCGGTGC |
| 4426 | RHO_P23H_ | LegRNA_RHO_P23H_ | GAGTACTGTGGGTACTCGAAGGTTTAAGAGCTAGAAATAGCAAG |
| | noPAM_160 | noPAM_160_Linker2 | TTTAAATAAGGCTAGTCCGTTATCAGCGTGAGTGTGGTACGCAGC |
| | | | CCCTTCGAGTACCCACAGAGAAACGCGGCACCGAGTCGGTGC |
| 4427 | RHO_P23H_ | LegRNA_RHO_P23H_ | GAGTACTGTGGGTACTCGAAGGTTTAAGAGCTAGAAATAGCAAG |
| | noPAM_160 | noPAM_160_Linker4 | TTTAAATAAGGCTAGTCCGTTATCAGCGTGAGTGTGGTACGCAGC |
| | | | CCCTTCGAGTACCCACAGCCGAAAACGCGGCACCGAGTCGGTGC |
| 4428 | RHO_P23H_ | LegRNA_RHO_P23H_ | GAGTACTGTGGGTACTCGAAGGTTTAAGAGCTAGAAATAGCAAG |
| | noPAM_160 | noPAM_160_Linker6 | TTTAAATAAGGCTAGTCCGTTATCAGCGTGAGTGTGGTACGCAGC |
| | | | CCCTTCGAGTACCCACAGATCATAAAACGCGGCACCGAGTCGGT |
| | | | GC |
| 4429 | RHO_P23H_ | LegRNA_RHO_P23H_ | GAGTACTGTGGGTACTCGAAGGTTTAAGAGCTAGAAATAGCAAG |
| | noPAM_160 | noPAM_160_Linker8 | TTTAAATAAGGCTAGTCCGTTATCAGCGTGAGTGTGGTACGCAGC |
| | | | CCCTTCGAGTACCCACAGATAGCCCAAAACGCGGCACCGAGTCG |
| | | | GTGC |
| 4430 | SLC37A4_ | LegRNA_SLC37A4_ | GCAGCTCTGGATCCTGGTATGTTTAAGAGCTAGAAATAGCAAGTT |
| | G339C_93 | G339C_93_Linker0 | TAAATAAGGCTAGTCCGTTATCAGCGTGAAGAAACCAAATACAG |
| | | | CTCCCAATACCAGGATCCAAACGCGGCACCGAGTCGGTGC |
| 4431 | SLC37A4_ | LegRNA_SLC37A4_ | GCAGCTCTGGATCCTGGTATGTTTAAGAGCTAGAAATAGCAAGTT |
| | G339C_93 | G339C_93_Linker2 | TAAATAAGGCTAGTCCGTTATCAGCGTGAAGAAACCAAATACAG |
| | | | CTCCCAATACCAGGATCCCCAAACGCGGCACCGAGTCGGTGC |
| 4432 | SLC37A4_ | LegRNA_SLC37A4_ | GCAGCTCTGGATCCTGGTATGTTTAAGAGCTAGAAATAGCAAGTT |
| | G339C_93 | G339C_93_Linker4 | TAAATAAGGCTAGTCCGTTATCAGCGTGAAGAAACCAAATACAG |
| | | | CTCCCAATACCAGGATCCCACAAAACGCGGCACCGAGTCGGTGC |
| 4433 | SLC37A4_ | LegRNA_SLC37A4_ | GCAGCTCTGGATCCTGGTATGTTTAAGAGCTAGAAATAGCAAGTT |
| | G339C_93 | G339C_93_Linker6 | TAAATAAGGCTAGTCCGTTATCAGCGTGAAGAAACCAAATACAG |
| | | | CTCCCAATACCAGGATCCCTATATAAACGCGGCACCGAGTCGGTG |
| | | | C |
| 4434 | SLC37A4_ | LegRNA_SLC37A4_ | GCAGCTCTGGATCCTGGTATGTTTAAGAGCTAGAAATAGCAAGTT |
| | G339C_93 | G339C_93_Linker8 | TAAATAAGGCTAGTCCGTTATCAGCGTGAAGAAACCAAATACAG |
| | | | CTCCCAATACCAGGATCCCCCGATCAAAACGCGGCACCGAGTCG |
| | | | GTGC |
| 4435 | SLC37A4_ | LegRNA_SLC37A4_ | GCAGCTCTGGATCCTGGTATGTTTAAGAGCTAGAAATAGCAAGTT |
| | G339C_85 | G339C_85_Linker0 | TAAATAAGGCTAGTCCGTTATCAGCGTGAGAAACCAAATACAGC |
| | | | TCCCAATACCAGGATCCAAAACGCGGCACCGAGTCGGTGC |
| 4436 | SLC37A4_ | LegRNA_SLC37A4_ | GCAGCTCTGGATCCTGGTATGTTTAAGAGCTAGAAATAGCAAGTT |
| | G339C_85 | G339C_85_Linker2 | TAAATAAGGCTAGTCCGTTATCAGCGTGAGAAACCAAATACAGC |
| | | | TCCCAATACCAGGATCCACCAAACGCGGCACCGAGTCGGTGC |
| 4437 | SLC37A4_ | LegRNA_SLC37A4_ | GCAGCTCTGGATCCTGGTATGTTTAAGAGCTAGAAATAGCAAGTT |
| | G339C_85 | G339C_85_Linker4 | TAAATAAGGCTAGTCCGTTATCAGCGTGAGAAACCAAATACAGC |
| | | | TCCCAATACCAGGATCCACCACAAACGCGGCACCGAGTCGGTGC |
| 4438 | SLC37A4_ | LegRNA_SLC37A4_ | GCAGCTCTGGATCCTGGTATGTTTAAGAGCTAGAAATAGCAAGTT |
| | G339C_85 | G339C_85_Linker6 | TAAATAAGGCTAGTCCGTTATCAGCGTGAGAAACCAAATACAGC |
| | | | TCCCAATACCAGGATCCACTTTCAAAACGCGGCACCGAGTCGGTG |
| | | | C |
| 4439 | SLC37A4_ | LegRNA_SLC37A4_ | GCAGCTCTGGATCCTGGTATGTTTAAGAGCTAGAAATAGCAAGTT |
| | G339C_85 | G339C_85_Linker8 | TAAATAAGGCTAGTCCGTTATCAGCGTGAGAAACCAAATACAGC |
| | | | TCCCAATACCAGGATCCACTTCGTTAAAACGCGGCACCGAGTCGG |
| | | | TGC |

Extended gRNA Cores

In some embodiments, a PEgRNA comprises a gRNA core that comprises one or more nucleotide insertions compared to a wild type CRISPR guide RNA scaffold sequence (e.g. a canonical SpCa9 guide RNA scaffold), i.e. an extended in length gRNA core. Potential advantages associated with such extended gRNA cores may include improved prime editing efficiency and/or improved manufacturing via a split synthesis scheme.

In some embodiments, the gRNA core comprises insertion of one or more nucleotides in the direct repeat compared to a wild type CRISPR guide RNA scaffold sequence as set forth in SEQ ID NO: 16. In some embodiments, the gRNA core comprises insertion of one or more nucleotides in the second stem loop compared to a canonical SpCas9 guide RNA scaffold sequence as set forth in SEQ ID NO: 16. Exemplary extended gRNA cores are provided in Tables 1 and 2. Although gRNA core sequences provided in Tables 1 and 2 are RNA sequences, “T” is used instead of “U” in the sequences for consistency with the ST.26 standard.

Components of a PEgRNA, e.g., an extended PEgRNA, may be synthesized by split synthesis, which refers to synthesizing two (or more) portions of a PEgRNA (e.g., a 5′ half of the PEgRNA and a 3′ half of the PEgRNA) separately and ligating the first half to a second half to form a full length PEgRNA. Exemplary “split” positions between the 5′ half and the 3′ half, e.g., in the direct repeat or in the second stem loop of a gRNA core, are shown in FIG. 8. Exemplary gRNA core sequences and corresponding first half and second half portions for split synthesis are shown in Table 2.

In certain embodiments, PEgRNAs provided herein comprise: i) a first sequence comprising a spacer that comprises a region of complementarity to a search target sequence in target strand of a double stranded target DNA, and a first half of a gRNA core; and ii) a second sequence comprising a second half of the gRNA core, an editing template that comprises an intended edit compared to the double stranded target DNA; a primer binding site (PBS) that comprises a region of complementarity to a region upstream of a nick site in a non-target strand of the double stranded target DNA; and, wherein the gRNA core comprises a direct repeat, a first stem loop, and a second stem loop.

In certain embodiments, PEgRNAs provided herein comprise i) a first sequence comprising an editing template that comprises an intended edit compared to the double stranded target DNA; a primer binding site (PBS) that comprises a region of complementarity to a region upstream of a nick site in a non-target strand of the double stranded target DNA; a spacer that comprises a region of complementarity to a search target sequence in target strand of a double stranded target DNA; and a first half of a gRNA core; and ii) a second sequence comprising a second half of a gRNA core, wherein the gRNA core comprises a direct repeat, a first stem loop, and a second stem loop.

In some embodiments, the first sequence is on a first RNA molecule and the second sequence is on a second RNA molecule. In some embodiments, the spacer and the first sequence and the second sequence are on the same RNA molecule. In some embodiments, the first half of the gRNA core and the second half of the gRNA core are selected from the paired first half gRNA core sequences and second half gRNA sequences provided in Table 2.

It should be appreciated that the first half and second half of the gRNA core may or may not be equal in length. In some embodiments, the first half of the gRNA core is at least five, at least 10, at least 15, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, or at least 75 nucleotides in length. In some embodiments, the second half of the gRNA core is at least five, at least 10, at least 15, at least 20 nucleotides, at least 25 nucleotides, at least 30 nucleotides, at least 35 nucleotides, at least 40 nucleotides, at least 45 nucleotides, at least 50 nucleotides, at least 55 nucleotides, at least 60 nucleotides, at least 65 nucleotides, at least 70 nucleotides, or at least 75 nucleotides in length.

In some embodiments, the first half of the gRNA core is at least 80%, at least 85%, at least 90%, at least 95%, at least 99% identical to a sequence provided in Table 2. In some embodiments, the first half of the gRNA core is identical to a sequence provided in Table 2. In some embodiments, the second half of the gRNA core is at least 80%, at least 85%, at least 90%, at least 95%, at least 99% identical to a sequence provided in Table 2. In some embodiments, the second half of the gRNA core is identical to a sequence provided in Table 2.

As previously discussed, the gRNA core may comprise a direct repeat and/or one or multiple stem loops. In some embodiments, gRNA cores synthesize using split synthesis comprise a first half of a gRNA core comprising a first half of the direct repeat and a second half of a gRNA core comprising the second half of the direct repeat. In some embodiments, gRNA cores synthesizes using split synthesis comprises a first half of a gRNA core comprising a first half of the second stem loop and a second half of a gRNA core comprising the second half of the second stem loop.

TABLE 2
Exemplary gRNA core sequences

| SEQ | | | SEQ | | SEQ | | | Base | |
|---|---|---|---|---|---|---|---|---|---|
| ID | | | ID | Exemplary First half | ID | Exemplary Second half | Extended | Pairs of | Lenth |
| NO. | Name | gRNA core sequence | NO: | of the gRNA core | NO: | of the gRNA core | feature | Extension | (nts) |
| 3860 | L2_e3_ | GTTTAAGAGCTAGAA | 6378 | GTTTAAGAGCTAGA | 6536 | CTCGAAAGAGACGC | Stem loop | 3 | 82 |
| | 1 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCTCGAAAGA | | TTATCAGCGT | | | | | |
| | | GACGCGGCACCGAGT | | | | | | | |
| | | CGGTGC | | | | | | | |
| 3861 | L2_e3_ | GTTTAAGAGCTAGAA | 6379 | GTTTAAGAGCTAGA | 6537 | GCAGAAATGCACGC | Stem loop | 3 | 82 |
| | 2 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGCAGAAATG | | TTATCAGCGT | | | | | |
| | | CACGCGGCACCGAGT | | | | | | | |
| | | CGGTGC | | | | | | | |
| 3862 | L2_e3_ | GTTTAAGAGCTAGAA | 6380 | GTTTAAGAGCTAGA | 6538 | CCCGAAAGGGACGC | Stem loop | 3 | 82 |
| | 3 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCCCGAAAGG | | TTATCAGCGT | | | | | |
| | | GACGCGGCACCGAGT | | | | | | | |
| | | CGGTGC | | | | | | | |
| 3863 | L2_e3_ | GTTTAAGAGCTAGAA | 6381 | GTTTAAGAGCTAGA | 6539 | TGCGAAAGCAACGC | Stem loop | 3 | 82 |
| | 4 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTTGCGAAAGC | | TTATCAGCGT | | | | | |
| | | AACGCGGCACCGAGT | | | | | | | |
| | | CGGTGC | | | | | | | |
| 3864 | L2_e3_ | GTTTAAGAGCTAGAA | 6382 | GTTTAAGAGCTAGA | 6540 | CACGAAAGTGACGC | Stem loop | 3 | 82 |
| | 5 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCACGAAAGT | | TTATCAGCGT | | | | | |
| | | GACGCGGCACCGAGT | | | | | | | |
| | | CGGTGC | | | | | | | |
| 3865 | L2_e3_ | GTTTAAGAGCTAGAA | 6383 | GTTTAAGAGCTAGA | 6541 | CCAGAAATGGACGC | Stem loop | 3 | 82 |
| | 6 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCCAGAAATG | | TTATCAGCGT | | | | | |
| | | GACGCGGCACCGAGT | | | | | | | |
| | | CGGTGC | | | | | | | |
| 3866 | L2_e3_ | GTTTAAGAGCTAGAA | 6384 | GTTTAAGAGCTAGA | 6542 | GCTGAAAAGCACGC | Stem loop | 3 | 82 |
| | 7 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGCTGAAAAG | | TTATCAGCGT | | | | | |
| | | CACGCGGCACCGAGT | | | | | | | |
| | | CGGTGC | | | | | | | |
| 3867 | L2_e3_ | GTTTAAGAGCTAGAA | 6385 | GTTTAAGAGCTAGA | 6543 | GTCGAAAGGCACGC | Stem loop | 3 | 82 |
| | 8 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGTCGAAAGG | | TTATCAGCGT | | | | | |
| | | CACGCGGCACCGAGT | | | | | | | |
| | | CGGTGC | | | | | | | |
| 3868 | L2_e3_ | GTTTAAGAGCTAGAA | 6386 | GTTTAAGAGCTAGA | 6544 | CGGGAAACCGACGC | Stem loop | 3 | 82 |
| | 9 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCGGGAAACC | | TTATCAGCGT | | | | | |
| | | GACGCGGCACCGAGT | | | | | | | |
| | | CGGTGC | | | | | | | |
| 3869 | L2_e3_ | GTTTAAGAGCTAGAA | 6387 | GTTTAAGAGCTAGA | 6545 | CAGGAAACTGACGC | Stem loop | 3 | 82 |
| | 10 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCAGGAAACT | | TTATCAGCGT | | | | | |
| | | GACGCGGCACCGAGT | | | | | | | |
| | | CGGTGC | | | | | | | |
| 3870 | L2_e3_ | GTTTAAGAGCTAGAA | 6388 | GTTTAAGAGCTAGA | 6546 | TCCGAAAGGAACGC | Stem loop | 3 | 82 |
| | 11 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTTCCGAAAGG | | TTATCAGCGT | | | | | |
| | | AACGCGGCACCGAGT | | | | | | | |
| | | CGGTGC | | | | | | | |
| 3871 | L2_e3_ | GTTTAAGAGCTAGAA | 6389 | GTTTAAGAGCTAGA | 6547 | GGGGAAACCCACGC | Stem loop | 3 | 82 |
| | 12 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGGGGAAACC | | TTATCAGCGT | | | | | |
| | | CACGCGGCACCGAGT | | | | | | | |
| | | CGGTGC | | | | | | | |
| 3872 | L2_e3_ | GTTTAAGAGCTAGAA | 6390 | GTTTAAGAGCTAGA | 6548 | AGCGAAAGCTACGC | Stem loop | 3 | 82 |
| | 13 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTAGCGAAAGC | | TTATCAGCGT | | | | | |
| | | TACGCGGCACCGAGT | | | | | | | |
| | | CGGTGC | | | | | | | |
| 3873 | L2_e3_ | GTTTAAGAGCTAGAA | 6391 | GTTTAAGAGCTAGA | 6549 | GTGGAAACACACGC | Stem loop | 3 | 82 |
| | 14 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGTGGAAACA | | TTATCAGCGT | | | | | |
| | | CACGCGGCACCGAGT | | | | | | | |
| | | CGGTGC | | | | | | | |
| 3874 | L2_e3_ | GTTTAAGAGCTAGAA | 6392 | GTTTAAGAGCTAGA | 6550 | GCCGAAAGGCACGC | Stem loop | 3 | 82 |
| | 15 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGCCGAAAGG | | TTATCAGCGT | | | | | |
| | | CACGCGGCACCGAGT | | | | | | | |
| | | CGGTGC | | | | | | | |
| 3875 | L2_e3_ | GTTTAAGAGCTAGAA | 6393 | GTTTAAGAGCTAGA | 6551 | CGTGAAAACGACGC | Stem loop | 3 | 82 |
| | 16 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCGTGAAAAC | | TTATCAGCGT | | | | | |
| | | GACGCGGCACCGAGT | | | | | | | |
| | | CGGTGC | | | | | | | |
| 3876 | L2_e3_ | GTTTAAGAGCTAGAA | 6394 | GTTTAAGAGCTAGA | 6552 | ACCGAAAGGTACGC | Stem loop | 3 | 82 |
| | 17 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTACCGAAAGG | | TTATCAGCGT | | | | | |
| | | TACGCGGCACCGAGT | | | | | | | |
| | | CGGTGC | | | | | | | |
| 3877 | L2_e3_ | GTTTAAGAGCTAGAA | 6395 | GTTTAAGAGCTAGA | 6553 | CCTGAAAAGGACGC | Stem loop | 3 | 82 |
| | 18 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCCTGAAAAG | | TTATCAGCGT | | | | | |
| | | GACGCGGCACCGAGT | | | | | | | |
| | | CGGTGC | | | | | | | |
| 3878 | L2_e3_ | GTTTAAGAGCTAGAA | 6396 | GTTTAAGAGCTAGA | 6554 | CCGGAAACGGACGC | Stem loop | 3 | 82 |
| | 19 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCCGGAAACG | | TTATCAGCGT | | | | | |
| | | GACGCGGCACCGAGT | | | | | | | |
| | | CGGTGC | | | | | | | |
| 3879 | L2_e3_ | GTTTAAGAGCTAGAA | 6397 | GTTTAAGAGCTAGA | 6555 | ACGGAAACGTACGC | Stem loop | 3 | 82 |
| | 20 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTACGGAAACG | | TTATCAGCGT | | | | | |
| | | TACGCGGCACCGAGT | | | | | | | |
| | | CGGTGC | | | | | | | |
| 3880 | L2_e3_ | GTTTAAGAGCTAGAA | 6398 | GTTTAAGAGCTAGA | 6556 | GCGGAAACGCACGC | Stem loop | 3 | 82 |
| | 21 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGCGGAAACG | | TTATCAGCGT | | | | | |
| | | CACGCGGCACCGAGT | | | | | | | |
| | | CGGTGC | | | | | | | |
| 3881 | L2_e3_ | GTTTAAGAGCTAGAA | 6399 | GTTTAAGAGCTAGA | 6557 | CTGGAAACAGACGC | Stem loop | 3 | 82 |
| | 22 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCTGGAAACA | | TTATCAGCGT | | | | | |
| | | GACGCGGCACCGAGT | | | | | | | |
| | | CGGTGC | | | | | | | |
| 3882 | L2_e3 | GTTTAAGAGCTAGAA | 6400 | GTTTAAGAGCTAGA | 6558 | GGGGAAACTCACGC | Stem loop | 3 | 82 |
| | 23 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTGGGGAAACT | | TTATCAGCGT | | | | | |
| | | CACGCGGCACCGAGT | | | | | | | |
| | | CGGTGC | | | | | | | |
| 3883 | L2_e3_ | GTTTAAGAGCTAGAA | 6401 | GTTTAAGAGCTAGA | 6559 | CCGGAAATGGACGC | Stem loop | 3 | 82 |
| | 24 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCCGGAAATG | | TTATCAGCGT | | | | | |
| | | GACGCGGCACCGAGT | | | | | | | |
| | | CGGTGC | | | | | | | |
| 3884 | L2_e3_ | GTTTAAGAGCTAGAA | 6402 | GTTTAAGAGCTAGA | 6560 | CGTGAAAGCGACGC | Stem loop | 3 | 82 |
| | 25 | ATAGCAAGTTTAAAT | | AATAGCAAGTTTAA | | GGCACCGAGTCGGT | 2 | | |
| | | AAGGCTAGTCCGTTAT | | ATAAGGCTAGTCCG | | GC | | | |
| | | CAGCGTCGTGAAAGC | | TTATCAGCGT | | | | | |
| | | GACGCGGCACCGAGT | | | | | | | |
| | | CGGTGC | | | | | | | |
| 3885 | L2_e4_ | GTTTAAGAGCTAGAA | 6403 | GTTTAAGAGCTAGA | 6561 | GTGGAAACACAACG | Stem loop | 4 | 84 |
| | 1 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGTGGAAAC | | TTATCAGCGTt | | | | | |
| | | ACAACGCGGCACCGA | | | | | | | |
| | | GTCGGTGC | | | | | | | |
| 3886 | L2_e4_ | GTTTAAGAGCTAGAA | 6404 | GTTTAAGAGCTAGA | 6562 | CAGGAAACTGTACG | Stem loop | 4 | 84 |
| | 2 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACAGGAAAC | | TTATCAGCGTa | | | | | |
| | | TGTACGCGGCACCGA | | | | | | | |
| | | GTCGGTGC | | | | | | | |
| 3887 | L2_e4_ | GTTTAAGAGCTAGAA | 6405 | GTTTAAGAGCTAGA | 6563 | GAAGAAATTCGACG | Stem loop | 4 | 84 |
| | 3 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGAAGAAAT | | TTATCAGCGTc | | | | | |
| | | TCGACGCGGCACCGA | | | | | | | |
| | | GTCGGTGC | | | | | | | |
| 3888 | L2_e4_ | GTTTAAGAGCTAGAA | 6406 | GTTTAAGAGCTAGA | 6564 | ACCGAAAGGTAACG | Stem loop | 4 | 84 |
| | 4 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTACCGAAAG | | TTATCAGCGTt | | | | | |
| | | GTAACGCGGCACCGA | | | | | | | |
| | | GTCGGTGC | | | | | | | |
| 3889 | L2_e4_ | GTTTAAGAGCTAGAA | 6407 | GTTTAAGAGCTAGA | 6565 | CCGGAAACGGCACG | Stem loop | 4 | 84 |
| | 5 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGCCGGAAAC | | TTATCAGCGTg | | | | | |
| | | GGCACGCGGCACCGA | | | | | | | |
| | | GTCGGTGC | | | | | | | |
| 3890 | L2_e4_ | GTTTAAGAGCTAGAA | 6408 | GTTTAAGAGCTAGA | 6566 | AACGAAAGTTGACG | Stem loop | 4 | 84 |
| | 6 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCAACGAAAG | | TTATCAGCGTc | | | | | |
| | | TTGACGCGGCACCGA | | | | | | | |
| | | GTCGGTGC | | | | | | | |
| 3891 | L2_e4_ | GTTTAAGAGCTAGAA | 6409 | GTTTAAGAGCTAGA | 6567 | GGCGAAAGCTAACG | Stem loop | 4 | 84 |
| | 7 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGGCGAAAG | | TTATCAGCGTt | | | | | |
| | | CTAACGCGGCACCGA | | | | | | | |
| | | GTCGGTGC | | | | | | | |
| 3892 | L2_e4_ | GTTTAAGAGCTAGAA | 6410 | GTTTAAGAGCTAGA | 6568 | AGCGAAAGCTAACG | Stem loop | 4 | 84 |
| | 8 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTAGCGAAAG | | TTATCAGCGTt | | | | | |
| | | CTAACGCGGCACCGA | | | | | | | |
| | | GTCGGTGC | | | | | | | |
| 3893 | L2_e4_ | GTTTAAGAGCTAGAA | 6411 | GTTTAAGAGCTAGA | 6569 | GTCGAAAGACCACG | Stem loop | 4 | 84 |
| | 9 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGGTCGAAAG | | TTATCAGCGTg | | | | | |
| | | ACCACGCGGCACCGA | | | | | | | |
| | | GTCGGTGC | | | | | | | |
| 3894 | L2_e4_ | GTTTAAGAGCTAGAA | 6412 | GTTTAAGAGCTAGA | 6570 | CGCGAAAGCGTACG | Stem loop | 4 | 84 |
| | 10 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACGCGAAAG | | TTATCAGCGTa | | | | | |
| | | CGTACGCGGCACCGA | | | | | | | |
| | | GTCGGTGC | | | | | | | |
| 3895 | L2_e4_ | GTTTAAGAGCTAGAA | 6413 | GTTTAAGAGCTAGA | 6571 | GTGGAAACATCACG | Stem loop | 4 | 84 |
| | 11 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGGTGGAAAC | | TTATCAGCGTg | | | | | |
| | | ATCACGCGGCACCGA | | | | | | | |
| | | GTCGGTGC | | | | | | | |
| 3896 | L2_e4_ | GTTTAAGAGCTAGAA | 6414 | GTTTAAGAGCTAGA | 6572 | GTGGAAACACGACG | Stem loop | 4 | 84 |
| | 12 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGTGGAAAC | | TTATCAGCGTc | | | | | |
| | | ACGACGCGGCACCGA | | | | | | | |
| | | GTCGGTGC | | | | | | | |
| 3897 | L2_e4_ | GTTTAAGAGCTAGAA | 6415 | GTTTAAGAGCTAGA | 6573 | ACTGAAAAGTCACG | Stem loop | 4 | 84 |
| | 13 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGACTGAAAA | | TTATCAGCGTg | | | | | |
| | | GTCACGCGGCACCGA | | | | | | | |
| | | GTCGGTGC | | | | | | | |
| 3898 | L2_e4_ | GTTTAAGAGCTAGAA | 6416 | GTTTAAGAGCTAGA | 6574 | GCAGAAATGCTACG | Stem loop | 4 | 84 |
| | 14 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTAGCAGAAAT | | TTATCAGCGTa | | | | | |
| | | GCTACGCGGCACCGA | | | | | | | |
| | | GTCGGTGC | | | | | | | |
| 3899 | L2_e4_ | GTTTAAGAGCTAGAA | 6417 | GTTTAAGAGCTAGA | 6575 | TGCGAAAGCAGACG | Stem loop | 4 | 84 |
| | 15 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTTGCGAAAG | | TTATCAGCGTt | | | | | |
| | | CAGACGCGGCACCGA | | | | | | | |
| | | GTCGGTGC | | | | | | | |
| 3900 | L2_e4_ | GTTTAAGAGCTAGAA | 6418 | GTTTAAGAGCTAGA | 6576 | TTCGAAAGAGGACG | Stem loop | 4 | 84 |
| | 16 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCTTCGAAAG | | TTATCAGCGTc | | | | | |
| | | AGGACGCGGCACCGA | | | | | | | |
| | | GTCGGTGC | | | | | | | |
| 3901 | L2_e4_ | GTTTAAGAGCTAGAA | 6419 | GTTTAAGAGCTAGA | 6577 | GGGGAAACCTGACG | Stem loop | 4 | 84 |
| | 17 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGGGGAAAC | | TTATCAGCGTc | | | | | |
| | | CTGACGCGGCACCGA | | | | | | | |
| | | GTCGGTGC | | | | | | | |
| 3902 | L2_e4_ | GTTTAAGAGCTAGAA | 6420 | GTTTAAGAGCTAGA | 6578 | GCGGAAACGCTACG | Stem loop | 4 | 84 |
| | 18 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTAGCGGAAAC | | TTATCAGCGTa | | | | | |
| | | GCTACGCGGCACCGA | | | | | | | |
| | | GTCGGTGC | | | | | | | |
| 3903 | L2_e4_ | GTTTAAGAGCTAGAA | 6421 | GTTTAAGAGCTAGA | 6579 | ACCGAAAGGTGACG | Stem loop | 4 | 84 |
| | 19 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCACCGAAAG | | TTATCAGCGTc | | | | | |
| | | GTGACGCGGCACCGA | | | | | | | |
| | | GTCGGTGC | | | | | | | |
| 3904 | L2_e4_ | GTTTAAGAGCTAGAA | 6422 | GTTTAAGAGCTAGA | 6580 | GCGGAAACGCCACG | Stem loop | 4 | 84 |
| | 20 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGGCGGAAAC | | TTATCAGCGTg | | | | | |
| | | GCCACGCGGCACCGA | | | | | | | |
| | | GTCGGTGC | | | | | | | |
| 3905 | L2_e4_ | GTTTAAGAGCTAGAA | 6423 | GTTTAAGAGCTAGA | 6581 | TACGAAAGTAGACG | Stem loop | 4 | 84 |
| | 21 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCTACGAAAG | | TTATCAGCGTc | | | | | |
| | | TAGACGCGGCACCGA | | | | | | | |
| | | GTCGGTGC | | | | | | | |
| 3906 | L2_e4 | GTTTAAGAGCTAGAA | 6424 | GTTTAAGAGCTAGA | 6582 | CCTGAAAAGGCACG | Stem loop | 4 | 84 |
| | 22 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGCCTGAAAA | | TTATCAGCGTg | | | | | |
| | | GGCACGCGGCACCGA | | | | | | | |
| | | GTCGGTGC | | | | | | | |
| 3907 | L2_e4_ | GTTTAAGAGCTAGAA | 6425 | GTTTAAGAGCTAGA | 6583 | TGCGAAAGCACACG | Stem loop | 4 | 84 |
| | 23 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGTGCGAAAG | | TTATCAGCGTg | | | | | |
| | | CACACGCGGCACCGA | | | | | | | |
| | | GTCGGTGC | | | | | | | |
| 3908 | L2_e4_ | GTTTAAGAGCTAGAA | 6426 | GTTTAAGAGCTAGA | 6584 | TGAGAAATCACACG | Stem loop | 4 | 84 |
| | 24 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGTGAGAAAT | | TTATCAGCGTg | | | | | |
| | | CACACGCGGCACCGA | | | | | | | |
| | | GTCGGTGC | | | | | | | |
| 3909 | L2_e4_ | GTTTAAGAGCTAGAA | 6427 | GTTTAAGAGCTAGA | 6585 | CAGGAAACTGCACG | Stem loop | 4 | 84 |
| | 25 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGCAGGAAAC | | TTATCAGCGTg | | | | | |
| | | TGCACGCGGCACCGA | | | | | | | |
| | | GTCGGTGC | | | | | | | |
| 3910 | L2_e4_ | GTTTAAGAGCTAGAA | 6428 | GTTTAAGAGCTAGA | 6586 | CTCGAAAGAGAACG | Stem loop | 4 | 84 |
| | 26 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTCTCGAAAG | | TTATCAGCGTt | | | | | |
| | | AGAACGCGGCACCGA | | | | | | | |
| | | GTCGGTGC | | | | | | | |
| 3911 | L2_e4_ | GTTTAAGAGCTAGAA | 6429 | GTTTAAGAGCTAGA | 6587 | CCCGAAAGGGCACG | Stem loop | 4 | 84 |
| | 27 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGCCCGAAAG | | TTATCAGCGTg | | | | | |
| | | GGCACGCGGCACCGA | | | | | | | |
| | | GTCGGTGC | | | | | | | |
| 3912 | L2_e4_ | GTTTAAGAGCTAGAA | 6430 | GTTTAAGAGCTAGA | 6588 | ACCGAAAGGTTACG | Stem loop | 4 | 84 |
| | 28 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTAACCGAAAG | | TTATCAGCGTa | | | | | |
| | | GTTACGCGGCACCGA | | | | | | | |
| | | GTCGGTGC | | | | | | | |
| 3913 | L2_e4_ | GTTTAAGAGCTAGAA | 6431 | GTTTAAGAGCTAGA | 6589 | CTGGAAACAGTACG | Stem loop | 4 | 84 |
| | 29 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACTGGAAAC | | TTATCAGCGTa | | | | | |
| | | AGTACGCGGCACCGA | | | | | | | |
| | | GTCGGTGC | | | | | | | |
| 3914 | L2_e4_ | GTTTAAGAGCTAGAA | 6432 | GTTTAAGAGCTAGA | 6590 | GGCGAAAGCCGACG | Stem loop | 4 | 84 |
| | 30 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGGCGAAAG | | TTATCAGCGTc | | | | | |
| | | CCGACGCGGCACCGA | | | | | | | |
| | | GTCGGTGC | | | | | | | |
| 3915 | L2_e4_ | GTTTAAGAGCTAGAA | 6433 | GTTTAAGAGCTAGA | 6591 | GCCGAAAGGCTACG | Stem loop | 4 | 84 |
| | 31 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTAGCCGAAAG | | TTATCAGCGTa | | | | | |
| | | GCTACGCGGCACCGA | | | | | | | |
| | | GTCGGTGC | | | | | | | |
| 3916 | L2_e4_ | GTTTAAGAGCTAGAA | 6434 | GTTTAAGAGCTAGA | 6592 | ACAGAAATGTCACG | Stem loop | 4 | 84 |
| | 32 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGACAGAAAT | | TTATCAGCGTg | | | | | |
| | | GTCACGCGGCACCGA | | | | | | | |
| | | GTCGGTGC | | | | | | | |
| 3917 | L2_e4_ | GTTTAAGAGCTAGAA | 6435 | GTTTAAGAGCTAGA | 6593 | GTCGAAAGACGACG | Stem loop | 4 | 84 |
| | 33 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGTCGAAAG | | TTATCAGCGTt | | | | | |
| | | ACGACGCGGCACCGA | | | | | | | |
| | | GTCGGTGC | | | | | | | |
| 3918 | L2_e4_ | GTTTAAGAGCTAGAA | 6436 | GTTTAAGAGCTAGA | 6594 | CACGAAAGTGGACG | Stem loop | 4 | 84 |
| | 34 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCCACGAAAG | | TTATCAGCGTc | | | | | |
| | | TGGACGCGGCACCGA | | | | | | | |
| | | GTCGGTGC | | | | | | | |
| 3919 | L2_e4_ | GTTTAAGAGCTAGAA | 6437 | GTTTAAGAGCTAGA | 6595 | ACGGAAACGTCACG | Stem loop | 4 | 84 |
| | 35 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGACGGAAAC | | TTATCAGCGTg | | | | | |
| | | GTCACGCGGCACCGA | | | | | | | |
| | | GTCGGTGC | | | | | | | |
| 3920 | L2_e4_ | GTTTAAGAGCTAGAA | 6438 | GTTTAAGAGCTAGA | 6596 | GTGGAAACACCACG | Stem loop | 4 | 84 |
| | 36 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGGTGGAAAC | | TTATCAGCGTg | | | | | |
| | | ACCACGCGGCACCGA | | | | | | | |
| | | GTCGGTGC | | | | | | | |
| 3921 | L2_e4_ | GTTTAAGAGCTAGAA | 6439 | GTTTAAGAGCTAGA | 6597 | AAGGAAACTTCACG | Stem loop | 4 | 84 |
| | 37 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGAAGGAAAC | | TTATCAGCGTg | | | | | |
| | | TTCACGCGGCACCGA | | | | | | | |
| | | GTCGGTGC | | | | | | | |
| 3922 | L2_e4_ | GTTTAAGAGCTAGAA | 6440 | GTTTAAGAGCTAGA | 6598 | CCCGAAAGGGGACG | Stem loop | 4 | 84 |
| | 38 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCCCCGAAAG | | TTATCAGCGTc | | | | | |
| | | GGGACGCGGCACCGA | | | | | | | |
| | | GTCGGTGC | | | | | | | |
| 3923 | L2_e4_ | GTTTAAGAGCTAGAA | 6441 | GTTTAAGAGCTAGA | 6599 | TGTGAAAACGCACG | Stem loop | 4 | 84 |
| | 39 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGTGTGAAAA | | TTATCAGCGTg | | | | | |
| | | CGCACGCGGCACCGA | | | | | | | |
| | | GTCGGTGC | | | | | | | |
| 3924 | L2_e4_ | GTTTAAGAGCTAGAA | 6442 | GTTTAAGAGCTAGA | 6600 | CGGGAAACCGCACG | Stem loop | 4 | 84 |
| | 40 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGCGGGAAAC | | TTATCAGCGTg | | | | | |
| | | CGCACGCGGCACCGA | | | | | | | |
| | | GTCGGTGC | | | | | | | |
| 3925 | L2_e4_ | GTTTAAGAGCTAGAA | 6443 | GTTTAAGAGCTAGA | 6601 | GAGGAAACTCAACG | Stem loop | 4 | 84 |
| | 41 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGAGGAAAC | | TTATCAGCGTt | | | | | |
| | | TCAACGCGGCACCGA | | | | | | | |
| | | GTCGGTGC | | | | | | | |
| 3926 | L2_e4_ | GTTTAAGAGCTAGAA | 6444 | GTTTAAGAGCTAGA | 6602 | CGGGAAACCGTACG | Stem loop | 4 | 84 |
| | 42 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACGGGAAAC | | TTATCAGCGTa | | | | | |
| | | CGTACGCGGCACCGA | | | | | | | |
| | | GTCGGTGC | | | | | | | |
| 3927 | L2_e4_ | GTTTAAGAGCTAGAA | 6445 | GTTTAAGAGCTAGA | 6603 | GACGAAAGTCTACG | Stem loop | 4 | 84 |
| | 43 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTAGACGAAAG | | TTATCAGCGTa | | | | | |
| | | TCTACGCGGCACCGA | | | | | | | |
| | | GTCGGTGC | | | | | | | |
| 3928 | L2_e4_ | GTTTAAGAGCTAGAA | 6446 | GTTTAAGAGCTAGA | 6604 | TTCGAAAGAAGACG | Stem loop | 4 | 84 |
| | 44 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCTTCGAAAG | | TTATCAGCGTc | | | | | |
| | | AAGACGCGGCACCGA | | | | | | | |
| | | GTCGGTGC | | | | | | | |
| 3929 | L2_e4_ | GTTTAAGAGCTAGAA | 6447 | GTTTAAGAGCTAGA | 6605 | TGGGAAACCATACG | Stem loop | 4 | 84 |
| | 45 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTATGGGAAAC | | TTATCAGCGTa | | | | | |
| | | CATACGCGGCACCGA | | | | | | | |
| | | GTCGGTGC | | | | | | | |
| 3930 | L2_e4_ | GTTTAAGAGCTAGAA | 6448 | GTTTAAGAGCTAGA | 6606 | TGGGAAACCACACG | Stem loop | 4 | 84 |
| | 46 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGTGGGAAAC | | TTATCAGCGTg | | | | | |
| | | CACACGCGGCACCGA | | | | | | | |
| | | GTCGGTGC | | | | | | | |
| 3931 | L2_e4_ | GTTTAAGAGCTAGAA | 6449 | GTTTAAGAGCTAGA | 6590 | GGCGAAAGCCGACG | Stem loop | 4 | 84 |
| | 47 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGGCGAAAG | | TTATCAGCGTt | | | | | |
| | | CCGACGCGGCACCGA | | | | | | | |
| | | GTCGGTGC | | | | | | | |
| 3932 | L2_e4_ | GTTTAAGAGCTAGAA | 6450 | GTTTAAGAGCTAGA | 6607 | AGGGAAACCTTACG | Stem loop | 4 | 84 |
| | 48 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTAAGGGAAAC | | TTATCAGCGTa | | | | | |
| | | CTTACGCGGCACCGA | | | | | | | |
| | | GTCGGTGC | | | | | | | |
| 3933 | L2_e4_ | GTTTAAGAGCTAGAA | 6451 | GTTTAAGAGCTAGA | 6608 | AGCGAAAGCTGACG | Stem loop | 4 | 84 |
| | 49 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCAGCGAAAG | | TTATCAGCGTc | | | | | |
| | | CTGACGCGGCACCGA | | | | | | | |
| | | GTCGGTGC | | | | | | | |
| 3934 | L2_e4_ | GTTTAAGAGCTAGAA | 6452 | GTTTAAGAGCTAGA | 6609 | CACGAAAGTGTACG | Stem loop | 4 | 84 |
| | 50 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACACGAAAG | | TTATCAGCGTa | | | | | |
| | | TGTACGCGGCACCGA | | | | | | | |
| | | GTCGGTGC | | | | | | | |
| 3935 | L2_e4_ | GTTTAAGAGCTAGAA | 6453 | GTTTAAGAGCTAGA | 6610 | GTCGAAAGGCGACG | Stem loop | 4 | 84 |
| | 51 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGTCGAAAG | | TTATCAGCGTc | | | | | |
| | | GCGACGCGGCACCGA | | | | | | | |
| | | GTCGGTGC | | | | | | | |
| 3936 | L2_e4_ | GTTTAAGAGCTAGAA | 6454 | GTTTAAGAGCTAGA | 6611 | GCCGAAAGGCAACG | Stem loop | 4 | 84 |
| | 52 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGCCGAAAG | | TTATCAGCGTt | | | | | |
| | | GCAACGCGGCACCGA | | | | | | | |
| | | GTCGGTGC | | | | | | | |
| 3937 | L2_e4_ | GTTTAAGAGCTAGAA | 6455 | GTTTAAGAGCTAGA | 6612 | GGAGAAATCCAACG | Stem loop | 4 | 84 |
| | 53 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGGAGAAAT | | TTATCAGCGTt | | | | | |
| | | CCAACGCGGCACCGA | | | | | | | |
| | | GTCGGTGC | | | | | | | |
| 3938 | L2_e4_ | GTTTAAGAGCTAGAA | 6456 | GTTTAAGAGCTAGA | 6613 | CTCGAAAGAGTACG | Stem loop | 4 | 84 |
| | 54 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACTCGAAAG | | TTATCAGCGTa | | | | | |
| | | AGTACGCGGCACCGA | | | | | | | |
| | | GTCGGTGC | | | | | | | |
| 3939 | L2_e4_ | GTTTAAGAGCTAGAA | 6457 | GTTTAAGAGCTAGA | 6614 | CGTGAAAACGTACG | Stem loop | 4 | 84 |
| | 55 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACGTGAAAA | | TTATCAGCGTa | | | | | |
| | | CGTACGCGGCACCGA | | | | | | | |
| | | GTCGGTGC | | | | | | | |
| 3940 | L2_e4_ | GTTTAAGAGCTAGAA | 6458 | GTTTAAGAGCTAGA | 6615 | ACGGAAACGTTACG | Stem loop | 4 | 84 |
| | 56 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTAACGGAAAC | | TTATCAGCGTa | | | | | |
| | | GTTACGCGGCACCGA | | | | | | | |
| | | GTCGGTGC | | | | | | | |
| 3941 | L2_e4_ | GTTTAAGAGCTAGAA | 6459 | GTTTAAGAGCTAGA | 6616 | ATCGAAAGGTGACG | Stem loop | 4 | 84 |
| | 57 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCATCGAAAG | | TTATCAGCGTc | | | | | |
| | | GTGACGCGGCACCGA | | | | | | | |
| | | GTCGGTGC | | | | | | | |
| 3942 | L2_e4_ | GTTTAAGAGCTAGAA | 6460 | GTTTAAGAGCTAGA | 6617 | TGGGAAACCAGACG | Stem loop | 4 | 84 |
| | 58 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTTGGGAAAC | | TTATCAGCGTt | | | | | |
| | | CAGACGCGGCACCGA | | | | | | | |
| | | GTCGGTGC | | | | | | | |
| 3943 | L2_e4_ | GTTTAAGAGCTAGAA | 6461 | GTTTAAGAGCTAGA | 6618 | AAGGAAACTTGACG | Stem loop | 4 | 84 |
| | 59 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCAAGGAAAC | | TTATCAGCGTc | | | | | |
| | | TTGACGCGGCACCGA | | | | | | | |
| | | GTCGGTGC | | | | | | | |
| 3944 | L2_e4_ | GTTTAAGAGCTAGAA | 6462 | GTTTAAGAGCTAGA | 6619 | GGTGAAAGCCAACG | Stem loop | 4 | 84 |
| | 60 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGGTGAAAG | | TTATCAGCGTt | | | | | |
| | | CCAACGCGGCACCGA | | | | | | | |
| | | GTCGGTGC | | | | | | | |
| 3945 | L2_e4_ | GTTTAAGAGCTAGAA | 6463 | GTTTAAGAGCTAGA | 6620 | CGAGAAATCGGACG | Stem loop | 4 | 84 |
| | 61 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTCGAGAAAT | | TTATCAGCGTt | | | | | |
| | | CGGACGCGGCACCGA | | | | | | | |
| | | GTCGGTGC | | | | | | | |
| 3946 | L2_e4_ | GTTTAAGAGCTAGAA | 6464 | GTTTAAGAGCTAGA | 6621 | GGTGAAAACCCACG | Stem loop | 4 | 84 |
| | 62 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGGGTGAAAA | | TTATCAGCGTg | | | | | |
| | | CCCACGCGGCACCGA | | | | | | | |
| | | GTCGGTGC | | | | | | | |
| 3947 | L2_e4_ | GTTTAAGAGCTAGAA | 6465 | GTTTAAGAGCTAGA | 6622 | ATCGAAAGATCACG | Stem loop | 4 | 84 |
| | 63 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGATCGAAAG | | TTATCAGCGTg | | | | | |
| | | ATCACGCGGCACCGA | | | | | | | |
| | | GTCGGTGC | | | | | | | |
| 3948 | L2_e4_ | GTTTAAGAGCTAGAA | 6466 | GTTTAAGAGCTAGA | 6623 | GCGGAAACGCAACG | Stem loop | 4 | 84 |
| | 64 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGCGGAAAC | | TTATCAGCGTt | | | | | |
| | | GCAACGCGGCACCGA | | | | | | | |
| | | GTCGGTGC | | | | | | | |
| 3949 | L2_e4_ | GTTTAAGAGCTAGAA | 6467 | GTTTAAGAGCTAGA | 6624 | TCGGAAACGAGACG | Stem loop | 4 | 84 |
| | 65 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTTCGGAAAC | | TTATCAGCGTt | | | | | |
| | | GAGACGCGGCACCGA | | | | | | | |
| | | GTCGGTGC | | | | | | | |
| 3950 | L2_e4_ | GTTTAAGAGCTAGAA | 6468 | GTTTAAGAGCTAGA | 6625 | TCAGAAATGGCACG | Stem loop | 4 | 84 |
| | 66 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTGTCAGAAAT | | TTATCAGCGTg | | | | | |
| | | GGCACGCGGCACCGA | | | | | | | |
| | | GTCGGTGC | | | | | | | |
| 3951 | L2_e4_ | GTTTAAGAGCTAGAA | 6469 | GTTTAAGAGCTAGA | 6626 | GCCGAAAGGTAACG | Stem loop | 4 | 84 |
| | 67 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTTGCCGAAAG | | TTATCAGCGTt | | | | | |
| | | GTAACGCGGCACCGA | | | | | | | |
| | | GTCGGTGC | | | | | | | |
| 3952 | L2_e4_ | GTTTAAGAGCTAGAA | 6470 | GTTTAAGAGCTAGA | 6627 | GAGGAAACTCGACG | Stem loop | 4 | 84 |
| | 68 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGAGGAAAC | | TTATCAGCGTc | | | | | |
| | | TCGACGCGGCACCGA | | | | | | | |
| | | GTCGGTGC | | | | | | | |
| 3953 | L2_e4_ | GTTTAAGAGCTAGAA | 6471 | GTTTAAGAGCTAGA | 6628 | GCAGAAATGCGACG | Stem loop | 4 | 84 |
| | 69 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGCAGAAAT | | TTATCAGCGTc | | | | | |
| | | GCGACGCGGCACCGA | | | | | | | |
| | | GTCGGTGC | | | | | | | |
| 3954 | L2_e4_ | GTTTAAGAGCTAGAA | 6472 | GTTTAAGAGCTAGA | 6629 | CTTGAAAAAGGACG | Stem loop | 4 | 84 |
| | 70 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCCTTGAAAA | | TTATCAGCGTc | | | | | |
| | | AGGACGCGGCACCGA | | | | | | | |
| | | GTCGGTGC | | | | | | | |
| 3955 | L2_e4_ | GTTTAAGAGCTAGAA | 6473 | GTTTAAGAGCTAGA | 6630 | GATGAAAATCGACG | Stem loop | 4 | 84 |
| | 71 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGATGAAAA | | TTATCAGCGTc | | | | | |
| | | TCGACGCGGCACCGA | | | | | | | |
| | | GTCGGTGC | | | | | | | |
| 3956 | L2_e4_ | GTTTAAGAGCTAGAA | 6474 | GTTTAAGAGCTAGA | 6593 | GTCGAAAGACGACG | Stem loop | 4 | 84 |
| | 72 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCGTCGAAAG | | TTATCAGCGTc | | | | | |
| | | ACGACGCGGCACCGA | | | | | | | |
| | | GTCGGTGC | | | | | | | |
| 3957 | L2_e4_ | GTTTAAGAGCTAGAA | 6475 | GTTTAAGAGCTAGA | 6631 | ACTGAAAAGTGACG | Stem loop | 4 | 84 |
| | 73 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCACTGAAAA | | TTATCAGCGTc | | | | | |
| | | GTGACGCGGCACCGA | | | | | | | |
| | | GTCGGTGC | | | | | | | |
| 3958 | L2_e4_ | GTTTAAGAGCTAGAA | 6476 | GTTTAAGAGCTAGA | 6632 | CCAGAAATGGTACG | Stem loop | 4 | 84 |
| | 74 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTACCAGAAAT | | TTATCAGCGTa | | | | | |
| | | GGTACGCGGCACCGA | | | | | | | |
| | | GTCGGTGC | | | | | | | |
| 3959 | L2_e4_ | GTTTAAGAGCTAGAA | 6477 | GTTTAAGAGCTAGA | 6633 | TCCGAAAGGAGACG | Stem loop | 4 | 84 |
| | 75 | ATAGCAAGTTTAAAT | | AATAGCAAGTTTAA | | CGGCACCGAGTCGG | 2 | | |
| | | AAGGCTAGTCCGTTAT | | ATAAGGCTAGTCCG | | TGC | | | |
| | | CAGCGTCTCCGAAAG | | TTATCAGCGTc | | | | | |
| | | GAGACGCGGCACCGA | | | | | | | |
| | | GTCGGTGC | | | | | | | |
| 3960 | L2_e5_ | GTTTAAGAGCTAGAA | 6478 | GTTTAAGAGCTAGA | 6634 | GGCGAAAGCCTCAC | Stem loop | 5 | 86 |
| | 1 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGGGGCGAAA | | TTATCAGCGTgg | | | | | |
| | | GCCTCACGCGGCACC | | | | | | | |
| | | GAGTCGGTGC | | | | | | | |
| 3961 | L2_e5_ | GTTTAAGAGCTAGAA | 6479 | GTTTAAGAGCTAGA | 6635 | CAGGAAACTGAGAC | Stem loop | 5 | 86 |
| | 2 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTCAGGAAA | | TTATCAGCGTct | | | | | |
| | | CTGAGACGCGGCACC | | | | | | | |
| | | GAGTCGGTGC | | | | | | | |
| 3962 | L2_e5_ | GTTTAAGAGCTAGAA | 6480 | GTTTAAGAGCTAGA | 6636 | GCCGAAAGGCCGAC | Stem loop | 5 | 86 |
| | 3 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCGGCCGAAA | | TTATCAGCGTcg | | | | | |
| | | GGCCGACGCGGCACC | | | | | | | |
| | | GAGTCGGTGC | | | | | | | |
| 3963 | L2_e5_ | GTTTAAGAGCTAGAA | 6481 | GTTTAAGAGCTAGA | 6637 | GATGAAAGTCGCAC | Stem loop | 5 | 86 |
| | 4 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCGATGAAA | | TTATCAGCGTgc | | | | | |
| | | GTCGCACGCGGCACC | | | | | | | |
| | | GAGTCGGTGC | | | | | | | |
| 3964 | L2_e5_ | GTTTAAGAGCTAGAA | 6482 | GTTTAAGAGCTAGA | 6638 | TGGGAAACCACTAC | Stem loop | 5 | 86 |
| | 5 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAGTGGGAAA | | TTATCAGCGTag | | | | | |
| | | CCACTACGCGGCACC | | | | | | | |
| | | GAGTCGGTGC | | | | | | | |
| 3965 | L2_e5_ | GTTTAAGAGCTAGAA | 6483 | GTTTAAGAGCTAGA | 6639 | GCCGAAAGGCGAAC | Stem loop | 5 | 86 |
| | 6 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTCGCCGAAA | | TTATCAGCGTtc | | | | | |
| | | GGCGAACGCGGCACC | | | | | | | |
| | | GAGTCGGTGC | | | | | | | |
| 3966 | L2_e5_ | GTTTAAGAGCTAGAA | 6484 | GTTTAAGAGCTAGA | 6640 | CGAGAAATTGGCAC | Stem loop | 5 | 86 |
| | 7 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCCGAGAAA | | TTATCAGCGTgc | | | | | |
| | | TTGGCACGCGGCACC | | | | | | | |
| | | GAGTCGGTGC | | | | | | | |
| 3967 | L2_e5_ | GTTTAAGAGCTAGAA | 6485 | GTTTAAGAGCTAGA | 6641 | CTCGAAAGAGCTAC | Stem loop | 5 | 86 |
| | 8 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAGCTCGAAA | | TTATCAGCGTag | | | | | |
| | | GAGCTACGCGGCACC | | | | | | | |
| | | GAGTCGGTGC | | | | | | | |
| 3968 | L2_e5_ | GTTTAAGAGCTAGAA | 6486 | GTTTAAGAGCTAGA | 6642 | GGAGAAATCCTGAC | Stem loop | 5 | 86 |
| | 9 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCAGGAGAAA | | TTATCAGCGTca | | | | | |
| | | TCCTGACGCGGCACC | | | | | | | |
| | | GAGTCGGTGC | | | | | | | |
| 3969 | L2_e5_ | GTTTAAGAGCTAGAA | 6487 | GTTTAAGAGCTAGA | 6643 | GGGGAAACCCGTAC | Stem loop | 5 | 86 |
| | 10 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACGGGGAAA | | TTATCAGCGTac | | | | | |
| | | CCCGTACGCGGCACC | | | | | | | |
| | | GAGTCGGTGC | | | | | | | |
| 3970 | L2_e5_ | GTTTAAGAGCTAGAA | 6488 | GTTTAAGAGCTAGA | 6644 | GGTGAAAGCCTGAC | Stem loop | 5 | 86 |
| | 11 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCAGGTGAAA | | TTATCAGCGTca | | | | | |
| | | GCCTGACGCGGCACC | | | | | | | |
| | | GAGTCGGTGC | | | | | | | |
| 3971 | L2_e5_ | GTTTAAGAGCTAGAA | 6489 | GTTTAAGAGCTAGA | 6645 | CAGGAAACTGGAAC | Stem loop | 5 | 86 |
| | 12 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTCCAGGAAA | | TTATCAGCGTtc | | | | | |
| | | CTGGAACGCGGCACC | | | | | | | |
| | | GAGTCGGTGC | | | | | | | |
| 3972 | L2_e5_ | GTTTAAGAGCTAGAA | 6490 | GTTTAAGAGCTAGA | 6646 | GAGGAAACTCTCAC | Stem loop | 5 | 86 |
| | 13 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGAGAGGAAA | | TTATCAGCGTga | | | | | |
| | | CTCTCACGCGGCACCG | | | | | | | |
| | | AGTCGGTGC | | | | | | | |
| 3973 | L2_e5_ | GTTTAAGAGCTAGAA | 6491 | GTTTAAGAGCTAGA | 6647 | CTCGAAAGAGTGAC | Stem loop | 5 | 86 |
| | 14 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCACTCGAAA | | TTATCAGCGTca | | | | | |
| | | GAGTGACGCGGCACC | | | | | | | |
| | | GAGTCGGTGC | | | | | | | |
| 3974 | L2_e5_ | GTTTAAGAGCTAGAA | 6492 | GTTTAAGAGCTAGA | 6648 | TGCGAAAGCAGCAC | Stem loop | 5 | 86 |
| | 15 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTTGCGAAA | | TTATCAGCGTgt | | | | | |
| | | GCAGCACGCGGCACC | | | | | | | |
| | | GAGTCGGTGC | | | | | | | |
| 3975 | L2_e5_ | GTTTAAGAGCTAGAA | 6493 | GTTTAAGAGCTAGA | 6649 | AGGGAAACCTGCAC | Stem loop | 5 | 86 |
| | 16 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTAGGGAAA | | TTATCAGCGTgt | | | | | |
| | | CCTGCACGCGGCACC | | | | | | | |
| | | GAGTCGGTGC | | | | | | | |
| 3976 | L2_e5_ | GTTTAAGAGCTAGAA | 6494 | GTTTAAGAGCTAGA | 6650 | CCTGAAAGGGACAC | Stem loop | 5 | 86 |
| | 17 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTCCTGAAA | | TTATCAGCGTgt | | | | | |
| | | GGGACACGCGGCACC | | | | | | | |
| | | GAGTCGGTGC | | | | | | | |
| 3977 | L2_e5_ | GTTTAAGAGCTAGAA | 6495 | GTTTAAGAGCTAGA | 6651 | CCCGAAAGGGTTAC | Stem loop | 5 | 86 |
| | 18 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAACCCGAAA | | TTATCAGCGTaa | | | | | |
| | | GGGTTACGCGGCACC | | | | | | | |
| | | GAGTCGGTGC | | | | | | | |
| 3978 | L2_e5_ | GTTTAAGAGCTAGAA | 6496 | GTTTAAGAGCTAGA | 6652 | CCGGAAACGGGTAC | Stem loop | 5 | 86 |
| | 19 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACCCGGAAA | | TTATCAGCGTac | | | | | |
| | | CGGGTACGCGGCACC | | | | | | | |
| | | GAGTCGGTGC | | | | | | | |
| 3979 | L2_e5_ | GTTTAAGAGCTAGAA | 6497 | GTTTAAGAGCTAGA | 6653 | GTTGAAAGACGCAC | Stem loop | 5 | 86 |
| | 20 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCGTTGAAA | | TTATCAGCGTgc | | | | | |
| | | GACGCACGCGGCACC | | | | | | | |
| | | GAGTCGGTGC | | | | | | | |
| 3980 | L2_e5_ | GTTTAAGAGCTAGAA | 6498 | GTTTAAGAGCTAGA | 6654 | CGCGAAAGCGGCAC | Stem loop | 5 | 86 |
| | 21 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCCGCGAAA | | TTATCAGCGTgc | | | | | |
| | | GCGGCACGCGGCACC | | | | | | | |
| | | GAGTCGGTGC | | | | | | | |
| 3981 | L2_e5_ | GTTTAAGAGCTAGAA | 6499 | GTTTAAGAGCTAGA | 6655 | GGGGAAATCCCAAC | Stem loop | 5 | 86 |
| | 22 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTGGGGGAAA | | TTATCAGCGTtg | | | | | |
| | | TCCCAACGCGGCACC | | | | | | | |
| | | GAGTCGGTGC | | | | | | | |
| 3982 | L2_e5_ | GTTTAAGAGCTAGAA | 6500 | GTTTAAGAGCTAGA | 6656 | AGAGAAATCTGCAC | Stem loop | 5 | 86 |
| | 23 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCAGAGAAA | | TTATCAGCGTgc | | | | | |
| | | TCTGCACGCGGCACC | | | | | | | |
| | | GAGTCGGTGC | | | | | | | |
| 3983 | L2_e5_ | GTTTAAGAGCTAGAA | 6501 | GTTTAAGAGCTAGA | 6657 | CGTGAAAACGACAC | Stem loop | 5 | 86 |
| | 24 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTCGTGAAA | | TTATCAGCGTgt | | | | | |
| | | ACGACACGCGGCACC | | | | | | | |
| | | GAGTCGGTGC | | | | | | | |
| 3984 | L2_e5_ | GTTTAAGAGCTAGAA | 6502 | GTTTAAGAGCTAGA | 6658 | TACGAAAGTACGAC | Stem loop | 5 | 86 |
| | 25 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCGTACGAAA | | TTATCAGCGTcg | | | | | |
| | | GTACGACGCGGCACC | | | | | | | |
| | | GAGTCGGTGC | | | | | | | |
| 3985 | L2_e5_ | GTTTAAGAGCTAGAA | 6503 | GTTTAAGAGCTAGA | 6659 | CGCGAAAGCGTGAC | Stem loop | 5 | 86 |
| | 26 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTACGCGAAA | | TTATCAGCGTta | | | | | |
| | | GCGTGACGCGGCACC | | | | | | | |
| | | GAGTCGGTGC | | | | | | | |
| 3986 | L2_e5_ | GTTTAAGAGCTAGAA | 6504 | GTTTAAGAGCTAGA | 6660 | GCGGAAACGTTGAC | Stem loop | 5 | 86 |
| | 27 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCAGCGGAAA | | TTATCAGCGTca | | | | | |
| | | CGTTGACGCGGCACC | | | | | | | |
| | | GAGTCGGTGC | | | | | | | |
| 3987 | L2_e5_ | GTTTAAGAGCTAGAA | 6505 | GTTTAAGAGCTAGA | 6661 | GACGAAAGTCAGAC | Stem loop | 5 | 86 |
| | 28 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTGACGAAA | | TTATCAGCGTct | | | | | |
| | | GTCAGACGCGGCACC | | | | | | | |
| | | GAGTCGGTGC | | | | | | | |
| 3988 | L2_e5_ | GTTTAAGAGCTAGAA | 6506 | GTTTAAGAGCTAGA | 6662 | CGCGAAAGCGAGAC | Stem loop | 5 | 86 |
| | 29 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTCGCGAAA | | TTATCAGCGTct | | | | | |
| | | GCGAGACGCGGCACC | | | | | | | |
| | | GAGTCGGTGC | | | | | | | |
| 3989 | L2_e5_ | GTTTAAGAGCTAGAA | 6507 | GTTTAAGAGCTAGA | 6663 | GGCGAAAGCCTAAC | Stem loop | 5 | 86 |
| | 30 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTGGGCGAAA | | TTATCAGCGTtg | | | | | |
| | | GCCTAACGCGGCACC | | | | | | | |
| | | GAGTCGGTGC | | | | | | | |
| 3990 | L2_e5_ | GTTTAAGAGCTAGAA | 6508 | GTTTAAGAGCTAGA | 6664 | AGGGAAACCTGGAC | Stem loop | 5 | 86 |
| | 31 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCAGGGAAA | | TTATCAGCGTcc | | | | | |
| | | CCTGGACGCGGCACC | | | | | | | |
| | | GAGTCGGTGC | | | | | | | |
| 3991 | L2_e5_ | GTTTAAGAGCTAGAA | 6509 | GTTTAAGAGCTAGA | 6665 | CCTGAAAGGGGTAC | Stem loop | 5 | 86 |
| | 32 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACCCTGAAA | | TTATCAGCGTac | | | | | |
| | | GGGGTACGCGGCACC | | | | | | | |
| | | GAGTCGGTGC | | | | | | | |
| 3992 | L2_e5_ | GTTTAAGAGCTAGAA | 6510 | GTTTAAGAGCTAGA | 6666 | CCGGAAACGGGGAC | Stem loop | 5 | 86 |
| | 33 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTCCCGGAAA | | TTATCAGCGTtc | | | | | |
| | | CGGGGACGCGGCACC | | | | | | | |
| | | GAGTCGGTGC | | | | | | | |
| 3993 | L2_e5_ | GTTTAAGAGCTAGAA | 6511 | GTTTAAGAGCTAGA | 6667 | ACGGAAACGTGTAC | Stem loop | 5 | 86 |
| | 34 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACACGGAAA | | TTATCAGCGTac | | | | | |
| | | CGTGTACGCGGCACC | | | | | | | |
| | | GAGTCGGTGC | | | | | | | |
| 3994 | L2_e5_ | GTTTAAGAGCTAGAA | 6512 | GTTTAAGAGCTAGA | 6668 | ACGGAAACGTGGAC | Stem loop | 5 | 86 |
| | 35 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCACGGAAA | | TTATCAGCGTcc | | | | | |
| | | CGTGGACGCGGCACC | | | | | | | |
| | | GAGTCGGTGC | | | | | | | |
| 3995 | L2_e5_ | GTTTAAGAGCTAGAA | 6513 | GTTTAAGAGCTAGA | 6669 | CATGAAAGTGGCAC | Stem loop | 5 | 86 |
| | 36 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCCATGAAA | | TTATCAGCGTgc | | | | | |
| | | GTGGCACGCGGCACC | | | | | | | |
| | | GAGTCGGTGC | | | | | | | |
| 3996 | L2_e5_ | GTTTAAGAGCTAGAA | 6514 | GTTTAAGAGCTAGA | 6670 | CTCGAAAGAGGTAC | Stem loop | 5 | 86 |
| | 37 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACCTCGAAA | | TTATCAGCGTac | | | | | |
| | | GAGGTACGCGGCACC | | | | | | | |
| | | GAGTCGGTGC | | | | | | | |
| 3997 | L2_e5_ | GTTTAAGAGCTAGAA | 6515 | GTTTAAGAGCTAGA | 6671 | CTCGAAAGAGGCAC | Stem loop | 5 | 86 |
| | 38 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTCTCGAAA | | TTATCAGCGTgt | | | | | |
| | | GAGGCACGCGGCACC | | | | | | | |
| | | GAGTCGGTGC | | | | | | | |
| 3998 | L2_e5_ | GTTTAAGAGCTAGAA | 6516 | GTTTAAGAGCTAGA | 6672 | ACCGAAAGGTCGAC | Stem loop | 5 | 86 |
| | 39 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCGACCGAAA | | TTATCAGCGTcg | | | | | |
| | | GGTCGACGCGGCACC | | | | | | | |
| | | GAGTCGGTGC | | | | | | | |
| 3999 | L2_e5_ | GTTTAAGAGCTAGAA | 6517 | GTTTAAGAGCTAGA | 6673 | GACGAAAGTCTGAC | Stem loop | 5 | 86 |
| | 40 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCAGACGAAA | | TTATCAGCGTca | | | | | |
| | | GTCTGACGCGGCACC | | | | | | | |
| | | GAGTCGGTGC | | | | | | | |
| 4000 | L2_e5_ | GTTTAAGAGCTAGAA | 6518 | GTTTAAGAGCTAGA | 6674 | TGGGAAACCGTCAC | Stem loop | 5 | 86 |
| | 41 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGATGGGAAA | | TTATCAGCGTga | | | | | |
| | | CCGTCACGCGGCACC | | | | | | | |
| | | GAGTCGGTGC | | | | | | | |
| 4001 | L2_e5_ | GTTTAAGAGCTAGAA | 6519 | GTTTAAGAGCTAGA | 6675 | GGGGAAACCCCTAC | Stem loop | 5 | 86 |
| | 42 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAGGGGGAAA | | TTATCAGCGTag | | | | | |
| | | CCCCTACGCGGCACC | | | | | | | |
| | | GAGTCGGTGC | | | | | | | |
| 4002 | L2_e5_ | GTTTAAGAGCTAGAA | 6520 | GTTTAAGAGCTAGA | 6676 | CACGAAAGTGGTAC | Stem loop | 5 | 86 |
| | 43 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACCACGAAA | | TTATCAGCGTac | | | | | |
| | | GTGGTACGCGGCACC | | | | | | | |
| | | GAGTCGGTGC | | | | | | | |
| 4003 | L2_e5_ | GTTTAAGAGCTAGAA | 6521 | GTTTAAGAGCTAGA | 6677 | GACGAAAGTCGCAC | Stem loop | 5 | 86 |
| | 44 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCGACGAAA | | TTATCAGCGTgc | | | | | |
| | | GTCGCACGCGGCACC | | | | | | | |
| | | GAGTCGGTGC | | | | | | | |
| 4004 | L2_e5_ | GTTTAAGAGCTAGAA | 6522 | GTTTAAGAGCTAGA | 6678 | GGGGAAACCCCGAC | Stem loop | 5 | 86 |
| | 45 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCGGGGGAAA | | TTATCAGCGTcg | | | | | |
| | | CCCCGACGCGGCACC | | | | | | | |
| | | GAGTCGGTGC | | | | | | | |
| 4005 | L2_e5_ | GTTTAAGAGCTAGAA | 6523 | GTTTAAGAGCTAGA | 6679 | CGAGAAATCGGTAC | Stem loop | 5 | 86 |
| | 46 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACCGAGAAA | | TTATCAGCGTac | | | | | |
| | | TCGGTACGCGGCACC | | | | | | | |
| | | GAGTCGGTGC | | | | | | | |
| 4006 | L2_e5_ | GTTTAAGAGCTAGAA | 6524 | GTTTAAGAGCTAGA | 6680 | TCGGAAACGACGAC | Stem loop | 5 | 86 |
| | 47 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTGTCGGAAA | | TTATCAGCGTtg | | | | | |
| | | CGACGACGCGGCACC | | | | | | | |
| | | GAGTCGGTGC | | | | | | | |
| 4007 | L2_e5_ | GTTTAAGAGCTAGAA | 6525 | GTTTAAGAGCTAGA | 6681 | ACGGAAATGTCCAC | Stem loop | 5 | 86 |
| | 48 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGGACGGAAA | | TTATCAGCGTgg | | | | | |
| | | TGTCCACGCGGCACC | | | | | | | |
| | | GAGTCGGTGC | | | | | | | |
| 4008 | L2_e5_ | GTTTAAGAGCTAGAA | 6526 | GTTTAAGAGCTAGA | 6682 | GGCGAAAGCCTGAC | Stem loop | 5 | 86 |
| | 49 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCAGGCGAAA | | TTATCAGCGTca | | | | | |
| | | GCCTGACGCGGCACC | | | | | | | |
| | | GAGTCGGTGC | | | | | | | |
| 4009 | L2_e5_ | GTTTAAGAGCTAGAA | 6527 | GTTTAAGAGCTAGA | 6683 | TCCGAAAGGAAGAC | Stem loop | 5 | 86 |
| | 50 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTTCCGAAA | | TTATCAGCGTct | | | | | |
| | | GGAAGACGCGGCACC | | | | | | | |
| | | GAGTCGGTGC | | | | | | | |
| 4010 | L2_e5_ | GTTTAAGAGCTAGAA | 6528 | GTTTAAGAGCTAGA | 6684 | CGAGAAATCGGAAC | Stem loop | 5 | 86 |
| | 51 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTCCGAGAAA | | TTATCAGCGTtc | | | | | |
| | | TCGGAACGCGGCACC | | | | | | | |
| | | GAGTCGGTGC | | | | | | | |
| 4011 | L2_e5_ | GTTTAAGAGCTAGAA | 6529 | GTTTAAGAGCTAGA | 6685 | GCCGAAAGGTGTAC | Stem loop | 5 | 86 |
| | 52 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACGCCGAAA | | TTATCAGCGTac | | | | | |
| | | GGTGTACGCGGCACC | | | | | | | |
| | | GAGTCGGTGC | | | | | | | |
| 4012 | L2_e5_ | GTTTAAGAGCTAGAA | 6530 | GTTTAAGAGCTAGA | 6686 | CGTGAAAACGCTAC | Stem loop | 5 | 86 |
| | 53 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGGCGTGAAA | | TTATCAGCGTgg | | | | | |
| | | ACGCTACGCGGCACC | | | | | | | |
| | | GAGTCGGTGC | | | | | | | |
| 4013 | L2_e5_ | GTTTAAGAGCTAGAA | 6531 | GTTTAAGAGCTAGA | 6687 | GACGAAAGTCCTAC | Stem loop | 5 | 86 |
| | 54 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGGGACGAAA | | TTATCAGCGTgg | | | | | |
| | | GTCCTACGCGGCACC | | | | | | | |
| | | GAGTCGGTGC | | | | | | | |
| 4014 | L2_e5_ | GTTTAAGAGCTAGAA | 6532 | GTTTAAGAGCTAGA | 6688 | GGGGAAACCCAGAC | Stem loop | 5 | 86 |
| | 55 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTGGGGAAA | | TTATCAGCGTct | | | | | |
| | | CCCAGACGCGGCACC | | | | | | | |
| | | GAGTCGGTGC | | | | | | | |
| 4015 | L2_e5_ | GTTTAAGAGCTAGAA | 6533 | GTTTAAGAGCTAGA | 6689 | ATCGAAAGATGCAC | Stem loop | 5 | 86 |
| | 56 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCATCGAAA | | TTATCAGCGTgc | | | | | |
| | | GATGCACGCGGCACC | | | | | | | |
| | | GAGTCGGTGC | | | | | | | |
| 4016 | L2_e5_ | GTTTAAGAGCTAGAA | 6534 | GTTTAAGAGCTAGA | 6690 | CGCGAAAGCGTTAC | Stem loop | 5 | 86 |
| | 57 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAACGCGAAA | | TTATCAGCGTaa | | | | | |
| | | GCGTTACGCGGCACC | | | | | | | |
| | | GAGTCGGTGC | | | | | | | |
| 4017 | L2_e5_ | GTTTAAGAGCTAGAA | 6535 | GTTTAAGAGCTAGA | 6691 | CAGGAAACTGGCAC | Stem loop | 5 | 86 |
| | 58 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCCAGGAAA | | TTATCAGCGTgc | | | | | |
| | | CTGGCACGCGGCACC | | | | | | | |
| | | GAGTCGGTGC | | | | | | | |
| 4018 | L2_e5_ | GTTTAAGAGCTAGAA | 6536 | GTTTAAGAGCTAGA | 6692 | ACGGAAACGTGAAC | Stem loop | 5 | 86 |
| | 59 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTCACGGAAA | | TTATCAGCGTtc | | | | | |
| | | CGTGAACGCGGCACC | | | | | | | |
| | | GAGTCGGTGC | | | | | | | |
| 4019 | L2_e5_ | GTTTAAGAGCTAGAA | 6537 | GTTTAAGAGCTAGA | 6693 | GACGAAAGTCGTAC | Stem loop | 5 | 86 |
| | 60 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACGACGAAA | | TTATCAGCGTac | | | | | |
| | | GTCGTACGCGGCACC | | | | | | | |
| | | GAGTCGGTGC | | | | | | | |
| 4020 | L2_e5_ | GTTTAAGAGCTAGAA | 6538 | GTTTAAGAGCTAGA | 6694 | TGGGAAACCAGCAC | Stem loop | 5 | 86 |
| | 61 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCTGGGAAA | | TTATCAGCGTgc | | | | | |
| | | CCAGCACGCGGCACC | | | | | | | |
| | | GAGTCGGTGC | | | | | | | |
| 4021 | L2_e5_ | GTTTAAGAGCTAGAA | 6539 | GTTTAAGAGCTAGA | 6695 | CCAGAAATGGGGAC | Stem loop | 5 | 86 |
| | 62 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTCCAGAAA | | TTATCAGCGTct | | | | | |
| | | TGGGGACGCGGCACC | | | | | | | |
| | | GAGTCGGTGC | | | | | | | |
| 4022 | L2_e5_ | GTTTAAGAGCTAGAA | 6540 | GTTTAAGAGCTAGA | 6696 | GGGGAAACCCCAAC | Stem loop | 5 | 86 |
| | 63 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTGGGGGAAA | | TTATCAGCGTtg | | | | | |
| | | CCCCAACGCGGCACC | | | | | | | |
| | | GAGTCGGTGC | | | | | | | |
| 4023 | L2_e5_ | GTTTAAGAGCTAGAA | 6541 | GTTTAAGAGCTAGA | 6697 | CCCGAAAGGGCTAC | Stem loop | 5 | 86 |
| | 64 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAGCCCGAAA | | TTATCAGCGTag | | | | | |
| | | GGGCTACGCGGCACC | | | | | | | |
| | | GAGTCGGTGC | | | | | | | |
| 4024 | L2_e5_ | GTTTAAGAGCTAGAA | 6542 | GTTTAAGAGCTAGA | 6698 | CCAGAAATGGGTAC | Stem loop | 5 | 86 |
| | 65 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTACCCAGAAA | | TTATCAGCGTac | | | | | |
| | | TGGGTACGCGGCACC | | | | | | | |
| | | GAGTCGGTGC | | | | | | | |
| 4025 | L2_e5_ | GTTTAAGAGCTAGAA | 6543 | GTTTAAGAGCTAGA | 6699 | CGTGAAAGCGCTAC | Stem loop | 5 | 86 |
| | 66 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAGCGTGAAA | | TTATCAGCGTag | | | | | |
| | | GCGCTACGCGGCACC | | | | | | | |
| | | GAGTCGGTGC | | | | | | | |
| 4026 | L2_e5_ | GTTTAAGAGCTAGAA | 6544 | GTTTAAGAGCTAGA | 6700 | CTAGAAATAGGCAC | Stem loop | 5 | 86 |
| | 67 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCCTAGAAA | | TTATCAGCGTgc | | | | | |
| | | TAGGCACGCGGCACC | | | | | | | |
| | | GAGTCGGTGC | | | | | | | |
| 4027 | L2_e5_ | GTTTAAGAGCTAGAA | 6545 | GTTTAAGAGCTAGA | 6701 | GTCGAAAGACAGAC | Stem loop | 5 | 86 |
| | 68 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTGTCGAAA | | TTATCAGCGTct | | | | | |
| | | GACAGACGCGGCACC | | | | | | | |
| | | GAGTCGGTGC | | | | | | | |
| 4028 | L2_e5_ | GTTTAAGAGCTAGAA | 6546 | GTTTAAGAGCTAGA | 6649 | AGGGAAACCTGCAC | Stem loop | 5 | 86 |
| | 69 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCAGGGAAA | | TTATCAGCGTgc | | | | | |
| | | CCTGCACGCGGCACC | | | | | | | |
| | | GAGTCGGTGC | | | | | | | |
| 4029 | L2_e5_ | GTTTAAGAGCTAGAA | 6547 | GTTTAAGAGCTAGA | 6702 | AGGGAAACCTACAC | Stem loop | 5 | 86 |
| | 70 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTAGGGAAA | | TTATCAGCGTgt | | | | | |
| | | CCTACACGCGGCACC | | | | | | | |
| | | GAGTCGGTGC | | | | | | | |
| 4030 | L2_e5_ | GTTTAAGAGCTAGAA | 6548 | GTTTAAGAGCTAGA | 6703 | GCTGAAAGGCGGAC | Stem loop | 5 | 86 |
| | 71 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCGCTGAAA | | TTATCAGCGTcc | | | | | |
| | | GGCGGACGCGGCACC | | | | | | | |
| | | GAGTCGGTGC | | | | | | | |
| 4031 | L2_e5_ | GTTTAAGAGCTAGAA | 6549 | GTTTAAGAGCTAGA | 6704 | GTCGAAAGGCACAC | Stem loop | 5 | 86 |
| | 72 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTGTCGAAA | | TTATCAGCGTgt | | | | | |
| | | GGCACACGCGGCACC | | | | | | | |
| | | GAGTCGGTGC | | | | | | | |
| 4032 | L2_e5_ | GTTTAAGAGCTAGAA | 6550 | GTTTAAGAGCTAGA | 6705 | TACGAAAGTGGGAC | Stem loop | 5 | 86 |
| | 73 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCTACGAAA | | TTATCAGCGTcc | | | | | |
| | | GTGGGACGCGGCACC | | | | | | | |
| | | GAGTCGGTGC | | | | | | | |
| 4033 | L2_e5_ | GTTTAAGAGCTAGAA | 6551 | GTTTAAGAGCTAGA | 6706 | GGGGAAACCCACAC | Stem loop | 5 | 86 |
| | 74 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTGGGGAAA | | TTATCAGCGTgt | | | | | |
| | | CCCACACGCGGCACC | | | | | | | |
| | | GAGTCGGTGC | | | | | | | |
| 4034 | L2_e5_ | GTTTAAGAGCTAGAA | 6552 | GTTTAAGAGCTAGA | 6707 | GGTGAAAACTCGAC | Stem loop | 5 | 86 |
| | 75 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCGGGTGAAA | | TTATCAGCGTcg | | | | | |
| | | ACTCGACGCGGCACC | | | | | | | |
| | | GAGTCGGTGC | | | | | | | |
| 4035 | L2_e5_ | GTTTAAGAGCTAGAA | 6553 | GTTTAAGAGCTAGA | 6708 | TCGGAAACGACAAC | Stem loop | 5 | 86 |
| | 76 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTGTCGGAAA | | TTATCAGCGTtg | | | | | |
| | | CGACAACGCGGCACC | | | | | | | |
| | | GAGTCGGTGC | | | | | | | |
| 4036 | L2_e5_ | GTTTAAGAGCTAGAA | 6554 | GTTTAAGAGCTAGA | 6709 | CCCGAAAGGGGGAC | Stem loop | 5 | 86 |
| | 77 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCCCCGAAA | | TTATCAGCGTcc | | | | | |
| | | GGGGGACGCGGCACC | | | | | | | |
| | | GAGTCGGTGC | | | | | | | |
| 4037 | L2_e5_ | GTTTAAGAGCTAGAA | 6555 | GTTTAAGAGCTAGA | 6710 | GGCGAAAGCCCAAC | Stem loop | 5 | 86 |
| | 78 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTGGGCGAAA | | TTATCAGCGTtg | | | | | |
| | | GCCCAACGCGGCACC | | | | | | | |
| | | GAGTCGGTGC | | | | | | | |
| 4038 | L2_e5_ | GTTTAAGAGCTAGAA | 6556 | GTTTAAGAGCTAGA | 6711 | ACCGAAAGGTTCAC | Stem loop | 5 | 86 |
| | 79 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGGACCGAAA | | TTATCAGCGTgg | | | | | |
| | | GGTTCACGCGGCACC | | | | | | | |
| | | GAGTCGGTGC | | | | | | | |
| 4039 | L2_e5_ | GTTTAAGAGCTAGAA | 6557 | GTTTAAGAGCTAGA | 6712 | CGCGAAAGCGATAC | Stem loop | 5 | 86 |
| | 80 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTCGCGAAA | | TTATCAGCGTgt | | | | | |
| | | GCGATACGCGGCACC | | | | | | | |
| | | GAGTCGGTGC | | | | | | | |
| 4040 | L2_e5_ | GTTTAAGAGCTAGAA | 6558 | GTTTAAGAGCTAGA | 6713 | TGCGAAAGCAGGAC | Stem loop | 5 | 86 |
| | 81 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCTGCGAAA | | TTATCAGCGTcc | | | | | |
| | | GCAGGACGCGGCACC | | | | | | | |
| | | GAGTCGGTGC | | | | | | | |
| 4041 | L2_e5_ | GTTTAAGAGCTAGAA | 6559 | GTTTAAGAGCTAGA | 6714 | CCTGAAAAGGTCAC | Stem loop | 5 | 86 |
| | 82 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGACCTGAAA | | TTATCAGCGTga | | | | | |
| | | AGGTCACGCGGCACC | | | | | | | |
| | | GAGTCGGTGC | | | | | | | |
| 4042 | L2_e5_ | GTTTAAGAGCTAGAA | 6560 | GTTTAAGAGCTAGA | 6715 | ACCGAAAGGTGCAC | Stem loop | 5 | 86 |
| | 83 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGCACCGAAA | | TTATCAGCGTgc | | | | | |
| | | GGTGCACGCGGCACC | | | | | | | |
| | | GAGTCGGTGC | | | | | | | |
| 4043 | L2_e5_ | GTTTAAGAGCTAGAA | 6561 | GTTTAAGAGCTAGA | 6716 | GGCGAAAGCCCTAC | Stem loop | 5 | 86 |
| | 84 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTAGGGCGAAA | | TTATCAGCGTag | | | | | |
| | | GCCCTACGCGGCACC | | | | | | | |
| | | GAGTCGGTGC | | | | | | | |
| 4044 | L2_e5_ | GTTTAAGAGCTAGAA | 6562 | GTTTAAGAGCTAGA | 6717 | TCGGAAACGGTCAC | Stem loop | 5 | 86 |
| | 85 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGATCGGAAA | | TTATCAGCGTga | | | | | |
| | | CGGTCACGCGGCACC | | | | | | | |
| | | GAGTCGGTGC | | | | | | | |
| 4045 | L2_e5_ | GTTTAAGAGCTAGAA | 6563 | GTTTAAGAGCTAGA | 6718 | CGTGAAAGCGTGAC | Stem loop | 5 | 86 |
| | 86 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCGCGTGAAA | | TTATCAGCGTcg | | | | | |
| | | GCGTGACGCGGCACC | | | | | | | |
| | | GAGTCGGTGC | | | | | | | |
| 4046 | L2_e5_ | GTTTAAGAGCTAGAA | 6564 | GTTTAAGAGCTAGA | 6712 | CGCGAAAGCGATAC | Stem loop | 5 | 86 |
| | 87 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTATCGCGAAA | | TTATCAGCGTat | | | | | |
| | | GCGATACGCGGCACC | | | | | | | |
| | | GAGTCGGTGC | | | | | | | |
| 4047 | L2_e5_ | GTTTAAGAGCTAGAA | 6565 | GTTTAAGAGCTAGA | 6719 | CAGGAAACTGGGAC | Stem loop | 5 | 86 |
| | 88 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCCAGGAAA | | TTATCAGCGTcc | | | | | |
| | | CTGGGACGCGGCACC | | | | | | | |
| | | GAGTCGGTGC | | | | | | | |
| 4048 | L2_e5_ | GTTTAAGAGCTAGAA | 6566 | GTTTAAGAGCTAGA | 6720 | GCCGAAAGGCGGAC | Stem loop | 5 | 86 |
| | 89 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCCGCCGAAA | | TTATCAGCGTcc | | | | | |
| | | GGCGGACGCGGCACC | | | | | | | |
| | | GAGTCGGTGC | | | | | | | |
| 4049 | L2_e5_ | GTTTAAGAGCTAGAA | 6567 | GTTTAAGAGCTAGA | 6659 | CGCGAAAGCGTGAC | Stem loop | 5 | 86 |
| | 90 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCACGCGAAA | | TTATCAGCGTca | | | | | |
| | | GCGTGACGCGGCACC | | | | | | | |
| | | GAGTCGGTGC | | | | | | | |
| 4050 | L2_e5_ | GTTTAAGAGCTAGAA | 6568 | GTTTAAGAGCTAGA | 6721 | GCAGAAATGCAGAC | Stem loop | 5 | 86 |
| | 91 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTCTGCAGAAA | | TTATCAGCGTct | | | | | |
| | | TGCAGACGCGGCACC | | | | | | | |
| | | GAGTCGGTGC | | | | | | | |
| 4051 | L2_e5_ | GTTTAAGAGCTAGAA | 6569 | GTTTAAGAGCTAGA | 6722 | TAGGAAACTACCAC | Stem loop | 5 | 86 |
| | 92 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGGTAGGAAA | | TTATCAGCGTgg | | | | | |
| | | CTACCACGCGGCACC | | | | | | | |
| | | GAGTCGGTGC | | | | | | | |
| 4052 | L2_e5_ | GTTTAAGAGCTAGAA | 6570 | GTTTAAGAGCTAGA | 6723 | CCTGAAAAGGCCAC | Stem loop | 5 | 86 |
| | 93 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGGCCTGAAA | | TTATCAGCGTgg | | | | | |
| | | AGGCCACGCGGCACC | | | | | | | |
| | | GAGTCGGTGC | | | | | | | |
| 4053 | L2_e5_ | GTTTAAGAGCTAGAA | 6571 | GTTTAAGAGCTAGA | 6724 | GACGAAAGTCTCAC | Stem loop | 5 | 86 |
| | 94 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGAGACGAAA | | TTATCAGCGTga | | | | | |
| | | GTCTCACGCGGCACC | | | | | | | |
| | | GAGTCGGTGC | | | | | | | |
| 4054 | L2_e5_ | GTTTAAGAGCTAGAA | 6572 | GTTTAAGAGCTAGA | 6725 | ACCGAAAGGTGGAC | Stem loop | 5 | 86 |
| | 95 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTCACCGAAA | | TTATCAGCGTtc | | | | | |
| | | GGTGGACGCGGCACC | | | | | | | |
| | | GAGTCGGTGC | | | | | | | |
| 4055 | L2_e5_ | GTTTAAGAGCTAGAA | 6573 | GTTTAAGAGCTAGA | 6726 | TCCGAAAGGAACAC | Stem loop | 5 | 86 |
| | 96 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTTCCGAAA | | TTATCAGCGTgt | | | | | |
| | | GGAACACGCGGCACC | | | | | | | |
| | | GAGTCGGTGC | | | | | | | |
| 4056 | L2_e5_ | GTTTAAGAGCTAGAA | 6574 | GTTTAAGAGCTAGA | 672.7 | GCGGAAACGCGTAC | Stem loop | 5 | 86 |
| | 97 | ATAGCAAGTTTAAAT | | AATAGCAAGTTTAA | | GCGGCACCGAGTCG | 7 | | |
| | | AAGGCTAGTCCGTTAT | | ATAAGGCTAGTCCG | | GTGC | | | |
| | | CAGCGTGCGCGGAAA | | TTATCAGCGTor | | | | | |
| | | CGCGTACGCGGCACC | | | | | | | |
| | | GAGTOGGTGC | | | | | | | |
| 4057 | L2_e5_ | GTTTAAGAGCTAGAA | 6575 | GTTTAAGAGCTAGA | 6728 | ACCGAAAGGTGAAC | Stem loop | 5 | 86 |
| | 98 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTTCACCGAAA | | TTATCAGCGTtc | | | | | |
| | | GGTGAACGCGGCACC | | | | | | | |
| | | GAGTCGGTGC | | | | | | | |
| 4058 | L2_e5_ | GTTTAAGAGCTAGAA | 6576 | GTTTAAGAGCTAGA | 6651 | CCCGAAAGGGTTAC | Stem loop | 5 | 86 |
| | 99 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGACCCGAAA | | TTATCAGCGTga | | | | | |
| | | GGGTTACGCGGCACC | | | | | | | |
| | | GAGTCGGTGC | | | | | | | |
| 4059 | L2_e5_ | GTTTAAGAGCTAGAA | 6577 | GTTTAAGAGCTAGA | 6729 | ACGGAAACGTGCAC | Stem loop | 5 | 86 |
| | 100 | ATAGCAAGTTTAAAT | | AATAGCAAGTTTAA | | GCGGCACCGAGTCG | 2 | | |
| | | AAGGCTAGTCCGTTAT | | ATAAGGCTAGTCCG | | GTGC | | | |
| | | CAGCGTGTACGGAAA | | TTATCAGCGTgt | | | | | |
| | | CGTGCACGCGGCACC | | | | | | | |
| | | GAGTCGGTGC | | | | | | | |
| 4060 | L2_e6_ | GTTTAAGAGCTAGAA | 6578 | GTTTAAGAGCTAGA | 6730 | GCGGAAACGCATGA | Stem loop | 6 | 88 |
| | 1 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCATGCGGAA | | TTATCAGCGTcat | | | | | |
| | | ACGCATGACGCGGCA | | | | | | | |
| | | CCGAGTCGGTGC | | | | | | | |
| 4061 | L2_e6_ | GTTTAAGAGCTAGAA | 6579 | GTTTAAGAGCTAGA | 6731 | GTTGAAAAACCCCA | Stem loop | 6 | 88 |
| | 2 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGGGTTGAA | | TTATCAGCGTggg | | | | | |
| | | AAACCCCACGCGGCA | | | | | | | |
| | | CCGAGTCGGTGC | | | | | | | |
| 4062 | L2_e6_ | GTTTAAGAGCTAGAA | 6580 | GTTTAAGAGCTAGA | 6732 | AGCGAAAGCTGTCA | Stem loop | 6 | 88 |
| | 3 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGACAGCGAA | | TTATCAGCGTgac | | | | | |
| | | AGCTGTCACGCGGCA | | | | | | | |
| | | CCGAGTCGGTGC | | | | | | | |
| 4063 | L2_e6_ | GTTTAAGAGCTAGAA | 6581 | GTTTAAGAGCTAGA | 6733 | GTAGAAATACGCTA | Stem loop | 6 | 88 |
| | 4 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGCGTAGAA | | TTATCAGCGTggc | | | | | |
| | | ATACGCTACGCGGCA | | | | | | | |
| | | CCGAGTCGGTGC | | | | | | | |
| 4064 | L2_e6_ | GTTTAAGAGCTAGAA | 6582 | GTTTAAGAGCTAGA | 6734 | ACGGAAACGTTGCA | Stem loop | 6 | 88 |
| | 5 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGTAACGGAA | | TTATCAGCGTgta | | | | | |
| | | ACGTTGCACGCGGCA | | | | | | | |
| | | CCGAGTCGGTGC | | | | | | | |
| 4065 | L2_e6_ | GTTTAAGAGCTAGAA | 6583 | GTTTAAGAGCTAGA | 6735 | CACGAAAGTGACGA | Stem loop | 6 | 88 |
| | 6 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCGTCACGAA | | TTATCAGCGTcgt | | | | | |
| | | AGTGACGACGCGGCA | | | | | | | |
| | | CCGAGTCGGTGC | | | | | | | |
| 4066 | L2_e6_ | GTTTAAGAGCTAGAA | 6584 | GTTTAAGAGCTAGA | 6736 | ACCGAAAGGTGTGA | Stem loop | 6 | 88 |
| | 7 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTACACCGAA | | TTATCAGCGTtac | | | | | |
| | | AGGTGTGACGCGGCA | | | | | | | |
| | | CCGAGTCGGTGC | | | | | | | |
| 4067 | L2_e6_ | GTTTAAGAGCTAGAA | 6585 | GTTTAAGAGCTAGA | 6737 | GCGGAAACGCGTCA | Stem loop | 6 | 88 |
| | 8 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGACGCGGAA | | TTATCAGCGTgac | | | | | |
| | | ACGCGTCACGCGGCA | | | | | | | |
| | | CCGAGTCGGTGC | | | | | | | |
| 4068 | L2_e6_ | GTTTAAGAGCTAGAA | 6586 | GTTTAAGAGCTAGA | 6738 | GCCGAAAGGCAACA | Stem loop | 6 | 88 |
| | 9 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGTTGCCGAA | | TTATCAGCGTgtt | | | | | |
| | | AGGCAACACGCGGCA | | | | | | | |
| | | CCGAGTCGGTGC | | | | | | | |
| 4069 | L2_e6_ | GTTTAAGAGCTAGAA | 6587 | GTTTAAGAGCTAGA | 6739 | GGCGAAAGCCTTAA | Stem loop | 6 | 88 |
| | 10 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTAAGGCGAA | | TTATCAGCGTtaa | | | | | |
| | | AGCCTTAACGCGGCA | | | | | | | |
| | | CCGAGTCGGTGC | | | | | | | |
| 4070 | L2_e6_ | GTTTAAGAGCTAGAA | 6588 | GTTTAAGAGCTAGA | 6740 | CGGGAAACCGGGGA | Stem loop | 6 | 88 |
| | 11 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCTCCGGGAA | | TTATCAGCGTctc | | | | | |
| | | ACCGGGGACGCGGCA | | | | | | | |
| | | CCGAGTCGGTGC | | | | | | | |
| 4071 | L2_e6_ | GTTTAAGAGCTAGAA | 6589 | GTTTAAGAGCTAGA | 6741 | TCGGAAACGATAGA | Stem loop | 6 | 88 |
| | 12 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCTATCGGAA | | TTATCAGCGTcta | | | | | |
| | | ACGATAGACGCGGCA | | | | | | | |
| | | CCGAGTCGGTGC | | | | | | | |
| 4072 | L2_e6_ | GTTTAAGAGCTAGAA | 6590 | GTTTAAGAGCTAGA | 6742 | GGTGAAAGCCGGGA | Stem loop | 6 | 88 |
| | 13 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCCGGTGAA | | TTATCAGCGTccc | | | | | |
| | | AGCCGGGACGCGGCA | | | | | | | |
| | | CCGAGTCGGTGC | | | | | | | |
| 4073 | L2_e6_ | GTTTAAGAGCTAGAA | 6591 | GTTTAAGAGCTAGA | 6743 | TATGAAAATAGGGA | Stem loop | 6 | 88 |
| | 14 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCCTATGAA | | TTATCAGCGTccc | | | | | |
| | | AATAGGGACGCGGCA | | | | | | | |
| | | CCGAGTCGGTGC | | | | | | | |
| 4074 | L2_e6_ | GTTTAAGAGCTAGAA | 6592 | GTTTAAGAGCTAGA | 6744 | GCGGAAACGCTTGA | Stem loop | 6 | 88 |
| | 15 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCAAGCGGAA | | TTATCAGCGTcaa | | | | | |
| | | ACGCTTGACGCGGCA | | | | | | | |
| | | CCGAGTCGGTGC | | | | | | | |
| 4075 | L2_e6_ | GTTTAAGAGCTAGAA | 6593 | GTTTAAGAGCTAGA | 6745 | GCGGAAACGCTCTA | Stem loop | 6 | 88 |
| | 16 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGGGCGGAA | | TTATCAGCGTagg | | | | | |
| | | ACGCTCTACGCGGCA | | | | | | | |
| | | CCGAGTCGGTGC | | | | | | | |
| 4076 | L2_e6_ | GTTTAAGAGCTAGAA | 6594 | GTTTAAGAGCTAGA | 6740 | CGGGAAACCGGGGA | Stem loop | 6 | 88 |
| | 17 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCTCGGGAA | | TTATCAGCGTcct | | | | | |
| | | ACCGGGGACGCGGCA | | | | | | | |
| | | CCGAGTCGGTGC | | | | | | | |
| 4077 | L2_e6_ | GTTTAAGAGCTAGAA | 6595 | GTTTAAGAGCTAGA | 6746 | ACGGAAACGTGGAA | Stem loop | 6 | 88 |
| | 18 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTCCACGGAA | | TTATCAGCGTtcc | | | | | |
| | | ACGTGGAACGCGGCA | | | | | | | |
| | | CCGAGTCGGTGC | | | | | | | |
| 4078 | L2_e6_ | GTTTAAGAGCTAGAA | 6596 | GTTTAAGAGCTAGA | 6747 | ATCGAAAGATCCCA | Stem loop | 6 | 88 |
| | 19 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGGATCGAA | | TTATCAGCGTggg | | | | | |
| | | AGATCCCACGCGGCA | | | | | | | |
| | | CCGAGTCGGTGC | | | | | | | |
| 4079 | L2_e6_ | GTTTAAGAGCTAGAA | 6597 | GTTTAAGAGCTAGA | 6748 | CTTGAAAGAGGCCA | Stem loop | 6 | 88 |
| | 20 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGTCTTGAA | | TTATCAGCGTggt | | | | | |
| | | AGAGGCCACGCGGCA | | | | | | | |
| | | CCGAGTCGGTGC | | | | | | | |
| 4080 | L2_e6_ | GTTTAAGAGCTAGAA | 6598 | GTTTAAGAGCTAGA | 6749 | GACGAAAGTTCTCA | Stem loop | 6 | 88 |
| | 21 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGAGGACGAA | | TTATCAGCGTgag | | | | | |
| | | AGTTCTCACGCGGCAC | | | | | | | |
| | | CGAGTCGGTGC | | | | | | | |
| 4081 | L2_e6_ | GTTTAAGAGCTAGAA | 6599 | GTTTAAGAGCTAGA | 6750 | GCAGAAATGCGGTA | Stem loop | 6 | 88 |
| | 22 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCCGCAGAA | | TTATCAGCGTgcc | | | | | |
| | | ATGCGGTACGCGGCA | | | | | | | |
| | | CCGAGTCGGTGC | | | | | | | |
| 4082 | L2_e6_ | GTTTAAGAGCTAGAA | 6600 | GTTTAAGAGCTAGA | 6751 | ACCGAAAGGTCTTA | Stem loop | 6 | 88 |
| | 23 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAAGACCGAA | | TTATCAGCGTaag | | | | | |
| | | AGGTCTTACGCGGCA | | | | | | | |
| | | CCGAGTCGGTGC | | | | | | | |
| 4083 | L2_e6_ | GTTTAAGAGCTAGAA | 6601 | GTTTAAGAGCTAGA | 6752 | ACCGAAAGGTCCAA | Stem loop | 6 | 88 |
| | 24 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTGGACCGAA | | TTATCAGCGTtgg | | | | | |
| | | AGGTCCAACGCGGCA | | | | | | | |
| | | CCGAGTCGGTGC | | | | | | | |
| 4084 | L2_e6_ | GTTTAAGAGCTAGAA | 6602 | GTTTAAGAGCTAGA | 6753 | GCTGAAAGGCGTGA | Stem loop | 6 | 88 |
| | 25 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCACGCTGAA | | TTATCAGCGTcac | | | | | |
| | | AGGCGTGACGCGGCA | | | | | | | |
| | | CCGAGTCGGTGC | | | | | | | |
| 4085 | L2_e6_ | GTTTAAGAGCTAGAA | 6603 | GTTTAAGAGCTAGA | 6754 | CGAGAAATCGAGTA | Stem loop | 6 | 88 |
| | 26 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTACTCGAGAA | | TTATCAGCGTact | | | | | |
| | | ATCGAGTACGCGGCA | | | | | | | |
| | | CCGAGTCGGTGC | | | | | | | |
| 4086 | L2_e6 | GTTTAAGAGCTAGAA | 6604 | GTTTAAGAGCTAGA | 6755 | CGTGAAAACGGGGA | Stem loop | 6 | 88 |
| | 27 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCCCGTGAA | | TTATCAGCGTecc | | | | | |
| | | AACGGGGACGCGGCA | | | | | | | |
| | | CCGAGTCGGTGC | | | | | | | |
| 4087 | L2_e6_ | GTTTAAGAGCTAGAA | 6605 | GTTTAAGAGCTAGA | 6756 | GGAGAAATCCGGTA | Stem loop | 6 | 88 |
| | 28 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTACCGGAGAA | | TTATCAGCGTacc | | | | | |
| | | ATCCGGTACGCGGCA | | | | | | | |
| | | CCGAGTCGGTGC | | | | | | | |
| 4088 | L2_e6_ | GTTTAAGAGCTAGAA | 6606 | GTTTAAGAGCTAGA | 6757 | GAAGAAATTCCCAA | Stem loop | 6 | 88 |
| | 29 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTGGGAAGAA | | TTATCAGCGTtgg | | | | | |
| | | ATTCCCAACGCGGCA | | | | | | | |
| | | CCGAGTCGGTGC | | | | | | | |
| 4089 | L2_e6_ | GTTTAAGAGCTAGAA | 6607 | GTTTAAGAGCTAGA | 6758 | GCGGAAACGCTAGA | Stem loop | 6 | 88 |
| | 30 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTTGGCGGAA | | TTATCAGCGTttg | | | | | |
| | | ACGCTAGACGCGGCA | | | | | | | |
| | | CCGAGTCGGTGC | | | | | | | |
| 4090 | L2_e6_ | GTTTAAGAGCTAGAA | 6608 | GTTTAAGAGCTAGA | 6759 | TCAGAAATGAGTCA | Stem loop | 6 | 88 |
| | 31 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGCTCAGAA | | TTATCAGCGTggc | | | | | |
| | | ATGAGTCACGCGGCA | | | | | | | |
| | | CCGAGTCGGTGC | | | | | | | |
| 4091 | L2_e6_ | GTTTAAGAGCTAGAA | 6609 | GTTTAAGAGCTAGA | 6760 | CGAGAAATCGTGAA | Stem loop | 6 | 88 |
| | 32 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTCACGAGAA | | TTATCAGCGTtca | | | | | |
| | | ATCGTGAACGCGGCA | | | | | | | |
| | | CCGAGTCGGTGC | | | | | | | |
| 4092 | L2_e6_ | GTTTAAGAGCTAGAA | 6610 | GTTTAAGAGCTAGA | 6761 | TACGAAAGTAAGGA | Stem loop | 6 | 88 |
| | 33 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCTTACGAA | | TTATCAGCGTcct | | | | | |
| | | AGTAAGGACGCGGCA | | | | | | | |
| | | CCGAGTCGGTGC | | | | | | | |
| 4093 | L2_e6_ | GTTTAAGAGCTAGAA | 6611 | GTTTAAGAGCTAGA | 6762 | GAGGAAACTCACCA | Stem loop | 6 | 88 |
| | 34 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGTGAGGAA | | TTATCAGCGTggt | | | | | |
| | | ACTCACCACGCGGCA | | | | | | | |
| | | CCGAGTCGGTGC | | | | | | | |
| 4094 | L2_e6_ | GTTTAAGAGCTAGAA | 6612 | GTTTAAGAGCTAGA | 6763 | CTGGAAACAGTGCA | Stem loop | 6 | 88 |
| | 35 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCACTGGAA | | TTATCAGCGTgca | | | | | |
| | | ACAGTGCACGCGGCA | | | | | | | |
| | | CCGAGTCGGTGC | | | | | | | |
| 4095 | L2_e6_ | GTTTAAGAGCTAGAA | 6613 | GTTTAAGAGCTAGA | 6764 | CCAGAAATGGAGCA | Stem loop | 6 | 88 |
| | 36 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCTCCAGAA | | TTATCAGCGTgct | | | | | |
| | | ATGGAGCACGCGGCA | | | | | | | |
| | | CCGAGTCGGTGC | | | | | | | |
| 4096 | L2_e6_ | GTTTAAGAGCTAGAA | 6614 | GTTTAAGAGCTAGA | 6765 | AGAGAAATCTCCTA | Stem loop | 6 | 88 |
| | 37 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGGAGAGAA | | TTATCAGCGTagg | | | | | |
| | | ATCTCCTACGCGGCAC | | | | | | | |
| | | CGAGTCGGTGC | | | | | | | |
| 4097 | L2_e6_ | GTTTAAGAGCTAGAA | 6615 | GTTTAAGAGCTAGA | 6766 | GCGGAAACGCTGTA | Stem loop | 6 | 88 |
| | 38 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTACAGCGGAA | | TTATCAGCGTaca | | | | | |
| | | ACGCTGTACGCGGCA | | | | | | | |
| | | CCGAGTCGGTGC | | | | | | | |
| 4098 | L2_e6_ | GTTTAAGAGCTAGAA | 6616 | GTTTAAGAGCTAGA | 6767 | GCGGAAACGCTACA | Stem loop | 6 | 88 |
| | 39 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGTAGCGGAA | | TTATCAGCGTgta | | | | | |
| | | ACGCTACACGCGGCA | | | | | | | |
| | | CCGAGTCGGTGC | | | | | | | |
| 4099 | L2_e6_ | GTTTAAGAGCTAGAA | 6617 | GTTTAAGAGCTAGA | 6740 | CGGGAAACCGGGGA | Stem loop | 6 | 88 |
| | 40 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCCCGGGAA | | TTATCAGCGTccc | | | | | |
| | | ACCGGGGACGCGGCA | | | | | | | |
| | | CCGAGTCGGTGC | | | | | | | |
| 4100 | L2_e6_ | GTTTAAGAGCTAGAA | 6618 | GTTTAAGAGCTAGA | 6768 | CCTGAAAAGGATGA | Stem loop | 6 | 88 |
| | 41 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCATCCTGAA | | TTATCAGCGTcat | | | | | |
| | | AAGGATGACGCGGCA | | | | | | | |
| | | CCGAGTCGGTGC | | | | | | | |
| 4101 | L2_e6_ | GTTTAAGAGCTAGAA | 6619 | GTTTAAGAGCTAGA | 6769 | TAGGAAACTAGGAA | Stem loop | 6 | 88 |
| | 42 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTCCTAGGAA | | TTATCAGCGTtcc | | | | | |
| | | ACTAGGAACGCGGCA | | | | | | | |
| | | CCGAGTCGGTGC | | | | | | | |
| 4102 | L2_e6_ | GTTTAAGAGCTAGAA | 6620 | GTTTAAGAGCTAGA | 6770 | AGCGAAAGCTCGGA | Stem loop | 6 | 88 |
| | 43 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCTGAGCGAA | | TTATCAGCGTctg | | | | | |
| | | AGCTCGGACGCGGCA | | | | | | | |
| | | CCGAGTCGGTGC | | | | | | | |
| 4103 | L2_e6_ | GTTTAAGAGCTAGAA | 6621 | GTTTAAGAGCTAGA | 6771 | TTCGAAAGGAGGGA | Stem loop | 6 | 88 |
| | 44 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCCTTCGAAA | | TTATCAGCGTccc | | | | | |
| | | GGAGGGACGCGGCAC | | | | | | | |
| | | CGAGTCGGTGC | | | | | | | |
| 4104 | L2_e6_ | GTTTAAGAGCTAGAA | 6622 | GTTTAAGAGCTAGA | 6772 | GCGGAAACGCGTGA | Stem loop | 6 | 88 |
| | 45 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCACGCGGAA | | TTATCAGCGTcac | | | | | |
| | | ACGCGTGACGCGGCA | | | | | | | |
| | | CCGAGTCGGTGC | | | | | | | |
| 4105 | L2_e6_ | GTTTAAGAGCTAGAA | 6623 | GTTTAAGAGCTAGA | 6773 | CTGGAAACAGTGAA | Stem loop | 6 | 88 |
| | 46 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTCGCTGGAA | | TTATCAGCGTtcg | | | | | |
| | | ACAGTGAACGCGGCA | | | | | | | |
| | | CCGAGTCGGTGC | | | | | | | |
| 4106 | L2_e6_ | GTTTAAGAGCTAGAA | 6624 | GTTTAAGAGCTAGA | 6774 | CTCGAAAGAGGCCA | Stem loop | 6 | 88 |
| | 47 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGTCTCGAA | | TTATCAGCGTggt | | | | | |
| | | AGAGGCCACGCGGCA | | | | | | | |
| | | CCGAGTCGGTGC | | | | | | | |
| 4107 | L2_e6_ | GTTTAAGAGCTAGAA | 6625 | GTTTAAGAGCTAGA | 6775 | GTAGAAATGCGGGA | Stem loop | 6 | 88 |
| | 48 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTCCGTAGAA | | TTATCAGCGTtcc | | | | | |
| | | ATGCGGGACGCGGCA | | | | | | | |
| | | CCGAGTCGGTGC | | | | | | | |
| 4108 | L2_e6_ | GTTTAAGAGCTAGAA | 6626 | GTTTAAGAGCTAGA | 6776 | CCGGAAACGGTACA | Stem loop | 6 | 88 |
| | 49 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGTACCGGAA | | TTATCAGCGTgta | | | | | |
| | | ACGGTACACGCGGCA | | | | | | | |
| | | CCGAGTCGGTGC | | | | | | | |
| 4109 | L2_e6_ | GTTTAAGAGCTAGAA | 6627 | GTTTAAGAGCTAGA | 6777 | CTCGAAAGGGACTA | Stem loop | 6 | 88 |
| | 50 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGTCTCGAA | | TTATCAGCGTagt | | | | | |
| | | AGGGACTACGCGGCA | | | | | | | |
| | | CCGAGTCGGTGC | | | | | | | |
| 4110 | L2_e6 | GTTTAAGAGCTAGAA | 6628 | GTTTAAGAGCTAGA | 6778 | CTTGAAAAAGGTCA | Stem loop | 6 | 88 |
| | 51 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGCCTTGAA | | TTATCAGCGTggc | | | | | |
| | | AAAGGTCACGCGGCA | | | | | | | |
| | | CCGAGTCGGTGC | | | | | | | |
| 4111 | L2_e6_ | GTTTAAGAGCTAGAA | 6629 | GTTTAAGAGCTAGA | 6779 | CCGGAAACGGGGCA | Stem loop | 6 | 88 |
| | 52 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCTCCGGAA | | TTATCAGCGTgct | | | | | |
| | | ACGGGGCACGCGGCA | | | | | | | |
| | | CCGAGTCGGTGC | | | | | | | |
| 4112 | L2_e6_ | GTTTAAGAGCTAGAA | 6630 | GTTTAAGAGCTAGA | 6780 | TCTGAAAAGGCAGA | Stem loop | 6 | 88 |
| | 53 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCTGTCTGAAA | | TTATCAGCGTctg | | | | | |
| | | AGGCAGACGCGGCAC | | | | | | | |
| | | CGAGTCGGTGC | | | | | | | |
| 4113 | L2_e6_ | GTTTAAGAGCTAGAA | 6631 | GTTTAAGAGCTAGA | 6781 | CCTGAAAAGGCTCA | Stem loop | 6 | 88 |
| | 54 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGAGCCTGAA | | TTATCAGCGTgag | | | | | |
| | | AAGGCTCACGCGGCA | | | | | | | |
| | | CCGAGTCGGTGC | | | | | | | |
| 4114 | L2_e6_ | GTTTAAGAGCTAGAA | 6632 | GTTTAAGAGCTAGA | 6782 | TAGGAAACTGGGCA | Stem loop | 6 | 88 |
| | 55 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCCTAGGAA | | TTATCAGCGTgcc | | | | | |
| | | ACTGGGCACGCGGCA | | | | | | | |
| | | CCGAGTCGGTGC | | | | | | | |
| 4115 | L2_e6_ | GTTTAAGAGCTAGAA | 6633 | GTTTAAGAGCTAGA | 6783 | GCCGAAAGGCTCAA | Stem loop | 6 | 88 |
| | 56 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTGAGCCGAA | | TTATCAGCGTtga | | | | | |
| | | AGGCTCAACGCGGCA | | | | | | | |
| | | CCGAGTCGGTGC | | | | | | | |
| 4116 | L2_e6_ | GTTTAAGAGCTAGAA | 6634 | GTTTAAGAGCTAGA | 6784 | AGGGAAACCTGTTA | Stem loop | 6 | 88 |
| | 57 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGCAGGGAA | | TTATCAGCGTagc | | | | | |
| | | ACCTGTTACGCGGCAC | | | | | | | |
| | | CGAGTCGGTGC | | | | | | | |
| 4117 | L2_e6_ | GTTTAAGAGCTAGAA | 6635 | GTTTAAGAGCTAGA | 6785 | ACTGAAAAGTGTCA | Stem loop | 6 | 88 |
| | 58 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGACACTGAA | | TTATCAGCGTgac | | | | | |
| | | AAGTGTCACGCGGCA | | | | | | | |
| | | CCGAGTCGGTGC | | | | | | | |
| 4118 | L2_e6_ | GTTTAAGAGCTAGAA | 6636 | GTTTAAGAGCTAGA | 6786 | TCAGAAATGAGGGA | Stem loop | 6 | 88 |
| | 59 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCCTCAGAA | | TTATCAGCGTccc | | | | | |
| | | ATGAGGGACGCGGCA | | | | | | | |
| | | CCGAGTCGGTGC | | | | | | | |
| 4119 | L2_e6_ | GTTTAAGAGCTAGAA | 6637 | GTTTAAGAGCTAGA | 6787 | TTCGAAAGAACCTA | Stem loop | 6 | 88 |
| | 60 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGGTTCGAA | | TTATCAGCGTggg | | | | | |
| | | AGAACCTACGCGGCA | | | | | | | |
| | | CCGAGTCGGTGC | | | | | | | |
| 4120 | L2_e6_ | GTTTAAGAGCTAGAA | 6638 | GTTTAAGAGCTAGA | 6788 | TCGGAAACGAAACA | Stem loop | 6 | 88 |
| | 61 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGTTTCGGAA | | TTATCAGCGTgtt | | | | | |
| | | ACGAAACACGCGGCA | | | | | | | |
| | | CCGAGTCGGTGC | | | | | | | |
| 4121 | L2_e6_ | GTTTAAGAGCTAGAA | 6639 | GTTTAAGAGCTAGA | 6789 | TCCGAAAGGAAGAA | Stem loop | 6 | 88 |
| | 62 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTCTTCCGAAA | | TTATCAGCGTtct | | | | | |
| | | GGAAGAACGCGGCAC | | | | | | | |
| | | CGAGTCGGTGC | | | | | | | |
| 4122 | L2_e6_ | GTTTAAGAGCTAGAA | 6640 | GTTTAAGAGCTAGA | 6790 | TCCGAAAGGGGATA | Stem loop | 6 | 88 |
| | 63 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTATCTCCGAA | | TTATCAGCGTatc | | | | | |
| | | AGGGGATACGCGGCA | | | | | | | |
| | | CCGAGTCGGTGC | | | | | | | |
| 4123 | L2_e6_ | GTTTAAGAGCTAGAA | 6641 | GTTTAAGAGCTAGA | 6791 | CACGAAAGTGTGTA | Stem loop | 6 | 88 |
| | 64 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTACACACGAA | | TTATCAGCGTaca | | | | | |
| | | AGTGTGTACGCGGCA | | | | | | | |
| | | CCGAGTCGGTGC | | | | | | | |
| 4124 | L2_e6_ | GTTTAAGAGCTAGAA | 6642 | GTTTAAGAGCTAGA | 6792 | TTTGAAAAAAGCCA | Stem loop | 6 | 88 |
| | 65 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGCTTTGAA | | TTATCAGCGTggc | | | | | |
| | | AAAAGCCACGCGGCA | | | | | | | |
| | | CCGAGTCGGTGC | | | | | | | |
| 4125 | L2_e6_ | GTTTAAGAGCTAGAA | 6643 | GTTTAAGAGCTAGA | 6793 | GCCGAAAGGCTTTA | Stem loop | 6 | 88 |
| | 66 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGAGCCGAA | | TTATCAGCGTaga | | | | | |
| | | AGGCTTTACGCGGCA | | | | | | | |
| | | CCGAGTCGGTGC | | | | | | | |
| 4126 | L2_e6_ | GTTTAAGAGCTAGAA | 6644 | GTTTAAGAGCTAGA | 6794 | CCAGAAATGGACCA | Stem loop | 6 | 88 |
| | 67 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGTCCAGAA | | TTATCAGCGTggt | | | | | |
| | | ATGGACCACGCGGCA | | | | | | | |
| | | CCGAGTCGGTGC | | | | | | | |
| 4127 | L2_e6_ | GTTTAAGAGCTAGAA | 6645 | GTTTAAGAGCTAGA | 6795 | CTAGAAATAGGCAA | Stem loop | 6 | 88 |
| | 68 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTGCCTAGAA | | TTATCAGCGTtgc | | | | | |
| | | ATAGGCAACGCGGCA | | | | | | | |
| | | CCGAGTCGGTGC | | | | | | | |
| 4128 | L2_e6_ | GTTTAAGAGCTAGAA | 6646 | GTTTAAGAGCTAGA | 6796 | AACGAAAGTTGCTA | Stem loop | 6 | 88 |
| | 69 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGCAACGAA | | TTATCAGCGTagc | | | | | |
| | | AGTTGCTACGCGGCA | | | | | | | |
| | | CCGAGTCGGTGC | | | | | | | |
| 4129 | L2_e6_ | GTTTAAGAGCTAGAA | 6647 | GTTTAAGAGCTAGA | 6797 | TCAGAAATGGCCAA | Stem loop | 6 | 88 |
| | 70 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTGGTCAGAA | | TTATCAGCGTtgg | | | | | |
| | | ATGGCCAACGCGGCA | | | | | | | |
| | | CCGAGTCGGTGC | | | | | | | |
| 4130 | L2_e6_ | GTTTAAGAGCTAGAA | 6648 | GTTTAAGAGCTAGA | 6798 | AGGGAAACTTTGGA | Stem loop | 6 | 88 |
| | 71 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCAAGGGAA | | TTATCAGCGTcca | | | | | |
| | | ACTTTGGACGCGGCA | | | | | | | |
| | | CCGAGTCGGTGC | | | | | | | |
| 4131 | L2_e6_ | GTTTAAGAGCTAGAA | 6649 | GTTTAAGAGCTAGA | 6799 | CACGAAAGTGGAAA | Stem loop | 6 | 88 |
| | 72 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTTCCACGAA | | TTATCAGCGTttc | | | | | |
| | | AGTGGAAACGCGGCA | | | | | | | |
| | | CCGAGTCGGTGC | | | | | | | |
| 4132 | L2_e6_ | GTTTAAGAGCTAGAA | 6650 | GTTTAAGAGCTAGA | 6800 | CGGGAAACCGCGCA | Stem loop | 6 | 88 |
| | 73 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCGCGGGAA | | TTATCAGCGTgcg | | | | | |
| | | ACCGCGCACGCGGCA | | | | | | | |
| | | CCGAGTCGGTGC | | | | | | | |
| 4133 | L2_e6_ | GTTTAAGAGCTAGAA | 6651 | GTTTAAGAGCTAGA | 6801 | TGAGAAATTAGGGA | Stem loop | 6 | 88 |
| | 74 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCCTGAGAA | | TTATCAGCGTccc | | | | | |
| | | ATTAGGGACGCGGCA | | | | | | | |
| | | CCGAGTCGGTGC | | | | | | | |
| 4134 | L2_e6_ | GTTTAAGAGCTAGAA | 6652 | GTTTAAGAGCTAGA | 6802 | GTGGAAACACGTGA | Stem loop | 6 | 88 |
| | 75 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCACGTGGAA | | TTATCAGCGTcac | | | | | |
| | | ACACGTGACGCGGCA | | | | | | | |
| | | CCGAGTCGGTGC | | | | | | | |
| 4135 | L2_e6_ | GTTTAAGAGCTAGAA | 6653 | GTTTAAGAGCTAGA | 6803 | CTCGAAAGAGGGCA | Stem loop | 6 | 88 |
| | 76 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCCCTCGAA | | TTATCAGCGTgcc | | | | | |
| | | AGAGGGCACGCGGCA | | | | | | | |
| | | CCGAGTCGGTGC | | | | | | | |
| 4136 | L2_e6_ | GTTTAAGAGCTAGAA | 6654 | GTTTAAGAGCTAGA | 6804 | GGTGAAAGCCTGCA | Stem loop | 6 | 88 |
| | 77 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCGGGTGAA | | TTATCAGCGTgcg | | | | | |
| | | AGCCTGCACGCGGCA | | | | | | | |
| | | CCGAGTCGGTGC | | | | | | | |
| 4137 | L2_e6_ | GTTTAAGAGCTAGAA | 6655 | GTTTAAGAGCTAGA | 6805 | GCAGAAATGCGCTA | Stem loop | 6 | 88 |
| | 78 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGCGCAGAA | | TTATCAGCGTagc | | | | | |
| | | ATGCGCTACGCGGCA | | | | | | | |
| | | CCGAGTCGGTGC | | | | | | | |
| 4138 | L2_e6_ | GTTTAAGAGCTAGAA | 6656 | GTTTAAGAGCTAGA | 6806 | GGGGAAACCCTGGA | Stem loop | 6 | 88 |
| | 79 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCAGGGGAA | | TTATCAGCGTcca | | | | | |
| | | ACCCTGGACGCGGCA | | | | | | | |
| | | CCGAGTCGGTGC | | | | | | | |
| 4139 | L2_e6_ | GTTTAAGAGCTAGAA | 6657 | GTTTAAGAGCTAGA | 6807 | CGAGAAATTGGCTA | Stem loop | 6 | 88 |
| | 80 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGCCGAGAA | | TTATCAGCGTagc | | | | | |
| | | ATTGGCTACGCGGCA | | | | | | | |
| | | CCGAGTCGGTGC | | | | | | | |
| 4140 | L2_e6_ | GTTTAAGAGCTAGAA | 6658 | GTTTAAGAGCTAGA | 6808 | GCGGAAACGCGTAA | Stem loop | 6 | 88 |
| | 81 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTACGCGGAA | | TTATCAGCGTtac | | | | | |
| | | ACGCGTAACGCGGCA | | | | | | | |
| | | CCGAGTCGGTGC | | | | | | | |
| 4141 | L2_e6_ | GTTTAAGAGCTAGAA | 6659 | GTTTAAGAGCTAGA | 6809 | CTCGAAAGGGTGTA | Stem loop | 6 | 88 |
| | 82 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTACACTCGAA | | TTATCAGCGTaca | | | | | |
| | | AGGGTGTACGCGGCA | | | | | | | |
| | | CCGAGTCGGTGC | | | | | | | |
| 4142 | L2_e6_ | GTTTAAGAGCTAGAA | 6660 | GTTTAAGAGCTAGA | 6810 | ACCGAAAGGTGCTA | Stem loop | 6 | 88 |
| | 83 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGTACCGAA | | TTATCAGCGTagt | | | | | |
| | | AGGTGCTACGCGGCA | | | | | | | |
| | | CCGAGTCGGTGC | | | | | | | |
| 4143 | L2_e6_ | GTTTAAGAGCTAGAA | 6661 | GTTTAAGAGCTAGA | 6811 | GGGGAAACCCCCAA | Stem loop | 6 | 88 |
| | 84 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTGGGGGGAA | | TTATCAGCGTtgg | | | | | |
| | | ACCCCCAACGCGGCA | | | | | | | |
| | | CCGAGTCGGTGC | | | | | | | |
| 4144 | L2_e6_ | GTTTAAGAGCTAGAA | 6662 | GTTTAAGAGCTAGA | 6812 | GGTGAAAGCCACCA | Stem loop | 6 | 88 |
| | 85 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGTGGTGAA | | TTATCAGCGTggt | | | | | |
| | | AGCCACCACGCGGCA | | | | | | | |
| | | CCGAGTCGGTGC | | | | | | | |
| 4145 | L2_e6 | GTTTAAGAGCTAGAA | 6663 | GTTTAAGAGCTAGA | 6813 | GAGGAAACTCTCCA | Stem loop | 6 | 88 |
| | 86 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGGGAGGAA | | TTATCAGCGTggg | | | | | |
| | | ACTCTCCACGCGGCAC | | | | | | | |
| | | CGAGTCGGTGC | | | | | | | |
| 4146 | L2_e6 | GTTTAAGAGCTAGAA | 6664 | GTTTAAGAGCTAGA | 6814 | TGGGAAACCAAACA | Stem loop | 6 | 88 |
| | 87 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGTTTGGGAA | | TTATCAGCGTgtt | | | | | |
| | | ACCAAACACGCGGCA | | | | | | | |
| | | CCGAGTCGGTGC | | | | | | | |
| 4147 | L2_e6_ | GTTTAAGAGCTAGAA | 6665 | GTTTAAGAGCTAGA | 6815 | AACGAAAGTTGGAA | Stem loop | 6 | 88 |
| | 88 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTCCAACGAA | | TTATCAGCGTtec | | | | | |
| | | AGTTGGAACGCGGCA | | | | | | | |
| | | CCGAGTCGGTGC | | | | | | | |
| 4148 | L2_e6_ | GTTTAAGAGCTAGAA | 6666 | GTTTAAGAGCTAGA | 6816 | CGCGAAAGCGCGTA | Stem loop | 6 | 88 |
| | 89 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCGCGCGAA | | TTATCAGCGTgcg | | | | | |
| | | AGCGCGTACGCGGCA | | | | | | | |
| | | CCGAGTCGGTGC | | | | | | | |
| 4149 | L2_e6_ | GTTTAAGAGCTAGAA | 6667 | GTTTAAGAGCTAGA | 6817 | GTCGAAAGGCAGCA | Stem loop | 6 | 88 |
| | 90 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGCTGTCGAA | | TTATCAGCGTgct | | | | | |
| | | AGGCAGCACGCGGCA | | | | | | | |
| | | CCGAGTCGGTGC | | | | | | | |
| 4150 | L2_e6_ | GTTTAAGAGCTAGAA | 6668 | GTTTAAGAGCTAGA | 6818 | GTCGAAAGACGAGA | Stem loop | 6 | 88 |
| | 91 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCTCGTCGAA | | TTATCAGCGTctc | | | | | |
| | | AGACGAGACGCGGCA | | | | | | | |
| | | CCGAGTCGGTGC | | | | | | | |
| 4151 | L2_e6_ | GTTTAAGAGCTAGAA | 6669 | GTTTAAGAGCTAGA | 6819 | CAGGAAACTGGTCA | Stem loop | 6 | 88 |
| | 92 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGACCAGGAA | | TTATCAGCGTgac | | | | | |
| | | ACTGGTCACGCGGCA | | | | | | | |
| | | CCGAGTCGGTGC | | | | | | | |
| 4152 | L2_e6_ | GTTTAAGAGCTAGAA | 6670 | GTTTAAGAGCTAGA | 6820 | GTGGAAACACGCCA | Stem loop | 6 | 88 |
| | 93 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGTGTGGAA | | TTATCAGCGTggt | | | | | |
| | | ACACGCCACGCGGCA | | | | | | | |
| | | CCGAGTCGGTGC | | | | | | | |
| 4153 | L2_e6_ | GTTTAAGAGCTAGAA | 6671 | GTTTAAGAGCTAGA | 6821 | GCCGAAAGGCAAAA | Stem loop | 6 | 88 |
| | 94 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTTTGCCGAAA | | TTATCAGCGTttt | | | | | |
| | | GGCAAAACGCGGCAC | | | | | | | |
| | | CGAGTCGGTGC | | | | | | | |
| 4154 | L2_e6_ | GTTTAAGAGCTAGAA | 6672 | GTTTAAGAGCTAGA | 6822 | GACGAAAGTCACTA | Stem loop | 6 | 88 |
| | 95 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTAGTGACGAA | | TTATCAGCGTagt | | | | | |
| | | AGTCACTACGCGGCA | | | | | | | |
| | | CCGAGTCGGTGC | | | | | | | |
| 4155 | L2_e6_ | GTTTAAGAGCTAGAA | 6673 | GTTTAAGAGCTAGA | 6823 | AAGGAAACTTAGGA | Stem loop | 6 | 88 |
| | 96 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTCCTAAGGAA | | TTATCAGCGTcct | | | | | |
| | | ACTTAGGACGCGGCA | | | | | | | |
| | | CCGAGTCGGTGC | | | | | | | |
| 4156 | L2_e6_ | GTTTAAGAGCTAGAA | 6674 | GTTTAAGAGCTAGA | 6824 | CTAGAAATAGGCTA | Stem loop | 6 | 88 |
| | 97 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGCCTAGAA | | TTATCAGCGTggc | | | | | |
| | | ATAGGCTACGCGGCA | | | | | | | |
| | | CCGAGTCGGTGC | | | | | | | |
| 4157 | L2_e6_ | GTTTAAGAGCTAGAA | 6675 | GTTTAAGAGCTAGA | 6825 | AGCGAAAGCTCATA | Stem loop | 6 | 88 |
| | 98 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTATGAGCGAA | | TTATCAGCGTatg | | | | | |
| | | AGCTCATACGCGGCA | | | | | | | |
| | | CCGAGTCGGTGC | | | | | | | |
| 4158 | L2_e6_ | GTTTAAGAGCTAGAA | 6676 | GTTTAAGAGCTAGA | 6826 | ATCGAAAGATCCAA | Stem loop | 6 | 88 |
| | 99 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTTGGATCGAA | | TTATCAGCGTtgg | | | | | |
| | | AGATCCAACGCGGCA | | | | | | | |
| | | CCGAGTCGGTGC | | | | | | | |
| 4159 | L2_e6_ | GTTTAAGAGCTAGAA | 6677 | GTTTAAGAGCTAGA | 6827 | CAAGAAATTGCCCA | Stem loop | 6 | 88 |
| | 100 | ATAGCAAGTTTAAAT | | AATAGCAAGTTTAA | | CGCGGCACCGAGTC | 2 | | |
| | | AAGGCTAGTCCGTTAT | | ATAAGGCTAGTCCG | | GGTGC | | | |
| | | CAGCGTGGGCAAGAA | | TTATCAGCGTggg | | | | | |
| | | ATTGCCCACGCGGCA | | | | | | | |
| | | CCGAGTCGGTGC | | | | | | | |
| 4160 | L2_e7_ | GTTTAAGAGCTAGAA | 6678 | GTTTAAGAGCTAGA | 6828 | CGCGAAAGCGACGA | Stem loop | 7 | 90 |
| | 1 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTCGTCGCGA | | TTATCAGCGTtcgt | | | | | |
| | | AAGCGACGAACGCGG | | | | | | | |
| | | CACCGAGTCGGTGC | | | | | | | |
| 4161 | L2_e7_ | GTTTAAGAGCTAGAA | 6679 | GTTTAAGAGCTAGA | 6829 | CGCGAAAGCGCACT | Stem loop | 7 | 90 |
| | 2 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGGTGCGCGA | | TTATCAGCGTggtg | | | | | |
| | | AAGCGCACTACGCGG | | | | | | | |
| | | CACCGAGTCGGTGC | | | | | | | |
| 4162 | L2_e7_ | GTTTAAGAGCTAGAA | 6680 | GTTTAAGAGCTAGA | 6830 | CGGGAAACTGTGTC | Stem loop | 7 | 90 |
| | 3 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGGCACGGGA | | TTATCAGCGTggca | | | | | |
| | | AACTGTGTCACGCGG | | | | | | | |
| | | CACCGAGTCGGTGC | | | | | | | |
| 4163 | L2_e7_ | GTTTAAGAGCTAGAA | 6681 | GTTTAAGAGCTAGA | 6831 | GGGGAAACCCTTAG | Stem loop | 7 | 90 |
| | 4 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCTAAGGGGA | | TTATCAGCGTctaa | | | | | |
| | | AACCCTTAGACGCGG | | | | | | | |
| | | CACCGAGTCGGTGC | | | | | | | |
| 4164 | L2_e7_ | GTTTAAGAGCTAGAA | 6682 | GTTTAAGAGCTAGA | 6832 | TCGGAAACGAGTCT | Stem loop | 7 | 90 |
| | 5 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTAGACTCGGA | | TTATCAGCGTagac | | | | | |
| | | AACGAGTCTACGCGG | | | | | | | |
| | | CACCGAGTCGGTGC | | | | | | | |
| 4165 | L2_e7_ | GTTTAAGAGCTAGAA | 6683 | GTTTAAGAGCTAGA | 6833 | GACGAAAGTCGGCG | Stem loop | 7 | 90 |
| | 6 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGCTGACGA | | TTATCAGCGTcgct | | | | | |
| | | AAGTCGGCGACGCGG | | | | | | | |
| | | CACCGAGTCGGTGC | | | | | | | |
| 4166 | L2_e7_ | GTTTAAGAGCTAGAA | 6684 | GTTTAAGAGCTAGA | 6834 | TCGGAAACGACTGT | Stem loop | 7 | 90 |
| | 7 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTACAGTCGGA | | TTATCAGCGTacag | | | | | |
| | | AACGACTGTACGCGG | | | | | | | |
| | | CACCGAGTCGGTGC | | | | | | | |
| 4167 | L2_e7_ | GTTTAAGAGCTAGAA | 6685 | GTTTAAGAGCTAGA | 6835 | CTGGAAACAGGCCA | Stem loop | 7 | 90 |
| | 8 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTGGCCTGGA | | TTATCAGCGTtggc | | | | | |
| | | AACAGGCCAACGCGG | | | | | | | |
| | | CACCGAGTCGGTGC | | | | | | | |
| 4168 | L2_e7_ | GTTTAAGAGCTAGAA | 6686 | GTTTAAGAGCTAGA | 6836 | GTCGAAAGACGGGC | Stem loop | 7 | 90 |
| | 9 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCCCGTCGA | | TTATCAGCGTgccc | | | | | |
| | | AAGACGGGCACGCGG | | | | | | | |
| | | CACCGAGTCGGTGC | | | | | | | |
| 4169 | L2_e7_ | GTTTAAGAGCTAGAA | 6687 | GTTTAAGAGCTAGA | 6837 | GCTGAAAGGCCCGG | Stem loop | 7 | 90 |
| | 10 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCGGGCTGA | | TTATCAGCGTccgg | | | | | |
| | | AAGGCCCGGACGCGG | | | | | | | |
| | | CACCGAGTCGGTGC | | | | | | | |
| 4170 | L2_e7_ | GTTTAAGAGCTAGAA | 6688 | GTTTAAGAGCTAGA | 6838 | GTGGAAATACGCTG | Stem loop | 7 | 90 |
| | 11 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGGCGTGGA | | TTATCAGCGTcggc | | | | | |
| | | AATACGCTGACGCGG | | | | | | | |
| | | CACCGAGTCGGTGC | | | | | | | |
| 4171 | L2_e7_ | GTTTAAGAGCTAGAA | 6689 | GTTTAAGAGCTAGA | 6839 | CACGAAAGTGCCCC | Stem loop | 7 | 90 |
| | 12 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGGGGCACGA | | TTATCAGCGTgggg | | | | | |
| | | AAGTGCCCCACGCGG | | | | | | | |
| | | CACCGAGTCGGTGC | | | | | | | |
| 4172 | L2_e7_ | GTTTAAGAGCTAGAA | 6690 | GTTTAAGAGCTAGA | 6840 | CCGGAAATGGACCC | Stem loop | 7 | 90 |
| | 13 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGGGTCCGGA | | TTATCAGCGTgggt | | | | | |
| | | AATGGACCCACGCGG | | | | | | | |
| | | CACCGAGTCGGTGC | | | | | | | |
| 4173 | L2_e7_ | GTTTAAGAGCTAGAA | 6691 | GTTTAAGAGCTAGA | 6841 | ACGGAAACGTGTTG | Stem loop | 7 | 90 |
| | 14 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCAACACGGA | | TTATCAGCGTcaac | | | | | |
| | | AACGTGTTGACGCGG | | | | | | | |
| | | CACCGAGTCGGTGC | | | | | | | |
| 4174 | L2_e7_ | GTTTAAGAGCTAGAA | 6692 | GTTTAAGAGCTAGA | 6842 | GCTGAAAAGCCACA | Stem loop | 7 | 90 |
| | 15 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTGTGGCTGA | | TTATCAGCGTtgtg | | | | | |
| | | AAAGCCACAACGCGG | | | | | | | |
| | | CACCGAGTCGGTGC | | | | | | | |
| 4175 | L2_e7_ | GTTTAAGAGCTAGAA | 6693 | GTTTAAGAGCTAGA | 6843 | ACCGAAAGGTAACG | Stem loop | 7 | 90 |
| | 16 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGTTACCGA | | TTATCAGCGTcgtt | | | | | |
| | | AAGGTAACGACGCGG | | | | | | | |
| | | CACCGAGTCGGTGC | | | | | | | |
| 4176 | L2_e7_ | GTTTAAGAGCTAGAA | 6694 | GTTTAAGAGCTAGA | 6844 | AGCGAAAGCTAGGC | Stem loop | 7 | 90 |
| | 17 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCTTAGCGA | | TTATCAGCGTgctt | | | | | |
| | | AAGCTAGGCACGCGG | | | | | | | |
| | | CACCGAGTCGGTGC | | | | | | | |
| 4177 | L2_e7_ | GTTTAAGAGCTAGAA | 6695 | GTTTAAGAGCTAGA | 6845 | GCAGAAATGCGGTT | Stem loop | 7 | 90 |
| | 18 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTAACCGCAGA | | TTATCAGCGTaacc | | | | | |
| | | AATGCGGTTACGCGG | | | | | | | |
| | | CACCGAGTCGGTGC | | | | | | | |
| 4178 | L2_e7_ | GTTTAAGAGCTAGAA | 6696 | GTTTAAGAGCTAGA | 6846 | TGGGAAACCGGTGT | Stem loop | 7 | 90 |
| | 19 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTACACTGGGA | | TTATCAGCGTacac | | | | | |
| | | AACCGGTGTACGCGG | | | | | | | |
| | | CACCGAGTCGGTGC | | | | | | | |
| 4179 | L2_e7_ | GTTTAAGAGCTAGAA | 6697 | GTTTAAGAGCTAGA | 6847 | GGGGAAATCCCCGC | Stem loop | 7 | 90 |
| | 20 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCGGGGGGA | | TTATCAGCGTgcgg | | | | | |
| | | AATCCCCGCACGCGG | | | | | | | |
| | | CACCGAGTCGGTGC | | | | | | | |
| 4180 | L2_e7_ | GTTTAAGAGCTAGAA | 6698 | GTTTAAGAGCTAGA | 6848 | TTCGAAAGGACGGA | Stem loop | 7 | 90 |
| | 21 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTCCGTTCGAA | | TTATCAGCGTtccg | | | | | |
| | | AGGACGGAACGCGGC | | | | | | | |
| | | ACCGAGTCGGTGC | | | | | | | |
| 4181 | L2_e7_ | GTTTAAGAGCTAGAA | 6699 | GTTTAAGAGCTAGA | 6849 | GCCGAAAGGCCAGG | Stem loop | 7 | 90 |
| | 22 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCTGGCCGA | | TTATCAGCGTcctg | | | | | |
| | | AAGGCCAGGACGCGG | | | | | | | |
| | | CACCGAGTCGGTGC | | | | | | | |
| 4182 | L2_e7_ | GTTTAAGAGCTAGAA | 6700 | GTTTAAGAGCTAGA | 6850 | ACCGAAAGGTCCTG | Stem loop | 7 | 90 |
| | 23 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCAGGACCGA | | TTATCAGCGTcagg | | | | | |
| | | AAGGTCCTGACGCGG | | | | | | | |
| | | CACCGAGTCGGTGC | | | | | | | |
| 4183 | L2_e7_ | GTTTAAGAGCTAGAA | 6701 | GTTTAAGAGCTAGA | 6851 | GGAGAAATCCGGGG | Stem loop | 7 | 90 |
| | 24 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTTCCGGAGA | | TTATCAGCGTttec | | | | | |
| | | AATCCGGGGACGCGG | | | | | | | |
| | | CACCGAGTCGGTGC | | | | | | | |
| 4184 | L2_e7_ | GTTTAAGAGCTAGAA | 6702 | GTTTAAGAGCTAGA | 6852 | CGAGAAATCGACTC | Stem loop | 7 | 90 |
| | 25 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGAGTCGAGA | | TTATCAGCGTgagt | | | | | |
| | | AATCGACTCACGCGG | | | | | | | |
| | | CACCGAGTCGGTGC | | | | | | | |
| 4185 | L2_e7_ | GTTTAAGAGCTAGAA | 6703 | GTTTAAGAGCTAGA | 6853 | GTTGAAAGACAGGG | Stem loop | 7 | 90 |
| | 26 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCCTGTTGAA | | TTATCAGCGTccct | | | | | |
| | | AGACAGGGACGCGGC | | | | | | | |
| | | ACCGAGTCGGTGC | | | | | | | |
| 4186 | L2_e7_ | GTTTAAGAGCTAGAA | 6704 | GTTTAAGAGCTAGA | 6854 | CCGGAAACGGTGGT | Stem loop | 7 | 90 |
| | 27 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTATCACCGGA | | TTATCAGCGTatca | | | | | |
| | | AACGGTGGTACGCGG | | | | | | | |
| | | CACCGAGTCGGTGC | | | | | | | |
| 4187 | L2_e7_ | GTTTAAGAGCTAGAA | 6705 | GTTTAAGAGCTAGA | 6855 | CTGGAAACAGCGTC | Stem loop | 7 | 90 |
| | 28 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGACGCTGGA | | TTATCAGCGTgacg | | | | | |
| | | AACAGCGTCACGCGG | | | | | | | |
| | | CACCGAGTCGGTGC | | | | | | | |
| 4188 | L2_e7_ | GTTTAAGAGCTAGAA | 6706 | GTTTAAGAGCTAGA | 6856 | GCGGAAACGCGCGA | Stem loop | 7 | 90 |
| | 29 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTCGCGCGGA | | TTATCAGCGTtcgc | | | | | |
| | | AACGCGCGAACGCGG | | | | | | | |
| | | CACCGAGTCGGTGC | | | | | | | |
| 4189 | L2_e7_ | GTTTAAGAGCTAGAA | 6707 | GTTTAAGAGCTAGA | 6857 | ATGGAAACATCCCG | Stem loop | 7 | 90 |
| | 30 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGGGATGGA | | TTATCAGCGTcggg | | | | | |
| | | AACATCCCGACGCGG | | | | | | | |
| | | CACCGAGTCGGTGC | | | | | | | |
| 4190 | L2_e7_ | GTTTAAGAGCTAGAA | 6708 | GTTTAAGAGCTAGA | 6858 | CGCGAAAGCGGTGC | Stem loop | 7 | 90 |
| | 31 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGTACCGCGA | | TTATCAGCGTgtac | | | | | |
| | | AAGCGGTGCACGCGG | | | | | | | |
| | | CACCGAGTCGGTGC | | | | | | | |
| 4191 | L2_e7_ | GTTTAAGAGCTAGAA | 6709 | GTTTAAGAGCTAGA | 6859 | GCTGAAAAGCGCGG | Stem loop | 7 | 90 |
| | 32 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCGCGCTGA | | TTATCAGCGTccgc | | | | | |
| | | AAAGCGCGGACGCGG | | | | | | | |
| | | CACCGAGTCGGTGC | | | | | | | |
| 4192 | L2_e7_ | GTTTAAGAGCTAGAA | 6710 | GTTTAAGAGCTAGA | 6860 | GAGGAAACTCTCGT | Stem loop | 7 | 90 |
| | 33 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCGAGAGGA | | TTATCAGCGTgcga | | | | | |
| | | AACTCTCGTACGCGGC | | | | | | | |
| | | ACCGAGTCGGTGC | | | | | | | |
| 4193 | L2_e7_ | GTTTAAGAGCTAGAA | 6711 | GTTTAAGAGCTAGA | 6861 | CTCGAAAGAGTGTC | Stem loop | 7 | 90 |
| | 34 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGACGCTCGA | | TTATCAGCGTgacg | | | | | |
| | | AAGAGTGTCACGCGG | | | | | | | |
| | | CACCGAGTCGGTGC | | | | | | | |
| 4194 | L2_e7_ | GTTTAAGAGCTAGAA | 6712 | GTTTAAGAGCTAGA | 6862 | CTCGAAAGAGGCAT | Stem loop | 7 | 90 |
| | 35 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGTGCCTCGA | | TTATCAGCGTgtgc | | | | | |
| | | AAGAGGCATACGCGG | | | | | | | |
| | | CACCGAGTCGGTGC | | | | | | | |
| 4195 | L2_e7_ | GTTTAAGAGCTAGAA | 6713 | GTTTAAGAGCTAGA | 6863 | CGGGAAACCGTTGC | Stem loop | 7 | 90 |
| | 36 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCAACGGGA | | TTATCAGCGTgcaa | | | | | |
| | | AACCGTTGCACGCGG | | | | | | | |
| | | CACCGAGTCGGTGC | | | | | | | |
| 4196 | L2_e7_ | GTTTAAGAGCTAGAA | 6714 | GTTTAAGAGCTAGA | 6864 | TCGGAAACGATCGG | Stem loop | 7 | 90 |
| | 37 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCGATCGGA | | TTATCAGCGTccga | | | | | |
| | | AACGATCGGACGCGG | | | | | | | |
| | | CACCGAGTCGGTGC | | | | | | | |
| 4197 | L2_e7_ | GTTTAAGAGCTAGAA | 6715 | GTTTAAGAGCTAGA | 6865 | TGCGAAAGCAACAG | Stem loop | 7 | 90 |
| | 38 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCTGTTGCGAA | | TTATCAGCGTctgt | | | | | |
| | | AGCAACAGACGCGGC | | | | | | | |
| | | ACCGAGTCGGTGC | | | | | | | |
| 4198 | L2_e7_ | GTTTAAGAGCTAGAA | 6716 | GTTTAAGAGCTAGA | 6866 | TTCGAAAGAATCCC | Stem loop | 7 | 90 |
| | 39 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGGGATTCGA | | TTATCAGCGTggga | | | | | |
| | | AAGAATCCCACGCGG | | | | | | | |
| | | CACCGAGTCGGTGC | | | | | | | |
| 4199 | L2_e7_ | GTTTAAGAGCTAGAA | 6717 | GTTTAAGAGCTAGA | 6867 | CAGGAAACTGGGTA | Stem loop | 7 | 90 |
| | 40 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTACCCAGGA | | TTATCAGCGTtacc | | | | | |
| | | AACTGGGTAACGCGG | | | | | | | |
| | | CACCGAGTCGGTGC | | | | | | | |
| 4200 | L2_e7_ | GTTTAAGAGCTAGAA | 6718 | GTTTAAGAGCTAGA | 6868 | TCCGAAAGGAAGGT | Stem loop | 7 | 90 |
| | 41 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTACCTTCCGAA | | TTATCAGCGTacct | | | | | |
| | | AGGAAGGTACGCGGC | | | | | | | |
| | | ACCGAGTCGGTGC | | | | | | | |
| 4201 | L2_e7_ | GTTTAAGAGCTAGAA | 6719 | GTTTAAGAGCTAGA | 6869 | GCAGAAATGCCGAG | Stem loop | 7 | 90 |
| | 42 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCTCGGCAGA | | TTATCAGCGTctcg | | | | | |
| | | AATGCCGAGACGCGG | | | | | | | |
| | | CACCGAGTCGGTGC | | | | | | | |
| 4202 | L2_e7_ | GTTTAAGAGCTAGAA | 6720 | GTTTAAGAGCTAGA | 6870 | GCCGAAAGGCTTAG | Stem loop | 7 | 90 |
| | 43 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCTGAGCCGA | | TTATCAGCGTctga | | | | | |
| | | AAGGCTTAGACGCGG | | | | | | | |
| | | CACCGAGTCGGTGC | | | | | | | |
| 4203 | L2_e7_ | GTTTAAGAGCTAGAA | 6721 | GTTTAAGAGCTAGA | 6871 | CTCGAAAGAGGGCC | Stem loop | 7 | 90 |
| | 44 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGGCCCTCGA | | TTATCAGCGTggcc | | | | | |
| | | AAGAGGGCCACGCGG | | | | | | | |
| | | CACCGAGTCGGTGC | | | | | | | |
| 4204 | L2_e7_ | GTTTAAGAGCTAGAA | 6722 | GTTTAAGAGCTAGA | 6872 | GGAGAAATCCTGCG | Stem loop | 7 | 90 |
| | 45 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGCAGGAGA | | TTATCAGCGTcgca | | | | | |
| | | AATCCTGCGACGCGG | | | | | | | |
| | | CACCGAGTCGGTGC | | | | | | | |
| 4205 | L2_e7_ | GTTTAAGAGCTAGAA | 6723 | GTTTAAGAGCTAGA | 6873 | CGTGAAAACGGCTA | Stem loop | 7 | 90 |
| | 46 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTAGCCGTGA | | TTATCAGCGTtagc | | | | | |
| | | AAACGGCTAACGCGG | | | | | | | |
| | | CACCGAGTCGGTGC | | | | | | | |
| 4206 | L2_e7_ | GTTTAAGAGCTAGAA | 6724 | GTTTAAGAGCTAGA | 6874 | GTGGAAACACCTTG | Stem loop | 7 | 90 |
| | 47 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCAAGGTGGA | | TTATCAGCGTcaag | | | | | |
| | | AACACCTTGACGCGG | | | | | | | |
| | | CACCGAGTCGGTGC | | | | | | | |
| 4207 | L2_e7_ | GTTTAAGAGCTAGAA | 6725 | GTTTAAGAGCTAGA | 6875 | CCGGAAACGGTTGG | Stem loop | 7 | 90 |
| | 48 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCAACCGGA | | TTATCAGCGTccaa | | | | | |
| | | AACGGTTGGACGCGG | | | | | | | |
| | | CACCGAGTCGGTGC | | | | | | | |
| 4208 | L2_e7_ | GTTTAAGAGCTAGAA | 6726 | GTTTAAGAGCTAGA | 6876 | GAGGAAACTTGTGC | Stem loop | 7 | 90 |
| | 49 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCACGAGGA | | TTATCAGCGTgcac | | | | | |
| | | AACTTGTGCACGCGG | | | | | | | |
| | | CACCGAGTCGGTGC | | | | | | | |
| 4209 | L2_e7 | GTTTAAGAGCTAGAA | 6727 | GTTTAAGAGCTAGA | 6877 | TGTGAAAGCACACG | Stem loop | 7 | 90 |
| | 50 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGTGTGTGA | | TTATCAGCGTcgtg | | | | | |
| | | AAGCACACGACGCGG | | | | | | | |
| | | CACCGAGTCGGTGC | | | | | | | |
| 4210 | L2_e7_ | GTTTAAGAGCTAGAA | 6728 | GTTTAAGAGCTAGA | 6878 | TGCGAAAGCACCAG | Stem loop | 7 | 90 |
| | 51 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCTGGTGCGA | | TTATCAGCGTctgg | | | | | |
| | | AAGCACCAGACGCGG | | | | | | | |
| | | CACCGAGTCGGTGC | | | | | | | |
| 4211 | L2_e7_ | GTTTAAGAGCTAGAA | 6729 | GTTTAAGAGCTAGA | 6879 | AGGGAAACCTGACA | Stem loop | 7 | 90 |
| | 52 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTGTCAGGGA | | TTATCAGCGTtgtc | | | | | |
| | | AACCTGACAACGCGG | | | | | | | |
| | | CACCGAGTCGGTGC | | | | | | | |
| 4212 | L2_e7_ | GTTTAAGAGCTAGAA | 6730 | GTTTAAGAGCTAGA | 6880 | GATGAAAATCCCGG | Stem loop | 7 | 90 |
| | 53 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCGGGATGA | | TTATCAGCGTccgg | | | | | |
| | | AAATCCCGGACGCGG | | | | | | | |
| | | CACCGAGTCGGTGC | | | | | | | |
| 4213 | L2_e7_ | GTTTAAGAGCTAGAA | 6731 | GTTTAAGAGCTAGA | 6881 | CCGGAAACGGATCT | Stem loop | 7 | 90 |
| | 54 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTAGATCCGGA | | TTATCAGCGTagat | | | | | |
| | | AACGGATCTACGCGG | | | | | | | |
| | | CACCGAGTCGGTGC | | | | | | | |
| 4214 | L2_e7_ | GTTTAAGAGCTAGAA | 6732 | GTTTAAGAGCTAGA | 6882 | GGCGAAAGCCAAGT | Stem loop | 7 | 90 |
| | 55 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTACTTGGCGA | | TTATCAGCGTactt | | | | | |
| | | AAGCCAAGTACGCGG | | | | | | | |
| | | CACCGAGTCGGTGC | | | | | | | |
| 4215 | L2_e7_ | GTTTAAGAGCTAGAA | 6733 | GTTTAAGAGCTAGA | 6883 | GCCGAAAGGCCATT | Stem loop | 7 | 90 |
| | 56 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGATGGCCGA | | TTATCAGCGTgatg | | | | | |
| | | AAGGCCATTACGCGG | | | | | | | |
| | | CACCGAGTCGGTGC | | | | | | | |
| 4216 | L2_e7_ | GTTTAAGAGCTAGAA | 6734 | GTTTAAGAGCTAGA | 6884 | CGCGAAAGTGGTGG | Stem loop | 7 | 90 |
| | 57 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCCATCGCGA | | TTATCAGCGTccat | | | | | |
| | | AAGTGGTGGACGCGG | | | | | | | |
| | | CACCGAGTCGGTGC | | | | | | | |
| 4217 | L2_e7_ | GTTTAAGAGCTAGAA | 6735 | GTTTAAGAGCTAGA | 6885 | CCCGAAAGGGGGTC | Stem loop | 7 | 90 |
| | 58 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGACCCCCGA | | TTATCAGCGTgacc | | | | | |
| | | AAGGGGGTCACGCGG | | | | | | | |
| | | CACCGAGTCGGTGC | | | | | | | |
| 4218 | L2_e7_ | GTTTAAGAGCTAGAA | 6736 | GTTTAAGAGCTAGA | 6886 | ACGGAAACGTTCCG | Stem loop | 7 | 90 |
| | 59 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGGAACGGA | | TTATCAGCGTogga | | | | | |
| | | AACGTTCCGACGCGG | | | | | | | |
| | | CACCGAGTCGGTGC | | | | | | | |
| 4219 | L2_e7_ | GTTTAAGAGCTAGAA | 6737 | GTTTAAGAGCTAGA | 6887 | GCGGAAACGCTCCA | Stem loop | 7 | 90 |
| | 60 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTGGAGCGGA | | TTATCAGCGTtgga | | | | | |
| | | AACGCTCCAACGCGG | | | | | | | |
| | | CACCGAGTCGGTGC | | | | | | | |
| 4220 | L2_e7_ | GTTTAAGAGCTAGAA | 6738 | GTTTAAGAGCTAGA | 6888 | TCCGAAAGGAACGT | Stem loop | 7 | 90 |
| | 61 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTACGTTCCGA | | TTATCAGCGTacgt | | | | | |
| | | AAGGAACGTACGCGG | | | | | | | |
| | | CACCGAGTCGGTGC | | | | | | | |
| 4221 | L2_e7_ | GTTTAAGAGCTAGAA | 6739 | GTTTAAGAGCTAGA | 6889 | CTAGAAATAGCGAC | Stem loop | 7 | 90 |
| | 62 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGTCGCTAGA | | TTATCAGCGTgtcg | | | | | |
| | | AATAGCGACACGCGG | | | | | | | |
| | | CACCGAGTCGGTGC | | | | | | | |
| 4222 | L2_e7_ | GTTTAAGAGCTAGAA | 6740 | GTTTAAGAGCTAGA | 6890 | AGCGAAAGCTCTTC | Stem loop | 7 | 90 |
| | 63 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGAAGAGCGA | | TTATCAGCGTgaag | | | | | |
| | | AAGCTCTTCACGCGGC | | | | | | | |
| | | ACCGAGTCGGTGC | | | | | | | |
| 4223 | L2_e7_ | GTTTAAGAGCTAGAA | 6741 | GTTTAAGAGCTAGA | 6891 | GGGGAAACTCGCTT | Stem loop | 7 | 90 |
| | 64 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTAAGCGGGGA | | TTATCAGCGTaagc | | | | | |
| | | AACTCGCTTACGCGGC | | | | | | | |
| | | ACCGAGTCGGTGC | | | | | | | |
| 4224 | L2_e7_ | GTTTAAGAGCTAGAA | 6742 | GTTTAAGAGCTAGA | 6892 | TTCGAAAGGGCAGC | Stem loop | 7 | 90 |
| | 65 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCTGTTCGAA | | TTATCAGCGTgctg | | | | | |
| | | AGGGCAGCACGCGGC | | | | | | | |
| | | ACCGAGTCGGTGC | | | | | | | |
| 4225 | L2_e7_ | GTTTAAGAGCTAGAA | 6743 | GTTTAAGAGCTAGA | 6893 | CAGGAAACTGGGCT | Stem loop | 7 | 90 |
| | 66 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGGTCCAGGA | | TTATCAGCGTggtc | | | | | |
| | | AACTGGGCTACGCGG | | | | | | | |
| | | CACCGAGTCGGTGC | | | | | | | |
| 4226 | L2_e7_ | GTTTAAGAGCTAGAA | 6744 | GTTTAAGAGCTAGA | 6894 | CCAGAAATGGAGGC | Stem loop | 7 | 90 |
| | 67 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCTTCCAGA | | TTATCAGCGTgctt | | | | | |
| | | AATGGAGGCACGCGG | | | | | | | |
| | | CACCGAGTCGGTGC | | | | | | | |
| 4227 | L2_e7_ | GTTTAAGAGCTAGAA | 6745 | GTTTAAGAGCTAGA | 6895 | GGTGAAAACCGGCT | Stem loop | 7 | 90 |
| | 68 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTAGCCGGTGA | | TTATCAGCGTagcc | | | | | |
| | | AAACCGGCTACGCGG | | | | | | | |
| | | CACCGAGTCGGTGC | | | | | | | |
| 4228 | L2_e7_ | GTTTAAGAGCTAGAA | 6746 | GTTTAAGAGCTAGA | 6896 | GCGGAAACGCCGTC | Stem loop | 7 | 90 |
| | 69 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGACGGCGGA | | TTATCAGCGTgacg | | | | | |
| | | AACGCCGTCACGCGG | | | | | | | |
| | | CACCGAGTCGGTGC | | | | | | | |
| 4229 | L2_e7_ | GTTTAAGAGCTAGAA | 6747 | GTTTAAGAGCTAGA | 6897 | GCCGAAAGGCGAAG | Stem loop | 7 | 90 |
| | 70 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCTTCGCCGAA | | TTATCAGCGTette | | | | | |
| | | AGGCGAAGACGCGGC | | | | | | | |
| | | ACCGAGTCGGTGC | | | | | | | |
| 4230 | L2_e7_ | GTTTAAGAGCTAGAA | 6748 | GTTTAAGAGCTAGA | 6898 | CACGAAAGTGAGGG | Stem loop | 7 | 90 |
| | 71 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCTCTCACGAA | | TTATCAGCGTctct | | | | | |
| | | AGTGAGGGACGCGGC | | | | | | | |
| | | ACCGAGTCGGTGC | | | | | | | |
| 4231 | L2_e7_ | GTTTAAGAGCTAGAA | 6749 | GTTTAAGAGCTAGA | 6899 | GGCGAAAGCCTACT | Stem loop | 7 | 90 |
| | 72 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTAGTAGGCGA | | TTATCAGCGTagta | | | | | |
| | | AAGCCTACTACGCGG | | | | | | | |
| | | CACCGAGTCGGTGC | | | | | | | |
| 4232 | L2_e7_ | GTTTAAGAGCTAGAA | 6750 | GTTTAAGAGCTAGA | 6900 | TCAGAAATGACCTG | Stem loop | 7 | 90 |
| | 73 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTCGGGTCAGA | | TTATCAGCGTcggg | | | | | |
| | | AATGACCTGACGCGG | | | | | | | |
| | | CACCGAGTCGGTGC | | | | | | | |
| 4233 | L2_e7_ | GTTTAAGAGCTAGAA | 6751 | GTTTAAGAGCTAGA | 6901 | TCGGAAACGAGCTA | Stem loop | 7 | 90 |
| | 74 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTTAGCTCGGA | | TTATCAGCGTtagc | | | | | |
| | | AACGAGCTAACGCGG | | | | | | | |
| | | CACCGAGTCGGTGC | | | | | | | |
| 4234 | L2_e7_ | GTTTAAGAGCTAGAA | 6752 | GTTTAAGAGCTAGA | 6902 | CCAGAAATGGGTGC | Stem loop | 7 | 90 |
| | 75 | ATAGCAAGTTTAAAT | | AATAGCAAGTTTAA | | ACGCGGCACCGAGT | 2 | | |
| | | AAGGCTAGTCCGTTAT | | ATAAGGCTAGTCCG | | CGGTGC | | | |
| | | CAGCGTGCGCCCAGA | | TTATCAGCGTgcgc | | | | | |
| | | AATGGGTGCACGCGG | | | | | | | |
| | | CACCGAGTCGGTGC | | | | | | | |
| 4235 | L2_e8_ | GTTTAAGAGCTAGAA | 6753 | GTTTAAGAGCTAGA | 6903 | CTTGAAAGAGTGGG | Stem loop | 8 | 92 |
| | 1 | ATAGCAAGTTTAAAT | | AATAGCAAGTTTAA | | AACGCGGCACCGAG | 2 | | |
| | | AAGGCTAGTCCGTTAT | | ATAAGGCTAGTCCG | | TCGGTGC | | | |
| | | CAGCGTTCCCACTTGA | | TTATCAGCGTtccca | | | | | |
| | | AAGAGTGGGAACGCG | | | | | | | |
| | | GCACCGAGTCGGTGC | | | | | | | |
| 4236 | L2_e8_ | GTTTAAGAGCTAGAA | 6754 | GTTTAAGAGCTAGA | 6904 | CCCGAAAGGGGGAT | Stem loop | 8 | 92 |
| | 2 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCGTCCCCCGA | | TTATCAGCGTegtcc | | | | | |
| | | AAGGGGGATGACGCG | | | | | | | |
| | | GCACCGAGTCGGTGC | | | | | | | |
| 4237 | L2_e8_ | GTTTAAGAGCTAGAA | 6755 | GTTTAAGAGCTAGA | 6905 | GGAGAAATCCCTGC | Stem loop | 8 | 92 |
| | 3 | ATAGCAAGTTTAAAT | | AATAGCAAGTTTAA | | AACGCGGCACCGAG | 2 | | |
| | | AAGGCTAGTCCGTTAT | | ATAAGGCTAGTCCG | | TCGGTGC | | | |
| | | CAGCGTTGCAGGGAG | | TTATCAGCGTtgcag | | | | | |
| | | AAATCCCTGCAACGC | | | | | | | |
| | | GGCACCGAGTCGGTG | | | | | | | |
| | | C | | | | | | | |
| 4238 | L2_e8_ | GTTTAAGAGCTAGAA | 6756 | GTTTAAGAGCTAGA | 6906 | AAGGAAATTTGGCG | Stem loop | 8 | 92 |
| | 4 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTTCGCCAAGG | | TTATCAGCGTtegcc | | | | | |
| | | AAATTTGGCGGACGC | | | | | | | |
| | | GGCACCGAGTCGGTG | | | | | | | |
| | | C | | | | | | | |
| 4239 | L2_e8_ | GTTTAAGAGCTAGAA | 6757 | GTTTAAGAGCTAGA | 6907 | GCCGAAAGGCCCCG | Stem loop | 8 | 92 |
| | 5 | ATAGCAAGTTTAAAT | | AATAGCAAGTTTAA | | AACGCGGCACCGAG | 2 | | |
| | | AAGGCTAGTCCGTTAT | | ATAAGGCTAGTCCG | | TCGGTGC | | | |
| | | CAGCGTTCGGGGCCG | | TTATCAGCGTtcggg | | | | | |
| | | AAAGGCCCCGAACGC | | | | | | | |
| | | GGCACCGAGTCGGTG | | | | | | | |
| | | C | | | | | | | |
| 4240 | L2_e8_ | GTTTAAGAGCTAGAA | 6758 | GTTTAAGAGCTAGA | 6908 | TGGGAAACTACCTG | Stem loop | 8 | 92 |
| | 6 | ATAGCAAGTTTAAAT | | AATAGCAAGTTTAA | | AACGCGGCACCGAG | 2 | | |
| | | AAGGCTAGTCCGTTAT | | ATAAGGCTAGTCCG | | TCGGTGC | | | |
| | | CAGCGTTCGGGTGGG | | TTATCAGCGTtcggg | | | | | |
| | | AAACTACCTGAACGC | | | | | | | |
| | | GGCACCGAGTCGGTG | | | | | | | |
| | | C | | | | | | | |
| 4241 | L2_e8_ | GTTTAAGAGCTAGAA | 6759 | GTTTAAGAGCTAGA | 6909 | CTGGAAACGGGTGC | Stem loop | 8 | 92 |
| | 7 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTTGCACCTGG | | TTATCAGCGTtgcac | | | | | |
| | | AAACGGGTGCGACGC | | | | | | | |
| | | GGCACCGAGTCGGTG | | | | | | | |
| | | C | | | | | | | |
| 4242 | L2_e8_ | GTTTAAGAGCTAGAA | 6760 | GTTTAAGAGCTAGA | 6910 | CGGGAAACTGTAGA | Stem loop | 8 | 92 |
| | 8 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCTCTACGGG | | TTATCAGCGTctcta | | | | | |
| | | AAACTGTAGAGACGC | | | | | | | |
| | | GGCACCGAGTCGGTG | | | | | | | |
| | | C | | | | | | | |
| 4243 | L2_e8_ | GTTTAAGAGCTAGAA | 6761 | GTTTAAGAGCTAGA | 6911 | TCTGAAAAGGCGGC | Stem loop | 8 | 92 |
| | 9 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCGCCGTCTGA | | TTATCAGCGTcgccg | | | | | |
| | | AAAGGCGGCGACGCG | | | | | | | |
| | | GCACCGAGTCGGTGC | | | | | | | |
| 4244 | L2_e8_ | GTTTAAGAGCTAGAA | 6762 | GTTTAAGAGCTAGA | 6912 | GATGAAAATCCCGT | Stem loop | 8 | 92 |
| | 10 | ATAGCAAGTTTAAAT | | AATAGCAAGTTTAA | | AACGCGGCACCGAG | 2 | | |
| | | AAGGCTAGTCCGTTAT | | ATAAGGCTAGTCCG | | TCGGTGC | | | |
| | | CAGCGTTACGGGATG | | TTATCAGCGTtacgg | | | | | |
| | | AAAATCCCGTAACGC | | | | | | | |
| | | GGCACCGAGTCGGTG | | | | | | | |
| | | C | | | | | | | |
| 4245 | L2_e8_ | GTTTAAGAGCTAGAA | 6763 | GTTTAAGAGCTAGA | 6913 | CCGGAAACGGCAGC | Stem loop | 8 | 92 |
| | 11 | ATAGCAAGTTTAAAT | | AATAGCAAGTTTAA | | TACGCGGCACCGAG | 2 | | |
| | | AAGGCTAGTCCGTTAT | | ATAAGGCTAGTCCG | | TCGGTGC | | | |
| | | CAGCGTAGCTGCCGG | | TTATCAGCGTagctg | | | | | |
| | | AAACGGCAGCTACGC | | | | | | | |
| | | GGCACCGAGTCGGTG | | | | | | | |
| | | C | | | | | | | |
| 4246 | L2_e8_ | GTTTAAGAGCTAGAA | 6764 | GTTTAAGAGCTAGA | 6914 | TCTGAAAAGGCCAG | Stem loop | 8 | 92 |
| | 12 | ATAGCAAGTTTAAAT | | AATAGCAAGTTTAA | | TACGCGGCACCGAG | 2 | | |
| | | AAGGCTAGTCCGTTAT | | ATAAGGCTAGTCCG | | TCGGTGC | | | |
| | | CAGCGTACTGGTCTGA | | TTATCAGCGTactgg | | | | | |
| | | AAAGGCCAGTACGCG | | | | | | | |
| | | GCACCGAGTCGGTGC | | | | | | | |
| 4247 | L2_e8_ | GTTTAAGAGCTAGAA | 6765 | GTTTAAGAGCTAGA | 6915 | GCAGAAATGTCAGC | Stem loop | 8 | 92 |
| | 13 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCGCTGGCAG | | TTATCAGCGTcgctg | | | | | |
| | | AAATGTCAGCGACGC | | | | | | | |
| | | GGCACCGAGTCGGTG | | | | | | | |
| | | C | | | | | | | |
| 4248 | L2_e8_ | GTTTAAGAGCTAGAA | 6766 | GTTTAAGAGCTAGA | 6916 | CCTGAAAAGGACAG | Stem loop | 8 | 92 |
| | 14 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCCTGTCCTGA | | TTATCAGCGTcctgt | | | | | |
| | | AAAGGACAGGACGCG | | | | | | | |
| | | GCACCGAGTCGGTGC | | | | | | | |
| 4249 | L2_e8_ | GTTTAAGAGCTAGAA | 6767 | GTTTAAGAGCTAGA | 6917 | GAGGAAACTCGGTA | Stem loop | 8 | 92 |
| | 15 | ATAGCAAGTTTAAAT | | AATAGCAAGTTTAA | | TACGCGGCACCGAG | 2 | | |
| | | AAGGCTAGTCCGTTAT | | ATAAGGCTAGTCCG | | TCGGTGC | | | |
| | | CAGCGTATACCGAGG | | TTATCAGCGTatacc | | | | | |
| | | AAACTCGGTATACGC | | | | | | | |
| | | GGCACCGAGTCGGTG | | | | | | | |
| | | C | | | | | | | |
| 4250 | L2_e8_ | GTTTAAGAGCTAGAA | 6768 | GTTTAAGAGCTAGA | 6918 | CGCGAAAGCGACCT | Stem loop | 8 | 92 |
| | 16 | ATAGCAAGTTTAAAT | | AATAGCAAGTTTAA | | AACGCGGCACCGAG | 2 | | |
| | | AAGGCTAGTCCGTTAT | | ATAAGGCTAGTCCG | | TCGGTGC | | | |
| | | CAGCGTTAGGTCGCG | | TTATCAGCGTtaggt | | | | | |
| | | AAAGCGACCTAACGC | | | | | | | |
| | | GGCACCGAGTCGGTG | | | | | | | |
| | | C | | | | | | | |
| 4251 | L2_e8_ | GTTTAAGAGCTAGAA | 6769 | GTTTAAGAGCTAGA | 6919 | AACGAAAGTTTCGG | Stem loop | 8 | 92 |
| | 17 | ATAGCAAGTTTAAAT | | AATAGCAAGTTTAA | | CACGCGGCACCGAG | 2 | | |
| | | AAGGCTAGTCCGTTAT | | ATAAGGCTAGTCCG | | TCGGTGC | | | |
| | | CAGCGTGCTGAAACG | | TTATCAGCGTgctga | | | | | |
| | | AAAGTTTCGGCACGC | | | | | | | |
| | | GGCACCGAGTCGGTG | | | | | | | |
| | | C | | | | | | | |
| 4252 | L2_e8_ | GTTTAAGAGCTAGAA | 6770 | GTTTAAGAGCTAGA | 6920 | AAAGAAATTTGCCG | Stem loop | 8 | 92 |
| | 18 | ATAGCAAGTTTAAAT | | AATAGCAAGTTTAA | | TACGCGGCACCGAG | 2 | | |
| | | AAGGCTAGTCCGTTAT | | ATAAGGCTAGTCCG | | TCGGTGC | | | |
| | | CAGCGTACGGCAAAG | | TTATCAGCGTacggc | | | | | |
| | | AAATTTGCCGTACGCG | | | | | | | |
| | | GCACCGAGTCGGTGC | | | | | | | |
| 4253 | L2_e8_ | GTTTAAGAGCTAGAA | 6771 | GTTTAAGAGCTAGA | 6921 | CCTGAAAAGGCTCA | Stem loop | 8 | 92 |
| | 19 | ATAGCAAGTTTAAAT | | AATAGCAAGTTTAA | | TACGCGGCACCGAG | 2 | | |
| | | AAGGCTAGTCCGTTAT | | ATAAGGCTAGTCCG | | TCGGTGC | | | |
| | | CAGCGTGTGAGCCTG | | TTATCAGCGTgtgag | | | | | |
| | | AAAAGGCTCATACGC | | | | | | | |
| | | GGCACCGAGTCGGTG | | | | | | | |
| | | C | | | | | | | |
| 4254 | L2_e8_ | GTTTAAGAGCTAGAA | 6772 | GTTTAAGAGCTAGA | 6922 | AAAGAAATTTTGCC | Stem loop | 8 | 92 |
| | 20 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCGGCAAAAG | | TTATCAGCGTcggca | | | | | |
| | | AAATTTTGCCGACGCG | | | | | | | |
| | | GCACCGAGTCGGTGC | | | | | | | |
| 4255 | L2_e8_ | GTTTAAGAGCTAGAA | 6773 | GTTTAAGAGCTAGA | 6923 | AGCGAAAGTTAGCG | Stem loop | 8 | 92 |
| | 21 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCCGCTAGCG | | TTATCAGCGTccgct | | | | | |
| | | AAAGTTAGCGGACGC | | | | | | | |
| | | GGCACCGAGTCGGTG | | | | | | | |
| | | C | | | | | | | |
| 4256 | L2_e8_ | GTTTAAGAGCTAGAA | 6774 | GTTTAAGAGCTAGA | 6924 | ATCGAAAGATGGTG | Stem loop | 8 | 92 |
| | 22 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCCACCATCG | | TTATCAGCGTccacc | | | | | |
| | | AAAGATGGTGGACGC | | | | | | | |
| | | GGCACCGAGTCGGTG | | | | | | | |
| | | C | | | | | | | |
| 4257 | L2_e8_ | GTTTAAGAGCTAGAA | 6775 | GTTTAAGAGCTAGA | 6925 | TGGGAAACCGGGAC | Stem loop | 8 | 92 |
| | 23 | ATAGCAAGTTTAAAT | | AATAGCAAGTTTAA | | TACGCGGCACCGAG | 2 | | |
| | | AAGGCTAGTCCGTTAT | | ATAAGGCTAGTCCG | | TCGGTGC | | | |
| | | CAGCGTAGTCCTGGG | | TTATCAGCGTagtcc | | | | | |
| | | AAACCGGGACTACGC | | | | | | | |
| | | GGCACCGAGTCGGTG | | | | | | | |
| | | C | | | | | | | |
| 4258 | L2_e8_ | GTTTAAGAGCTAGAA | 6776 | GTTTAAGAGCTAGA | 6926 | TACGAAAGTAATGC | Stem loop | 8 | 92 |
| | 24 | ATAGCAAGTTTAAAT | | AATAGCAAGTTTAA | | GACGCGGCACCGAG | 2 | | |
| | | AAGGCTAGTCCGTTAT | | ATAAGGCTAGTCCG | | TCGGTGC | | | |
| | | CAGCGTCGCATTACG | | TTATCAGCGTcgcat | | | | | |
| | | AAAGTAATGCGACGC | | | | | | | |
| | | GGCACCGAGTCGGTG | | | | | | | |
| | | C | | | | | | | |
| 4259 | L2_e8_ | GTTTAAGAGCTAGAA | 6777 | GTTTAAGAGCTAGA | 6927 | CACGAAAGTGTCAT | Stem loop | 8 | 92 |
| | 25 | ATAGCAAGTTTAAAT | | AATAGCAAGTTTAA | | CACGCGGCACCGAG | 2 | | |
| | | AAGGCTAGTCCGTTAT | | ATAAGGCTAGTCCG | | TCGGTGC | | | |
| | | CAGCGTGGTGACACG | | TTATCAGCGTggtga | | | | | |
| | | AAAGTGTCATCACGC | | | | | | | |
| | | GGCACCGAGTCGGTG | | | | | | | |
| | | C | | | | | | | |
| 4260 | T_e1a_ | GTTTAAGAGCCCGGA | 6778 | GTTTAAGAG | 6928 | CCCGGAAACGGGCA | Tetraloop | 1 | 78 |
| | 1 | AACGGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4261 | T_e1a_ | GTTTAAGAGCCCCGA | 6779 | GTTTAAGAG | 6929 | CCCCGAAAGGGGCA | Tetraloop | 1 | 78 |
| | 2 | AAGGGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4262 | T_e1a_ | GTTTAAGAGCCGGGA | 6780 | GTTTAAGAG | 6930 | CCGGGAAACCGGCA | Tetraloop | 1 | 78 |
| | 3 | AACCGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4263 | T_e1a_ | GTTTAAGAGCCGCGA | 6781 | GTTTAAGAG | 6931 | CCGCGAAAGCGGCA | Tetraloop | 1 | 78 |
| | 4 | AAGCGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4264 | T_e1a_ | GTTTAAGAGCGGCGA | 6782 | GTTTAAGAG | 6932 | CGGCGAAAGCCGCA | Tetraloop | 1 | 78 |
| | 5 | AAGCCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4265 | T_e1a_ | GTTTAAGAGCGCCGA | 6783 | GTTTAAGAG | 6933 | CGCCGAAAGGCGCA | Tetraloop | 1 | 78 |
| | 6 | AAGGCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4266 | T_e1a_ | GTTTAAGAGCGCGGA | 6784 | GTTTAAGAG | 6934 | CGCGGAAACGCGCA | Tetraloop | 1 | 78 |
| | 7 | AACGCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4267 | T_e1a_ | GTTTAAGAGCGGGGA | 6785 | GTTTAAGAG | 6935 | CGGGGAAACCCGCA | Tetraloop | 1 | 78 |
| | 8 | AACCCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4268 | T_e1b_ | GTTTAAGAGCCTGGA | 6786 | GTTTAAGAG | 6936 | CCTGGAAACAGGCA | Tetraloop | 1 | 78 |
| | 1 | AACAGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4269 | T_e1b_ | GTTTAAGAGCGAGGA | 6787 | GTTTAAGAG | 6937 | CGAGGAAACTCGCA | Tetraloop | 1 | 78 |
| | 2 | AACTCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4270 | T_e1b_ | GTTTAAGAGCGTCGA | 6788 | GTTTAAGAG | 6938 | CGTCGAAAGACGCA | Tetraloop | 1 | 78 |
| | 3 | AAGACGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4271 | T_e1b_ | GTTTAAGAGCAGCGA | 6789 | GTTTAAGAG | 6939 | CAGCGAAAGCTGCA | Tetraloop | 1 | 78 |
| | 4 | AAGCTGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4272 | T_e1b_ | GTTTAAGAGCCGGGA | 6790 | GTTTAAGAG | 6930 | CCGGGAAACCGGCA | Tetraloop | 1 | 78 |
| | 5 | AACCGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4273 | T_e1b_ | GTTTAAGAGCCGTGA | 6791 | GTTTAAGAG | 6940 | CCGTGAAAACGGCA | Tetraloop | 1 | 78 |
| | 6 | AAACGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4274 | T_e1b_ | GTTTAAGAGCTGGGA | 6792 | GTTTAAGAG | 6941 | CTGGGAAACCAGCA | Tetraloop | 1 | 78 |
| | 7 | AACCAGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4275 | T_e1b_ | GTTTAAGAGCCCTGA | 6793 | GTTTAAGAG | 6942 | CCCTGAAAAGGGCA | Tetraloop | 1 | 78 |
| | 8 | AAAGGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4276 | T_e1b_ | GTTTAAGAGCGACGA | 6794 | GTTTAAGAG | 6943 | CGACGAAAGTCGCA | Tetraloop | 1 | 78 |
| | 9 | AAGTCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4277 | T_e1b_ | GTTTAAGAGCGGAGA | 6795 | GTTTAAGAG | 6944 | CGGAGAAATCCGCA | Tetraloop | 1 | 78 |
| | 10 | AATCCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4278 | T_e1b_ | GTTTAAGAGCACCGA | 6796 | GTTTAAGAG | 6945 | CACCGAAAGGTGCA | Tetraloop | 1 | 78 |
| | 11 | AAGGTGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4279 | T_e1b_ | GTTTAAGAGCAGGGA | 6797 | GTTTAAGAG | 6946 | CAGGGAAACCTGCA | Tetraloop | 1 | 78 |
| | 12 | AACCTGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4280 | T_e1b_ | GTTTAAGAGCGGGGA | 6798 | GTTTAAGAG | 6947 | CGGGGAAATCCGCA | Tetraloop | 1 | 78 |
| | 13 | AATCCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4452 | T_e1b_ | GTTTAAGAGCGGGGA | 6799 | GTTTAAGAG | 6948 | CGGGGAAATCCGCA | Tetraloop | 1 | 78 |
| | 13_SL2 | AATCCGCAAGTTTAA | | | | AGTTTAAATAAGGC | | | |
| | | ATAAGGCTAGTCCGTT | | | | TAGTCCGTTATCAGC | | | |
| | | ATCAGCGTGAAAACG | | | | GTGAAAACGCGGCA | | | |
| | | CGGCACCGAGTCGGT | | | | CCGAGTCGGTGC | | | |
| | | GC | | | | | | | |
| 4281 | T_e1b_ | GTTTAAGAGCCGCGA | 6800 | GTTTAAGAG | 6931 | CCGCGAAAGCGGCA | Tetraloop | 1 | 78 |
| | 14 | AAGCGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4282 | T_e1b_ | GTTTAAGAGCCCCGA | 6801 | GTTTAAGAG | 6929 | CCCCGAAAGGGGCA | Tetraloop | 1 | 78 |
| | 15 | AAGGGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4283 | T_e1b_ | GTTTAAGAGCCTCGA | 6802 | GTTTAAGAG | 6949 | CCTCGAAAGGGGCA | Tetraloop | 1 | 78 |
| | 16 | AAGGGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4284 | T_e1b_ | GTTTAAGAGCGGGGA | 6803 | GTTTAAGAG | 6935 | CGGGGAAACCCGCA | Tetraloop | 1 | 78 |
| | 17 | AACCCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4285 | T_e1b_ | GTTTAAGAGCGCGGA | 6804 | GTTTAAGAG | 6934 | CGCGGAAACGCGCA | Tetraloop | 1 | 78 |
| | 18 | AACGCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4286 | T_e1b_ | GTTTAAGAGCTGCGA | 6805 | GTTTAAGAG | 6950 | CTGCGAAAGCAGCA | Tetraloop | 1 | 78 |
| | 19 | AAGCAGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4287 | T_e1b_ | GTTTAAGAGCACGGA | 6806 | GTTTAAGAG | 6951 | CACGGAAACGTGCA | Tetraloop | 1 | 78 |
| | 20 | AACGTGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4288 | T_e1b_ | GTTTAAGAGCGTGGA | 6807 | GTTTAAGAG | 6952 | CGTGGAAACACGCA | Tetraloop | 1 | 78 |
| | 21 | AACACGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4289 | T_e1b_ | GTTTAAGAGCGCCGA | 6808 | GTTTAAGAG | 6933 | CGCCGAAAGGCGCA | Tetraloop | 1 | 78 |
| | 22 | AAGGCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4290 | T_e1b_ | GTTTAAGAGCTCCGA | 6809 | GTTTAAGAG | 6953 | CTCCGAAAGGGGCA | Tetraloop | 1 | 78 |
| | 23 | AAGGGGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4291 | T_e1b_ | GTTTAAGAGCGGCGA | 6810 | GTTTAAGAG | 6932 | CGGCGAAAGCCGCA | Tetraloop | 1 | 78 |
| | 24 | AAGCCGCAAGTTTAA | | | | AGTTTAAATAAGGC | | | |
| | | ATAAGGCTAGTCCGTT | | | | TAGTCCGTTATCAAC | | | |
| | | ATCAACTTGAAAAAG | | | | TTGAAAAAGTGGCA | | | |
| | | TGGCACCGAGTCGGT | | | | CCGAGTCGGTGC | | | |
| | | GC | | | | | | | |
| 4292 | T_e2_1 | GTTTAAGAGCCACCG | 6811 | GTTTAAGAGC | 6954 | CACCGAAAGGTGGC | Tetraloop | 2 | 80 |
| | | AAAGGTGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4293 | T_e2_2 | GTTTAAGAGCCGTCG | 6812 | GTTTAAGAGC | 6955 | CGTCGAAAGACGGC | Tetraloop | 2 | 80 |
| | | AAAGACGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4294 | T_e2_3 | GTTTAAGAGCCAGCG | 6813 | GTTTAAGAGC | 6956 | CAGCGAAAGCTGGC | Tetraloop | 2 | 80 |
| | | AAAGCTGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4295 | T_e2_4 | GTTTAAGAGCGCGGG | 6814 | GTTTAAGAGC | 6957 | GCGGGAAACCGCGC | Tetraloop | 2 | 80 |
| | | AAACCGCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4296 | T_e2_5 | GTTTAAGAGCTCGCG | 6815 | GTTTAAGAGC | 6958 | TCGCGAAAGCGAGC | Tetraloop | 2 | 80 |
| | | AAAGCGAGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4297 | T_e2_6 | GTTTAAGAGCTCGGG | 6816 | GTTTAAGAGC | 6959 | TCGGGAAACCGAGC | Tetraloop | 2 | 80 |
| | | AAACCGAGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4298 | T_e2_7 | GTTTAAGAGCCCCTGA | 6817 | GTTTAAGAGC | 6960 | CCCTGAAAGGGGGC | Tetraloop | 2 | 80 |
| | | AAGGGGGCAAGTTTA | | | | AAGTTTAAATAAGG | | | |
| | | AATAAGGCTAGTCCG | | | | CTAGTCCGTTATCAA | | | |
| | | TTATCAACTTGAAAAA | | | | CTTGAAAAAGTGGC | | | |
| | | GTGGCACCGAGTCGG | | | | ACCGAGTCGGTGC | | | |
| | | TGC | | | | | | | |
| 4299 | T_e2_8 | GTTTAAGAGCCGTGG | 6818 | GTTTAAGAGC | 6961 | CGTGGAAACGCGGC | Tetraloop | 2 | 80 |
| | | AAACGCGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4300 | T_e2_9 | GTTTAAGAGCGACCG | 6819 | GTTTAAGAGC | 6962 | GACCGAAAGGTCGC | Tetraloop | 2 | 80 |
| | | AAAGGTCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4301 | T_e2_10 | GTTTAAGAGCACGCG | 6820 | GTTTAAGAGC | 6963 | ACGCGAAAGCGTGC | Tetraloop | 2 | 80 |
| | | AAAGCGTGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4302 | T_e2_11 | GTTTAAGAGCGAGCG | 6821 | GTTTAAGAGC | 6964 | GAGCGAAAGCTCGC | Tetraloop | 2 | 80 |
| | | AAAGCTCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4303 | T_e2_12 | GTTTAAGAGCTCCGG | 6822 | GTTTAAGAGC | 6965 | TCCGGAAACGGAGC | Tetraloop | 2 | 80 |
| | | AAACGGAGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4304 | T_e2_13 | GTTTAAGAGCGCCAG | 6823 | GTTTAAGAGC | 6966 | GCCAGAAATGGCGC | Tetraloop | 2 | 80 |
| | | AAATGGCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4305 | T_e2_14 | GTTTAAGAGCCCGTG | 6824 | GTTTAAGAGC | 6967 | CCGTGAAAACGGGC | Tetraloop | 2 | 80 |
| | | AAAACGGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4306 | T_e2_15 | GTTTAAGAGCGTGCG | 6825 | GTTTAAGAGC | 6968 | GTGCGAAAGCACGC | Tetraloop | 2 | 80 |
| | | AAAGCACGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4307 | T_e2_16 | GTTTAAGAGCTGCCG | 6826 | GTTTAAGAGC | 6969 | TGCCGAAAGGCGGC | Tetraloop | 2 | 80 |
| | | AAAGGCGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4308 | T_e2_17 | GTTTAAGAGCCCTGG | 6827 | GTTTAAGAGC | 6970 | CCTGGAAACAGGGC | Tetraloop | 2 | 80 |
| | | AAACAGGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4309 | T_e2_18 | GTTTAAGAGCGCTCG | 6828 | GTTTAAGAGC | 6971 | GCTCGAAAGGGCGC | Tetraloop | 2 | 80 |
| | | AAAGGGCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4310 | T_e2_19 | GTTTAAGAGCGCCCG | 6829 | GTTTAAGAGC | 6972 | GCCCGAAAGGGTGC | Tetraloop | 2 | 80 |
| | | AAAGGGTGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4311 | T_e2_20 | GTTTAAGAGCTGGGG | 6830 | GTTTAAGAGC | 6973 | TGGGGAAACCCGGC | Tetraloop | 2 | 80 |
| | | AAACCCGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4312 | T_e2_21 | GTTTAAGAGCGGTCG | 6831 | GTTTAAGAGC | 6974 | GGTCGAAAGGCCGC | Tetraloop | 2 | 80 |
| | | AAAGGCCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4313 | T_e2_22 | GTTTAAGAGCAGGCG | 6832 | GTTTAAGAGC | 6975 | AGGCGAAAGCCTGC | Tetraloop | 2 | 80 |
| | | AAAGCCTGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4314 | T_e2_23 | GTTTAAGAGCGTGGG | 6833 | GTTTAAGAGC | 6976 | GTGGGAAACCGCGC | Tetraloop | 2 | 80 |
| | | AAACCGCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4315 | T_e2_24 | GTTTAAGAGCGCTGG | 6834 | GTTTAAGAGC | 6977 | GCTGGAAACAGCGC | Tetraloop | 2 | 80 |
| | | AAACAGCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4316 | T_e2_25 | GTTTAAGAGCCTGGG | 6835 | GTTTAAGAGC | 6978 | CTGGGAAACCAGGC | Tetraloop | 2 | 80 |
| | | AAACCAGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4317 | T_e2_26 | GTTTAAGAGCCGCAG | 6836 | GTTTAAGAGC | 6979 | CGCAGAAATGCGGC | Tetraloop | 2 | 80 |
| | | AAATGCGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4318 | T_e2_27 | GTTTAAGAGCCCGGG | 6837 | GTTTAAGAGC | 6980 | CCGGGAAACCGGGC | Tetraloop | 2 | 80 |
| | | AAACCGGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4319 | T_e2_28 | GTTTAAGAGCCCGCG | 6838 | GTTTAAGAGC | 6981 | CCGCGAAAGCGGGC | Tetraloop | 2 | 80 |
| | | AAAGCGGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4320 | T_e2_29 | GTTTAAGAGCAGCCG | 6839 | GTTTAAGAGC | 6982 | AGCCGAAAGGCTGC | Tetraloop | 2 | 80 |
| | | AAAGGCTGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4321 | T_e2_30 | GTTTAAGAGCTGCCG | 6840 | GTTTAAGAGC | 6983 | TGCCGAAAGGCAGC | Tetraloop | 2 | 80 |
| | | AAAGGCAGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4322 | T_e2_31 | GTTTAAGAGCGGACG | 6841 | GTTTAAGAGC | 6984 | GGACGAAAGTCCGC | Tetraloop | 2 | 80 |
| | | AAAGTCCGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4323 | T_e2_32 | GTTTAAGAGCCCAGG | 6842 | GTTTAAGAGC | 6985 | CCAGGAAACTGGGC | Tetraloop | 2 | 80 |
| | | AAACTGGGCAAGTTT | | | | AAGTTTAAATAAGG | | | |
| | | AAATAAGGCTAGTCC | | | | CTAGTCCGTTATCAA | | | |
| | | GTTATCAACTTGAAAA | | | | CTTGAAAAAGTGGC | | | |
| | | AGTGGCACCGAGTCG | | | | ACCGAGTCGGTGC | | | |
| | | GTGC | | | | | | | |
| 4324 | T_e3_1 | GTTTAAGAGCGCGTG | 6843 | GTTTAAGAGCC | 6986 | GTTTAAGAGCGCGT | Tetraloop | 3 | 82 |
| | | GAAACATGCGCAAGT | | | | GGAAACATGCGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4325 | T_e3_2 | GTTTAAGAGCCTCCCG | 6844 | GTTTAAGAGCC | 6987 | TCCCGAAAGGGGGG | Tetraloop | 3 | 82 |
| | | AAAGGGGGGCAAGTT | | | | CAAGTTTAAATAAG | | | |
| | | TAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4326 | T_e3_3 | GTTTAAGAGCGCCAT | 6845 | GTTTAAGAGCC | 6988 | GTTTAAGAGCGCCA | Tetraloop | 3 | 82 |
| | | GAAAATGGCGCAAGT | | | | TGAAAATGGCGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4327 | T_e3_4 | GTTTAAGAGCGCAAC | 6846 | GTTTAAGAGCC | 6989 | GTTTAAGAGCGCAA | Tetraloop | 3 | 82 |
| | | GAAAGTTGCGCAAGT | | | | CGAAAGTTGCGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4328 | T_e3_5 | GTTTAAGAGCCCTGA | 6847 | GTTTAAGAGCC | 6990 | CTGAGAAATCAGGG | Tetraloop | 3 | 82 |
| | | GAAATCAGGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4329 | T_e3_6 | GTTTAAGAGCCTCCAG | 6848 | GTTTAAGAGCC | 6991 | TCCAGAAATGGAGG | Tetraloop | 3 | 82 |
| | | AAATGGAGGCAAGTT | | | | CAAGTTTAAATAAG | | | |
| | | TAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4330 | T_e3_7 | GTTTAAGAGCGCACA | 6849 | GTTTAAGAGCC | 6992 | GTTTAAGAGCGCAC | Tetraloop | 3 | 82 |
| | | GAAATGTGCGCAAGT | | | | AGAAATGTGCGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4331 | T_e3_8 | GTTTAAGAGCGAAGC | 6850 | GTTTAAGAGCC | 6993 | GTTTAAGAGCGAAG | Tetraloop | 3 | 82 |
| | | GAAAGCTTCGCAAGT | | | | CGAAAGCTTCGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4332 | T_e3_9 | GTTTAAGAGCGCCTCG | 6851 | GTTTAAGAGCC | 6994 | GTTTAAGAGCGCCTC | Tetraloop | 3 | 82 |
| | | AAAGAGGCGCAAGTT | | | | GAAAGAGGCGCAAG | | | |
| | | TAAATAAGGCTAGTC | | | | TTTAAATAAGGCTA | | | |
| | | CGTTATCAACTTGAAA | | | | GTCCGTTATCAACTT | | | |
| | | AAGTGGCACCGAGTC | | | | GAAAAAGTGGCACC | | | |
| | | GGTGC | | | | GAGTCGGTGC | | | |
| 4333 | T_e3_10 | GTTTAAGAGCCCGGG | 6852 | GTTTAAGAGCC | 6995 | CGGGGAAACTCGGG | Tetraloop | 3 | 82 |
| | | GAAACTCGGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4334 | T_e3_11 | GTTTAAGAGCGTCTGG | 6853 | GTTTAAGAGCC | 6996 | GTTTAAGAGCGTCTG | Tetraloop | 3 | 82 |
| | | AAACAGACGCAAGTT | | | | GAAACAGACGCAAG | | | |
| | | TAAATAAGGCTAGTC | | | | TTTAAATAAGGCTA | | | |
| | | CGTTATCAACTTGAAA | | | | GTCCGTTATCAACTT | | | |
| | | AAGTGGCACCGAGTC | | | | GAAAAAGTGGCACC | | | |
| | | GGTGC | | | | GAGTCGGTGC | | | |
| 4335 | T_e3_12 | GTTTAAGAGCGCAGG | 6854 | GTTTAAGAGCC | 6997 | GTTTAAGAGCGCAG | Tetraloop | 3 | 82 |
| | | GAAACCTGTGCAAGT | | | | GGAAACCTGTGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4336 | T_e3_13 | GTTTAAGAGCCGCCC | 6855 | GTTTAAGAGCC | 6998 | GCCCGAAAGGGCGG | Tetraloop | 3 | 82 |
| | | GAAAGGGCGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4337 | T_e3_14 | GTTTAAGAGCCCCGC | 6856 | GTTTAAGAGCC | 6999 | CCGCGAAAGCGGGG | Tetraloop | 3 | 82 |
| | | GAAAGCGGGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4338 | T_e3_15 | GTTTAAGAGCTGGCA | 6857 | GTTTAAGAGCC | 7000 | GTTTAAGAGCTGGC | Tetraloop | 3 | 82 |
| | | GAAATGCCAGCAAGT | | | | AGAAATGCCAGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4339 | T_e3_16 | GTTTAAGAGCGCCTTG | 6858 | GTTTAAGAGCC | 7001 | GTTTAAGAGCGCCTT | Tetraloop | 3 | 82 |
| | | AAAAAGGCGCAAGTT | | | | GAAAAAGGCGCAAG | | | |
| | | TAAATAAGGCTAGTC | | | | TTTAAATAAGGCTA | | | |
| | | CGTTATCAACTTGAAA | | | | GTCCGTTATCAACTT | | | |
| | | AAGTGGCACCGAGTC | | | | GAAAAAGTGGCACC | | | |
| | | GGTGC | | | | GAGTCGGTGC | | | |
| 4340 | T_e3_17 | GTTTAAGAGCGCCAG | 6859 | GTTTAAGAGCC | 7002 | GTTTAAGAGCGCCA | Tetraloop | 3 | 82 |
| | | GAAACTGGCGCAAGT | | | | GGAAACTGGCGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4341 | T_e3_18 | GTTTAAGAGCCCGCG | 6860 | GTTTAAGAGCC | 7003 | CGCGGAAACGCGGG | Tetraloop | 3 | 82 |
| | | GAAACGCGGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4342 | T_e3_19 | GTTTAAGAGCCGGCC | 6861 | GTTTAAGAGCC | 7004 | GGCCGAAAGGCCGG | Tetraloop | 3 | 82 |
| | | GAAAGGCCGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4343 | T_e3_2 | GTTTAAGAGCCCATG | 6862 | GTTTAAGAGCC | 7005 | CATGGAAACATGGG | Tetraloop | 3 | 82 |
| | 0 | GAAACATGGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4344 | T_e3_21 | GTTTAAGAGCAACCG | 6863 | GTTTAAGAGCC | 7006 | GTTTAAGAGCAACC | Tetraloop | 3 | 82 |
| | | GAAACGGTTGCAAGT | | | | GGAAACGGTTGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4345 | T_e3_22 | GTTTAAGAGCGTTCGG | 6864 | GTTTAAGAGCC | 7007 | GTTTAAGAGCGTTCG | Tetraloop | 3 | 82 |
| | | AAACGAACGCAAGTT | | | | GAAACGAACGCAAG | | | |
| | | TAAATAAGGCTAGTC | | | | TTTAAATAAGGCTA | | | |
| | | CGTTATCAACTTGAAA | | | | GTCCGTTATCAACTT | | | |
| | | AAGTGGCACCGAGTC | | | | GAAAAAGTGGCACC | | | |
| | | GGTGC | | | | GAGTCGGTGC | | | |
| 4346 | T_e3_23 | GTTTAAGAGCGAGGC | 6865 | GTTTAAGAGCC | 7008 | GTTTAAGAGCGAGG | Tetraloop | 3 | 82 |
| | | GAAAGTCTCGCAAGT | | | | CGAAAGTCTCGCAA | | | |
| | | TTAAATAAGGCTAGTC | | | | GTTTAAATAAGGCT | | | |
| | | CGTTATCAACTTGAAA | | | | AGTCCGTTATCAACT | | | |
| | | AAGTGGCACCGAGTC | | | | TGAAAAAGTGGCAC | | | |
| | | GGTGC | | | | CGAGTCGGTGC | | | |
| 4347 | T_e3_24 | GTTTAAGAGCCCGAG | 6866 | GTTTAAGAGCC | 7009 | CGAGGAAACTCGGG | Tetraloop | 3 | 82 |
| | | GAAACTCGGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4348 | T_e3_25 | GTTTAAGAGCCGCAG | 6867 | GTTTAAGAGCC | 7010 | GCAGGAAACTGCGG | Tetraloop | 3 | 82 |
| | | GAAACTGCGGCAAGT | | | | CAAGTTTAAATAAG | | | |
| | | TTAAATAAGGCTAGTC | | | | GCTAGTCCGTTATCA | | | |
| | | CGTTATCAACTTGAAA | | | | ACTTGAAAAAGTGG | | | |
| | | AAGTGGCACCGAGTC | | | | CACCGAGTCGGTGC | | | |
| | | GGTGC | | | | | | | |
| 4349 | T_e3_26 | GTTTAAGAGCTCCACG | 6868 | GTTTAAGAGCC | 7011 | GTTTAAGAGCTCCAC | Tetraloop | 3 | 82 |
| | | AAAGTGGAGCAAGTT | | | | GAAAGTGGAGCAAG | | | |
| | | TAAATAAGGCTAGTC | | | | TTTAAATAAGGCTA | | | |
| | | CGTTATCAACTTGAAA | | | | GTCCGTTATCAACTT | | | |
| | | AAGTGGCACCGAGTC | | | | GAAAAAGTGGCACC | | | |
| | | GGTGC | | | | GAGTCGGTGC | | | |
| 4350 | canonical | GTTTTAGAGCTAGAA | 6869 | | 7012 | GTTTTAGAGCTAGA | none | 0 | 76 |
| or 16 | SpCas9 | ATAGCAAGTTAAAAT | | | | AATAGCAAGTTAAA | | | |
| | | AAGGCTAGTCCGTTAT | | | | ATAAGGCTAGTCCG | | | |
| | | CAACTTGAAAAAGTG | | | | TTATCAACTTGAAAA | | | |
| | | GCACCGAGTCGGTGC | | | | AGTGGCACCGAGTC | | | |
| | | | | | | GGTGC | | | |
| 4351 | dnr6 | GTTTTAGAGCGCGGA | 6870 | | 7013 | GTTTTAGAGCGCGG | Tetraloop | 1 | 78 |
| | | AACGCGCAAGTTAAA | | | | AAACGCGCAAGTTA | | | |
| | | ATAAGGCTAGTCCGTT | | | | AAATAAGGCTAGTC | | | |
| | | ATCAACTTGAAAAAG | | | | CGTTATCAACTTGAA | | | |
| | | TGGCACCGAGTCGGT | | | | AAAGTGGCACCGAG | | | |
| | | GC | | | | TCGGTGC | | | |
| 4352 | dnr6_flip | GTTTAAGAGCGCGGA | 6871 | | 7014 | GTTTAAGAGCGCGG | Tetraloop | 1 | 78 |
| | | AACGCGCAAGTTTAA | | | | AAACGCGCAAGTTT | | | |
| | | ATAAGGCTAGTCCGTT | | | | AAATAAGGCTAGTC | | | |
| | | ATCAACTTGAAAAAG | | | | CGTTATCAACTTGAA | | | |
| | | TGGCACCGAGTCGGT | | | | AAAGTGGCACCGAG | | | |
| | | GC | | | | TCGGTGC | | | |
| 4353 | M4 | GTTTAAGAGCTAGAA | 6872 | | 7015 | GTTTAAGAGCTAGA | none | 0 | 76 |
| | | ATAGCAAGTTTAAAT | | | | AATAGCAAGTTTAA | | | |
| | | AAGGCTAGTCCGTTAT | | | | ATAAGGCTAGTCCG | | | |
| | | CAACTTGAAAAAGTG | | | | TTATCAACTTGAAAA | | | |
| | | GCACCGAGTCGGTGC | | | | AGTGGCACCGAGTC | | | |
| | | | | | | GGTGC | | | |
| 4354 | F + E | GTTTAAGAGCTATGCT | 6873 | | 7016 | GTTTAAGAGCTATGC | Tetraloop | 5 | 86 |
| | | GGAAACAGCATAGCA | | | | TGGAAACAGCATAG | | | |
| | | AGTTTAAATAAGGCT | | | | CAAGTTTAAATAAG | | | |
| | | AGTCCGTTATCAACTT | | | | GCTAGTCCGTTATCA | | | |
| | | GAAAAAGTGGCACCG | | | | ACTTGAAAAAGTGG | | | |
| | | AGTCGGTGC | | | | CACCGAGTCGGTGC | | | |
| 4355 | s12_flip | GTTTAAGAGCTAGAA | 6874 | | 7017 | GTTTAAGAGCTAGA | none | 0 | 76 |
| | | ATAGCAAGTTTAAAT | | | | AATAGCAAGTTTAA | | | |
| | | AAGGCTAGTCCGTTAT | | | | ATAAGGCTAGTCCG | | | |
| | | CAGCGTGAAAACGCG | | | | TTATCAGCGTGAAA | | | |
| | | GCACCGAGTCGGTGC | | | | ACGCGGCACCGAGT | | | |
| | | | | | | CGGTGC | | | |
| 4356 | FE_s12 | GTTTAAGAGCTATGCT | 6875 | | 7018 | GTTTAAGAGCTATGC | Tetraloop | 5 | 86 |
| | | GGAAACAGCATAGCA | | | | TGGAAACAGCATAG | | | |
| | | AGTTTAAATAAGGCT | | | | CAAGTTTAAATAAG | | | |
| | | AGTCCGTTATCAGCGT | | | | GCTAGTCCGTTATCA | | | |
| | | GAAAACGCGGCACCG | | | | GCGTGAAAACGCGG | | | |
| | | AGTCGGTGC | | | | CACCGAGTCGGTGC | | | |
| 4357 | flip_U46A | GTTTAAGAGCTAGAA | 6876 | | 7019 | GTTTAAGAGCTAGA | none | 0 | 76 |
| | | ATAGCAAGTTTAAAT | | | | AATAGCAAGTTTAA | | | |
| | | AAGGCTAGTCCGTTA | | | | ATAAGGCTAGTCCG | | | |
| | | ACAACTTGAAAAAGT | | | | TTAACAACTTGAAA | | | |
| | | GGCACCGAGTCGGTG | | | | AAGTGGCACCGAGT | | | |
| | | C | | | | CGGTGC | | | |
| 4358 | M4_GC | GTTTGAGAGCTAGAA | 6877 | | 7020 | GTTTGAGAGCTAGA | none | 0 | 76 |
| | | ATAGCAAGTTCAAAT | | | | AATAGCAAGTTCAA | | | |
| | | AAGGCTAGTCCGTTAT | | | | ATAAGGCTAGTCCG | | | |
| | | CAACTTGAAAAAGTG | | | | TTATCAACTTGAAAA | | | |
| | | GCACCGAGTCGGTGC | | | | AGTGGCACCGAGTC | | | |
| | | | | | | GGTGC | | | |
| 4359 | M4_CG | GTTTCAGAGCTAGAA | 6878 | | 7021 | GTTTCAGAGCTAGA | none | 0 | 76 |
| | | ATAGCAAGTTGAAAT | | | | AATAGCAAGTTGAA | | | |
| | | AAGGCTAGTCCGTTAT | | | | ATAAGGCTAGTCCG | | | |
| | | CAACTTGAAAAAGTG | | | | TTATCAACTTGAAAA | | | |
| | | GCACCGAGTCGGTGC | | | | AGTGGCACCGAGTC | | | |
| | | | | | | GGTGC | | | |

### Nucleotide Editing

Provided herein are exemplary PEgRNAs with modifications disclosed herein for nucleotide editing. An intended nucleotide edit in an editing template of a PEgRNA may comprise various types of alterations as compared to the target gene sequence. In some embodiments, the nucleotide edit is a single nucleotide substitution as compared to the target gene sequence. In some embodiments, the nucleotide edit is a deletion as compared to the target gene sequence. In some embodiments, the nucleotide edit is an insertion as compared to the target gene sequence. In some embodiments, the editing template comprises one to ten intended nucleotide edits as compared to the target gene sequence. In some embodiments, the editing template comprises one or more intended nucleotide edits as compared to the target gene sequence. In some embodiments, the editing template comprises two or more intended nucleotide edits as compared to the target gene sequence. In some embodiments, the editing template comprises three or more intended nucleotide edits as compared to the target gene sequence. In some embodiments, the editing template comprises four or more, five or more, or six or more intended nucleotide edits as compared to the target gene sequence. In some embodiments, the editing template comprises two single nucleotide substitutions, insertions, deletions, or any combination thereof, as compared to the target gene sequence. In some embodiments, the editing template comprises three single nucleotide substitutions, insertions, deletions, or any combination thereof, as compared to the target gene sequence. In some embodiments, the editing template comprises four, five, or six single nucleotide substitutions, insertions, deletions, or any combination thereof, as compared to the target gene sequence. In some embodiments, a nucleotide substitution comprises an adenine (A)-to-thymine (T) substitution. In some embodiments, a nucleotide substitution comprises an A-to-guanine (G) substitution. In some embodiments, a nucleotide substitution comprises an A-to-cytosine (C) substitution. In some embodiments, a nucleotide substitution comprises a T-A substitution. In some embodiments, a nucleotide substitution comprises a T-G substitution. In some embodiments, a nucleotide substitution comprises a T-C substitution. In some embodiments, a nucleotide substitution comprises a G-to-A substitution. In some embodiments, a nucleotide substitution comprises a G-to-T substitution. In some embodiments, a nucleotide substitution comprises a G-to-C substitution. In some embodiments, a nucleotide substitution comprises a C-to-A substitution. In some embodiments, a nucleotide substitution comprises a C-to-T substitution. In some embodiments, a nucleotide substitution comprises a C-to-G substitution.

In some embodiments, a nucleotide insertion is at least 5 nucleotides, at least 6 nucleotides, at least 7 nucleotides, at least 8 nucleotides, at least 9 nucleotides, at least 10 nucleotides, at least 11 nucleotides, at least 12 nucleotides, at least 13 nucleotides, at least 14 nucleotides, at least 15 nucleotides, at least 16 nucleotides, at least 17 nucleotides, at least 18 nucleotides, at least 19 nucleotides, or at least 20 nucleotides in length. In some embodiments, a nucleotide insertion is from 1 to 2 nucleotides, from 1 to 3 nucleotides, from 1 to 4 nucleotides, from 1 to 5 nucleotides, form 2 to 5 nucleotides, from 3 to 5 nucleotides, from 3 to 6 nucleotides, from 3 to 8 nucleotides, from 4 to 9 nucleotides, from 5 to 10 nucleotides, from 6 to 11 nucleotides, from 7 to 12 nucleotides, from 8 to 13 nucleotides, from 9 to 14 nucleotides, from 10 to 15 nucleotides, from 11 to 16 nucleotides, from 12 to 17 nucleotides, from 13 to 18 nucleotides, from 14 to 19 nucleotides, from 15 to 20 nucleotides in length. In some embodiments, a nucleotide insertion is a single nucleotide insertion. In some embodiments, a nucleotide insertion comprises insertion of two nucleotides.

The editing template of a PEgRNA may comprise one or more intended nucleotide edits, compared to the gene to be edited. Position of the intended nucleotide edit(s) relevant to other components of the PEgRNA, or to particular nucleotides (e.g., mutations) in the target gene may vary. In some embodiments, the nucleotide edit is in a region of the PEgRNA corresponding to or homologous to the protospacer sequence. In some embodiments, the nucleotide edit is in a region of the PEgRNA corresponding to a region of the gene outside of the protospacer sequence.

In some embodiments, the position of a nucleotide edit incorporation in the target gene may be determined based on position of the protospacer adjacent motif (PAM). For instance, the intended nucleotide edit may be installed in a sequence corresponding to the protospacer adjacent motif (PAM) sequence. In some embodiments, a nucleotide edit in the editing template is at a position corresponding to the 5′ most nucleotide of the PAM sequence. In some embodiments, a nucleotide edit in the editing template is at a position corresponding to the 3′ most nucleotide of the PAM sequence. In some embodiments, position of an intended nucleotide edit in the editing template may be referred to by aligning the editing template with the partially complementary edit strand of the target gene, and referring to nucleotide positions on the editing strand where the intended nucleotide edit is incorporated. In some embodiments, a nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 basepairs upstream of the 5′ most nucleotide of the PAM sequence in the edit strand of the target gene. By 0 basepair upstream or downstream of a reference position, it is meant that the intended nucleotide is immediately upstream or downstream of the reference position. In some embodiments, a nucleotide edit is incorporated at a position corresponding to about 0 to 2 basepairs, 0 to 4 basepairs, 0 to 6 basepairs, 0 to 8 basepairs, 0 to 10 basepairs, 2 to 4 basepairs, 2 to 6 basepairs, 2 to 8 basepairs, 2 to 10 basepairs, 2 to 12 basepairs, 4 to 6 basepairs, 4 to 8 basepairs, 4 to 10 basepairs, 4 to 12 basepairs, 4 to 14 basepairs, 6 to 8 basepairs, 6 to 10 basepairs, 6 to 12 basepairs, 6 to 14 basepairs, 6 to 16 basepairs, 8 to 10 basepairs, 8 to 12 basepairs, 8 to 14 basepairs, 8 to 16 basepairs, 8 to 18 basepairs, 10 to 12 basepairs, 10 to 14 basepairs, 10 to 16 basepairs, 10 to 18 basepairs, 10 to 20 basepairs, 12 to 14 basepairs, 12 to 16 basepairs, 12 to 18 basepairs, 12 to 20 basepairs, 12 to 22 basepairs, 14 to 16 basepairs, 14 to 18 basepairs, 14 to 20 basepairs, 14 to 22 basepairs, 14 to 24 basepairs, 16 to 18 basepairs, 16 to 20 basepairs, 16 to 22 basepairs, 16 to 24 basepairs, 16 to 26 basepairs, 18 to 20 basepairs, 18 to 22 basepairs, 18 to 24 basepairs, 18 to 26 basepairs, 18 to 28 basepairs, 20 to 22 basepairs, 20 to 24 basepairs, 20 to 26 basepairs, 20 to 28 basepairs, or 20 to 30 basepairs upstream of the 5′ most nucleotide of the PAM sequence. In some embodiments, the nucleotide edit is incorporated at a position corresponding to 3 basepairs upstream of the 5′ most nucleotide of the PAM sequence. In some embodiments, the nucleotide edit in is incorporated at a position corresponding to 4 basepairs upstream of the 5′ most nucleotide of the PAM sequence. In some embodiments, the nucleotide edit is incorporated at a position corresponding to 5 basepairs upstream of the 5′ most nucleotide of the PAM sequence. In some embodiments, the nucleotide edit in the editing template is at a position corresponding to 6 basepairs upstream of the 5′ most nucleotide of the PAM sequence.

In some embodiments, an intended nucleotide edit is incorporated at a position corresponding to about 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 basepairs downstream of the 5′ most nucleotide of the PAM sequence in the edit strand of the target gene. In some embodiments, a nucleotide edit is incorporated at a position corresponding to about 0 to 2 basepairs, 0 to 4 basepairs, 0 to 6 basepairs, 0 to 8 basepairs, 0 to 10 basepairs, 2 to 4 basepairs, 2 to 6 basepairs, 2 to 8 basepairs, 2 to 10 basepairs, 2 to 12 basepairs, 4 to 6 basepairs, 4 to 8 basepairs, 4 to 10 basepairs, 4 to 12 basepairs, 4 to 14 basepairs, 6 to 8 basepairs, 6 to 10 basepairs, 6 to 12 basepairs, 6 to 14 basepairs, 6 to 16 basepairs, 8 to 10 basepairs, 8 to 12 basepairs, 8 to 14 basepairs, 8 to 16 basepairs, 8 to 18 basepairs, 10 to 12 basepairs, 10 to 14 basepairs, 10 to 16 basepairs, 10 to 18 basepairs, 10 to 20 basepairs, 12 to 14 basepairs, 12 to 16 basepairs, 12 to 18 basepairs, 12 to 20 basepairs, 12 to 22 basepairs, 14 to 16 basepairs, 14 to 18 basepairs, 14 to 20 basepairs, 14 to 22 basepairs, 14 to 24 basepairs, 16 to 18 basepairs, 16 to 20 basepairs, 16 to 22 basepairs, 16 to 24 basepairs, 16 to 26 basepairs, 18 to 20 basepairs, 18 to 22 basepairs, 18 to 24 basepairs, 18 to 26 basepairs, 18 to 28 basepairs, 20 to 22 basepairs, 20 to 24 basepairs, 20 to 26 basepairs, 20 to 28 basepairs, or 20 to 30 basepairs downstream of the 5′ most nucleotide of the PAM sequence. In some embodiments, a nucleotide edit is incorporated at a position corresponding to 3 basepairs downstream of the 5′ most nucleotide of the PAM sequence. In some embodiments, a nucleotide edit is incorporated at a position corresponding to 4 basepairs downstream of the 5′ most nucleotide of the PAM sequence. In some embodiments, a nucleotide edit is incorporated at a position corresponding to 5 basepairs downstream of the 5′ most nucleotide of the PAM sequence. In some embodiments, a nucleotide edit is incorporated at a position corresponding to 6 basepairs downstream of the 5′ most nucleotide of the PAM sequence. By “upstream” and “downstream” it is intended to define relevant positions at least two regions or sequences in a nucleic acid molecule orientated in a 5′-to-3′ direction. For example, a first sequence is upstream of a second sequence in a DNA molecule where the first sequence is positioned 5′ to the second sequence. Accordingly, the second sequence is downstream of the first sequence.

When referred to in the PEgRNA, positions of the one or more intended nucleotide edits may be referred to relevant to components of the PEgRNA. For example, an intended nucleotide edit may be 5′ or 3′ to the PBS. In some embodiments, a PEgRNA comprises the structure, from 5′ to 3′: a spacer, a gRNA core, an editing template, and a PBS. In some embodiments, the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 basepairs upstream to the 5′ most nucleotide of the PBS. In some embodiments, the intended nucleotide edit is 0 to 2 basepairs, 0 to 4 basepairs, 0 to 6 basepairs, 0 to 8 basepairs, 0 to 10 basepairs, 2 to 4 basepairs, 2 to 6 basepairs, 2 to 8 basepairs, 2 to 10 basepairs, 2 to 12 basepairs, 4 to 6 basepairs, 4 to 8 basepairs, 4 to 10 basepairs, 4 to 12 basepairs, 4 to 14 basepairs, 6 to 8 basepairs, 6 to 10 basepairs, 6 to 12 basepairs, 6 to 14 basepairs, 6 to 16 basepairs, 8 to 10 basepairs, 8 to 12 basepairs, 8 to 14 basepairs, 8 to 16 basepairs, 8 to 18 basepairs, 10 to 12 basepairs, 10 to 14 basepairs, 10 to 16 basepairs, 10 to 18 basepairs, 10 to 20 basepairs, 12 to 14 basepairs, 12 to 16 basepairs, 12 to 18 basepairs, 12 to 20 basepairs, 12 to 22 basepairs, 14 to 16 basepairs, 14 to 18 basepairs, 14 to 20 basepairs, 14 to 22 basepairs, 14 to 24 basepairs, 16 to 18 basepairs, 16 to 20 basepairs, 16 to 22 basepairs, 16 to 24 basepairs, 16 to 26 basepairs, 18 to 20 basepairs, 18 to 22 basepairs, 18 to 24 basepairs, 18 to 26 basepairs, 18 to 28 basepairs, 20 to 22 basepairs, 20 to 24 basepairs, 20 to 26 basepairs, 20 to 28 basepairs, or 20 to 30 basepairs upstream to the 5′ most nucleotide of the PBS.

The corresponding positions of the intended nucleotide edit incorporated in the target gene may also be referred to bases on the nicking position generated by a prime editor based on sequence homology and complementarity. For example, in embodiments, the distance between the nucleotide edit to be incorporated into the target gene and the nick generated by the prime editor may be determined when the spacer hybridizes with the search target sequence and the extension arm hybridizes with the editing target sequence. In certain embodiments, the position of the nucleotide edit can be in any position downstream of the nick site on the edit strand (or the PAM strand) generated by the prime editor, such that the distance between the nick site and the intended nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides upstream of the nick site on the edit strand. In some embodiments, the position of the nucleotide edit is 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 nucleotides downstream of the nick site on the edit strand. In some embodiments, the position of the nucleotide edit is 0 basepairs from the nick site on the edit strand, that is, the editing position is at the same position as the nick site. As used herein, the distance between the nick site and the nucleotide edit, for example, where the nucleotide edit comprises an insertion or deletion, refers to the 5′ most position of the nucleotide edit for a nick that creates a 3′ free end on the edit strand (i.e., the “near position” of the nucleotide edit to the nick site). Similarly, as used herein, the distance between the nick site and a PAM position edit, for example, where the nucleotide edit comprises an insertion, deletion, or substitution of two or more contiguous nucleotides, refers to the 5′ most position of the nucleotide edit and the 5′ most position of the PAM sequence.

A PEgRNA may also comprise optional modifiers, e.g., 3′ end modifier region and/or a 5′ end modifier region. In some embodiments, a PEgRNA comprises at least one nucleotide that is not part of a spacer, a gRNA core, or an extension arm. The optional sequence modifiers could be positioned within or between any of the other regions shown, and not limited to being located at the 3′ and 5′ ends. In certain embodiments, the PEgRNA comprises secondary RNA structure, such as, but not limited to, aptamers, hairpins, stem/loops, toeloops, and/or RNA-binding protein recruitment domains (e.g., the MS2 aptamer which recruits and binds to the MS2cp protein). In some embodiments, a PEgRNA comprises a short stretch of uracil at the 5′ end or the 3′ end. For example, in some embodiments, a PEgRNA comprising a 3′ extension arm comprises a “UUU” sequence at the 3′ end of the extension arm. In some embodiments, a PEgRNA comprises a toeloop sequence at the 3′ end. In some embodiments, the PEgRNA comprises a 3′ extension arm and a toeloop sequence at the 3′ end of the extension arm. In some embodiments, the PEgRNA comprises a 5′ extension arm and a toeloop sequence at the 5′ end of the extension arm. In some embodiments, the PEgRNA comprises a toeloop element having the sequence 5′-GAAANNNNN-3′, wherein N is any nucleobase. In some embodiments, the secondary RNA structure is positioned within the spacer. In some embodiments, the secondary structure is positioned within the extension arm. In some embodiments, the secondary structure is positioned within the gRNA core. In some embodiments, the secondary structure is positioned between the spacer and the gRNA core, between the gRNA core and the extension arm, or between the spacer and the extension arm. In some embodiments, the secondary structure is positioned between the PBS and the editing template. In some embodiments the secondary structure is positioned at the 3′ end or at the 5′ end of the PEgRNA. In some embodiments, the PEgRNA comprises a transcriptional termination signal at the 3′ end of the PEgRNA. In addition to secondary RNA structures, the PEgRNA may comprise a chemical linker or a poly(N) linker or tail, where “N” can be any nucleobase. In some embodiments, the chemical linker may function to prevent reverse transcription of the gRNA core.

In some embodiments, a prime editing system or composition further comprises a nick guide polynucleotide, such as a nick guide RNA (ngRNA). Without wishing to be bound by any particular theory, the non-edit strand of a double stranded target DNA in the target gene may be nicked by a CRISPR-Cas nickase directed by an ngRNA. In some embodiments, the nick on the non-edit strand directs endogenous DNA repair machinery to use the edit strand as a template for repair of the non-edit strand, which may increase efficiency of prime editing. In some embodiments, the non-edit strand is nicked by a prime editor localized to the non-edit strand by the ngRNA. Accordingly, also provided herein are PEgRNA systems comprising at least one PEgRNA and at least one ngRNA.

In some embodiments, the ngRNA is a guide RNA which contains a variable spacer sequence and a guide RNA scaffold or core region that interacts with the DNA binding domain, e.g., Cas9 of the prime editor. In some embodiments, the ngRNA comprises a spacer sequence (referred to herein as an ng spacer, or a second spacer) that is substantially complementary to a second search target sequence (or ng search target sequence), which is located on the edit strand, or the non-target strand. Thus, in some embodiments, the ng search target sequence recognized by the ng spacer and the search target sequence recognized by the spacer sequence of the PEgRNA are on opposite strands of the double stranded target DNA of target gene, e.g., the gene.

A prime editing system or composition that does not comprise a ngRNA may be referred to as a “PE2” prime editing system. A prime editing system or composition comprising a ngRNA may be referred to as a “PE3” prime editing system or PE3 prime editing complex.

In some embodiments, the ng search target sequence is located on the non-target strand, within 10 basepairs to 100 basepairs of an intended nucleotide edit incorporated by the PEgRNA on the edit strand. In some embodiments, the ng target search target sequence is within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp of an intended nucleotide edit incorporated by the PEgRNA on the edit strand. In some embodiments, the 5′ ends of the ng search target sequence and the PEgRNA search target sequence are within 1, 2, 3, 4, 5, 6, 7, 8, 9, or 10 bp apart from each other. In some embodiments, the 5′ ends of the ng search target sequence and the PEgRNA search target sequence are within 10 bp, 20 bp, 30 bp, 40 bp, 50 bp, 60 bp, 70 bp, 80 bp, 90 bp, 91 bp, 92 bp, 93 bp, 94 bp, 95 bp, 96 bp, 97 bp, 98 bp, 99 bp, or 100 bp apart from each other.

In some embodiments, an ng spacer sequence is complementary to, and may hybridize with the second search target sequence only after an intended nucleotide edit has been incorporated on the edit strand, by the editing template of a PEgRNA. Such a prime editing system may be referred to as a “PE3b” prime editing system or composition. In some embodiments, the ngRNA comprises a spacer sequence that matches only the edit strand after incorporation of the nucleotide edits, but not the endogenous target gene sequence on the edit strand. Accordingly, in some embodiments, an intended nucleotide edit is incorporated within the ng search target sequence. In some embodiments, the intended nucleotide edit is incorporated within about 1-10 nucleotides of the position corresponding to the PAM of the ng search target sequence.

A PEgRNA and/or an ngRNA of this disclosure, in some embodiments, may include modified nucleotides, e.g., chemically modified DNA or RNA nucleobases, and may include one or more nucleobase analogs (e.g., modifications which might add functionality, such as temperature resilience). In some embodiments, PEgRNAs and/or ngRNAs as described herein may be chemically modified. The phrase “chemical modifications,” as used herein, can include modifications which introduce chemistries which differ from those seen in naturally occurring DNA or RNAs, for example, covalent modifications such as the introduction of modified nucleotides, (e.g., nucleotide analogs, or the inclusion of pendant groups which are not naturally found in DNA or RNA molecules).

In some embodiments, the PEgRNAs and/or ngRNAs provided in this disclosure may have undergone a chemical or biological modifications. Modifications may be made at any position within a PEgRNA or ngRNA, and may include modification to a nucleobase or to a phosphate backbone of the PEgRNA or ngRNA. In some embodiments, chemical modifications can be a structure guided modifications. In some embodiments, a chemical modification is at the 5′ end and/or the 3′ end of a PEgRNA. In some embodiments, a chemical modification is at the 5′ end and/or the 3′ end of a ngRNA. In some embodiments, a chemical modification may be within the spacer sequence, the extension arm, the editing template sequence, or the primer binding site of a PEgRNA. In some embodiments, a chemical modification may be within the spacer sequence or the gRNA core of a PEgRNA or a ngRNA. In some embodiments, a chemical modification may be within the 3′ most nucleotides of a PEgRNA or ngRNA. In some embodiments, a chemical modification may be within the 3′ most end of a PEgRNA or ngRNA. In some embodiments, a chemical modification may be within the 5′ most end of a PEgRNA or ngRNA. In some embodiments, a PEgRNA or ngRNA comprises 1, 2, 3, 4, 5, 6, 7, 8, 9, 10 or more chemically modified nucleotides at the 3′ end. In some embodiments, a PEgRNA or ngRNA comprises 1, 2, 3, 4, 5, 6, 7, 8, 9, 10 or more chemically modified nucleotides at the 5′ end. In some embodiments, a PEgRNA or ngRNA comprises 1, 2, 3, 4, or 5 or more chemically modified nucleotides at the 3′ end. In some embodiments, a PEgRNA or ngRNA comprises 1, 2, 3, 4, or 5 more chemically modified nucleotides at the 5′ end. In some embodiments, a PEgRNA or ngRNA comprises 1, 2, or 3 or more chemically modified nucleotides at the 3′ end. In some embodiments, a PEgRNA or ngRNA comprises 1, 2, or 3 more chemically modified nucleotides at the 5′ end. In some embodiments, a PEgRNA or ngRNA comprises 1, 2, 3, 4, 5, 6, 7, 8, 9, 10 or more contiguous chemically modified nucleotides at the 3′ end. In some embodiments, a PEgRNA or ngRNA comprises 1, 2, 3, 4, 5, 6, 7, 8, 9, 10 or more contiguous chemically modified nucleotides at the 5′ end. In some embodiments, a PEgRNA or ngRNA comprises 1, 2, 3, 4, or 5 contiguous chemically modified nucleotides at the 3′ end. In some embodiments, a PEgRNA or ngRNA comprises 1, 2, 3, 4, or 5 contiguous chemically modified nucleotides at the 5′ end. In some embodiments, a PEgRNA or ngRNA comprises 1, 2, or 3 contiguous chemically modified nucleotides at the 3′ end. In some embodiments, a PEgRNA or ngRNA comprises 1, 2, or 3 contiguous chemically modified nucleotides at the 5′ end. In some embodiments, a PEgRNA or ngRNA comprises 3 contiguous chemically modified nucleotides at the 3′ end. In some embodiments, a PEgRNA or ngRNA comprises 1, 2, 3, 4, 5, or more chemically modified nucleotides near the 3′ end. In some embodiments, a PEgRNA or ngRNA comprises 3 contiguous chemically modified nucleotides at the 3′ end. In some embodiments, a PEgRNA or ngRNA comprises 3 contiguous chemically modified nucleotides at the 5′ end. In some embodiments, a PEgRNA or ngRNA comprises 1, 2, 3, 4, 5, or more chemically modified nucleotides near the 3′ end. In some embodiments, a PEgRNA or ngRNA comprises 1, 2, 3, 4, 5, or more contiguous chemically modified nucleotides near the 3′ end. In some embodiments, a PEgRNA or ngRNA comprises 1, 2, 3, 4, 5, or more chemically modified nucleotides near the 3′ end, where the 3′ most nucleotide is not modified, and the 1, 2, 3, 4, 5, or more chemically modified nucleotides precede the 3′ most nucleotide in a 5′-to-3′ order. In some embodiments, a PEgRNA or ngRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides near the 3′ end, where the 3′ most nucleotide is not modified, and the 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35 or more chemically modified nucleotides precede the 3′ most nucleotide in a 5′-to-3′ order.

In some embodiments, a PEgRNA or ngRNA comprises one or more chemical modified nucleotides in the gRNA core. As exemplified in FIG. 8, the gRNA core of a PEgRNA may comprise one or more regions of a basepaired lower stem, a basepaired upper stem, where the lower stem and upper stem may be connected by a bulge comprising unpaired RNAs. The gRNA core may further comprise a nexus distal from the spacer sequence. In some embodiments, the gRNA core comprises one or more chemically modified nucleotides in the lower stem, upper stem, and/or the hairpin regions. In some embodiments, all of the nucleotides in the lower stem, upper stem, and/or the hairpin regions are chemically modified.

A chemical modification to a PEgRNA or ngRNA can comprise a 2′-O-thionocarbamate-protected nucleoside phosphoramidite, a 2′-O-methyl (M), a 2′-O-methyl 3′phosphorothioate (MS), or a 2′-O-methyl 3′thioPACE (MSP), or any combination thereof. In some embodiments, a chemically modified PEgRNA and/or ngRNA can comprise a 2′-O-methyl (M) RNA, a 2′-O-methyl 3′phosphorothioate (MS) RNA, a 2′-O-methyl 3′thioPACE (MSP) RNA, a 2′-F RNA, a phosphorothioate bond modification, any other chemical modifications known in the art, or any combination thereof. A chemical modification may also include, for example, the incorporation of non-nucleotide linkages or modified nucleotides into the PEgRNA and/or ngRNA (e.g., modifications to one or both of the 3′ and 5′ ends of a guide RNA molecule). Such modifications can include the addition of bases to an RNA sequence, complexing the RNA with an agent (e.g., a protein or a complementary nucleic acid molecule), and inclusion of elements which change the structure of an RNA molecule (e.g., which form secondary structures).

### Prime Editor

The term “prime editor (PE)” refers to the polypeptide or polypeptide components involved in prime editing, or any polynucleotide(s) encoding the polypeptide or polypeptide components. In various embodiments, a prime editor includes a polypeptide domain having DNA binding activity and a polypeptide domain having DNA polymerase activity. In some embodiments, the prime editor further comprises a polypeptide domain having nuclease activity. In some embodiments, the polypeptide domain having DNA binding activity comprises a nuclease domain or nuclease activity. In some embodiments, the polypeptide domain having nuclease activity comprises a nickase, or a fully active nuclease. As used herein, the term “nickase” refers to a nuclease capable of cleaving only one strand of a double-stranded DNA target. In some embodiments, the prime editor comprises a polypeptide domain that is an inactive nuclease. In some embodiments, the polypeptide domain having programmable DNA binding activity comprises a nucleic acid guided DNA binding domain, for example, a CRISPR-Cas protein, for example, a Cas9 nickase, a Cpf1 nickase, or another CRISPR-Cas nuclease. In some embodiments, the polypeptide domain having DNA polymerase activity comprises a template-dependent DNA polymerase, for example, a DNA-dependent DNA polymerase or an RNA-dependent DNA polymerase. In some embodiments, the DNA polymerase is a reverse transcriptase. In some embodiments, the prime editor comprises additional polypeptides involved in prime editing, for example, a polypeptide domain having 5′ endonuclease activity, e.g., a 5′ endogenous DNA flap endonucleases (e.g., FEN1), for helping to drive the prime editing process towards the edited product formation. In some embodiments, the prime editor further comprises an RNA-protein recruitment polypeptide, for example, a MS2 coat protein.

A prime editor may be engineered. In some embodiments, the polypeptide components of a prime editor do not naturally occur in the same organism or cellular environment. In some embodiments, the polypeptide components of a prime editor may be of different origins or from different organisms. In some embodiments, a prime editor comprises a DNA binding domain and a DNA polymerase domain that are derived from different species. In some embodiments, a prime editor comprises a Cas polypeptide and a reverse transcriptase polypeptide that are derived from different species. For example, a prime editor may comprise a S. pyogenes Cas9 polypeptide and a Moloney murine leukemia virus (M-MLV) reverse transcriptase polypeptide.

In some embodiments, polypeptide domains of a prime editor may be fused or linked by a peptide linker to form a fusion protein. In other embodiments, a prime editor comprises one or more polypeptide domains provided in trans as separate proteins, which are capable of being associated to each other through non-peptide linkages or through aptamers or recruitment sequences. For example, a prime editor may comprise a DNA binding domain and a reverse transcriptase domain associated with each other by an RNA-protein recruitment aptamer, e.g., a MS2 aptamer, which may be linked to a PEgRNA. Prime editor polypeptide components may be encoded by one or more polynucleotides in whole or in part. In some embodiments, a single polynucleotide, construct, or vector encodes the prime editor fusion protein. In some embodiments, multiple polynucleotides, constructs, or vectors each encode a polypeptide domain or portion of a domain of a prime editor, or a portion of a prime editor fusion protein. For example, a prime editor fusion protein may comprise an N-terminal portion fused to an intein-N and a C-terminal portion fused to an intein-C, each of which is individually encoded by an AAV vector.

### Prime Editor Nucleotide Polymerase Domain

In some embodiments, a prime editor comprises a nucleotide polymerase domain, e.g., a DNA polymerase domain. The DNA polymerase domain may be a wild-type DNA polymerase domain, a full-length DNA polymerase protein domain, or may be a functional mutant, a functional variant, or a functional fragment thereof. In some embodiments, the polymerase domain is a template dependent polymerase domain. For example, the DNA polymerase may rely on a template polynucleotide strand, e.g., the editing template sequence, for new strand DNA synthesis. In some embodiments, the prime editor comprises a DNA-dependent DNA polymerase. For example, a prime editor having a DNA-dependent DNA polymerase can synthesize a new single stranded DNA using a PEgRNA editing template that comprises a DNA sequence as a template. In such cases, the PEgRNA is a chimeric or hybrid PEgRNA, and comprising an extension arm comprising a DNA strand. The chimeric or hybrid PEgRNA may comprise an RNA portion (including the spacer and the gRNA core) and a DNA portion (the extension arm comprising the editing template that includes a strand of DNA).

The DNA polymerases can be wild type polymerases from eukaryotic, prokaryotic, archael, or viral organisms, and/or the polymerases may be modified by genetic engineering, mutagenesis, or directed evolution-based processes. The polymerases can be a T7 DNA polymerase, T5 DNA polymerase, T4 DNA polymerase, Klenow fragment DNA polymerase, DNA polymerase III and the like. The polymerases can be thermostable, and can include Taq, Tne, Tma, Pfu, Tfl, Tth, Stoffel fragment, VENT® and DEEPVENT® DNA polymerases, KOD, Tgo, JDF3, and mutants, variants and derivatives thereof.

For synthesis of longer nucleic acid molecules (e.g., nucleic acid molecules longer than about 3-5 Kb in length), at least two DNA polymerases can be employed. In certain embodiments, one of the polymerases can be substantially lacking a 3′ exonuclease activity and the other may have a 3′ exonuclease activity. Such pairings may include polymerases that are the same or different. Examples of DNA polymerases substantially lacking in 3′ exonuclease activity include, but are not limited to, Taq, Tne(exo-), Tma(exo-), Pfu(exo-), Pwo(exo-), exo-KOD and Tth DNA polymerases, and any functional mutants, functional variants and functional fragments thereof.

In some embodiments, the DNA polymerase is a bacteriophage polymerase, for example, a T4, T7, or phi29 DNA polymerase. In some embodiments, the DNA polymerase is an archaeal polymerase, for example, pol I type archaeal polymerase or a pol II type archaeal polymerase. In some embodiments, the DNA polymerase comprises a thermostable archaeal DNA polymerase. In some embodiments, the DNA polymerase comprises a eubacterial DNA polymerase, for example, Pol I, Pol II, or Pol III polymerase. In some embodiments, the DNA polymerase is a Pol I family DNA polymerase. In some embodiments, the DNA polymerase is a E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol IV family DNA polymerase. In some embodiments, the DNA polymerase is an E. coli Pol IV DNA polymerase.

In some embodiments, the DNA polymerase comprises a eukaryotic DNA polymerase. In some embodiments, the DNA polymerase is a Pol-beta DNA polymerase, a Pol-lambda DNA polymerase, a Pol-sigma DNA polymerase, or a Pol-mu DNA polymerase. In some embodiments, the DNA polymerase is a Pol-alpha DNA polymerase. In some embodiments, the DNA polymerase is a POLA1 DNA polymerase. In some embodiments, the DNA polymerase is a POLA2 DNA polymerase. In some embodiments, the DNA polymerase is a Pol-delta DNA polymerase. In some embodiments, the DNA polymerase is a POLD1 DNA polymerase. In some embodiments, the DNA polymerase is a POLD2 DNA polymerase. In some embodiments, the DNA polymerase is a human POLD1 DNA polymerase. In some embodiments, the DNA polymerase is a human POLD2 DNA polymerase. In some embodiments, the DNA polymerase is a POLD3 DNA polymerase. In some embodiments, the DNA polymerase is a POLD4 DNA polymerase. In some embodiments, the DNA polymerase is a Pol-epsilon DNA polymerase. In some embodiments, the DNA polymerase is a POLE1 DNA polymerase. In some embodiments, the DNA polymerase is a POLE2 DNA polymerase. In some embodiments, the DNA polymerase is a POLE3 DNA polymerase. In some embodiments, the DNA polymerase is a Pol-eta (POLH) DNA polymerase. In some embodiments, the DNA polymerase is a Pol-iota (POLI) DNA polymerase. In some embodiments, the DNA polymerase is a Pol-kappa (POLK) DNA polymerase. In some embodiments, the DNA polymerase is a Rev1 DNA polymerase. In some embodiments, the DNA polymerase is a human Rev1 DNA polymerase. In some embodiments, the DNA polymerase is a viral DNA-dependent DNA polymerase. In some embodiments, the DNA polymerase is a B family DNA polymerases. In some embodiments, the DNA polymerase is a herpes simplex virus (HSV) UL30 DNA polymerase. In some embodiments, the DNA polymerase is a cytomegalovirus (CMV) UL54 DNA polymerase.

In some embodiments, the DNA polymerase is an archaeal polymerase. In some embodiments, the DNA polymerase is a Family B/pol I type DNA polymerase. For example, in some embodiments, the DNA polymerase is a homolog of Pfu from Pyrococcus furiosus. In some embodiments, the DNA polymerase is a pol II type DNA polymerase. For example, in some embodiments, the DNA polymerase is a homolog of P. furiosus DP1/DP2 2-subunit polymerase. In some embodiments, the DNA polymerase lacks 5′ to 3′ nuclease activity. Suitable DNA polymerases (pol I or pol II) can be derived from archaea with optimal growth temperatures that are similar to the desired assay temperatures.

In some embodiments, the DNA polymerase comprises a thermostable archaeal DNA polymerase. In some embodiments, the thermostable DNA polymerase is isolated or derived from Pyrococcus species (furiosus, species GB-D, woesii, abysii, horikoshii), Thermococcus species (kodakaraensis KOD1, litoralis, species 9 degrees North-7, species JDF-3, gorgonarius), Pyrodictium occultum, and Archaeoglobus fulgidus.

Polymerases may also be from eubacterial species. In some embodiments, the DNA polymerase is a Pol I family DNA polymerase. In some embodiments, the DNA polymerase is an E. coli Pol I DNA polymerase. In some embodiments, the DNA polymerase is a Pol II family DNA polymerase. In some embodiments, the DNA polymerase is a Pyrococcus furiosus (Pfu) Pol II DNA polymerase. In some embodiments, the DNA polymerase is a Pol III family DNA polymerase. In some embodiments, the DNA polymerase is a Pol IV family DNA polymerase. In some embodiments, the DNA polymerase is an E. coli Pol IV DNA polymerase. In some embodiments, the Pol I DNA polymerase is a DNA polymerase functional variant that lacks or has reduced 5′ to 3′ exonuclease activity.

Suitable thermostable pol I DNA polymerases can be isolated from a variety of thermophilic eubacteria, including Thermus species and Thermotoga maritima such as Thermus aquaticus (Taq), Thermus thermophilus (Tth) and Thermotoga maritima (Tma UITma).

In some embodiments, a prime editor comprises an RNA-dependent DNA polymerase domain, for example, a reverse transcriptase (RT). A RT or an RT domain may be a wild type RT domain, a full-length RT domain, or may be a functional mutant, a functional variant, or a functional fragment thereof. An RT or an RT domain of a prime editor may comprise a wild-type RT, or may be engineered or evolved to contain specific amino acid substitutions, truncations, or variants. An engineered RT may comprise sequences or amino acid changes different from a naturally occurring RT. In some embodiments, the engineered RT may have improved reverse transcription activity over a naturally occurring RT or RT domain. In some embodiments, the engineered RT may have improved features over a naturally occurring RT, for example, improved thermostability, reverse transcription efficiency, or target fidelity. In some embodiments, a prime editor comprising the engineered RT has improved prime editing efficiency over a prime editor having a reference naturally occurring RT.

In some embodiments, a prime editor comprises a virus RT, for example, a retrovirus RT. Non-limiting examples of virus RT include Moloney murine leukemia virus (M-MLV or MLVRT); human T-cell leukemia virus type 1 (HTLV-1) RT; bovine leukemia virus (BLV) RT; Rous Sarcoma Virus (RSV) RT; human immunodeficiency virus (HIV) RT, M-MFV RT, Avian Sarcoma-Leukosis Virus (ASLV) RT, Rous Sarcoma Virus (RSV) RT, Avian Myeloblastosis Virus (AMV) RT, Avian Erythroblastosis Virus (AEV) Helper Virus MCAV RT, Avian Myelocytomatosis Virus MC29 Helper Virus MCAV RT, Avian Reticuloendotheliosis Virus (REV-T) Helper Virus REV-A RT, Avian Sarcoma Virus UR2 Helper Virus (UR2AV) RT, Avian Sarcoma Virus Y73 Helper Virus YAV RT, Rous Associated Virus (RAV) RT, and Myeloblastosis Associated Virus (MAV) RT, all of which may be suitably used in the methods and composition described herein.

In some embodiments, the prime editor comprises a wild type M-MLV RT. An exemplary sequence of a wild type M-MLV RT is provided in SEQ ID NO: 4448.

In some embodiments, the prime editor comprises a M-MMLV RT comprising one or more of amino acid substitutions P51X, S67X, E69X, L139X, T197X, D200X, H204X, F209X, E302X, T306X, F309X, W313X, T330X, L345X, L435X, N454X, D524X, E562X, D583X, H594X, L603X, E607X, or D653X as compared to the wild type M-MMLV RT as set forth in SEQ ID NO: 4448, where X is any amino acid other than the wild type amino acid. In some embodiments, the prime editor comprises a M-MMLV RT comprising one or more of amino acid substitutions P51L, S67K, E69K, L139P, T197A, D200N, H204R, F209N, E302K, E302R, T306K, F309N, W313F, T330P, L345G, L435G, N454K, D524G, E562Q, D583N, H594Q, L603W, E607K, and D653N as compared to the wild type M-MMLV RT as set forth in SEQ ID NO: 4448. In some embodiments, the prime editor comprises a M-MLV RT comprising one or more amino acid substitutions D200N, T330P, L603W, T306K, and W313F as compared to the wild type M-MMLV RT as set forth in SEQ ID NO: 4448. In some embodiments, the prime editor comprises a M-MLV RT comprising amino acid substitutions D200N, T330P, L603W, T306K, and W313F as compared to the wild type M-MMLV RT as set forth in SEQ ID NO: 4448.

Exemplary wild type moloney murine leukemia virus reverse transcriptase:

TLNIEDEYRLHETSKEPDVSLGSTWLSDFPQAWAETGGMGLAVRQAPLIIPLKAT STPVSIKQYPMSQEARLGIKPHIQRLLDQGILVPCQSPWNTPLLPVKKPGTNDYRPVQDL REVNKRVEDIHPTVPNPYNLLSGLPPSHQWYTVLDLKDAFFCLRLHPTSQPLFAFEWRD PEMGISGQLTWTRLPQGFKNSPTLFDEALHRDLADFRIQHPDLILLQYVDDLLLAATSEL DCQQGTRALLQTLGNLGYRASAKKAQICQKQVKYLGYLLKEGQRWLTEARKETVMG QPTPKTPRQLREFLGTAGFCRLWIPGFAEMAAPLYPLTKTGTLFNWGPDQQKAYQEIKQ ALLTAPALGLPDLTKPFELFVDEKQGYAKGVLTQKLGPWRRPVAYLSKKLDPVAAGWP PCLRMVAAIAVLTKDAGKLTMGQPLVILAPHAVEALVKQPPDRWLSNARMTHYQALL LDTDRVQFGPVVALNPATLLPLPEEGLQHNCLDILAEAHGTRPDLTDQPLPDADHTWYT DGSSLLQEGQRKAGAAVTTETEVIWAKALPAGTSAQRAELIALTQALKMAEGKKLNVY TDSRYAFATAHIHGEIYRRRGLLTSEGKEIKNKDEILALLKALFLPKRLSIIHCPGHQKGH SAEARGNRMADQAARKAAITETPDTSTLLIENSSP (SEQ ID NO: 4448). In some embodiments, an RT variant may be a functional fragment of a reference RT that have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50, or up to 100, or up to 200, or up to 300, or up to 400, or up to 500 or more amino acid changes compared to a reference RT, e.g., a wild type RT. In some embodiments, the RT variant comprises a fragment of a reference RT, e.g., a wild type RT, such that the fragment is about 70% identical, about 80% identical, about 90% identical, about 95% identical, about 96% identical, about 97% identical, about 98% identical, about 99% identical, about 99.5% identical, or about 99.9% identical to the corresponding fragment of the reference RT. In some embodiments, the fragment is 30%, 35%, 40%, 45%, 50%, 55%, 60%, 65%, 70%, 75%, 80%, 85%, 90%, 95% identical, 96%, 97%, 98%, 99%, or 99.5% of the amino acid length of a corresponding wild type RT (M-MLV reverse transcriptase) (e.g., SEQ ID NO: 4448).

In some embodiments, the RT functional fragment is at least 100 amino acids in length. In some embodiments, the fragment is at least 100, 150, 200, 250, 300, 350, 400, 450, 500, 550, or up to 600 or more amino acids in length.

In still other embodiments, the functional RT variant is truncated at the N-terminus or the C-terminus, or both, by a certain number of amino acids which results in a truncated variant which still retains sufficient DNA polymerase function. In some embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the N-terminal end compared to a reference RT, e.g., a wild type RT. In some embodiments, the reference RT is a wild type M-MLV RT. In other embodiments, the RT truncated variant has a truncation of at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 30, 40, 50, 60, 70, 80, 90, 100, 110, 120, 130, 140, 150, 160, 170, 180, 190, 200, 210, 220, 230, 240, or 250 amino acids at the C-terminal end compared to a reference RT, e.g., a wild type RT. In some embodiments, the reference RT is a wild type M-MLV RT. In still other embodiments, the RT truncated variant has a truncation at the N-terminal and the C-terminal end compared to a reference RT, e.g., a wild type RT. In some embodiments, the N-terminal truncation and the C-terminal truncation are of the same length. In some embodiments, the N-terminal truncation and the C-terminal truncation are of different lengths.

For example, the prime editors disclosed herein may include a functional variant of a wild type M-MLV reverse transcriptase. In some embodiments, the prime editor comprises a functional variant of a wild type M-MLV RT, wherein the functional variant of M-MLV RT is truncated after amino acid position 502 compared to a wild type M-MLV RT as set forth in SEQ ID NO: 4448. In some embodiments, the functional variant of M-MLV RT further comprises a D200X, T306X, W313X, and/or T330X amino acid substitution compared to compared to a wild type M-MLV RT as set forth in SEQ ID NO: 4448, wherein X is any amino acid other than the original amino acid. In some embodiments, the functional variant of M-MLV RT further comprises a D200N, T306K, W313F, and/or T330P amino acid substitution compared to compared to a wild type M-MLV RT as set forth in SEQ ID NO: 4448, wherein X is any amino acid other than the original amino acid. A nucleic acid sequence encoding a prime editor comprising this truncated RT is 522 nt smaller than a nucleic acid sequence encoding a prime editor comprising a full-length M-MLV RT, and therefore makes its potentially useful for applications where delivery of the DNA sequence is challenging due to its size (i.e., adeno-associated virus and lentivirus delivery). In some embodiments, a prime editor comprises a M-MLV RT variant, wherein the M-MLV RT consists of the following amino acid sequence:

(SEQ ID NO: 7022)
TLNIEDEYRLHETSKEPDVSLGSTWLSDFPQAWAETGGMGLAVRQAPLII
PLKATSTPVSIKQYPMSQEARLGIKPHIQRLLDQGILVPCQSPWNTPLLP
VKKPGTNDYRPVQDLREVNKRVEDIHPTVPNPYNLLSGLPPSHQWYTVLD
LKDAFFCLRLHPTSQPLFAFEWRDPEMGISGQLTWTRLPQGFKNSPTLFN
EALHRDLADFRIQHPDLILLQYVDDLLLAATSELDCQQGTRALLQTLGNL
GYRASAKKAQICQKQVKYLGYLLKEGQRWLTEARKETVMGQPTPKTPRQL
REFLGKAGFCRLFIPGFAEMAAPLYPLTKPGTLFNWGPDQQKAYQEIKQA
LLTAPALGLPDLTKPFELFVDEKQGYAKGVLTQKLGPWRRPVAYLSKKLD
PVAAGWPPCLRMVAAIAVLTKDAGKLTMGQPLVILAPHAVEALVKQPPDR
WLSNARMTHYQALLLDTDRVQFGPVVALNPATLLPLPEEGLQHNCLDNSR
LIN.

In some embodiments, a prime editor comprises a eukaryotic RT, for example, a yeast, drosophila, rodent, or primate RT. In some embodiments, the prime editor comprises a Group II intron RT, for example, a. Geobacillus stearothermophilus Group II Intron (GsI-IIC) RT or a Eubacterium rectale group II intron (Eu.re.12) RT. In some embodiments, the prime editor comprises a retron RT.

### Programmable DNA Binding Domain

In some embodiments, the DNA-binding domain of a prime editor is a programmable DNA binding domain. A programmable DNA binding domain refers to a protein domain that is designed to bind a specific nucleic acid sequence, e.g., a target DNA or a target RNA. In some embodiments, the DNA-binding domain is a polynucleotide programmable DNA-binding domain that can associate with a guide polynucleotide (e.g., a PEgRNA) that guides the DNA-binding domain to a specific DNA sequence, e.g., a search target sequence in a target gene. In some embodiments, the DNA-binding domain comprises a Clustered Regularly Interspaced Short Palindromic Repeats (CRISPR) Associated (Cas) protein. A Cas protein may comprise any Cas protein described herein or a functional fragment or functional variant thereof. In some embodiments, a DNA-binding domain may also comprise a zinc-finger protein domain. In other cases, a DNA-binding domain comprises a transcription activator-like effector domain (TALE). In some embodiments, the DNA-binding domain comprises a DNA nuclease. For example, the DNA-binding domain of a prime editor may comprise an RNA-guided DNA endonuclease, e.g., a Cas protein. In some embodiments, the DNA-binding domain comprises a zinc finger nuclease (ZFN) or a transcription activator like effector domain nuclease (TALEN), where one or more zinc finger motifs or TALE motifs are associated with one or more nucleases, e.g., a Fok I nuclease domain.

In some embodiments, the DNA-binding domain comprise a nuclease activity. In some embodiments, the DNA-binding domain of a prime editor comprises an endonuclease domain having single strand DNA cleavage activity. For example, the endonuclease domain may comprise a FokI nuclease domain. In some embodiments, the DNA-binding domain of a prime editor comprises a nuclease having full nuclease activity. In some embodiments, the DNA-binding domain of a prime editor comprises a nuclease having modified or reduced nuclease activity as compared to a wild type endonuclease domain. For example, the endonuclease domain may comprise one or more amino acid substitutions as compared to a wild type endonuclease domain. In some embodiments, the DNA-binding domain of a prime editor has nickase activity. In some embodiments, the DNA-binding domain of a prime editor comprises a Cas protein domain that is a nickase. In some embodiments, compared to a wild type Cas protein, the Cas nickase comprises one or more amino acid substitutions in a nuclease domain that reduces or abolishes its double strand nuclease activity but retains DNA binding activity. In some embodiments, the Cas nickase comprises an amino acid substitution in a HNH domain. In some embodiments, the Cas nickase comprises an amino acid substitution in a RuvC domain.

In some embodiments, the DNA-binding domain comprises a CRISPR associated protein (Cas protein) domain. A Cas protein may be a Class 1 or a Class 2 Cas protein. A Cas protein can be a type I, type II, type III, type IV, type V Cas protein, or a type VI Cas protein. Non-limiting examples of Cas proteins include Cas9, Cas12a (Cpf1), Cas12e (CasX), Cas12d (CasY), Cas12b1 (C2c1), Cas12b2, Cas12c (C2c3), C2c4, C2c8, C2c5, C2c10, C2c9, Cas14a, Cas14b, Cas14c, Cas14d, Cas14e, Cas14f, Cas14g, Cas14h, Cas14u, Cns2, Cas Φ, and homologs, functional fragments, or modified versions thereof. A Cas protein can be a chimeric Cas protein that is fused to other proteins or polypeptides. A Cas protein can be a chimera of various Cas proteins, for example, comprising domains of Cas proteins from different organisms.

A Cas protein, e.g., Cas9, can be from any suitable organism. In some aspects, the organism is Streptococcus pyogenes (S. pyogenes). In some aspects, the organism is Staphylococcus aureus (S. aureus). In some aspects, the organism is Streptococcus thermophilus (S. thermophilus). In some embodiments, the organism is Staphylococcus lugdunensis.

A Cas protein, e.g., Cas9, can be a wild type or a modified form of a Cas protein. A Cas protein, e.g., Cas9, can be a nuclease active variant, nuclease inactive variant, a nickase, or a functional variant or functional fragment of a wild type Cas protein. A Cas protein, e.g., Cas9, can comprise an amino acid change such as a deletion, insertion, substitution, fusion, chimera, or any combination thereof relative to a wild-type version of the Cas protein. A Cas protein can be a polypeptide with at least about 50%, 60%, 70%, 80%, 90%, 91%, 92%, 93%, 94%, 95%, 96%, 97%, 98%, 99%, or 100% sequence identity or sequence similarity to a wild type exemplary Cas protein.

A Cas protein, e.g., Cas9, may comprise one or more domains. Non-limiting examples of Cas domains include, guide nucleic acid recognition and/or binding domain, nuclease domains (e.g., DNase or RNase domains, RuvC, HNH), DNA binding domain, RNA binding domain, helicase domains, protein-protein interaction domains, and dimerization domains. In various embodiments, a Cas protein comprises a guide nucleic acid recognition and/or binding domain can interact with a guide nucleic acid, and one or more nuclease domains that comprise catalytic activity for nucleic acid cleavage.

In some embodiments, a Cas protein, e.g., Cas9, comprises one or more nuclease domains. A Cas protein can comprise an amino acid sequence having at least about 50%, 60%, 70%, 80%, 90%, 91%, 92%, 93%, 94%, 95%, 96%, 97%, 98%, 99%, or 100% sequence identity to a nuclease domain (e.g., RuvC domain, HNH domain) of a wild-type Cas protein. In some embodiments, a Cas protein comprises a single nuclease domain. For example, a Cpf1 may comprise a RuvC domain but lacks HNH domain. In some embodiments, a Cas protein comprises two nuclease domains, e.g., a Cas9 protein can comprise an HNH nuclease domain and a RuvC nuclease domain.

In some embodiments, a prime editor comprises a Cas protein, e.g., Cas9, wherein all nuclease domains of the Cas protein are active. In some embodiments, a prime editor comprises a Cas protein having one or more inactive nuclease domains. One or a plurality of the nuclease domains (e.g., RuvC, HNH) of a Cas protein can be deleted or mutated so that they are no longer functional or comprise reduced nuclease activity. In some embodiments, a Cas protein, e.g., Cas9, comprising mutations in a nuclease domain has reduced (e.g., nickase) or abolished nuclease activity while maintaining its ability to target a nucleic acid locus at a search target sequence when complexed with a guide nucleic acid, e.g., a PEgRNA.

In some embodiments, a prime editor comprises a Cas nickase that can bind to the target gene in a sequence-specific manner and generate a single-strand break at a protospacer within double-stranded DNA in the target gene, but not a double-strand break. For example, the Cas nickase can cleave the edit strand or the non-edit strand of the target gene, but may not cleave both. In some embodiments, a prime editor comprises a Cas nickase comprising two nuclease domains (e.g., Cas9), with one of the two nuclease domains modified to lack catalytic activity or deleted. In some embodiments, the Cas nickase of a prime editor comprises a nuclease inactive RuvC domain and a nuclease active HNH domain. In some embodiments, the Cas nickase of a prime editor comprises a nuclease inactive HNH domain and a nuclease active RuvC domain. In some embodiments, a prime editor comprises a Cas9 nickase having an amino acid substitution in the RuvC domain. In some embodiments, the Cas9 nickase comprises a D10X amino acid substitution compared to a wild type S. pyogenes Cas9, wherein X is any amino acid other than D. In some embodiments, a prime editor comprises a Cas9 nickase having an amino acid substitution in the HNH domain. In some embodiments, the Cas9 nickase comprises a H840X amino acid substitution compared to a wild type S. pyogenes Cas9, wherein X is any amino acid other than H.

In some embodiments, a prime editor comprises a Cas protein that can bind to the target gene in a sequence-specific manner but lacks or has abolished nuclease activity and may not cleave either strand of a double stranded DNA in a target gene. Abolished activity or lacking activity can refer to an enzymatic activity less than 1%, less than 2%, less than 3%, less than 4%, less than 5%, less than 6%, less than 7%, less than 8%, less than 9%, or less than 10% activity compared to a wild-type exemplary activity (e.g., wild-type Cas9 nuclease activity). In some embodiments, a Cas protein of a prime editor completely lacks nuclease activity. A nuclease, e.g., Cas9, that lacks nuclease activity may be referred to as nuclease inactive or “nuclease dead” (abbreviated by “d”). A nuclease dead Cas protein (e.g., dCas, dCas9) can bind to a target polynucleotide but may not cleave the target polynucleotide. In some aspects, a dead Cas protein is a dead Cas9 protein. In some embodiments, a prime editor comprises a nuclease dead Cas protein wherein all of the nuclease domains (e.g., both RuvC and HNH nuclease domains in a Cas9 protein; RuvC nuclease domain in a Cpf1 protein) are mutated to lack catalytic activity, or are deleted.

A Cas protein can be modified. A Cas protein, e.g., Cas9, can be modified to increase or decrease nucleic acid binding affinity, nucleic acid binding specificity, and/or enzymatic activity. Cas proteins can also be modified to change any other activity or property of the protein, such as stability. For example, one or more nuclease domains of the Cas protein can be modified, deleted, or inactivated, or a Cas protein can be truncated to remove domains that are not essential for the function of the protein or to optimize (e.g., enhance or reduce) the activity of the Cas protein.

A Cas protein can be a fusion protein. For example, a Cas protein can be fused to a cleavage domain, an epigenetic modification domain, a transcriptional regulation domain, or a polymerase domain. A Cas protein can also be fused to a heterologous polypeptide providing increased or decreased stability. The fused domain or heterologous polypeptide can be located at the N-terminus, the C-terminus, or internally within the Cas protein.

In some embodiments, the Cas protein of a prime editor is a Class 2 Cas protein. In some embodiments, the Cas protein is a type II Cas protein. In some embodiments, the Cas protein is a Cas9 protein, a modified version of a Cas9 protein, a Cas9 protein homolog, mutant, variant, or a functional fragment thereof. As used herein, a Cas9, Cas9 protein, Cas9 polypeptide or a Cas9 nuclease refers to an RNA guided nuclease comprising one or more Cas9 nuclease domains and a Cas9 gRNA binding domain having the ability to bind a guide polynucleotide, e.g., a PEgRNA. A Cas9 protein may refer to a wild type Cas9 protein from any organism or a homolog, ortholog, or paralog from any organisms; any functional mutants or functional variants thereof; or any functional fragments or domains thereof. In some embodiments, a prime editor comprises a full-length Cas9 protein. In some embodiments, the Cas9 protein can generally comprises at least about 50%, 60%, 70%, 80%, 90%, 100% sequence identity to a wild type reference Cas9 protein (e.g., Cas9 from S. pyogenes). In some embodiments, the Cas9 comprises an amino acid change such as a deletion, insertion, substitution, fusion, chimera, or any combination thereof as compared to a wild type reference Cas9 protein.

In some embodiments, a Cas9 protein may comprise a Cas9 protein from Streptococcus pyogenes (Sp), Staphylococcus aureus (Sa), Streptococcus canis (Sc), Streptococcus thermophilus (St), Staphylococcus lugdunensis (Slu), Neisseria meningitidis (Nm), Campylobacter jejuni (Cj), Francisella novicida (Fn), or Treponema denticola (Td), or any Cas9 homolog or ortholog from an organism known in the art. In some embodiments, a Cas9 polypeptide is a SpCas9 polypeptide. In some embodiments, a Cas9 polypeptide is a SaCas9 polypeptide. In some embodiments, a Cas9 polypeptide is a ScCas9 polypeptide. In some embodiments, a Cas9 polypeptide is a StCas9 polypeptide. In some embodiments, a Cas9 polypeptide is a SluCas9 polypeptide. In some embodiments, a Cas9 polypeptide is a NmCas9 polypeptide. In some embodiments, a Cas9 polypeptide is a CjCas9 polypeptide. In some embodiments, a Cas9 polypeptide is a FnCas9 polypeptide. In some embodiments, a Cas9 polypeptide is a TdCas9 polypeptide. In some embodiments, a Cas9 polypeptide is a chimera comprising domains from two or more of the organisms described herein or those known in the art. In some embodiments, a Cas9 polypeptide is a Cas9 polypeptide from Streptococcus macacae. In some embodiments, a Cas9 polypeptide is a Cas9 polypeptide generated by replacing a PAM interaction domain of a SpCas9 with that of a Streptococcus macacae Cas9 (Spy-mac Cas9).

An exemplary Streptococcus pyogenes Cas9 (SpCas9) amino acid sequence is provided in SEQ ID NO: 4449.

Exemplary Streptococcus pyogenes Cas9 (SpCas9) amino acid sequence:

SEQ ID NO: 4449
MDKKYSIGLDIGTNSVGWAVITDEYKVPSKKFKVLGNTDRHSIKK
NLIGALLFDSGETAEATRLKRTARRRYTRRKNRICYLQEIFSNEM
AKVDDSFFHRLEESFLVEEDKKHERHPIFGNIVDEVAYHEKYPT
IYHLRKKLVDSTDKADLRLIYLALAHMIKFRGHFLIEGDLNPDNS
DVDKLFIQLVQTYNQLFEENPINASGVDAKAILSARLSKSRRLEN
LIAQLPGEKKNGLFGNLIALSLGLTPNFKSNFDLAEDAKLQLSKD
TYDDDLDNLLAQIGDQYADLFLAAKNLSDAILLSDILRVNTEITK
APLSASMIKRYDEHHQDLTLLKALVRQQLPEKYKEIFFDQSKNGY
AGYIDGGASQEEFYKFIKPILEKMDGTEELLVKLNREDLLRKQRT
FDNGSIPHQIHLGELHAILRRQEDFYPFLKDNREKIEKILTFRIP
YYVGPLARGNSRFAWMTRKSEETITPWNFEEVVDKGASAQSFIER
MTNFDKNLPNEKVLPKHSLLYEYFTVYNELTKVKYVTEGMRKPAF
LSGEQKKAIVDLLFKTNRKVTVKQLKEDYFKKIECFDSVEISGVE
DRFNASLGTYHDLLKIIKDKDFLDNEENEDILEDIVLTLTLFEDR
EMIEERLKTYAHLFDDKVMKQLKRRRYTGWGRLSRKLINGIRDKQ
SGKTILDFLKSDGFANRNFMQLIHDDSLTFKEDIQKAQVSGQGDS
LHEHIANLAGSPAIKKGILQTVKVVDELVKVMGRHKPENIVIEMA
RENQTTQKGQKNSRERMKRIEEGIKELGSQILKEHPVENTQLQNE
KLYLYYLQNGRDMYVDQELDINRLSDYDVDHIVPQSFLKDDSIDN
KVLTRSDKNRGKSDNVPSEEVVKKMKNYWRQLLNAKLITQRKFDN
LTKAERGGLSELDKAGFIKRQLVETRQITKHVAQILDSRMNTKYD
ENDKLIREVKVITLKSKLVSDFRKDFQFYKVREINNYHHAHDAYL
NAVVGTALIKKYPKLESEFVYGDYKVYDVRKMIAKSEQEIGKATA
KYFFYSNIMNFFKTEITLANGEIRKRPLIETNGETGEIVWDKGRD
FATVRKVLSMPQVNIVKKTEVQTGGFSKESILPKRNSDKLIARKK
DWDPKKYGGFDSPTVAYSVLVVAKVEKGKSKKLKSVKELLGITIM
ERSSFEKNPIDFLEAKGYKEVKKDLIIKLPKYSLFELENGRKRML
ASAGELQKGNELALPSKYVNFLYLASHYEKLKGSPEDNEQKQLFV
EQHKHYLDEIIEQISEFSKRVILADANLDKVLSAYNKHRDKPIRE
QAENIIHLFTLTNLGAPAAFKYFDTTIDRKRYTSTKEVLDATLIH
QSITGLYETRIDLSQLGGD.

In some embodiments, a prime editor comprises a Cas9 protein from Staphylococcus lugdunensis (Slu Cas9). An exemplary amino acid sequence of a Slu Cas9 is provided in SEQ ID NO: 4450.

Exemplary Staphylococcus lugdunensis Cas9 (Slu Cas9) amino acid sequence WP_002460848.1:

(SEQ ID NO: 4450)
MKRNYILGLDIGITSVGYGIIDYETRDVIDAGVRLFKEANVENNE
GRRSKRGARRLKRRRRHRIQRVKKLLFDYNLLTDHSELSGINPYE
ARVKGLSQKLSEEEFSAALLHLAKRRGVHNVNEVEEDTGNELSTK
EQISRNSKALEEKYVAELQLERLKKDGEVRGSINRFKTSDYVKEA
KQLLKVQKAYHQLDQSFIDTYIDLLETRRTYYEGPGEGSPFGWKD
IKEWYEMLMGHCTYFPEELRSVKYAYNADLYNALNDLNNLVITRD
ENEKLEYYEKFQIIENVFKQKKKPTLKQIAKEILVNEEDIKGYRV
TSTGKPEFTNLKVYHDIKDITARKEIIENAELLDQIAKILTIYQS
SEDIQEELTNLNSELTQEEIEQISNLKGYTGTHNLSLKAINLILD
ELWHTNDNQIAIFNRLKLVPKKVDLSQQKEIPTTLVDDFILSPVV
KRSFIQSIKVINAIIKKYGLPNDIIIELAREKNSKDAQKMINEMQ
KRNRQTNERIEEIIRTTGKENAKYLIEKIKLHDMQEGKCLYSLEA
IPLEDLLNNPFNYEVDHIIPRSVSFDNSFNNKVLVKQEEASKKGN
RTPFQYLSSSDSKISYETFKKHILNLAKGKGRISKTKKEYLLEER
DINRFSVQKDFINRNLVDTRYATRGLMNLLRSYFRVNNLDVKVKS
INGGFTSFLRRKWKFKKERNKGYKHHAEDALIIANADFIFKEWKK
LDKAKKVMENQMFEEKQAESMPEIETEQEYKEIFITPHQIKHIKD
FKDYKYSHRVDKKPNRELINDTLYSTRKDDKGNTLIVNNLNGLYD
KDNDKLKKLINKSPEKLLMYHHDPQTYQKLKLIMEQYGDEKNPLY
KYYEETGNYLTKYSKKDNGPVIKKIKYYGNKLNAHLDITDDYPNS
RNKVVKLSLKPYRFDVYLDNGVYKFVTVKNLDVIKKENYYEVNSK
CYEEAKKLKKISNQAEFIASFYNNDLIKINGELYRVIGVNNDLLN
RIEVNMIDITYREYLENMNDKRPPRIIKTIASKTQSIKKYSTDIL
GNLYEVKSKKHPQIIKK.

In some embodiments, a Cas9 protein comprises a variant Cas9 protein containing one or more amino acid substitutions. In some embodiments, a wildtype Cas9 protein comprises a RuvC domain and an HNH domain. In some embodiments, a prime editor comprises a nuclease active Cas9 protein that may cleave both strands of a double stranded target DNA sequence. In some embodiments, the nuclease active Cas9 protein comprises a functional RuvC domain and a functional HNH domain. In some embodiments, a prime editor comprises a Cas9 nickase that can bind to a guide polynucleotide and recognize a target DNA, but can cleave only one strand of a double stranded target DNA. In some embodiments, the Cas9 nickase comprises only one functional RuvC domain or one functional HNH domain. In some embodiments, a prime editor comprises a Cas9 that has a non-functional HNH domain and a functional RuvC domain. In some embodiments, the prime editor can cleave the edit strand (i.e., the PAM strand), but not the non-edit strand of a double stranded target DNA sequence. In some embodiments, a prime editor comprises a Cas9 having a non-functional RuvC domain that can cleave the target strand (i.e., the non-PAM strand), but not the edit strand of a double stranded target DNA sequence. In some embodiments, a prime editor comprises a Cas9 that has neither a functional RuvC domain nor a functional HNH domain, which may not cleave any strand of a double stranded target DNA sequence.

In some embodiments, a prime editor comprises a Cas9 having a mutation in the RuvC domain that reduces or abolishes the nuclease activity of the RuvC domain. In some embodiments, the Cas9 comprise a mutation at amino acid D10 as compared to a wild type SpCas9 as set forth in SEQ ID NO: 4449, or a corresponding mutation thereof. In some embodiments, the Cas9 comprise a D10A mutation as compared to a wild type SpCas9 as set forth in SEQ ID NO: 4449, or a corresponding mutation thereof. In some embodiments, the Cas9 polypeptide comprise a mutation at amino acid D10, G12, and/or G17 as compared to a wild type SpCas9 as set forth in SEQ ID NO: 4449, or a corresponding mutation thereof. In some embodiments, the Cas9 polypeptide comprise a D10A mutation, a G12A mutation, and/or a G17A mutation as compared to a wild type SpCas9 as set forth in SEQ ID NO: 4449, or a corresponding mutation thereof.

In some embodiments, a prime editor comprises a Cas9 polypeptide having a mutation in the HNH domain that reduces or abolishes the nuclease activity of the HNH domain. In some embodiments, the Cas9 polypeptide comprise a mutation at amino acid H840 as compared to a wild type SpCas9 as set forth in SEQ ID NO: 4449, or a corresponding mutation thereof. In some embodiments, the Cas9 polypeptide comprise a H840A mutation as compared to a wild type SpCas9 as set forth in SEQ ID NO: 4449, or a corresponding mutation thereof. In some embodiments, the Cas9 polypeptide comprise a mutation at amino acid E762, D839, H840, N854, N856, N863, H982, H983, A984, D986, and/or a A987 as compared to a wild type SpCas9 as set forth in SEQ ID NO: 4449, or a corresponding mutation thereof. In some embodiments, the Cas9 polypeptide comprise a E762A, D839A, H840A, N854A, N856A, N863A, H982A, H983A, A984A, and/or a D986A mutation as compared to a wild type SpCas9 as set forth in SEQ ID NO: 4449, or a corresponding mutation thereof.

In some embodiments, a prime editor comprises a Cas9 having one or more amino acid substitutions in both the HNH domain and the RuvC domain that reduce or abolish the nuclease activity of both the HNH domain and the RuvC domain. In some embodiments, the prime editor comprises a nuclease inactive Cas9, or a nuclease dead Cas9 (dCas9). In some embodiments, the dCas9 comprises a H840X substitution and a D10X mutation compared to a wild type SpCas9 as set forth in SEQ ID NO: 4449 or corresponding mutations thereof, wherein X is any amino acid other than H for the H840X substitution and any amino acid other than D for the D10X substitution. In some embodiments, the dead Cas9 comprises a H840A and a D10A mutation as compared to a wild type SpCas9 as set forth in SEQ ID NO: 4449, or corresponding mutations thereof.

In some embodiments, the N-terminal methionine is removed from a Cas9 nickase, or from any Cas9 variant, ortholog, or equivalent disclosed or contemplated herein. For example, methionine-minus Cas9 nickases include the following sequences, or a variant thereof having an amino acid sequence that has at least 80%, at least 85%, at least 90%, at least 95%, or at least 99% sequence identity thereto.

Besides dead Cas9 and Cas9 nickase variants, the Cas9 proteins used herein may also include other Cas9 variants having at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any reference Cas9 protein, including any wild type Cas9, or mutant Cas9 (e.g., a dead Cas9 or Cas9 nickase), or fragment Cas9, or circular permutant Cas9, or other variant of Cas9 disclosed herein or known in the art. In some embodiments, a Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a reference Cas9, e.g., a wild type Cas9. In some embodiments, the Cas9 variant comprises a fragment of a reference Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of the reference Cas9, e.g., a wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild type Cas9.

In some embodiments, a Cas9 fragment is a functional fragment that retains one or more Cas9 activities. In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length.

In some embodiments, a prime editor comprises a Cas protein, e.g., Cas9, containing modifications that allow altered PAM recognition. In prime editing using a Cas-protein-based prime editor, a “protospacer adjacent motif (PAM)”, PAM sequence, or PAM-like motif, may be used to refer to a short DNA sequence immediately following the protospacer sequence on the PAM strand of the target gene. In some embodiments, the PAM is recognized by the Cas nuclease in the prime editor during prime editing. In certain embodiments, the PAM is required for target binding of the Cas protein. The specific PAM sequence required for Cas protein recognition may depend on the specific type of the Cas protein. A PAM can be 1, 2, 3, 4, 5, 6, 7, 8, 9, 10 or more nucleotides in length. In some embodiments, a PAM is between 2-6 nucleotides in length. In some embodiments, the PAM can be a 5′ PAM (i.e., located upstream of the 5′ end of the protospacer). In other embodiments, the PAM can be a 3′ PAM (i.e., located downstream of the 5′ end of the protospacer). In some embodiments, the Cas protein of a prime editor recognizes a canonical PAM, for example, a SpCas9 recognizes 5′-NGG-3′ PAM. In some embodiments, the Cas protein of a prime editor has altered or non-canonical PAM specificities. Exemplary PAM sequences and corresponding Cas variants are described in Table 7 below. It should be appreciated that for each of the variants provided, the Cas protein comprises one or more of the amino acid substitutions as indicated compared to a wild type Cas protein sequence, for example, the Cas9 as set forth in SEQ ID NO: 4449. The PAM motifs as shown in Table 7 below are in the order of 5′ to 3′.

TABLE 7
Cas protein variants and corresponding PAM sequences

| Variant | PAM |
|---|---|
| spCas9 (wild type) | NGG, NGA, NAG, |
| | NGNGA |
| spCas9- VRVRFRR R1335V, L1111R, D1135V, | NG |
| G1218R, E1219F, A1322R, T1337R | |
| spCas9-VQR (D1135V, R1335Q, T1337R) | NGA |
| spCas9-EQR (D1135E, R1335Q, T1337R) | NGA |
| spCas9-VRER (D1135V, G1218R, R1335E, | NGCG |
| T1337R) | |
| spCas9-VRQR (D1135V, G1218R, R1335Q, | NGA |
| T1337R) | |
| Cas9-NG (L1111R, D1135V, G1218R, E1219F, | NGN |
| A1322R, T1337R, R1335V) | |
| SpG Cas9 (D1135L, S1136W, G1218K, E1219Q, | NGN |
| R1335Q, T1337R) | |
| SyRY Cas9 | NRN |
| (A61R, L1111R, N1317R, A1322R, and R1333P) | |
| xCas9 (E480K, E543D, E1219V, K294R, Q1256K, | NGN |
| A262T, S409I, M694I) | |
| SluCa9 | NNGG |
| sRGN1, sRGN2, sRGN4, sRGN3.1, sRGN3.3 | NNGG |
| saCas9 | NNGRRT, |
| | NNGRRN |
| saCas9-KKH (E782K, N968K, R1015H) | NNNRRT |
| spCas9-MQKSER (D1135M, S1136Q, G1218K, | NGCG/NGCN |
| E1219S, R1335E, T1337R) | |
| spCas9-LRKIQK (D1135L, S1136R, G1218K, | NGTN |
| E1219I, R1335Q, T1337K) | |
| spCas9-LRVSQK (D1135L, S1136R, G1218V, | NGTN |
| E1219S, R1335Q, T1337K) | |
| spCas9-LRVSQL(D1135L, S1136R, G1218V, | NGTN |
| E1219S, R1335Q, T1337L) | |
| Cpf1 | TTTV |
| Spy-Mac | NAA |
| NmCas9 | NNNNGATT |
| StCas9 | NNAGAAW |
| TdCas9 | NAAAAC |

In some embodiments, a prime editor comprises a Cas9 polypeptide comprising one or mutations selected from the group consisting of: A61R, L111R, D1135V, R221K, A262T, R324L, N394K, S4091, S4091, E427G, E480K, M495V, N497A, Y515N, K526E, F539S, E543D, R654L, R661A, R661L, R691A, N692A, M694A, M694I, Q695A, H698A, R753G, M763I, K848A, K890N, Q926A, K1003A, R1060A, L1111R, R1114G, D1135E, D1135L, D1135N, S1136W, V1139A, D1180G, G1218K, G1218R, G1218S, E1219Q, E1219V, E1219V, Q1221H, P1249S, E1253K, N1317R, A1320V, P1321S, A1322R, 11322V, D1332G, R1332N, A1332R, R1333K, R1333P, R1335L, R1335Q, R1335V, T1337N, T1337R, S1338T, H1349R, and any combinations thereof as compared to a wildtype SpCas9 polypeptide as set forth in SEQ ID NO: 4449.

In some embodiments, a prime editor comprises a SaCas9 polypeptide. In some embodiments, the SaCas9 polypeptide comprises one or more of mutations E782K, N968K, and R1015H as compared to a wild type SaCas9. In some embodiments, a prime editor comprises a FnCas9 polypeptide, for example, a wildtype FnCas9 polypeptide or a FnCas9 polypeptide comprising one or more of mutations E1369R, E1449H, or R1556A as compared to the wild type FnCas9. In some embodiments, a prime editor comprises a Sc Cas9, for example, a wild type ScCas9 or a ScCas9 polypeptide comprises one or more of mutations 1367K, G368D, 1369K, H371L, T375S, T376G, and T1227K as compared to the wild type ScCas9. In some embodiments, a prime editor comprises a St1 Cas9 polypeptide, a St3 Cas9 polypeptide, or a Slu Cas9 polypeptide.

In some embodiments, a prime editor comprises a Cas polypeptide that comprises a circular permutant Cas variant. For example, a Cas9 polypeptide of a prime editor may be engineered such that the N-terminus and the C-terminus of a Cas9 protein (e.g., a wild type Cas9 protein, or a Cas9 nickase) are topically rearranged to retain the ability to bind DNA when complexed with a guide RNA (gRNA). An exemplary circular permutant configuration may be N-terminus-[original C-terminus]-[original N-terminus]-C-terminus. Any of the Cas9 proteins described herein, including any variant, ortholog, or naturally occurring Cas9 or equivalent thereof, may be reconfigured as a circular permutant variant.

In various embodiments, the circular permutants of a Cas protein, e.g., a Cas9, may have the following structure: N-terminus-[original C-terminus]-[optional linker]-[original N-terminus]-C-terminus. In some embodiments, a circular permutant Cas9 comprises any one of the following structures:
N-terminus-[1268-1368]-[optional linker]-[1-1267]-C-terminus;
N-terminus-[1168-1368]-[optional linker]-[1-1167]-C-terminus;
N-terminus-[1068-1368]-[optional linker]-[1-1067]-C-terminus;
N-terminus-[968-1368]-[optional linker]-[1-967]-C-terminus;
N-terminus-[868-1368]-[optional linker]-[1-867]-C-terminus;
N-terminus-[768-1368]-[optional linker]-[1-767]-C-terminus;
N-terminus-[668-1368]-[optional linker]-[1-667]-C-terminus;
N-terminus-[568-1368]-[optional linker]-[1-567]-C-terminus;
N-terminus-[468-1368]-[optional linker]-[1-467]-C-terminus;
N-terminus-[368-1368]-[optional linker]-[1-367]-C-terminus;
N-terminus-[268-1368]-[optional linker]-[1-267]-C-terminus;
N-terminus-[168-1368]-[optional linker]-[1-167]-C-terminus;
N-terminus-[68-1368]-[optional linker]-[1-67]-C-terminus;
N-terminus-[10-1368]-[optional linker]-[1-9]-C-terminus, or the corresponding circular permutants of other Cas9 proteins (including other Cas9 orthologs, variants, etc.).

In some embodiments, a circular permutant Cas9 comprises any one of the following structures (amino acid positions as set forth in SEQ ID NO: 4449-1368 amino acids of UniProtKB-Q99ZW2:
N-terminus-[102-1368]-[optional linker]-[1-101]-C-terminus;
N-terminus-[1028-1368]-[optional linker]-[1-1027]-C-terminus;
N-terminus-[1041-1368]-[optional linker]-[1-1043]-C-terminus;
N-terminus-[1249-1368]-[optional linker]-[1-1248]-C-terminus; or
N-terminus-[1300-1368]-[optional linker]-[1-1299]-C-terminus, or the corresponding circular permutants of other Cas9 proteins (including other Cas9 orthologs, variants, etc).

In some embodiments, a circular permutant Cas9 comprises any one of the following structures (amino acid positions as set forth in SEQ ID NO: 4449-1368 amino acids of UniProtKB-Q99ZW2 N-terminus-[103-1368]-[optional linker]-[1-102]-C-terminus:
N-terminus-[1029-1368]-[optional linker]-[1-1028]-C-terminus;
N-terminus-[1042-1368]-[optional linker]-[1-1041]-C-terminus;
N-terminus-[1250-1368]-[optional linker]-[1-1249]-C-terminus; or
N-terminus-[1301-1368]-[optional linker]-[1-1300]-C-terminus, or the corresponding circular permutants of other Cas9 proteins (including other Cas9 orthologs, variants, etc).

In some embodiments, the circular permutant can be formed by linking a C-terminal fragment of a Cas9 to an N-terminal fragment of a Cas9, either directly or by using a linker, such as an amino acid linker. In some embodiments, The C-terminal fragment may correspond to the 95% or more of the C-terminal amino acids of a Cas9 (e.g., amino acids about 1300-1368 as set forth in SEQ ID No: 4449 or corresponding amino acid positions thereof), or the 90%, 85%, 80%, 75%, 70%, 65%, 60%, 55%, 50%, 45%, 40%, 35%, 30%, 25%, 20%, 15%, 10%, or 5% or more of the C-terminal amino acids of a Cas9 (e.g., SEQ ID No: 4449). The N-terminal portion may correspond to 95% or more of the N-terminal amino acids of a Cas9 (e.g., amino acids about 1-1300 as set forth in SEQ ID No: 4449 or corresponding amino acid positions thereof), or 90%, 85%, 80%, 75%, 70%, 65%, 60%, 55%, 50%, 45%, 40%, 35%, 30%, 25%, 20%, 15%, 10%, or 5% or more of the N terminal amino acids of a Cas9 (e.g., as set forth in SEQ ID No: 4449 or corresponding amino acid positions thereof).

In some embodiments, the circular permutant can be formed by linking a C-terminal fragment of a Cas9 to an N-terminal fragment of a Cas9, either directly or by using a linker, such as an amino acid linker. In some embodiments, the C-terminal fragment that is rearranged to the N-terminus includes or corresponds to the C-terminal 30% or less of the amino acids of a Cas9 (e.g., amino acids 1012-1368 as set forth in SEQ ID No: 4449 or corresponding amino acid positions thereof). In some embodiments, the C-terminal fragment that is rearranged to the N-terminus, includes or corresponds to the C-terminal 30%, 29%, 28%, 27%, 26%, 25%, 24%, 23%, 22%, 21%, 20%, 19%, 18%, 17%, 16%, 15%, 14%, 13%, 12%, 11%, 10%, 9%, 8%, 7%, 6%, 5%, 4%, 3%, 2%, or 1% of the amino acids of a Cas9 (e.g., as set forth in SEQ ID No: 4449 or corresponding amino acid positions thereof). In some embodiments, the C-terminal fragment that is rearranged to the N-terminus, includes or corresponds to the C-terminal 410 residues or less of a Cas9 (e.g., as set forth in SEQ ID No: 4449 or corresponding amino acid positions thereof). In some embodiments, the C-terminal portion that is rearranged to the N-terminus, includes or corresponds to the C-terminal 410, 400, 390, 380, 370, 360, 350, 340, 330, 320, 310, 300, 290, 280, 270, 260, 250, 240, 230, 220, 210, 200, 190, 180, 170, 160, 150, 140, 130, 120, 110, 100, 90, 80, 70, 60, 50, 40, 30, 20, or 10 residues of a Cas9 (e/g/as set forth in SEQ ID No: 4449 or corresponding amino acid positions thereof). In some embodiments, the C-terminal portion that is rearranged to the N-terminus includes or corresponds to the C-terminal 357, 341, 328, 120, or 69 residues of a Cas9 (e.g., as set forth in SEQ ID No: 4449 or corresponding amino acid positions thereof).

In other embodiments, circular permutant Cas9 variants may be a topological rearrangement of a Cas9 primary structure based on the following method, which is based on S. pyogenes Cas9 of SEQ ID NO: 4449: (a) selecting a circular permutant (CP) site corresponding to an internal amino acid residue of the Cas9 primary structure, which dissects the original protein into two halves: an N-terminal region and a C-terminal region; (b) modifying the Cas9 protein sequence (e.g., by genetic engineering techniques) by moving the original C-terminal region (comprising the CP site amino acid) to precede the original N-terminal region, thereby forming a new N-terminus of the Cas9 protein that now begins with the CP site amino acid residue. The CP site can be located in any domain of the Cas9 protein, including, for example, the helical-II domain, the RuvCIII domain, or the CTD domain. For example, the CP site may be located (as set forth in SEQ ID No: 4449 or corresponding amino acid positions thereof) at original amino acid residue 181, 199, 230, 270, 310, 1010, 1016, 1023, 1029, 1041, 1247, 1249, or 1282. Thus, once relocated to the N-terminus, original amino acid 181, 199, 230, 270, 310, 1010, 1016, 1023, 1029, 1041, 1247, 1249, or 1282 would become the new N-terminal amino acid. Nomenclature of these CP-Cas9 proteins may be referred to as Cas9-CP181, Cas9-CP199, Cas9-CP230, Cas9-CP270, Cas9-CP310, Cas9-CP1010, Cas9-CP1016, Cas9-CP1023, Cas9-CP1029, Cas9-CP1041, Cas9-CP1247, Cas9-CP1249, and Cas9-CP1282, respectively. This description is not meant to be limited to making CP variants from SEQ ID NO: 18, but may be implemented to make CP variants in any Cas9 sequence, either at CP sites that correspond to these positions, or at other CP sites entirely. This description is not meant to limit the specific CP sites in any way. Virtually any CP site may be used to form a CP-Cas9 variant.

In some embodiments, a prime editor comprises a Cas9 functional variant that is of smaller molecular weight than a wild type SpCas9 protein. In some embodiments, a smaller-sized Cas9 functional variant may facilitate delivery to cells, e.g., by an expression vector, nanoparticle, or other means of delivery. In certain embodiments, a smaller-sized Cas9 functional variant is a Class 2 Type II Cas protein. In certain embodiments, a smaller-sized Cas9 functional variant is a Class 2 Type V Cas protein. In certain embodiments, a smaller-sized Cas9 functional variant is a Class 2 Type VI Cas protein.

In some embodiments, a prime editor comprises a SpCas9 that is 1368 amino acids in length and has a predicted molecular weight of 158 kilodaltons. In some embodiments, a prime editor comprises a Cas9 functional variant or functional fragment that is less than 1300 amino acids, less than 1290 amino acids, than less than 1280 amino acids, less than 1270 amino acids, less than 1260 amino acid, less than 1250 amino acids, less than 1240 amino acids, less than 1230 amino acids, less than 1220 amino acids, less than 1210 amino acids, less than 1200 amino acids, less than 1190 amino acids, less than 1180 amino acids, less than 1170 amino acids, less than 1160 amino acids, less than 1150 amino acids, less than 1140 amino acids, less than 1130 amino acids, less than 1120 amino acids, less than 1110 amino acids, less than 1100 amino acids, less than 1050 amino acids, less than 1000 amino acids, less than 950 amino acids, less than 900 amino acids, less than 850 amino acids, less than 800 amino acids, less than 750 amino acids, less than 700 amino acids, less than 650 amino acids, less than 600 amino acids, less than 550 amino acids, or less than 500 amino acids, but at least larger than about 400 amino acids and retaining the one or more functions, e.g., DNA binding function, of the Cas9 protein.

In some embodiments, the Cas protein may include any CRISPR associated protein, including but not limited to, Cas12a, Cas12b1, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof, and preferably comprising a nickase mutation (e.g., a mutation corresponding to the D10A mutation of the wild type Cas9 polypeptide of SEQ ID NO: 18). In various other embodiments, the napDNAbp can be any of the following proteins: a Cas9, a Cas12a (Cpf1), a Cas12e (CasX), a Cas12d (CasY), a Cas12b1 (C2c1), a Cas13a (C2c2), a Cas12c (C2c3), a GeoCas9, a CjCas9, a Cas12g, a Cas12h, a Cas12i, a Cas13b, a Cas13c, a Cas13d, a Cas14, a Csn2, an xCas9, an SpCas9-NG, a circularly permuted Cas9, or an Argonaute (Ago) domain, or a functional variant or fragment thereof.

TABLE 8
Exemplary Cas proteins

| | |
|---|---|
| Legacy nomenclature | Current nomenclature |

type II CRISPR-Cas enzymes

| | |
|---|---|
| Cas9 | same |

type V CRISPR-Cas enzymes

| | |
|---|---|
| Cpf1 | Cas12a |
| CasX | Cas12e |
| C2c1 | Cas12b1 |
| Cas12b2 | same |
| C2c3 | Cas12c |
| CasY | Cas12d |
| C2c4 | same |
| C2c8 | same |
| C2c5 | same |
| C2c10 | same |
| C2c9 | same |

type VI CRISPR-Cas enzymes

| | |
|---|---|
| C2c2 | Cas13a |
| Cas13d | same |
| C2c7 | Cas13c |
| C2c6 | Cas13b |

In some embodiments, a prime editor as described herein may comprise a Cas12a (Cpf1) polypeptide or functional variants thereof. In some embodiments, the Cas12a polypeptide comprises a mutation that reduces or abolishes the endonuclease domain of the Cas12a polypeptide. In some embodiments, the Cas12a polypeptide is a Cas12a nickase. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12a polypeptide.

In some embodiments, a prime editor comprises a Cas protein that is a Cas12b (C2c1) or a Cas12c (C2c3) polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally occurring Cas12b (C2c1) or Cas12c (C2c3) protein. In some embodiments, the Cas protein is a Cas12b nickase or a Cas12c nickase. In some embodiments, the Cas protein is a Cas12e, a Cas12d, a Cas13, Cas14a, Cas14b, Cas14c, Cas14d, Cas14e, Cas14f, Cas14g, Cas14h, Cas14u, or a CasΦ polypeptide. In some embodiments, the Cas protein comprises an amino acid sequence that comprises at least about 50%, 60%, 70%, 80%, 90%, 91%, 92%, 93%, 94%, 95%, 96%, 97%, 98%, 99%, or 100% sequence identity to a naturally-occurring Cas12e, Cas12d, Cas13, Cas14a, Cas14b, Cas14c, Cas14d, Cas14e, Cas14f, Cas14g, Cas14h, Cas14u, or Cas @ protein. In some embodiments, the Cas protein is a Cas12e, Cas12d, Cas13, or Cas @ nickase.

### Flap Endonuclease

In some embodiments, a prime editor further comprises additional polypeptide components, for example, a flap endonuclease (FEN, e.g., FEN1). In some embodiments, the flap endonuclease excises the 5′ single stranded DNA of the edit strand of the target gene and assists incorporation of the intended nucleotide edit into the target gene. In some embodiments, the FEN is linked or fused to another component. In some embodiments, the FEN is provided in trans, for example, as a separate polypeptide or polynucleotide encoding the FEN.

In some embodiments, a prime editor or prime editing composition comprises a flap nuclease. In some embodiments, the flap nuclease is a FEN1, or any FEN1 functional variant, functional mutant, or functional fragment thereof. In some embodiments, the flap nuclease is a TREX2, EXO1, or any other flap nuclease known in the art, or any functional variant, functional mutant, or functional fragment thereof. In some embodiments, the flap nuclease has amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to any of the flap nucleases described herein or known in the art.

### Nuclear Localization Sequences

In some embodiments, a prime editor further comprises one or more nuclear localization sequence (NLS). In some embodiments, the NLS helps promote translocation of a protein into the cell nucleus. In some embodiments, a prime editor comprises a fusion protein, e.g., a fusion protein comprising a DNA binding domain and a DNA polymerase that comprises one or more NLSs. In some embodiments, one or more polypeptides of the prime editor are fused to or linked to one or more NLSs. In some embodiments, the prime editor comprises a DNA binding domain and a DNA polymerase domain that are provided in trans, wherein the DNA binding domain and/or the DNA polymerase domain is fused or linked to one or more NLSs.

In certain embodiments, a prime editor or prime editing complex comprises at least one NLS. In some embodiments, a prime editor or prime editing complex comprises at least two NLSs. In embodiments with at least two NLSs, the NLSs can be the same NLS, or they can be different NLSs.

In some instances, a prime editor may further comprise at least one nuclear localization sequence (NLS). In some cases, a prime editor may further comprise 1 NLS. In some cases, a prime editor may further comprise 2 NLSs. In other cases, a prime editor may further comprise 3 NLSs. In one case, a primer editor may further comprise more than 4, 5, 6, 7, 8, 9 or 10 NLSs.

In addition, the NLSs may be expressed as part of a prime editor complex. In some embodiments, a NLS can be positioned almost anywhere in a protein's amino acid sequence, and generally comprises a short sequence of three or more or four or more amino acids. The location of the NLS fusion can be at the N-terminus, the C-terminus, or positioned anywhere within a sequence of a prime editor or a component thereof (e.g., inserted between the DNA-binding domain and the DNA polymerase domain of a prime editor fusion protein, between the DNA binding domain and a linker sequence, between a DNA polymerase and a linker sequence, between two linker sequences of a prime editor fusion protein or a component thereof, in either N-terminus to C-terminus or C-terminus to N-terminus order). In some embodiments, a prime editor is fusion protein that comprises an NLS at the N terminus. In some embodiments, a prime editor is fusion protein that comprises an NLS at the C terminus. In some embodiments, a prime editor is fusion protein that comprises at least one NLS at both the N terminus and the C terminus. In some embodiments, the prime editor is a fusion protein that comprises two NLSs at the N terminus and/or the C terminus.

Any NLSs that are known in the art are also contemplated herein. The NLSs may be any naturally occurring NLS, or any non-naturally occurring NLS (e.g., an NLS with one or more mutations relative to a wild-type NLS). In some embodiments, the one or more NLSs of a prime editor comprise bipartite NLSs. In some embodiments, a nuclear localization signal (NLS) is predominantly basic. In some embodiments, the one or more NLSs of a prime editor are rich in lysine and arginine residues. In some embodiments, the one or more NLSs of a prime editor comprise proline residues. In some embodiments, a nuclear localization signal (NLS) comprises the sequence MDSLLMNRRKFLYQFKNVRWAKGRRETYLC (SEQ ID NO: 7023), KRTADGSEFESPKKKRKV (SEQ ID NO: 7024), KRTADGSEFEPKKKRKV (SEQ ID NO: 7025), NLSKRPAAIKKAGQAKKKK (SEQ ID NO: 7026), RQRRNELKRSF (SEQ ID NO: 7027), or NQSSNFGPMKGGNFGGRSSGPYGGGGQYFAKPRNQGGY (SEQ ID NO: 7028).

In some embodiments, a NLS is a monopartite NLS. For example, in some embodiments, a NLS is a SV40 large T antigen NLS PKKKRKV (SEQ ID NO: 7029). In some embodiments, a NLS is a bipartite NLS. In some embodiments, a bipartite NLS comprises two basic domains separated by a spacer sequence comprising a variable number of amino acids. In some embodiments, a NLS is a bipartite NLS. In some embodiments, a bipartite NLS consists of two basic domains separated by a spacer sequence comprising a variable number of amino acids. In some embodiments, the spacer amino acid sequence comprises the Xenopus nucleoplasmin sequence KRXXXXXXXXXXKKKL (SEQ ID NO: 4451) wherein X is any amino acid. In some embodiments, a NLS is a noncanonical sequences such as M9 of the hnRNP A1 protein, the influenza virus nucleoprotein NLS, and the yeast Gal4 protein NLS.

Other non-limiting examples of NLS sequences are provided in Table 9 below.

TABLE 9
Exemplary nuclear localization sequences

| | | SEQ |
|---|---|---|
| | | ID |
| Description | Sequence | NO: |
| NLS of SV40 | PKKKRKV | 7029 |
| Large T-AG | | |
| NLS | MKRTADGSEFESPKKKRKV | 7030 |
| NLS | MDSLLMNRRKFLYQFKNVR | 7023 |
| | WAKGRRETYLC | |
| NLS of | AVKRPAATKKAGQAKKKKLD | 7031 |
| Nucleoplasmin | | |
| NLS of EGL-13 | MSRRRKANPTKLSENAKKLA | 7032 |
| | KEVEN | |
| NLS of C-Myc | PAAKRVKLD | 7033 |
| NLS of Tus- | KLKIKRPVK | 7034 |
| protein | | |
| NLS of polyoma | VSRKRPRP | 7035 |
| large T-AG | | |
| NLS of Hepatitis | EGAPPAKRAR | 7036 |
| D virus | | |
| antigen | | |
| NLS of murine | PPQPKKKPLDGE | 7037 |
| p53 | | |
| NLS | SGGSKRTADGSE | 7038 |
| | FEPKKKRKV | |

### Additional Prime Editor Components

A prime editor described herein may comprise additional functional domains, for example, one or more domains that modify the folding, solubility, or charge of the prime editor. In some instances, the prime editor may comprise a solubility-enhancement (SET) domain.

In some embodiments, a split intein comprises two halves of an intein protein, which may be referred to as a N-terminal half of an intein, or intein-N, and a C-terminal half of an intein, or intein-C, respectively. In some embodiments, the intein-N and the intein-C may each be fused to a protein domain (the N-terminal and the C-terminal exteins). The exteins can be any protein or polypeptides, for example, any prime editor polypeptide component. In some embodiments, the intein-N and intein-C of a split intein can associate non-covalently to form an active intein and catalyze a-trans splicing reaction. In some embodiments, the trans splicing reaction excises the two intein sequences and links the two extein sequences with a peptide bond. As a result, the intein-N and the intein-C are spliced out, and a protein domain linked to the intein-N is fused to a protein domain linked to the intein-C essentially in same way as a contiguous intein does. In some embodiments, a split-intein is derived from a eukaryotic intein, a bacterial intein, or an archaeal intein. Preferably, the split intein so-derived will possess only the amino acid sequences essential for catalyzing trans-splicing reactions. In some embodiments, an intein-N or an intein-C further comprise one or more amino acid substitutions as compared to a wild type intein-N or wild type intein-C, for example, amino acid substitutions that enhances the trans-splicing activity of the split intein. In some embodiments, the intein-C comprises 4 to 7 contiguous amino acid residues, wherein at least 4 amino acids of which are from the last-strand of the intein from which it was derived. In some embodiments, the split intein is derived from a Ssp DnaE intein, e.g., Synechocytis sp. PCC6803, or any intein or split intein known in the art, or any functional variants or fragments thereof.

In some embodiments, a prime editor comprises one or more epitope tags. Non-limiting examples of epitope tags include histidine (His) tags, V5 tags, FLAG tags, influenza hemagglutinin (HA) tags, Myc tags, VSV-G tags, thioredoxin (Trx) tags, biotin carboxylase carrier protein (BCCP) tags, myc-tags, calmodulin-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g., Softag 1, Softag 3), strep-tags, biotin ligase tags, FlAsH tags, V5 tags, and SBP-tags. Additional suitable sequences will be apparent to those of skill in the art. In some embodiments, the fusion protein comprises one or more His tags.

In some embodiments, a prime editor comprises one or more polypeptide domains encoded by one or more reporter genes. Examples of reporter genes include, but are not limited to, glutathione-5-transferase (GST), horseradish peroxidase (HRP), chloramphenicol acetyltransferase (CAT), beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP).

In some embodiments, a prime editor comprises one or more polypeptide domains that binds DNA molecules or binds other cellular molecules. Examples of binding proteins or domains include, but are not limited to, maltose binding protein (MBP), S-tag, Lex A DNA binding domain (DBD) fusions, GAL4 DNA binding domain fusions, and herpes simplex virus (HSV) BP16 protein fusions.

In some embodiments, a prime editor comprises a protein domain that is capable of modifying the intracellular half-life of the prime editor.

In some embodiments, a prime editing complex comprises a fusion protein comprising a DNA binding domain (e.g., Cas9 (H840A)) and a reverse transcriptase (e.g., a variant MMLV RT) having the following structure: [NLS]-[Cas9 (H840A)]-[linker]-[MMLV_RT(D200N) (T330P) (L603W) (T306K) (W313F)], and a desired PEgRNA. In some embodiments, the prime editing complex comprises a prime editor fusion protein that has the amino acid sequence of SEQ ID NO: 4440.

Polypeptides comprising components of a prime editor may be fused via peptide linkers, or may be provided in trans relevant to each other. For example, a reverse transcriptase may be expressed, delivered, or otherwise provided as an individual component rather than as a part of a fusion protein with the DNA binding domain. In such cases, components of the prime editor may be associated through non-peptide linkages or co-localization functions. In some embodiments, a prime editor further comprises additional components capable of interacting with, associating with, or capable of recruiting other components of the prime editor or the prime editing system. For example, a prime editor may comprise an RNA-protein recruitment polypeptide that can associate with an RNA-protein recruitment RNA aptamer. In some embodiments, an RNA-protein recruitment polypeptide can recruit, or be recruited by, a specific RNA sequence. Non limiting examples of RNA-protein recruitment polypeptide and RNA aptamer pairs include a MS2 coat protein and a MS2 RNA hairpin, a PCP polypeptide and a PP7 RNA hairpin, a Com polypeptide and a Com RNA hairpin, a Ku protein and a telomerase Ku binding RNA motif, and a Sm7 protein and a telomerase Sm7 binding RNA motif. In some embodiments, the prime editor comprises a DNA binding domain fused or linked to an RNA-protein recruitment polypeptide. In some embodiments, the prime editor comprises a DNA polymerase domain fused or linked to an RNA-protein recruitment polypeptide. In some embodiments, the DNA binding domain and the DNA polymerase domain fused to the RNA-protein recruitment polypeptide, or the DNA binding domain fused to the RNA-protein recruitment polypeptide and the DNA polymerase domain are co-localized by the corresponding RNA-protein recruitment RNA aptamer of the RNA-protein recruitment polypeptide. In some embodiments, the corresponding RNA-protein recruitment RNA aptamer fused or linked to a portion of the PEgRNA or ngRNA. For example, an MS2 coat protein fused or linked to the DNA polymerase and a MS2 hairpin installed on the PEgRNA for co-localization of the DNA polymerase and the RNA-guided DNA binding domain (e.g., a Cas9 nickase).

In some embodiments, a prime editor comprises a polypeptide domain, an MS2 coat protein (MCP), that recognizes an MS2 hairpin. In some embodiments, the nucleotide sequence of the MS2 hairpin (or equivalently referred to as the “MS2 aptamer”) is: GCCAACATGAGGATCACCCATGTCTGCAGGGCC (SEQ ID NO: 4446). In some embodiments, the amino acid sequence of the MCP is:

(SEQ ID NO: 4447)
GSASNFTQFVLVDNGGTGDVTVAPSNFANGVAEWISSNSRSQAYK
VTCSVRQSSAQNRKYTIKVEVPKVATQTVGGEELPVAGWRSYLN
MELTIPIFATNSDCELIVKAMQGLLKDGNPIPSAIAANSGIY

In certain embodiments, components of a prime editor are directly fused to each other. In certain embodiments, components of a prime editor are associated to each other via a linker.

As used herein, a linker can be any chemical group or a molecule linking two molecules or moieties, e.g., a DNA binding domain and a polymerase domain of a prime editor. In some embodiments, a linker is an organic molecule, group, polymer, or chemical moiety. In some embodiments, the linker comprises a non-peptide moiety. The linker may be as simple as a covalent bond, or it may be a polymeric linker many atoms in length, for example, a polynucleotide sequence. In certain embodiments, the linker is a covalent bond (e.g., a carbon-carbon bond, disulfide bond, carbon-heteroatom bond, etc.).

In certain embodiments, two or more components of a prime editor are linked to each other by a peptide linker. In some embodiments, a peptide linker is 5-100 amino acids in length, for example, 2, 3, 4, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. In some embodiments, the peptide linker is 16 amino acids in length, 24 amino acids in length, 64 amino acids in length, or 96 amino acids in length.

In some embodiments, the linker comprises the amino acid sequence (GGGGS)n (SEQ ID NO: 7039), (G)n (SEQ ID NO: 7040), (EAAAK)n (SEQ ID NO: 7041), (GGS)n (SEQ ID NO: 7042), (SGGS)n (SEQ ID NO: 7043), (XP)n (SEQ ID NO: 7044), or any combination thereof, wherein n is independently an integer between 1 and 30, and wherein X is any amino acid. In some embodiments, the linker comprises the amino acid sequence (GGS) n, wherein n is 1, 3, or 7 (SEQ ID NO: 7045). In some embodiments, the linker comprises the amino acid sequence SGSETPGTSESATPES (SEQ ID NO: 7046). In some embodiments, the linker comprises the amino acid sequence SGGSSGGSSGSETPGTSESATPESSGGSSGGS (SEQ ID NO: 7047). In some embodiments, the linker comprises the amino acid sequence SGGSGGSGGS (SEQ ID NO: 7048). In some embodiments, the linker comprises the amino acid sequence SGGS (SEQ ID NO: 7049). In other embodiments, the linker comprises the amino acid sequence

(SEQ ID NO: 7050)
SGGSSGGSSGSETPGTSESATPESAGSYPYDVPDYAGSAAPAAKK
KKLDGSGSGGSSGG
S

In some embodiments, a linker comprises 1-100 amino acids. In some embodiments, the linker comprises the amino acid sequence SGSETPGTSESATPES (SEQ ID NO: 7046). In some embodiments, the linker comprises the amino acid sequence SGGSSGGSSGSETPGTSESATPESSGGSSGGS (SEQ ID NO: 7047). In some embodiments, the linker comprises the amino acid sequence SGGSGGSGGS (SEQ ID NO: 7048). In some embodiments, the linker comprises the amino acid sequence SGGS (SEQ ID NO: 7049). In some embodiments, the linker comprises the amino acid sequence GGSGGS (SEQ ID NO: 7051), GGSGGSGGS (SEQ ID NO: 7052),

(SEQ ID NO: 7050)
SGGSSGGSSGSETPGTSESATPESAGSYPYDVP
DYAGSAAPAAKKKKLDGSGSGGSSGGS,
or
(SEQ ID NO: 7053)
SGGSSGGSSGSETPGTSESATPESSGGSSGGSS

In certain embodiments, two or more components of a prime editor are linked to each other by a non-peptide linker. In some embodiments, the linker is a carbon-nitrogen bond of an amide linkage. In certain embodiments, the linker is a cyclic or acyclic, substituted or unsubstituted, branched or unbranched aliphatic or heteroaliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

Components of a prime editor may be connected to each other in any order. In some embodiments, the DNA binding domain and the DNA polymerase domain of a prime editor may be fused to form a fusion protein, or may be joined by a peptide or protein linker, in any order from the N terminus to the C terminus. In some embodiments, a prime editor comprises a DNA binding domain fused or linked to the C-terminal end of a DNA polymerase domain. In some embodiments, a prime editor comprises a DNA binding domain fused or linked to the N-terminal end of a DNA polymerase domain. In some embodiments, the prime editor comprises a fusion protein comprising the structure NH2-[DNA binding domain]-[polymerase]-COOH; or NH2-[polymerase]-[DNA binding domain]-COOH, wherein each instance of “]-[” indicates the presence of an optional linker sequence. In some embodiments, a prime editor comprises a fusion protein and a DNA polymerase domain provided in trans, wherein the fusion protein comprises the structure NH2-[DNA binding domain]-[RNA-protein recruitment polypeptide]-COOH. In some embodiments, a prime editor comprises a fusion protein and a DNA binding domain provided in trans, wherein the fusion protein comprises the structure NH2-[DNA polymerase domain]-[RNA-protein recruitment polypeptide]-COOH.

In some embodiments, a prime editor fusion protein, a polypeptide component of a prime editor, or a polynucleotide encoding the prime editor fusion protein or polypeptide component, may be split into an N-terminal half and a C-terminal half or polypeptides that encode the N-terminal half and the C terminal half, and provided to a target DNA in a cell separately. For example, in certain embodiments, a prime editor fusion protein may be split into a N-terminal and a C-terminal half for separate delivery in AAV vectors, and subsequently translated and colocalized in a target cell to reform the complete polypeptide or prime editor protein. In such cases, separate halves of a protein or a fusion protein may each comprise a split-intein to facilitate colocalization and reformation of the complete protein or fusion protein by the mechanism of intein facilitated trans splicing. In some embodiments, a prime editor comprises a N-terminal half fused to an intein-N, and a C-terminal half fused to an intein-C, or polynucleotides or vectors (e.g., AAV vectors) encoding each thereof. When delivered and/or expressed in a target cell, the intein-N and the intein-C can be excised via protein trans-splicing, resulting in a complete prime editor fusion protein in the target cell.

In some embodiments, a prime editor fusion protein comprises a Cas9 (H840A) nickase and a wild type M-MLV RT. In some embodiments, a prime editor fusion protein comprises one or more individual components of a prime editor fusion protein comprises a Cas9 (H840A) nickase and a wild type M-MLV RT. In some embodiments, a prime editor fusion protein comprises a Cas9 (H840A) nickase and a M-MLV RT that has amino acid substitutions D200N, T330P, T306K, W313F, and L603W compared to a wild type M-MLV RT. The amino acid sequence of an exemplary prime editor fusion protein comprises a Cas9 (H840A) nickase and an M-MLV RT that has amino acid substitutions D200N, T330P, T306K, W313F, and L603W and its individual components in shown in Table 4. In some embodiments, a prime editor fusion protein comprises the full amino acid sequence in Table 4. In some embodiments, a prime editor fusion protein comprises one or more individual components from Table 4.

In various embodiments, a prime editor fusion proteins comprises an amino acid sequence that is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to a prime editor fusion protein sequence described herein or known in the art.

TABLE 10
Amino acid sequence of an exemplary Prime Editor
fusion protein and its individual components

| SEQ | | |
|---|---|---|
| ID | | |
| NO. | DESCRIPTION | SEQUENCE |
| 4440 | Prime Editor | MKRTADGSEFESPKKKRKVDKKYSIGLDIGTNSVGWAVITD |
| | fusion protein | EYKVPSKKFKVLGNTDRHSIKKNLIGALLFDSGETAEATR |
| | CAS9(H840A)- | LKRTARRRYTRRKNRICYLQEIFSNEMAKVDDSFFHRLEE |
| | MMLV_RT | SFLVEEDKKHERHPIFGNIVDEVAYHEKYPTIYHLRKKLV |
| | D200N T330P | DSTDKADLRLIYLALAHMIKFRGHFLIEGDLNPDNSDVDK |
| | L603W T306K | LFIQLVQTYNQLFEENPINASGVDAKAILSARLSKSRRLE |
| | W313F | NLIAQLPGEKKNGLFGNLIALSLGLTPNFKSNFDLAEDAK |
| | | LQLSKDTYDDDLDNLLAQIGDQYADLFLAAKNLSDAILLS |
| | | DILRVNTEITKAPLSASMIKRYDEHHQDLTLLKALVRQQL |
| | | PEKYKEIFFDQSKNGYAGYIDGGASQEEFYKFIKPILEKM |
| | | DGTEELLVKLNREDLLRKQRTFDNGSIPHQIHLGELHAIL |
| | | RRQEDFYPFLKDNREKIEKILTFRIPYYVGPLARGNSRFA |
| | | WMTRKSEETITPWNFEEVVDKGASAQSFIERMTNFDKNLP |
| | | NEKVLPKHSLLYEYFTVYNELTKVKYVTEGMRKPAFLSGE |
| | | QKKAIVDLLFKTNRKVTVKQLKEDYFKKIECFDSVEISGV |
| | | EDRFNASLGTYHDLLKIIKDKDFLDNEENEDILEDIVLTL |
| | | TLFEDREMIEERLKTYAHLFDDKVMKQLKRRRYTGWGRLS |
| | | RKLINGIRDKQSGKTILDFLKSDGFANRNFMQLIHDDSLT |
| | | FKEDIQKAQVSGQGDSLHEHIANLAGSPAIKKGILQTVKV |
| | | VDELVKVMGRHKPENIVIEMARENQTTQKGQKNSRERMKR |
| | | IEEGIKELGSQILKEHPVENTQLQNEKLYLYYLQNGRDMY |
| | | VDQELDINRLSDYDVDAIVPQSFLKDDSIDNKVLTRSDKN |
| | | RGKSDNVPSEEVVKKMKNYWRQLLNAKLITQRKFDNLTKA |
| | | ERGGLSELDKAGFIKRQLVETRQITKHVAQILDSRMNTKY |
| | | DENDKLIREVKVITLKSKLVSDFRKDFQFYKVREINNYHH |
| | | AHDAYLNAVVGTALIKKYPKLESEFVYGDYKVYDVRKMIA |
| | | KSEQEIGKATAKYFFYSNIMNFFKTEITLANGEIRKRPLI |
| | | ETNGETGEIVWDKGRDFATVRKVLSMPQVNIVKKTEVQTG |
| | | GFSKESILPKRNSDKLIARKKDWDPKKYGGFDSPTVAYSV |
| | | LVVAKVEKGKSKKLKSVKELLGITIMERSSFEKNPIDFLE |
| | | AKGYKEVKKDLIIKLPKYSLFELENGRKRMLASAGELQKG |
| | | NELALPSKYVNFLYLASHYEKLKGSPEDNEQKQLFVEQHK |
| | | HYLDEIIEQISEFSKRVILADANLDKVLSAYNKHRDKPIR |
| | | EQAENIIHLFTLTNLGAPAAFKYFDTTIDRKRYTSTKEVL |
| | | DATLIHQSITGLYETRIDLSQLGGDSGGSSGGSSGSETPG |
| | | TSESATPESSGGSSGGSSTLNIEDEYRLHETSKEPDVSLG |
| | | STWLSDFPQAWAETGGMGLAVRQAPLIIPLKATSTPVSIK |
| | | QYPMSQEARLGIKPHIQRLLDQGILVPCQSPWNTPLLPVK |
| | | KPGTNDYRPVQDLREVNKRVEDIHPTVPNPYNLLSGLPPS |
| | | HQWYTVLDLKDAFFCLRLHPTSQPLFAFEWRDPEMGISGQ |
| | | LTWTRLPQGFKNSPTLFNEALHRDLADFRIQHPDLILLQY |
| | | VDDLLLAATSELDCQQGTRALLQTLGNLGYRASAKKAQIC |
| | | QKQVKYLGYLLKEGQRWLTEARKETVMGQPTPKTPRQLRE |
| | | FLGKAGFCRLFIPGFAEMAAPLYPLTKPGTLFNWGPDQQK |
| | | AYQEIKQALLTAPALGLPDLTKPFELFVDEKQGYAKGVLT |
| | | QKLGPWRRPVAYLSKKLDPVAAGWPPCLRMVAAIAVLTKD |
| | | AGKLTMGQPLVILAPHAVEALVKQPPDRWLSNARMTHYQA |
| | | LLLDTDRVQFGPVVALNPATLLPLPEEGLQHNCLDILAEA |
| | | HGTRPDLTDQPLPDADHTWYTDGSSLLQEGQRKAGAAVTT |
| | | ETEVIWAKALPAGTSAQRAELIALTQALKMAEGKKLNVYT |
| | | DSRYAFATAHIHGEIYRRRGWLTSEGKEIKNKDEILALLK |
| | | ALFLPKRLSIIHCPGHQKGHSAEARGNRMADQAARKAAIT |
| | | ETPDTSTLLIENSSPSGGSKRTADGSEFEPKKKRKV |
| | | KEY: |
| | | NUCLEAR LOCALIZATION SEQUENCE (NLS) |
| | | CAS9(H840A) |
| | | 33-AMINO ACID LINKER |
| | | M-MLV REVERSE TRANSCRIPTASE |
| 4441 | N-terminal NLS | MKRTADGSEFESPKKKRKV |
| 4442 | CAS9 (H840A) | DKKYSIGLDIGTNSVGWAVITDEYKVPSKKFKVLGNTDRH |
| | (MET MINUS) | SIKKNLIGALLFDSGETAEATRLKRTARRRYTRRKNRICY |
| | | LQEIFSNEMAKVDDSFFHRLEESFLVEEDKKHERHPIFGN |
| | | IVDEVAYHEKYPTIYHLRKKLVDSTDKADLRLIYLALAHM |
| | | IKFRGHFLIEGDLNPDNSDVDKLFIQLVQTYNQLFEENPI |
| | | NASGVDAKAILSARLSKSRRLENLIAQLPGEKKNGLFGNL |
| | | IALSLGLTPNFKSNFDLAEDAKLQLSKDTYDDDLDNLLAQ |
| | | IGDQYADLFLAAKNLSDAILLSDILRVNTEITKAPLSASM |
| | | IKRYDEHHQDLTLLKALVRQQLPEKYKEIFFDQSKNGYAG |
| | | YIDGGASQEEFYKFIKPILEKMDGTEELLVKLNREDLLRK |
| | | QRTFDNGSIPHQIHLGELHAILRRQEDFYPFLKDNREKIE |
| | | KILTFRIPYYVGPLARGNSRFAWMTRKSEETITPWNFEEV |
| | | VDKGASAQSFIERMTNFDKNLPNEKVLPKHSLLYEYFTVY |
| | | NELTKVKYVTEGMRKPAFLSGEQKKAIVDLLFKTNRKVTV |
| | | KQLKEDYFKKIECFDSVEISGVEDRFNASLGTYHDLLKII |
| | | KDKDFLDNEENEDILEDIVLTLTLFEDREMIEERLKTYAH |
| | | LFDDKVMKQLKRRRYTGWGRLSRKLINGIRDKQSGKTILD |
| | | FLKSDGFANRNFMQLIHDDSLTFKEDIQKAQVSGQGDSLH |
| | | EHIANLAGSPAIKKGILQTVKVVDELVKVMGRHKPENIVI |
| | | EMARENQTTQKGQKNSRERMKRIEEGIKELGSQILKEHPV |
| | | ENTQLQNEKLYLYYLQNGRDMYVDQELDINRLSDYDVDAI |
| | | VPQSFLKDDSIDNKVLTRSDKNRGKSDNVPSEEVVKKMKN |
| | | YWRQLLNAKLITQRKFDNLTKAERGGLSELDKAGFIKRQL |
| | | VETRQITKHVAQILDSRMNTKYDENDKLIREVKVITLKSK |
| | | LVSDFRKDFQFYKVREINNYHHAHDAYLNAVVGTALIKK |
| | | YPKLESEFVYGDYKVYDVRKMIAKSEQEIGKATAKYFFYS |
| | | NIMNFFKTEITLANGEIRKRPLIETNGETGEIVWDKGRDF |
| | | ATVRKVLSMPQVNIVKKTEVQTGGFSKESILPKRNSDKLI |
| | | ARKKDWDPKKYGGFDSPTVAYSVLVVAKVEKGKSKKLKSV |
| | | KELLGITIMERSSFEKNPIDFLEAKGYKEVKKDLIIKLPK |
| | | YSLFELENGRKRMLASAGELQKGNELALPSKYVNFLYLAS |
| | | HYEKLKGSPEDNEQKQLFVEQHKHYLDEIIEQISEFSKRV |
| | | ILADANLDKVLSAYNKHRDKPIREQAENIIHLFTLTNLGA |
| | | PAAFKYFDTTIDRKRYTSTKEVLDATLIHQSITGLYETRI |
| | | DLSQLGGD |
| 4443 | linker between | SGGSSGGSSGSETPGTSESATPESSGGSSGGSS |
| | CAS9 domain | |
| | and RT domain | |
| | (33 amino acids) | |
| 4444 | MMLV_RT | TLNIEDEYRLHETSKEPDVSLGSTWLSDFPQAWAETGGMG |
| | D200N T330P | LAVRQAPLIIPLKATSTPVSIKQYPMSQEARLGIKPHIQR |
| | L603W T306K | LLDQGILVPCQSPWNTPLLPVKKPGTNDYRPVQDLREVNK |
| | W313F | RVEDIHPTVPNPYNLLSGLPPSHQWYTVLDLKDAFFCLRL |
| | | HPTSQPLFAFEWRDPEMGISGQLTWTRLPQGFKNSPTLFN |
| | | EALHRDLADFRIQHPDLILLQYVDDLLLAATSELDCQQGT |
| | | RALLQTLGNLGYRASAKKAQICQKQVKYLGYLLKEGQRWL |
| | | TEARKETVMGQPTPKTPRQLREFLGKAGFCRLFIPGFAEM |
| | | AAPLYPLTKPGTLFNWGPDQQKAYQEIKQALLTAPALGLP |
| | | DLTKPFELFVDEKQGYAKGVLTQKLGPWRRPVAYLSKKLD |
| | | PVAAGWPPCLRMVAAIAVLTKDAGKLTMGQPLVILAPHAV |
| | | EALVKQPPDRWLSNARMTHYQALLLDTDRVQFGPVVALNP |
| | | ATLLPLPEEGLQHNCLDILAEAHGTRPDLTDQPLPDADHT |
| | | WYTDGSSLLQEGQRKAGAAVTTETEVIWAKALPAGTSAQR |
| | | AELIALTQALKMAEGKKLNVYTDSRYAFATAHIHGEIYRR |
| | | RGWLTSEGKEIKNKDEILALLKALFLPKRLSIIHCPGHQK |
| | | GHSAEARGNRMADQAARKAAITETPDTSTLLIENSSP |
| 4445 | C- terminal NLS | SGGSKRTADGSEFEPKKKRKV |

### Prime Editing Compositions

Disclosed herein, in some embodiments, are compositions, systems, and methods using a prime editing composition. The term “prime editing composition” or “prime editing system” refers to compositions involved in the method of prime editing as described herein. A prime editing composition may include a prime editor, e.g., a prime editor fusion protein, and a PEgRNA. A prime editing composition may further comprise additional elements, such as second strand nicking ngRNAs. Components of a prime editing composition may be combined to form a complex for prime editing, or may be kept separately, e.g., for administration purposes.

In some embodiments, a prime editing composition comprises a prime editor fusion protein complexed with a PEgRNA and optionally complexed with a ngRNA. In some embodiments, the prime editing composition comprises a prime editor comprising a DNA binding domain and a DNA polymerase domain associated with each other through a PEgRNA. For example, the prime editing composition may comprise a prime editor comprising a DNA binding domain and a DNA polymerase domain linked to each other by an RNA-protein recruitment aptamer RNA sequence, which is linked to a PEgRNA. In some embodiments, a prime editing composition comprises a PEgRNA and a polynucleotide, a polynucleotide construct, or a vector that encodes a prime editor fusion protein.

In some embodiments, a prime editing composition comprises a PEgRNA, a ngRNA, and a polynucleotide, a polynucleotide construct, or a vector that encodes a prime editor fusion protein. In some embodiments, a prime editing composition comprises multiple polynucleotides, polynucleotide constructs, or vectors, each of which encodes one or more prime editing composition components. In some embodiments, the PEgRNA of a prime editing composition is associated with the DNA binding domain, e.g., a Cas9 nickase, of the prime editor. In some embodiments, the PEgRNA of a prime editing composition complexes with the DNA binding domain of a prime editor and directs the prime editor to the target DNA.

In some embodiments, a prime editing composition comprises one or more polynucleotides that encode prime editor components and/or PEgRNA or ngRNAs. In some embodiments, a prime editing composition comprises a polynucleotide encoding a fusion protein comprising a DNA binding domain and a DNA polymerase domain. In some embodiments, a prime editing composition comprises (i) a polynucleotide encoding a fusion protein comprising a DNA binding domain and a DNA polymerase domain, and (ii) a PEgRNA or a polynucleotide encoding the PEgRNA. In some embodiments, a prime editing composition comprises (i) a polynucleotide encoding a fusion protein comprising a DNA binding domain and a DNA polymerase domain, (ii) a PEgRNA or a polynucleotide encoding the PEgRNA, and (iii) an ngRNA or a polynucleotide encoding the ngRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, and (iii) a PEgRNA or a polynucleotide encoding the PEgRNA. In some embodiments, a prime editing composition comprises (i) a polynucleotide encoding a DNA binding domain of a prime editor, e.g., a Cas9 nickase, (ii) a polynucleotide encoding a DNA polymerase domain of a prime editor, e.g., a reverse transcriptase, (iii) a PEgRNA or a polynucleotide encoding the PEgRNA, and (iv) an ngRNA or a polynucleotide encoding the ngRNA.

In some embodiments, the polynucleotide encoding the DNA biding domain or the polynucleotide encoding the DNA polymerase domain further encodes an additional polypeptide domain, e.g., an RNA-protein recruitment domain, such as a MS2 coat protein domain. In some embodiments, a prime editing composition comprises (i) a polynucleotide encoding a N-terminal half of a prime editor fusion protein and an intein-N and (ii) a polynucleotide encoding a C-terminal half of a prime editor fusion protein and an intein-C. In some embodiments, a prime editing composition comprises (i) a polynucleotide encoding a N-terminal half of a prime editor fusion protein and an intein-N (ii) a polynucleotide encoding a C-terminal half of a prime editor fusion protein and an intein-C, (iii) a PEgRNA or a polynucleotide encoding the PEgRNA, and/or (iv) an ngRNA or a polynucleotide encoding the ngRNA. In some embodiments, a prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain. In some embodiments, the DNA binding domain is a Cas protein domain, e.g., a Cas9 nickase. In some embodiments, the prime editing composition comprises (i) a polynucleotide encoding a N-terminal portion of a DNA binding domain and an intein-N, (ii) a polynucleotide encoding a C-terminal portion of the DNA binding domain, an intein-C, and a DNA polymerase domain, (iii) a PEgRNA or a polynucleotide encoding the PEgRNA, and/or (iv) a ngRNA or a polynucleotide encoding the ngRNA.

In some embodiments, a prime editing system comprises one or more polynucleotides encoding one or more prime editor polypeptides, wherein activity of the prime editing system may be temporally regulated by controlling the timing in which the vectors are delivered. For example, in some embodiments, a polynucleotide encoding the prime editor and a polynucleotide encoding a PEgRNA may be delivered simultaneously. For example, in some embodiments, a polynucleotide encoding the prime editor and a polynucleotide encoding a PEgRNA may be delivered sequentially.

In some embodiments, a polynucleotide encoding a component of a prime editing system may further comprise an element that is capable of modifying the intracellular half-life of the polynucleotide and/or modulating translational control. In some embodiments, the polynucleotide is a RNA, for example, an mRNA. In some embodiments, the half-life of the polynucleotide, e.g., the RNA may be increased. In some embodiments, the half-life of the polynucleotide, e.g., the RNA may be decreased. In some embodiments, the element may be capable of increasing the stability of the polynucleotide, e.g., the RNA. In some embodiments, the element may be capable of decreasing the stability of the polynucleotide, e.g., the RNA. In some embodiments, the element may be within the 3′ UTR of the RNA. In some embodiments, the element may include a polyadenylation signal (PA). In some embodiments, the element may include a cap, e.g., an upstream mRNA or PEgRNA end. In some embodiments, the RNA may comprise no PA such that it is subject to quicker degradation in the cell after transcription.

In some embodiments, the element may include at least one AU-rich element (ARE). The AREs may be bound by ARE binding proteins (ARE-BPs) in a manner that is dependent upon tissue type, cell type, timing, cellular localization, and environment. In some embodiments the destabilizing element may promote RNA decay, affect RNA stability, or activate translation. In some embodiments, the ARE may comprise 50 to 150 nucleotides in length. In some embodiments, the ARE may comprise at least one copy of the sequence AUUUA. In some embodiments, at least one ARE may be added to the 3′ UTR of the RNA. In some embodiments, the element may be a Woodchuck Hepatitis Virus Posttranscriptional Regulatory Element (WPRE). In further embodiments, the element is a modified and/or truncated WPRE sequence that is capable of enhancing expression from the transcript. In some embodiments, the WPRE or equivalent may be added to the 3′ UTR of the RNA. In some embodiments, the element may be selected from other RNA sequence motifs that are enriched in either fast- or slow-decaying transcripts. In some embodiments, the polynucleotide, e.g., a vector, encoding the PE or the PEgRNA may be self-destroyed via cleavage of a target sequence present on the polynucleotide, e.g., a vector. The cleavage may prevent continued transcription of a PE or a PEgRNA.

Polynucleotides encoding prime editing composition components can be DNA, RNA, or any combination thereof. In some embodiments, a polynucleotide encoding a prime editing composition component is an expression construct. In some embodiments, a polynucleotide encoding a prime editing composition component is a vector. In some embodiments, the vector is a DNA vector. In some embodiments, the vector is a plasmid. In some embodiments, the vector is a virus vector, e.g., a retroviral vector, adenoviral vector, lentiviral vector, herpesvirus vector, or an adeno-associated virus vector (AAV).

In some embodiments, polynucleotides encoding polypeptide components of a prime editing composition are codon optimized by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. In some embodiments, a polynucleotide encoding a polypeptide component of a prime editing composition are operably linked to one or more expression regulatory elements, for example, a promoter, a 3′ UTR, a 5′ UTR, or any combination thereof. In some embodiments, a polynucleotide encoding a prime editing composition component is a messenger RNA (mRNA). In some embodiments, the mRNA comprises a Cap at the 5′ end and/or a poly A tail at the 3′ end.

### Pharmaceutical Compositions

Disclosed herein are pharmaceutical compositions comprising any of the prime editing composition components, for example, prime editors, fusion proteins, polynucleotides encoding prime editor polypeptides, PEgRNAs, ngRNAs, and/or prime editing complexes described herein.

The term “pharmaceutical composition”, as used herein, refers to a composition formulated for pharmaceutical use. In some embodiments, the pharmaceutical composition further comprises a pharmaceutically acceptable carrier. In some embodiments, the pharmaceutical composition comprises additional agents, e.g., for specific delivery, increasing half-life, or other therapeutic compounds.

In some embodiments, a pharmaceutically-acceptable carrier comprises any vehicle, such as a liquid or solid filler, diluent, excipient, manufacturing aid (e.g., lubricant, talc magnesium, calcium or zinc stearate, or steric acid), or solvent encapsulating material, involved in carrying or transporting the compound from one site (e.g., the delivery site) of the body, to another site (e.g., organ, tissue or portion of the body). A pharmaceutically acceptable carrier is “acceptable” in the sense of being compatible with the other ingredients of the formulation and not injurious to the tissue of the subject (e.g., physiologically compatible, sterile, physiologic pH, etc.)

Formulations of the pharmaceutical compositions described herein can be prepared by any method known or hereafter developed in the art of pharmacology. In general, such preparatory methods include the step of bringing the active ingredient(s) into association with an excipient and/or one or more other accessory ingredients, and then, if necessary and/or desirable, shaping and/or packaging the product into a desired single- or multi-dose unit. Pharmaceutical formulations can additionally comprise a pharmaceutically acceptable excipient, which, as used herein, includes any and all solvents, dispersion media, diluents, or other liquid vehicles, dispersion or suspension aids, surface active agents, isotonic agents, thickening or emulsifying agents, preservatives, solid binders, lubricants and the like, as suited to the particular dosage form desired.

### Methods of Editing

The methods and compositions disclosed herein can be used to edit a target gene of interest by prime editing.

In some embodiments, the prime editing method comprises contacting a target gene, with a PEgRNA and a prime editor (PE) polypeptide described herein. In some embodiments, the target gene is double stranded, and comprises two strands of DNA complementary to each other. In some embodiments, the contacting with a PEgRNA and the contacting with a prime editor are performed sequentially. In some embodiments, the contacting with a prime editor is performed after the contacting with a PEgRNA. In some embodiments, the contacting with a PEgRNA is performed after the contacting with a prime editor. In some embodiments, the contacting with a PEgRNA, and the contacting with a prime editor are performed simultaneously. In some embodiments, the PEgRNA and the prime editor are associated in a complex prior to contacting a target gene.

In some embodiments, contacting the target gene with the prime editing composition results in binding of the PEgRNA to a target strand of the target gene. In some embodiments, contacting the target gene with the prime editing composition results in binding of the PEgRNA to a search target sequence on the target strand of the target gene upon contacting with the PEgRNA. In some embodiments, contacting the target gene with the prime editing composition results in binding of a spacer sequence of the PEgRNA to a search target sequence with the search target sequence on the target strand of the target gene upon said contacting of the PEgRNA.

In some embodiments, contacting the target gene with the prime editing composition results in binding of the prime editor to the target gene, e.g., the target gene, upon the contacting of the PE composition with the target gene. In some embodiments, the DNA binding domain of the PE associates with the PEgRNA. In some embodiments, the PE binds the target gene, directed by the PEgRNA. Accordingly, in some embodiments, the contacting of the target gene result in binding of a DNA binding domain of a prime editor of the target gene directed by the PEgRNA.

In some embodiments, contacting the target gene with the prime editing composition results in a nick in an edit strand of the target gene, by the prime editor upon contacting with the target gene, thereby generating a nicked on the edit strand of the target gene. In some embodiments, contacting the target gene with the prime editing composition results in a single-stranded DNA comprising a free 3′ end at the nick site of the edit strand of the target gene. In some embodiments, contacting the target gene with the prime editing composition results in a nick in the edit strand of the target gene by a DNA binding domain of the prime editor, thereby generating a single-stranded DNA comprising a free 3′ end at the nick site. In some embodiments, the DNA binding domain of the prime editor is a Cas domain. In some embodiments, the DNA binding domain of the prime editor is a Cas9. In some embodiments, the DNA binding domain of the prime editor is a Cas9 nickase.

In some embodiments, contacting the target gene with the prime editing composition results in hybridization of the PEgRNA with the 3′ end of the nicked single-stranded DNA, thereby priming DNA polymerization by a DNA polymerase domain of the prime editor. In some embodiments, the free 3′ end of the single-stranded DNA generated at the nick site hybridizes to a primer binding site sequence (PBS) of the contacted PEgRNA, thereby priming DNA polymerization. In some embodiments, the DNA polymerization is reverse transcription catalyzed by a reverse transcriptase domain of the prime editor. In some embodiments, the method comprises contacting the target gene with a DNA polymerase, e.g., a reverse transcriptase, as a part of a prime editor fusion protein or prime editing complex (in cis), or as a separate protein (in trans).

In some embodiments, contacting the target gene with the prime editing composition generates an edited single stranded DNA that is coded by the editing template of the PEgRNA by DNA polymerase mediated polymerization from the 3′ free end of the single-stranded DNA at the nick site. In some embodiments, the editing template of the PEgRNA comprises one or more intended nucleotide edits compared to endogenous sequence of the target gene. In some embodiments, the intended nucleotide edits are incorporated in the target gene, by excision of the 5′ single stranded DNA of the edit strand of the target gene generated at the nick site and DNA repair. In some embodiments, the intended nucleotide edits are incorporated in the target gene by excision of the editing target sequence and DNA repair. In some embodiments, excision of the 5″ single stranded DNA of the edit strand generated at the nick site is by a flap endonuclease. In some embodiments, the flap nuclease is FEN1. In some embodiments, the method further comprises contacting the target gene with a flap endonuclease. In some embodiments, the flap endonuclease is provided as a part of a prime editor fusion protein. In some embodiments, the flap endonuclease is provided in trans.

In some embodiments, contacting the target gene with the prime editing composition generates a mismatched heteroduplex comprising the edit strand of the target gene that comprises the edited single stranded DNA, and the unedited target strand of the target gene. Without being bound by theory, the endogenous DNA repair and replication may resolve the mismatched edited DNA to incorporate the nucleotide change(s) to form the desired edited target gene.

In some embodiments, the method further comprises contacting the target gene, with a nick guide (ngRNA) disclosed herein. In some embodiments, the ngRNA comprises a spacer that binds a second search target sequence on the edit strand of the target gene. In some embodiments, the contacted ngRNA directs the PE to introduce a nick in the target strand of the target gene. In some embodiments, the nick on the target strand (non-edit strand) results in endogenous DNA repair machinery to use the edit strand to repair the non-edit strand, thereby incorporating the intended nucleotide edit in both strand of the target gene and modifying the target gene. In some embodiments, the ngRNA comprises a spacer sequence that is complementary to, and may hybridize with, the second search target sequence on the edit strand only after the intended nucleotide edit(s) are incorporated in the edit strand of the target gene.

In some embodiments, the target gene is contacted by the ngRNA, the PEgRNA, and the PE simultaneously. In some embodiments, the ngRNA, the PEgRNA, and the PE form a complex when they contact the target gene. In some embodiments, the target gene is contacted with the ngRNA, the PEgRNA, and the prime editor sequentially. In some embodiments, the target gene is contacted with the ngRNA and/or the PEgRNA after contacting the target gene with the PE. In some embodiments, the target gene is contacted with the ngRNA and/or the PEgRNA before contacting the target gene with the prime editor.

In some embodiments, the target gene, is in a cell. Accordingly, also provided herein are methods of modifying a cell, such as a human cell, a human primary cell, and/or a human iPSC-derived cell.

In some embodiments, the prime editing method comprises introducing a PEgRNA, a prime editor, and/or a ngRNA into the cell that has the target gene. In some embodiments, the prime editing method comprises introducing into the cell that has the target gene with a prime editing composition comprising a PEgRNA, a prime editor polypeptide, and/or a ngRNA. In some embodiments, the PEgRNA, the prime editor polypeptide, and/or the ngRNA form a complex prior to the introduction into the cell. In some embodiments, the PEgRNA, the prime editor polypeptide, and/or the ngRNA form a complex after the introduction into the cell. The prime editors, PEgRNA and/or ngRNAs, and prime editing complexes may be introduced into the cell by any delivery approaches described herein or any delivery approach known in the art, including ribonucleoprotein (RNPs), lipid nanoparticles (LNPs), viral vectors, non-viral vectors, mRNA delivery, and physical techniques such as cell membrane disruption by a microfluidics device. The prime editors, PEgRNA and/or ngRNAs, and prime editing complexes may be introduced into the cell simultaneously or sequentially.

In some embodiments, the prime editing method comprises introducing into the cell a PEgRNA or a polynucleotide encoding the PEgRNA, a prime editor polynucleotide encoding a prime editor polypeptide, and optionally an ngRNA or a polynucleotide encoding the ngRNA. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell simultaneously. In some embodiments, the method comprises introducing the PEgRNA or the polynucleotide encoding the PEgRNA, the polynucleotide encoding the prime editor polypeptide, and/or the ngRNA or the polynucleotide encoding the ngRNA into the cell sequentially. In some embodiments, the method comprises introducing the polynucleotide encoding the prime editor polypeptide into the cell before introduction of the PEgRNA or the polynucleotide encoding the PEgRNA and/or the ngRNA or the polynucleotide encoding the ngRNA. In some embodiments, the polynucleotide encoding the prime editor polypeptide is introduced into and expressed in the cell before introduction of the PEgRNA or the polynucleotide encoding the PEgRNA and/or the ngRNA or the polynucleotide encoding the ngRNA into the cell. In some embodiments, the polynucleotide encoding the prime editor polypeptide is introduced into the cell after the PEgRNA or the polynucleotide encoding the PEgRNA and/or the ngRNA or the polynucleotide encoding the ngRNA are introduced into the cell. The polynucleotide encoding the prime editor polypeptide, the PEgRNA or the polynucleotide encoding the PEgRNA, and/or the ngRNA or the polynucleotide encoding the ngRNA, may be introduced into the cell by any delivery approaches described herein or any delivery approach known in the art, for example, by RNPs, LNPs, viral vectors, non-viral vectors, mRNA delivery, and physical delivery.

In some embodiments, the polynucleotide encoding the prime editor polypeptide, the polynucleotide encoding the PEgRNA, and/or the polynucleotide encoding the ngRNA integrate into the genome of the cell after being introduced into the cell. In some embodiments, the polynucleotide encoding the prime editor polypeptide, the polynucleotide encoding the PEgRNA, and/or the polynucleotide encoding the ngRNA are introduced into the cell for transient expression. Accordingly, also provided herein are cells modified by prime editing.

In some embodiments, the cell is a prokaryotic cell. In some embodiments, the cell is a eukaryotic cell. In some embodiments, the cell is a mammalian cell. In some embodiments, the cell is a non-human primate cell, bovine cell, porcine cell, rodent or mouse cell. In some embodiments, the cell is a human cell. In some embodiments, the cell is a primary cell. In some embodiments, the cell is a human primary cell. In some embodiments, the cell is a progenitor cell. In some embodiments, the cell is a human progenitor cell. In some embodiments, the cell is a human cell from an organ. In some embodiments, the cell is a primary human cell de

In some embodiments, the cell is a progenitor cell. In some embodiments, the cell is a stem cell. in some embodiments, the cell is an induced pluripotent stem cell. In some embodiments, the cell is an embryonic stem cell. In some embodiments, the cell is a retinal progenitor cell. In some embodiments, the cell is a retina precursor cell. In some embodiments, the cell is a fibroblast.

In some embodiments, the cell is a human stem cell. in some embodiments, the cell is an induced human pluripotent stem cell. In some embodiments, the cell is a human embryonic stem cell. In some embodiments, the cell is a human retinal progenitor cell. In some embodiments, the cell is a human retina precursor cell. In some embodiments, the cell is a human fibroblast.

In some embodiments, the cell is a primary cell. In some embodiments, the cell is a human primary cell. In some embodiments, the cell is a retina cell. In some embodiments, the cell is a photoreceptor. In some embodiments, the cell is a rod cell. In some embodiments, the cell is a cone cell. In some embodiments, the cell is a human cell from a retina. In some embodiments, the cell is a human photoreceptor. In some embodiments, the cell is a human rod cell. In some embodiments, the cell is a human cone cell. In some embodiments, the cell is a primary human photoreceptor derived from an induced human pluripotent stem cell (iPSC).

In some embodiments, the target gene edited by prime editing is in a chromosome of the cell. In some embodiments, the intended nucleotide edits incorporate in the chromosome of the cell and are inheritable by progeny cells. In some embodiments, the intended nucleotide edits introduced to the cell by the prime editing compositions and methods are such that the cell and progeny of the cell also include the intended nucleotide edits. In some embodiments, the cell is autologous, allogeneic, or xenogeneic to a subject. In some embodiments, the cell is from or derived from a subject. In some embodiments, the cell is from or derived from a human subject. In some embodiments, the cell is introduced back into the subject, e.g., a human subject, after incorporation of the intended nucleotide edits by prime editing.

In some embodiments, the method provided herein comprises introducing the prime editor polypeptide or the polynucleotide encoding the prime editor polypeptide, the PEgRNA or the polynucleotide encoding the PEgRNA, and/or the ngRNA or the polynucleotide encoding the ngRNA into a plurality or a population of cells that comprise the target gene. In some embodiments, the population of cells is of the same cell type. In some embodiments, the population of cells is of the same tissue or organ. In some embodiments, the population of cells is heterogeneous. In some embodiments, the population of cells is homogeneous. In some embodiments, the population of cells is from a single tissue or organ, and the cells are heterogeneous. In some embodiments, the introduction into the population of cells is ex vivo. In some embodiments, the introduction into the population of cells is in vivo, e.g., into a human subject.

In some embodiments, the target gene is in a genome of each cell of the population. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of one or more intended nucleotide edits in the target gene in at least one of the cells in the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in a plurality of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in each cell of the population of cells. In some embodiments, introduction of the prime editor polypeptide or the polynucleotide encoding the prime editor polypeptide, the PEgRNA or the polynucleotide encoding the PEgRNA, and/or the ngRNA or the polynucleotide encoding the ngRNA results in incorporation of the one or more intended nucleotide edits in the target gene in sufficient number of cells such that the disease or disorder is treated, prevented or ameliorated.

In some embodiments, editing efficiency of the prime editing compositions and method described herein can be measured by calculating the percentage of edited target genes in a population of cells introduced with the prime editing composition. In some embodiments, the editing efficiency is determined after 1 hour, 2 hours, 6 hours, 12 hours, 24 hours, 36 hours, 48 hours. 3 days, 4 days, 5 days, 7 days. 10 days, or 14 days of exposing a target gene within the genome of a cell) to a prime editing composition. In some embodiments, the population of cells introduced with the prime editing composition is ex vivo. In some embodiments, the population of cells introduced with the prime editing composition is in vitro. In some embodiments, the population of cells introduced with the prime editing composition is in vivo. In some embodiments, the prime editing methods disclosed herein have an editing efficiency of at least about 1%, at least about 5%, at least about 10%, at least about 20%, at least about 30%, at least about 40%, at least about 50%, at least about 60%, at least about 70%, at least about 80%, at least about 90%, at least about 95%, or at least about 99% relative to a suitable control. In some embodiments, the prime editing methods disclosed herein have an editing efficiency of at least 25% relative to a suitable control. In some embodiments, the prime editing methods disclosed herein have an editing efficiency of at least 35% relative to a suitable control. In some embodiments, a prime editing method disclosed herein has an editing efficiency of at least 30% relative to a suitable control. In some embodiments, the prime editing methods disclosed herein have an editing efficiency of at least 45% relative to a suitable control. In some embodiments, the prime editing methods disclosed herein have an editing efficiency of at least 50% relative to a suitable control.

In some embodiments, the methods disclosed herein have an editing efficiency of at least about 1%, at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a primary cell relative to a suitable control.

In some embodiments, the methods disclosed herein have an editing efficiency of at least about 5%, at least about 7.5%, at least about 10%, at least about 15%, at least about 20%, at least about 30%, at least about 40%, at least about 50%, at least about 60%, at least about 70%, at least about 80%, at least about 90%, or at least about 95% of editing a hepatocyte relative to a corresponding control hepatocyte. In some embodiments, the hepatocyte is a human hepatocyte.

In some embodiments, the prime editing compositions provided herein are capable of incorporated one or more intended nucleotide edits without generating a significant proportion of indels. The term “indel(s)”, as used herein, refers to the insertion or deletion of a nucleotide base within a polynucleotide, for example, a target gene. Such insertions or deletions can lead to frame shift mutations within a coding region of a gene. Indel frequency of editing can be calculated by methods known in the art. In some embodiments, indel frequency can be calculated based on sequence alignment such as the CRISPResso 2 algorithm as described in Clement et al., Nat. Biotechnol. 37 (3): 224-226 (2019), which is incorporated herein in its entirety. In some embodiments, the methods disclosed herein can have an indel frequency of less than 20%, less than 10%, less than 9%, less than 8%, less than 7%, less than 6%, less than 5%, less than 4%, less than 3%, less than 2%, less than 1.5%, or less than 1%. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a gene within the genome of a cell) to a prime editing composition.

In some embodiments, the prime editing compositions provided herein are capable of incorporated one or more intended nucleotide edits efficiently without generating a significant proportion of indels. In some embodiments, the prime editing methods disclosed herein have an editing efficiency of at least about 1% and an indel frequency of less than 1% in a target cell, e.g., a human primary cell, human iPSC, or human fibroblast. In some embodiments, the prime editing methods disclosed herein have an editing efficiency of at least about 1% and an indel frequency of less than 0.5% in a target cell, e.g., a human primary cell, human iPSC, or human fibroblast. In some embodiments, the prime editing methods disclosed herein have an editing efficiency of at least about 1% and an indel frequency of less than 0.1% in a target cell, e.g., a human primary cell, a human iPSC, or a human fibroblast. In some embodiments, the prime editing methods disclosed herein have an editing efficiency of at least about 5% and an indel frequency of less than 1% in a target cell, e.g., a human primary cell, a human iPSC, or a human fibroblast. In some embodiments, the prime editing methods disclosed herein have an editing efficiency of at least about 5% and an indel frequency of less than 0.5% in a target cell, e.g., a human primary cell, a human iPSC, or a human fibroblast. In some embodiments, the prime editing methods disclosed herein have an editing efficiency of at least about 5% and an indel frequency of less than 0.1% in a target cell, e.g., a human primary cell, a human iPSC, or a human fibroblast.

In some embodiments, the prime editing methods disclosed herein have an editing efficiency of at least about 7.5% and an indel frequency of less than 1% in a target cell, e.g., a human primary cell, a human iPSC, or a human fibroblast. In some embodiments, the prime editing methods disclosed herein have an editing efficiency of at least about 7.5% and an indel frequency of less than 0.5% in a target cell, e.g., a human primary cell, a human iPSC, or a human fibroblast. In some embodiments, the prime editing methods disclosed herein have an editing efficiency of at least about 7.5% and an indel frequency of less than 0.1% in a target cell, e.g., a human primary cell, a human iPSC, or a human fibroblast.

In some embodiments, the prime editing methods disclosed herein have an editing efficiency of at least about 10% and an indel frequency of less than 1% in a target cell, e.g., a human primary cell, a human iPSC, or a human fibroblast. In some embodiments, the prime editing methods disclosed herein have an editing efficiency of at least about 10% and an indel frequency of less than 0.5% in a target cell, e.g., a human primary cell, a human iPSC, or a human fibroblast. In some embodiments, the prime editing methods disclosed herein have an editing efficiency of at least about 10% and an indel frequency of less than 0.1% in a target cell, e.g., a human primary cell, a human iPSC, or a human fibroblast.

In some embodiments, the prime editing methods disclosed herein have an editing efficiency of at least about 15% and an indel frequency of less than 1% in a target cell, e.g., a human primary cell, a human iPSC, or a human fibroblast. In some embodiments, the prime editing methods disclosed herein have an editing efficiency of at least about 15% and an indel frequency of less than 0.5% in a target cell, e.g., a human primary cell, a human iPSC, or a human fibroblast. In some embodiments, the prime editing methods disclosed herein have an editing efficiency of at least about 15% and an indel frequency of less than 0.1% in a target cell, e.g., a human primary cell, a human iPSC, or a human fibroblast.

In some embodiments, the prime editing methods disclosed herein have an editing efficiency of at least about 20% and an indel frequency of less than 1% in a target cell, e.g., a human primary cell, a human iPSC, or a human fibroblast. In some embodiments, the prime editing methods disclosed herein have an editing efficiency of at least about 20% and an indel frequency of less than 0.5% in a target cell, e.g., a human primary cell, a human iPSC, or a human fibroblast. In some embodiments, the prime editing methods disclosed herein have an editing efficiency of at least about 20% and an indel frequency of less than 0.1% in a target cell, e.g., a human primary cell, a human iPSC, or a human fibroblast.

In some embodiments, the prime editing methods disclosed herein have an editing efficiency of at least about 30% and an indel frequency of less than 1% in a target cell, e.g., a human primary cell, a human iPSC, or a human fibroblast. In some embodiments, the prime editing methods disclosed herein have an editing efficiency of at least about 30% and an indel frequency of less than 0.5% in a target cell, e.g., a human primary cell, a human iPSC, or a human fibroblast. In some embodiments, the prime editing methods disclosed herein have an editing efficiency of at least about 30% and an indel frequency of less than 0.1% in a target cell, e.g., a human primary cell, a human iPSC, or a human fibroblast.

In some embodiments, the prime editing methods disclosed herein have an editing efficiency of at least about 40% and an indel frequency of less than 1% in a target cell, e.g., a human primary cell, a human iPSC, or a human fibroblast. In some embodiments, the prime editing methods disclosed herein have an editing efficiency of at least about 40% and an indel frequency of less than 0.5% in a target cell, e.g., a human primary cell, a human iPSC, or a human fibroblast. In some embodiments, the prime editing methods disclosed herein have an editing efficiency of at least about 40% and an indel frequency of less than 0.1% in a target cell, e.g., a human primary cell, a human iPSC, or a human fibroblast.

In some embodiments, the prime editing methods disclosed herein have an editing efficiency of at least about 50% and an indel frequency of less than 1% in a target cell, e.g., a human primary cell, a human iPSC, or a human fibroblast. In some embodiments, the prime editing methods disclosed herein have an editing efficiency of at least about 50% and an indel frequency of less than 0.5% in a target cell, e.g., a human primary cell, a human iPSC, or a human fibroblast. In some embodiments, the prime editing methods disclosed herein have an editing efficiency of at least about 50% and an indel frequency of less than 0.1% in a target cell, e.g., a human primary cell, a human iPSC, or a human fibroblast.

In some embodiments, the prime editing methods disclosed herein have an editing efficiency of at least about 60% and an indel frequency of less than 1% in a target cell, e.g., a human primary cell, a human iPSC, or a human fibroblast. In some embodiments, the prime editing methods disclosed herein have an editing efficiency of at least about 60% and an indel frequency of less than 0.5% in a target cell, e.g., a human primary cell, a human iPSC, or a human fibroblast. In some embodiments, the prime editing methods disclosed herein have an editing efficiency of at least about 60% and an indel frequency of less than 0.1% in a target cell, e.g., a human primary cell, a human iPSC, or a human fibroblast.

In some embodiments, the prime editing methods disclosed herein have an editing efficiency of at least about 70% and an indel frequency of less than 1% in a target cell, e.g., a human primary cell, a human iPSC, or a human fibroblast. In some embodiments, the prime editing methods disclosed herein have an editing efficiency of at least about 70% and an indel frequency of less than 0.5% in a target cell, e.g., a human primary cell, a human iPSC, or a human fibroblast. In some embodiments, the prime editing methods disclosed herein have an editing efficiency of at least about 70% and an indel frequency of less than 0.1% in a target cell, e.g., a human primary cell, a human iPSC, or a human fibroblast.

In some embodiments, the prime editing methods disclosed herein have an editing efficiency of at least about 80% and an indel frequency of less than 1% in a target cell, e.g., a human primary cell, a human iPSC, or a human fibroblast. In some embodiments, the prime editing methods disclosed herein have an editing efficiency of at least about 80% and an indel frequency of less than 0.5% in a target cell, e.g., a human primary cell, a human iPSC, or a human fibroblast. In some embodiments, the prime editing methods disclosed herein have an editing efficiency of at least about 80% and an indel frequency of less than 0.1% in a target cell, e.g., a human primary cell, a human iPSC, or a human fibroblast.

In some embodiments, the prime editing methods disclosed herein have an editing efficiency of at least about 90% and an indel frequency of less than 1% in a target cell, e.g., a human primary cell, a human iPSC, or a human fibroblast. In some embodiments, the prime editing methods disclosed herein have an editing efficiency of at least about 90% and an indel frequency of less than 0.5% in a target cell, e.g., a human primary cell, a human iPSC, or a human fibroblast. In some embodiments, the prime editing methods disclosed herein have an editing efficiency of at least about 90% and an indel frequency of less than 0.1% in a target cell, e.g., a human primary cell, a human iPSC, or a human fibroblast.

In some embodiments, the prime editing methods disclosed herein have an editing efficiency of at least about 95% and an indel frequency of less than 1% in a target cell, e.g., a human primary cell, a human iPSC, or a human fibroblast. In some embodiments, the prime editing methods disclosed herein have an editing efficiency of at least about 95% and an indel frequency of less than 0.5% in a target cell, e.g., a human primary cell, a human iPSC, or a human fibroblast. In some embodiments, the prime editing methods disclosed herein have an editing efficiency of at least about 95% and an indel frequency of less than 0.1% in a target cell, e.g., a human primary cell, a human iPSC, or a human fibroblast. In some embodiments, any number of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a gene within the genome of a cell) to a prime editing composition. In some embodiments, the editing efficiency is determined after 1 hour, 2 hours, 6 hours, 12 hours, 24 hours, 36 hours, 48 hours, 3 days, 4 days, 5 days, 7 days, 10 days, or 14 days of exposing a target gene (e.g., a gene within the genome of a cell) to a prime editing composition.

In some embodiments, the prime editing composition described herein result in less than 50%, less than 40%, less than 30%, less than 20%, less than 19%, less than 18%, less than 17%, less than 16%, less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, less than 0.9%, less than 0.8%, less than 0.7%, less than 0.6%, less than 0.5%, less than 0.4%, less than 0.3%, less than 0.2%, less than 0.1%, less than 0.09%, less than 0.08%, less than 0.07%, less than 0.06%, less than 0.05%, less than 0.04%, less than 0.03%, less than 0.02%, or less than 0.01% off-target editing in a chromosome that includes the target gene. In some embodiments, off-target editing is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a target gene (e.g., a nucleic acid within the genome of a cell) to a prime editing composition.

In some embodiments, the prime editing compositions (e.g., PEgRNAs and prime editors as described herein) and prime editing methods disclosed herein can be used to edit a target gene. In some embodiments, the target gene comprises a mutation compared to a wild type gene. In some embodiments, the mutation is associated a disease. In some embodiments, the target gene comprises an editing target sequence that contains the mutation associated with a disease. In some embodiments, the mutation is in a coding region of the target gene. In some embodiments, the mutation is in an exon of the target gene. In some embodiments, the prime editing method comprises contacting a target gene with a prime editing composition comprising a prime editor, a PEgRNA, and/or a ngRNA. In some embodiments, contacting the target gene with the prime editing composition results in incorporation of one or more intended nucleotide edits in the target gene. In some embodiments, the incorporation is in a region of the target gene that corresponds to an editing target sequence in the gene. In some embodiments, the one or more intended nucleotide edits comprises a single nucleotide substitution, an insertion, a deletion, or any combination thereof, compared to the endogenous sequence of the target gene. In some embodiments, incorporation of the one or more intended nucleotide edits results in replacement of one or more mutations with the corresponding sequence that encodes a wild type protein. In some embodiments, incorporation of the one or more intended nucleotide edits results in replacement of the one or more mutations with the corresponding sequence in a wild type gene. In some embodiments, incorporation of the one more intended nucleotide edits results in correction of a mutation in the target gene. In some embodiments, the target gene comprises an editing template sequence that contains the mutation. In some embodiments, contacting the target gene with the prime editing composition results in incorporation of one or more intended nucleotide edits in the target gene, which corrects the mutation in the editing target sequence (or a double stranded region comprising the editing target sequence and the complementary sequence to the editing target sequence on a target strand) in the target gene.

In some embodiments, incorporation of the one more intended nucleotide edits results in correction of a mutation in the target gene. In some embodiments, incorporation of the one more intended nucleotide edits results in correction of a gene sequence and restores wild type expression and function of the protein.

In some embodiments, the target gene is in a target cell. Accordingly, in one aspect provided herein is a method of editing a target cell comprising a target gene that encodes a polypeptide that comprises one or more mutations relative to a wild type gene. In some embodiments, the methods of the present disclosure comprise introducing a prime editing composition comprising a PEgRNA, a prime editor polypeptide, and/or a ngRNA into the target cell that has the target gene to edit the target gene, thereby generating an edited cell. In some embodiments, the target cell is a mammalian cell. In some embodiments, the target cell is a human cell. In some embodiments, the target cell is a primary cell. In some embodiments, the target cell is a human primary cell. In some embodiments, the target cell is a progenitor cell. In some embodiments, the target cell is a human progenitor cell. In some embodiments, the target cell is a stem cell. In some embodiments, the target cell is a human stem cell. In some embodiments, the target cell is a hepatocyte. In some embodiments, the target cell is a human hepatocyte. In some embodiments, the target cell is a primary human hepatocyte derived from an induced human pluripotent stem cell (iPSC). In some embodiments, the cell is a neuron. In some embodiments, the cell is a neuron from basal ganglia. In some embodiments, the cell is a neuron from basal ganglia of a subject. In some embodiments, the cell is a neuron in the basal ganglia of a subject.

In some embodiments, components of a prime editing composition described herein are provided to a target cell in vitro. In some embodiments, components of a prime editing composition described herein are provided to a target cell ex vivo. In some embodiments, components of a prime editing composition described herein are provided to a target cell in vivo.

In some embodiments, incorporation of the one or more intended nucleotide edits in the target gene that comprises one or more mutations restores wild type expression and function of protein encoded by the gene. In some embodiments, the target gene encodes at least one mutation as compared to the wild type protein prior to incorporation of the one or more intended nucleotide edits. In some embodiments, expression and/or function of protein may be measured when expressed in a target cell. In some embodiments, incorporation of the one or more intended nucleotide edits in the target gene comprising one or more mutations lead to a fold change in a level of gene expression, protein expression, or a combination thereof. In some embodiments, a change in the level of gene expression can comprise a fold change of, e.g., 2-fold, 3-fold. 4-fold, 5-fold, 6-fold, 7-fold, 8-fold, 9-fold, 10-fold, 15-fold, 20-fold, 25-fold, 30-fold, 40-fold, 50-fold, 60-fold, 70-fold, 80-fold, 90-fold, 100-fold or greater as compared to expression in a suitable control cell not introduced with a prime editing composition described herein. In some embodiments, incorporation of the one or more intended nucleotide edits in the target gene that comprises one or more mutations restores wild type expression of protein by at least 10%, 20%, 30%, 40%, 50%, 60%, 70%, 80%, 90%, 95%, 099% or more as compared to wild type expression of the protein in a suitable control cell that comprises a wild type gene.

In some embodiments, an expression increase can be measured by a functional assay. In some embodiments, protein expression can be measured using a protein assay. In some embodiments, protein expression can be measured using antibody testing. In some embodiments, protein expression can be measured using ELISA, mass spectrometry, Western blot, sodium dodecyl sulfate polyacrylamide gel electrophoresis (SDS-PAGE), high performance liquid chromatography (HPLC), electrophoresis, or any combination thereof. In some embodiments, a protein assay can comprise SDS-PAGE and densitometric analysis of a Coomassie Blue-stained gel.

### Delivery

Prime editing compositions described herein can be delivered to a cellular environment with any approach known in the art. Components of a prime editing composition can be delivered to a cell by the same mode or different modes. For example, in some embodiments, a prime editor can be delivered as a polypeptide or a polynucleotide (DNA or RNA) encoding the polypeptide. In some embodiments, a PEgRNA can be delivered directly as an RNA or as a DNA encoding the PEgRNA.

In some embodiments, a prime editing composition component is encoded by a polynucleotide, a vector, or a construct. In some embodiments, a prime editor polypeptide, a PEgRNA and/or a ngRNA is encoded by a polynucleotide. In some embodiments, the polynucleotide encodes a prime editor fusion protein comprising a DNA binding domain and a DNA polymerase domain. In some embodiments, the polynucleotide encodes a DNA polymerase domain of a prime editor. In some embodiments, the polynucleotide encodes a DNA polymerase domain of a prime editor. In some embodiments, the polynucleotide encodes a portion of a prime editor protein, for example, a N-terminal portion of a prime editor fusion protein connected to an intein-N. In some embodiments, the polynucleotide encodes a portion of a prime editor protein, for example, a C-terminal portion of a prime editor fusion protein connected to an intein-C. In some embodiments, the polynucleotide encodes a PEgRNA and/or a ngRNA. In some embodiments, the polypeptide encodes two or more components of a prime editing composition, for example, a prime editor fusion protein and a PEgRNA.

In some embodiments, the polynucleotide encoding one or more prime editing composition components is delivered to a target cell is integrated into the genome of the cell for long-term expression, for example, by a retroviral vector. In some embodiments, the polynucleotide delivered to a target cell is expressed transiently. For example, the polynucleotide may be delivered in the form of a mRNA, or a non-integrating vector (non-integrating virus, plasmids, minicircle DNAs) for episomal expression.

In some embodiments, a polynucleotide encoding one or more prime editing system components can be operably linked to a regulatory element, e.g., a transcriptional control element, such as a promoter. In some embodiments, the polynucleotide is operably linked to multiple control elements. Depending on the expression system utilized, any of a number of suitable transcription and translation control elements, including constitutive and inducible promoters, transcription enhancer elements, transcription terminators, etc. may be used in the expression vector (e.g., U6 promoter, H1 promoter).

In some embodiments, the polynucleotide encoding one or more prime editing composition components is a part of, or is encoded by, a vector. In some embodiments, the vector is a viral vector. In some embodiments, the vector is a non-viral vector.

Non-viral vector delivery systems can include DNA plasmids, RNA (e.g., a transcript of a vector described herein), naked nucleic acid, and nucleic acid complexed with a delivery vehicle, such as a liposome. In some embodiments, the polynucleotide is provided as an RNA, e.g., a mRNA or a transcript. Any RNA of the prime editing systems, for example a guide RNA or a base editor-encoding mRNA, can be delivered in the form of RNA. In some embodiments, one or more components of the prime editing system that are RNAs is produced by direct chemical synthesis or may be transcribed in vitro from a DNA. In some embodiments, a mRNA that encodes a prime editor polypeptide is generated using in vitro transcription. Guide polynucleotides (e.g., PEgRNA or ngRNA) can also be transcribed using in vitro transcription from a cassette containing a T7 promoter, followed by the sequence “GG”, and guide polynucleotide sequence. In some embodiments, the prime editor encoding mRNA, PEgRNA, and/or ngRNA are synthesized in vitro using an RNA polymerase enzyme (e.g., T7 polymerase, T3 polymerase, SP6 polymerase, etc.). Once synthesized, the RNA can directly contact a target gene or can be introduced into a cell using any suitable technique for introducing nucleic acids into cells (e.g., microinjection, electroporation, transfection). In some embodiments, the prime editor-coding sequences, the PEgRNAs, and/or the ngRNAs are modified to include one or more modified nucleoside e.g., using pseudo-U or 5-Methyl-C.

Methods of non-viral delivery of nucleic acids can include lipofection, nucleofection, microinjection, biolistics, virosomes, liposomes, immunoliposomes, polycation or lipid:nucleic acid conjugates, naked DNA, artificial virions, cell membrane disruption by a microfluidics device, and agent-enhanced uptake of DNA. Cationic and neutral lipids that are suitable for efficient receptor-recognition lipofection of polynucleotides can be used. Delivery can be to cells (e.g., in vitro or ex vivo administration) or target tissues (e.g., in vivo administration). The preparation of lipid:nucleic acid complexes, including targeted liposomes such as immunolipid complexes, can be used.

Viral vector delivery systems can include DNA and RNA viruses, which can have either episomal or integrated genomes after delivery to the cell. RNA or DNA viral based systems can be used to target specific cells and trafficking the viral payload to an organelle of the cell. Viral vectors can be administered directly (in vivo) or they can be used to treat cells in vitro, and the modified cells can optionally be administered (ex vivo).

In some embodiments, the viral vector is a retroviral, lentiviral, adenoviral, adeno-associated viral or herpes simplex viral vector. Retroviral vectors can include those based upon murine leukemia virus (MuLV), gibbon ape leukemia virus (GaLV), simian immunodeficiency virus (SIV), human immunodeficiency virus (HIV), and combinations thereof. In some embodiments, the retroviral vector is a lentiviral vector. In some embodiments, the retroviral vector is a gamma retroviral vector. In some embodiments, the viral vector is an adenoviral vector. In some embodiments, the viral vector is an adeno-associated virus (“AAV”) vector.

In some embodiments, polynucleotides encoding one or more prime editing composition components are packaged in a virus particle. Packaging cells can be used to form virus particles that can infect a target cell. Such cells can include 293 cells, (e.g., for packaging adenovirus), and ψ2 cells or PA317 cells (e.g., for packaging retrovirus). Viral vectors can be generated by producing a cell line that packages a nucleic acid vector into a viral particle. The vectors can contain the minimal viral sequences required for packaging and subsequent integration into a host. The vectors can contain other viral sequences being replaced by an expression cassette for the polynucleotide(s) to be expressed. The missing viral functions can be supplied in trans by the packaging cell line. For example, AAV vectors can comprise ITR sequences from the AAV genome which are required for packaging and integration into the host genome.

In some embodiments, dual AAV vectors are generated by splitting a large transgene expression cassette in two separate halves (5′ and 3′ ends that encode N-terminal portion and C-terminal portion of, e.g., a prime editor polypeptide), where each half of the cassette is no more than 5 kb in length, optionally no more than 4.7 kb in length, and is packaged in a single AAV vector. In some embodiments, the full-length transgene expression cassette is reassembled upon co-infection of the same cell by both dual AAV vectors. In some embodiments, a portion or fragment of a prime editor polypeptide, e.g., a Cas9 nickase, is fused to an intein. The portion or fragment of the polypeptide can be fused to the N-terminus or the C-terminus of the intein. In some embodiments, a N-terminal portion of the polypeptide is fused to an intein-N, and a C-terminal portion of the polypeptide is separately fused to an intein-C. In some embodiments, a portion or fragment of a prime editor fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, a polynucleotide encoding a prime editor fusion protein is split in two separate halves, each encoding a portion of the prime editor fusion protein and separately fused to an intein. In some embodiments, each of the two halves of the polynucleotide is packaged in an individual AAV vector of a dual AAV vector system. In some embodiments, each of the two halves of the polynucleotide is no more than 5 kb in length, optionally no more than 4.7 kb in length. In some embodiments, the full-length prime editor fusion protein is reassembled upon co-infection of the same cell by both dual AAV vectors, expression of both halves of the prime editor fusion protein, and self-excision of the inteins.

A target cell can be transiently or non-transiently transfected with one or more vectors described herein. A cell can be transfected as it naturally occurs in a subject. A cell can be taken or derived from a subject and transfected. A cell can be derived from cells taken from a subject, such as a cell line. In some embodiments, a cell transfected with one or more vectors described herein can be used to establish a new cell line comprising one or more vector-derived sequences. In some embodiments, a cell transiently transfected with the compositions of the disclosure (such as by transient transfection of one or more vectors, or transfection with RNA), and modified through the activity of a prime editor, can be used to establish a new cell line comprising cells containing the modification but lacking any other exogenous sequence. Any suitable vector compatible with the host cell can be used with the methods of the disclosure. Non-limiting examples of vectors include pXT1, pSG5, pSVK3, pBPV, pMSG, and pSVLSV40.

In some embodiments, a prime editor protein can be provided to cells as a polypeptide. In some embodiments, the prime editor protein is fused to a polypeptide domain that increases solubility of the protein. In some embodiments, the prime editor protein is formulated to improve solubility of the protein.

In some embodiment, a prime editor polypeptide is fused to a polypeptide permeant domain to promote uptake by the cell. In some embodiments, the permeant domain is a including peptide, a peptidomimetic, or a non-peptide carrier. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide can comprise the HIV-1 that basic region amino acid sequence, which may include, for example, amino acids 49-57 of naturally-occurring that protein. Other permeant domains can include poly-arginine motifs, for example, the region of amino acids 34-56 of HIV-1 rev protein, nona-arginine, and octa-arginine. The nona-arginine (R9) sequence can be used. The site at which the fusion can be made may be selected in order to optimize the biological activity, secretion or binding characteristics of the polypeptide.

In some embodiments, a prime editor polypeptide is produced in vitro or by host cells, and it may be further processed by unfolding, e.g., heat denaturation, DTT reduction, etc. and may be further refolded. In some embodiments, a prime editor polypeptide is prepared by in vitro synthesis. Various commercial synthetic apparatuses can be used. By using synthesizers, naturally occurring amino acids can be substituted with unnatural amino acids. In some embodiments, a prime editor polypeptide is isolated and purified in accordance with recombinant synthesis methods, for example, by expression in a host cell and the lysate purified using HPLC, exclusion chromatography, gel electrophoresis, affinity chromatography, or other purification technique.

In some embodiments, a prime editing composition, for example, prime editor polypeptide components and PEgRNA/ngRNA are introduced to a target cell by nanoparticles. In some embodiments, the prime editor polypeptide components and the PEgRNA and/or ngRNA form a complex in the nanoparticle. Any suitable nanoparticle design can be used to deliver genome editing system components or nucleic acids encoding such components. In some embodiments, the nanoparticle is inorganic. In some embodiments, the nanoparticle is organic. In some embodiments, a prime editing composition is delivered to a target cell, e.g., a hepatocyte, in an organic nanoparticle, e.g., a lipid nanoparticle (LNP) or polymer nanoparticle.

In some embodiments, LNPs are formulated from cationic, anionic, neutral lipids, or combinations thereof. In some embodiments, neutral lipids, such as the fusogenic phospholipid DOPE or the membrane component cholesterol, are included to enhance transfection activity and nanoparticle stability. In some embodiments, LNPs are formulated with hydrophobic lipids, hydrophilic lipids, or combinations thereof. Lipids may be formulated in a wide range of molar ratios to produce an LNP. Any lipid or combination of lipids that are known in the art can be used to produce an LNP. Exemplary lipids used to produce LNPs are provided in Table 11 below.

In some embodiments, components of a prime editing composition form a complex prior to delivery to a target cell. For example, a prime editor fusion protein, a PEgRNA, and/or a ngRNA can form a complex prior to delivery to the target cell. In some embodiments, a prime editing polypeptide (e.g., a prime editor fusion protein) and a guide polynucleotide (e.g., a PEgRNA or ngRNA) form a ribonucleoprotein (RNP) for delivery to a target cell. In some embodiments, the RNP comprises a prime editor fusion protein in complex with a PEgRNA. RNPs may be delivered to cells using known methods, such as electroporation, nucleofection, or cationic lipid-mediated methods, or any other approaches known in the art. In some embodiments, delivery of a prime editing composition or complex to the target cell does not require the delivery of foreign DNA into the cell. In some embodiments, the RNP comprising the prime editing complex is degraded over time in the target cell. Exemplary lipids for use in nanoparticle formulations and/or gene transfer are shown in Table 11 below.

TABLE 11
Exemplary lipids for nanoparticle formulation or gene transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N41-(2,3-Dioleyloxy)prophyliN,N,N-trimethylammonium | DOTMA | Cationic |
| chloride | | |
| 1,2-Dioleoyloxy-3-trimethylammonium-propane | DOGS | Cationic |
| Dioctadecylamidoglycylspermine | | |
| N-(3-Aminopropy1)-N,N-dimethy1-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl omithinate | LHON | Cationic |
| 1-(2,3-Dioleoyloxypropy1)-2,4,6-trimethylpyridinium | 2Oc | Cationic |
| 2,3-Dioleyloxy-N-P(spenninecarboxamido-ethy1J-N,Ndimethyl- | DOSPA | Cationic |
| 1-propanatninium trifluoroacetate | | |
| 1,2-Dioley1-3-trimethylamtnonium-propane | DOPA | Cationic |
| N-(2-Hydroxyethyl)-N,N-dimethy1-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′,N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermy1)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]- | CLIP-1 | Cationic |
| dimethylammonium chloride | | |
| rac-[2(2,3-Dihexadecyloxypropyloxymethyloxy) | CLIP-6 | Cationic |
| ethyl]trimethylammoniun bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylpho sphocholine | DSEPC | Cationic |
| N-Palmitoyl D-erythro-sphingosyl carbamoyl-spenmine | CCS | Cationic |
| N-t-Butyl-N0-tetradecyl-3-tetradecylaminopropionamidine | diC14- | Cationic |
| | amidine | |
| Octadecenolyoxy[ethyl-2-heptadecenyl-3 hydroxyethyl] | DOTIM | Cationic |
| imidazolinium chloride | | |
| N1-Cholesteryloxycarbonyl-3,7-diazanonane-1,9-diamine | CDAN | Cationic |
| 2-(3-Bis(3-amino-propy1)-amino]propylamino)- | RPR209120 | Cationic |
| Nditetradecylcarbamoylme-ethyl-acetamide | | |
| 1,2-dilinoleyloxy-3-dimethylaminopropane | DLinDMA | Cationic |
| 2,2-dilinoley1-4-dimethylaminoethyl-[1,3]-dioxolane | DLin-KC2- | Cationic |
| | DMA | |
| dilinoleyl-methyl-4-dimethylaminobutyrate | DLin-MC3- | Cationic |
| | DMA | |

Exemplary polymers for use in nanoparticle formulations and/or gene transfer are shown in Table 12 below.

TABLE 12
Exemplary lipids for nanoparticle formulation or gene transfer

| Polymer | Abbreviation |
|---|---|
| Poly(ethylene)glycol | PEG |
| Polyethylenimine | PEI |
| Dithiobis (succinimidylpropionate) | DSP |
| Dimethyl-3,3′-dithiobispropionimidate | DTBP |
| Poly(ethylene imine)biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amidoethylenimine) | SS_PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | pHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-dodacylated chitosam | |
| Histone | |
| Collagen | |
| Dextran-spermine | D-SPM |

Exemplary delivery methods for polynucleotides encoding prime editing composition components are shown in Table 13 below.

TABLE 13
Exemplary polynucleotide delivery methods

| | | Delivery | | | |
|---|---|---|---|---|---|
| | | into Non- | | | Type of |
| | | Dividing | Duration of | Genome | Molecule |
| Delivery | Vector/Mode | Cells | Expression | Integration | Delivered |
| Physical | (e.g., | YES | Transient | NO | Nucleic |
| | electroporation, | | | | Acids and |
| | particle gun, | | | | Proteins |
| | Calcium phosphate | | | | |
| | transfection) | | | | |
| Viral | Retrovirus | NO | Stable | YES | RNA |
| | Lentivirus | YES | Stable | YES/NO with | RNA |
| | | | | modification | |
| | Adenovirus | YES | Transient | NO | DNA |
| | Adeno-Associated | YES | Stable | NO | DNA |
| | Virus (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic |
| | | | | what is | acids and |
| | | | | delivered | Proteins |
| | Polymeric | YES | Transient | NO | Nucleic |
| | Nanoparticles | | | | Acids |
| Biological | Attenuated | YES | Transient | NO | Nucleic |
| | Bacteria | | | | Acids |
| Non-Viral | Engineered | YES | Transient | NO | Nucleic |
| Delivery | Bacteriophages | | | | Acids |
| Vehicles | Mammalian | YES | Transient | NO | Nucleic |
| | Virus-like | | | | Acids |
| | Particles | | | | |
| | Biological | YES | Transient | NO | Nucleic |
| | liposomes: | | | | Acids |
| | Erythrocyte | | | | |
| | Ghosts and | | | | |
| | Exosomes | | | | |

The prime editing compositions of the disclosure, whether introduced as polynucleotides or polypeptides, can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject cells one or more times, e.g., one time, twice, three times, or more than three times, and the cells allowed to incubate with the agent(s) for some amount of time following each contacting event e.g., 16-24 hours. In cases in which two or more different prime editing system components, e.g., two different polynucleotide constructs are provided to the cell (e.g., different components of the same prim editing system, or two different guide nucleic acids that are complementary to different sequences within the same or different target genes), the compositions may be delivered simultaneously (e.g., as two polypeptides and/or nucleic acids). Alternatively, they may be provided sequentially, e.g., one composition being provided first, followed by a second composition.

The prime editing compositions and pharmaceutical compositions of the disclosure, whether introduced as polynucleotides or polypeptides, can be administered to subjects in need thereof for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which can be repeated with a frequency of about every day to about every 4 days, e.g., every 1.5 days, every 2 days, every 3 days, or any other frequency from about every day to about every four days. The compositions may be provided to the subject one or more times, e.g., one time, twice, three times, or more than three times. In cases in which two or more different prime editing system components, e.g., two different polynucleotide constructs are administered to the subject (e.g., different components of the same prim editing system, or two different guide nucleic acids that are complementary to different sequences within the same or different target genes), the compositions may be administered simultaneously (e.g., as two polypeptides and/or nucleic acids). Alternatively, they may be provided sequentially, e.g., one composition being provided first, followed by a second composition.

### EXAMPLES

### Example 1—Experimental Protocol of PEgRNA Screening Library

This experimental protocol of pegRNA screening library builds from the published method for screening PEgRNA variants in a pooled format using lentivirus libraries (Kim, H. K., Yu, G., Park, J. et al. Predicting the efficiency of prime editing guide RNAs in human cells. Nat Biotechnol (2020)).

The PEgRNAs described herein were tested with lentivirus constructed libraries. Lentiviral vectors were designed and constructed such that each lentiviral vector contains a sequence that encodes a single pegRNA and a corresponding target sequence. The lentiviral vectors were used to transduce HEK293T cells at a low MOI to allow for ˜ 1 virus integrant per infected cell. The cells were grown under antibiotic selection to enrich for cells containing the editing construct. After selection, the enriched cells are transfected with a plasmid encoding a prime editor fusion protein. 3 days after transfection, genomic DNA was harvested, and the target sequences were amplified and sequenced to examine editing efficiency.

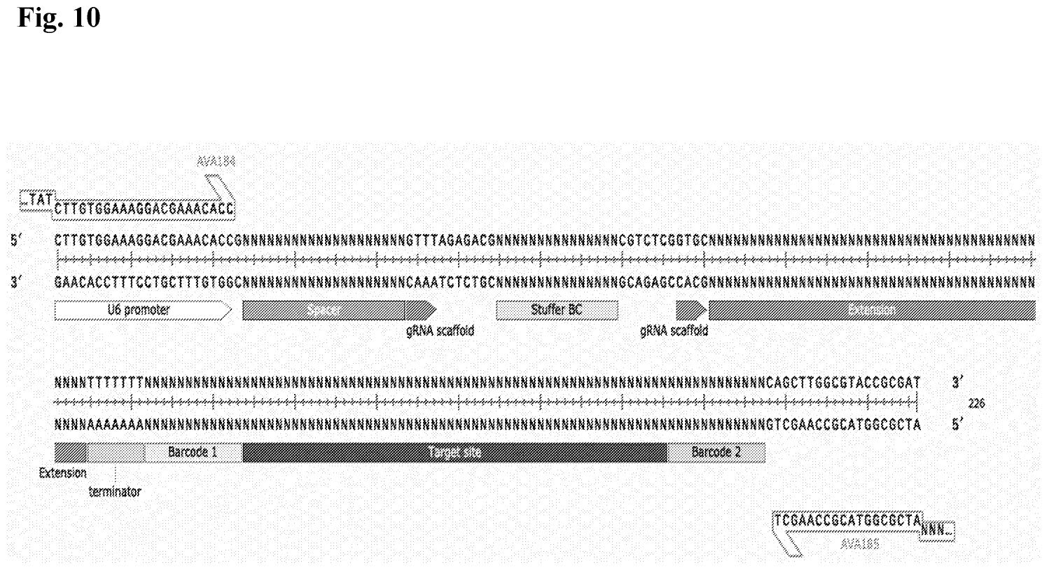

The overall design of the oligonucleotide library is shown in FIG. 10.

The oligonucleotides contain, in a 5′ to 3′ order, homology to a U6 promoter, a pegRNA spacer sequence, either a partial scaffold fragment with internal BsmBI site or a full scaffold sequence (depending on the library design), a 7-nt polyT terminator, a 12-nt barcode 1, the DNA target site, a 12-nt barcode 2, and a 3′ primer binding site. By having the target site flanked by two barcodes, matched at the stage of synthesis, crossover of PCR during amplification from gDNA were identified and removed from sequence analysis.

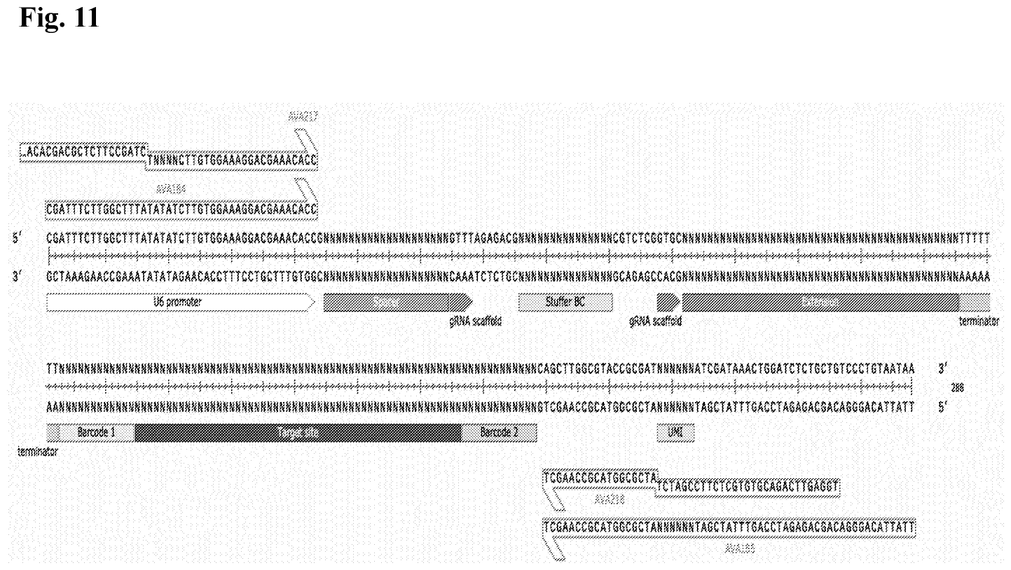

The product of PCR amplification with AVA184 and AVA185 produces the construct in FIG. 11.

### The Oligo Library was Amplified Using the Following Primers:

Forward primer (AVA184):
(SEQ ID NO: 7054)
CGATTTCTTGGCTTTATATATCTTGTGGAAAGGACGAAAC
ACC
(SEQ ID NO: 7055)
TTATTACAGGGACAGCAGAGATCCAGTTTATCGATNNNNN
NATCGCGGTACGCCAAGCT

### Gibson Assembly:

Lentiviral vectors were constructed using Gibson assembly method as described below. An acceptor vector was digested with Esp3I or BsmBI. The linearized product was purified and used for Gibson assembly with a 1:1-1:7 molar ratio to the oligo library. Electrocompetent NEB stable cells were transformed. The next day, the bacteria cells were harvested, and DNA was extracted with Qiagen plasmid plus midiprep using manufacturer's standard protocol. Clones were sequenced with next generation sequencing to ensure that the library as designed is represented in the clones.

### Make Lentivirus:

Lentivirus was prepared with a 3-plasmid system: On day 0, HEK293T cells were plated in a T150 flask. On day 1, 18 μg of lenti transfer vector, 18 μg of psPAX2, 12 μg of pMD2.g, and opti-mem up to 1250 uL were mixed. A separate solution containing lipofectamine 2000 was freshly prepared. Then, the two solutions were mixed and incubated. After 48 hours of incubation (day 3), the supernatant media was collected. 5× peg solution was added to the lentivirus solution to a final concentration of 1×, the solution was mixed well, and the mixture was left at 4° C. overnight. The next day, the precipitated lentivirus) was pelleted and resuspended and stored at −80° C.

### Full Scale Tissue Culture Transduction, Transfection:

Cells were plated in a T150 flask on day −1. On day 0, infect cells were predetermined with virus concentration condition to achieve ˜30% survival after antibiotic selection. The cells were transfected on day 6. On days 8-13, cells were harvested and genomic DNA was extracted for NGS prep. Forward and reverse primers as provided below are used to determine the optimal gDNA concentration to use per PCR reaction. Unique barcoding primers were used to uniquely identify editing at each target sequence. Sequencing results were analyzed, after removing reads that 1) do not contain barcodes or 2) contain crossover events (i.e. mismatch between the barcodes and the spacer sequences), with CRISPRESSO as described in Clement, K. et al., Nat. Biotechnol. 37, 224-226 (2019).

TABLE 14

| Forward primer | sequence |
|---|---|
| dsw50 | ACACTCTTTCCCTACACGACGCTCTTCC |
| | GATCTNNCTTGTGGAAAGGACGAAACA |
| | CC (SEQ ID NO: 7056) |
| dsw51 | ACACTCTTTCCCTACACGACGCTCTTCC |
| | GATCTNNNCTTGTGGAAAGGACGAAAC |
| | ACC (SEQ ID NO: 7057) |
| dsw52 | ACACTCTTTCCCTACACGACGCTCTTCC |
| | GATCTNNNNCTTGTGGAAAGGACGAA |
| | ACACC (SEQ ID NO: 7058) |
| dsw53 | ACACTCTTTCCCTACACGACGCTCTTCC |
| | GATCTNNNNNCTTGTGGAAAGGACGA |
| | AACACC (SEQ ID NO: 7059) |

TABLE 15

| Reverse primer | sequence |
|---|---|
| dsw5 | TGGAGTTCAGACGTGTGCTCTTCCGAT |
| | CTNGGACAGCAGAGATCCAGTTTATCG |
| | AT (SEQ ID NO: 7060) |
| dsw6 | TGGAGTTCAGACGTGTGCTCTTCCGAT |
| | CTNNGGACAGCAGAGATCCAGTTTATC |
| | GAT (SEQ ID NO: 7061) |
| dsw7 | TGGAGTTCAGACGTGTGCTCTTCCGAT |
| | CTNNNGGACAGCAGAGATCCAGTTTAT |
| | CGAT (SEQ ID NO: 7062) |

### Example 2—Prime Editing of Human Gene Targets with Modified PEgRNA Sequences

To examine modifications in the gRNA core of PEgRNAs, PEgRNAs were designed for prime editing in seven target loci in six human genes (ATP7B, SLC37A4, EMX1, FANCF, HEK3, and NCF1) as indicated in Table 17 below. For ATP7B, SLC37A4, and NCF1, the mutations to be corrected by PEgRNA for each gene are also indicated. Each PEgRNA contained one or more nucleotide sequence modifications as indicated in Table 17. PEgRNAs and screening library were generated editing efficiency was determined as described in Example 1 above. For each PEgRNA, prime editing efficiency was normalized to a corresponding control PEgRNA having the same spacer, PBS, and editing template for the target locus and with a gRNA core as set forth in SEQ ID NO: 16. The normalized editing results are shown in Table 17, where each number indicates fold change in editing efficiency as compared to the control PEgRNA. Sequences of the modified gRNA core are provided in Table 1: for each modification indicated in the “Variant” column in Table 17, the variant name after “Scaffold” corresponds to the gRNA core name in Table 1. For example, variant “Scaffold_F+E” corresponds to gRNA core name “F+E” in Table 1. The “modification includes M4” column indicates whether the PEgRNA includes a M4 modification besides the modification indicated by the “variant” name, where “Scaffold_M4” only has the M4 modification and no other modifications.

TABLE 17
gRNA variant core results

| | gRNA core | | | SLC37A | | | | NCF1- | | average fold editing |
|---|---|---|---|---|---|---|---|---|---|---|
| gRNA core | SEQ ID | ATP7B- | ATP7B- | 4 | EMX1_{—} | FANCF_{—} | HEK3_{—} | c.73_74 | Modification | relative to canonical |
| name | No. | H1069Q | R778L | G339C | TGC | ACC | GC | delGT | includes M4 | SpCas9 gRNA core |
| F + E | 24 | 2.25 | 2.11 | 4.17 | 1.14 | 2.4 | 3.23 | 0.59 | Y | 2.27 |
| sl2_flip | 25 | 1.8 | 2.93 | 3.24 | 1.14 | 1.88 | 3.35 | 1.38 | Y | 2.25 |
| TetraLoop_L1 | 27 | 2.39 | 1.66 | 4.06 | 1.26 | 1.79 | 2.69 | 1.01 | Y | 2.12 |
| TetraLoop_L2 | 28 | 2.42 | 1.66 | 3.85 | 1.31 | 2.18 | 2.58 | 0.81 | Y | 2.12 |
| TetraLoop_L8 | 34 | 2.03 | 1.92 | 3.67 | 1.02 | 1.76 | 2.81 | 0.8 | Y | 2 |
| TetraLoop_L9 | 35 | 1.98 | 1.18 | 3.95 | 1.32 | 2.5 | 2.4 | 0.66 | Y | 2 |
| TetraLoop_L10 | 36 | 1.54 | 1.65 | 4.15 | 1.56 | 2.07 | 1.98 | 0.94 | Y | 1.98 |
| Loop2_L3 | 41 | 2.02 | 1.87 | 1.957 | 1.18 | 2.4 | 2.98 | 1.23 | Y | 1.96 |
| TetraLoop_L0 | 26 | 2.16 | 1.95 | 3.34 | 0.65 | 2.43 | 1.75 | 0.83 | Y | 1.87 |
| TetraLoop_L11 | 37 | 1.84 | 1.54 | 3.459 | 0.88 | 2.44 | 2.08 | 0.81 | Y | 1.86 |
| Loop2_L2 | 40 | 2 | 2.05 | 1.85 | 1.4 | 2.05 | 2.72 | 0.85 | Y | 1.85 |
| TetraLoop_L5 | 31 | 1.64 | 1.94 | 2.82 | 0.73 | 2.53 | 2.35 | 0.84 | Y | 1.84 |
| Loop2_L0 | 38 | 1.94 | 1.98 | 2.58 | 0.95 | 2.88 | 1.15 | 1.16 | Y | 1.81 |
| TetraLoop_L3 | 29 | 1.39 | 0.85 | 3.28 | 0.98 | 2.94 | 2.39 | 0.69 | Y | 1.79 |
| TetraLoop_L4 | 30 | 2.3 | 1.58 | 2.39 | 1.39 | 1.78 | 2.1 | 0.88 | Y | 1.78 |
| M4 | 20 | 1.75 | 1.29 | 2.62 | 1.58 | 1.93 | 2.5 | 0.74 | Y(M4 alone) | 1.77 |
| E5 | 23 | 1.42 | 1.45 | 1.81 | 0.74 | 2.61 | 2.47 | 1.65 | no | 1.74 |
| TetraLoop_L6 | 32 | 1.69 | 1.62 | 2.18 | 0.82 | 1.78 | 2.32 | 0.91 | Y | 1.62 |
| TetraLoop_L7 | 33 | 1.38 | 1.41 | 1.69 | 0.94 | 2.52 | 2.42 | 0.8 | Y | 1.59 |
| Loop2_L8 | 46 | 2.44 | 1.83 | 1.33 | 0.74 | 1.39 | 2.04 | 1.06 | Y | 1.55 |
| Loop2_L5 | 43 | 1.61 | 1.42 | 1.87 | 1.24 | 2.13 | 1.45 | 0.96 | Y | 1.53 |
| Loop2_L10 | 48 | 1.11 | 1.74 | 1.35 | 1.04 | 2.63 | 1.49 | 0.91 | Y | 1.47 |
| Loop2_L1 | 39 | 1.87 | 1.22 | 1.13 | 1.17 | 2.18 | 1.72 | 0.89 | Y | 1.45 |
| M3 | 19 | 0.89 | 0.94 | 2.16 | 1.08 | 1.46 | 2.74 | 0.62 | no | 1.41 |
| Loop2_L9 | 47 | 1.39 | 1.12 | 1.46 | 0.91 | 2.09 | 1.82 | 0.85 | Y | 1.38 |
| Loop2_L4 | 42 | 1.87 | 1.54 | 1.39 | 0.66 | 2.55 | 0.93 | 0.64 | Y | 1.37 |
| M1 | 17 | 0.72 | 0.85 | 1.78 | 1.19 | 2.56 | 1.97 | 0.36 | no | 1.34 |
| Loop2_L6 | 44 | 0.96 | 1.76 | 1.76 | 1 | 1.45 | 1.08 | 0.78 | Y | 1.26 |
| Loop2_L7 | 45 | 0.99 | 1.14 | 1.28 | 1.52 | 2.16 | 1.06 | 0.59 | Y | 1.25 |
| M2 | 18 | 0.76 | 1.71 | 2.08 | 0.6 | 1.07 | 1.96 | 0.55 | no | 1.25 |
| sl2 cg | 22 | 1.48 | 1.08 | 0.92 | 0.87 | 1.22 | 2 | 1.02 | no | 1.23 |
| sl2 gc | 21 | 1.27 | 1.53 | 1.42 | 0.65 | 1.16 | 1.46 | 0.7 | no | 1.17 |
| Loop2_L11 | 49 | 1.08 | 0.6 | 1.26 | 1.03 | 1.52 | 1.19 | 0.88 | Y | 1.08 |
| Canonical | 16 | 1 | 1 | 1 | 1 | 1 | 1 | 1 | no | 1 |
| SpCas9 gRNA | | | | | | | | | | |
| core | | | | | | | | | | |
| Loop3_L11 | 61 | NA | 0.1 | 0.08 | 0 | 0.39 | 0.063 | 0 | Y | 0.1 |
| Loop3_L4 | 54 | 0.23 | 0.23 | 0.27 | 0.35 | 0.69 | 0.36 | 0.44 | Y | 0.37 |
| Loop3_L7 | 57 | 0.38 | 0.12 | 0.07 | 0.03 | 0.45 | 0.15 | 0.04 | Y | 0.18 |
| Loop3_L6 | 56 | 0.26 | 0.04 | 0.13 | 0.08 | 0.39 | 0.21 | 0.05 | Y | 0.16 |
| Loop3_L0 | 50 | 0.12 | 0.09 | 0.07 | 0.12 | 0.37 | NA | 0.14 | Y | 0.15 |
| Loop3_L10 | 60 | 0.35 | 0.03 | 0 | 0.04 | 0.35 | 0.06 | 0 | Y | 0.12 |
| Loop3_L9 | 59 | NA | 0 | 0 | 0.07 | 0.09 | 0.15 | 0.35 | Y | 0.1. |
| Loop3_L2 | 52 | 0.15 | 0.05 | 0 | 0.03 | 0.19 | 0.08 | 0.12 | Y | 0.09 |
| Loop3_L3 | 53 | 0.22 | 0.05 | 0.03 | 0.03 | 0.04 | 0.15 | 0.07 | Y | 0.08 |
| Loop3_L1 | 51 | 0 | 0.28 | 0 | 0.16 | 0 | 0.11 | 0 | Y | 0.08 |
| Loop3_L5 | 55 | 0.26 | 0.07 | 0 | 0 | 0.05 | 0.079 | 0.05 | Y | 0.07 |
| Loop3_L8 | 58 | 0.1 | 0.04 | 0.16 | 0 | 0.09 | 0.09 | 0 | Y | 0.07 |

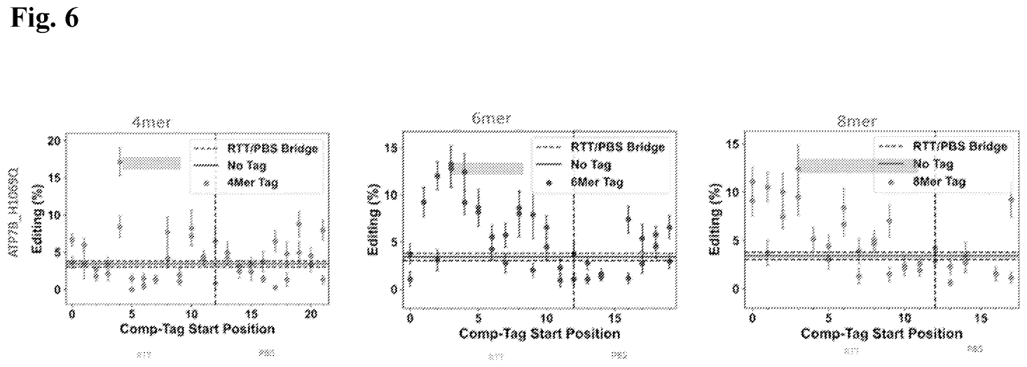

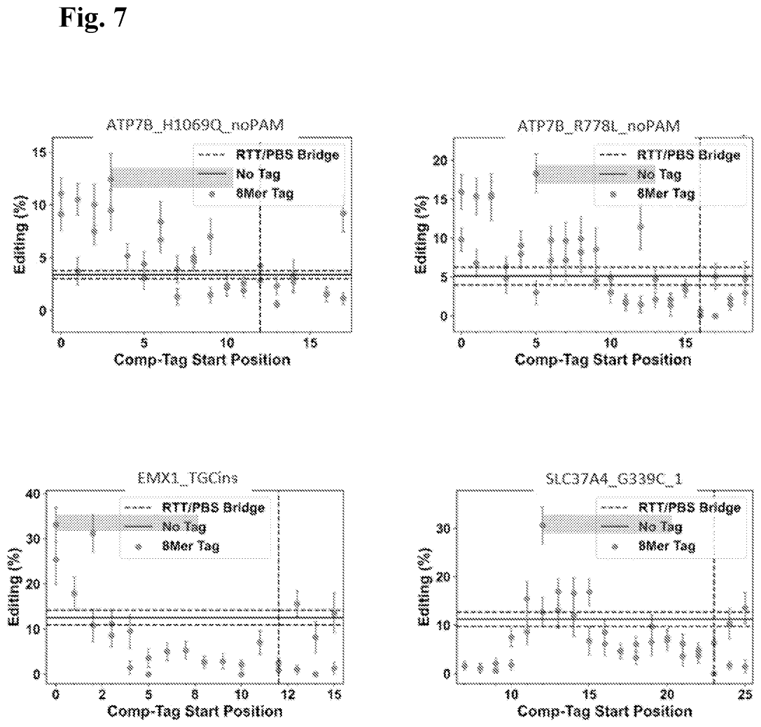

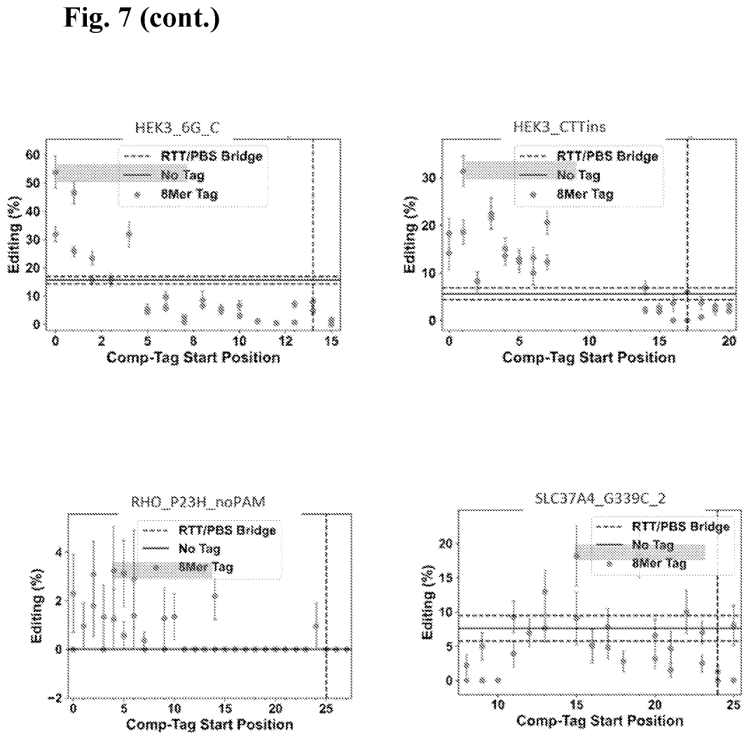

To examine tag sequence modification of PEgRNAs, PEgRNAs were designed for prime editing of five human genes (ATP7B, EMX1, SLC37A4, HEK3, and RHO). Seven target loci were tested: (correction of H1069Q or R778L for ATP, G-C substitution and CTT insertion for HEK3, correction of P23H for RHO, correction of G339C for SLC37A4, and insertion for EMX1) as indicated in FIGS. 6 and 7. For each PEgRNA, a tag sequence of various length and complementarity position with the editing template and/or PBS was added at the 3′ end of the PBS, such that the PEgRNA contains, from 5′ to 3′: the spacer, the gRNA core, the editing template, the PBS, and the tag sequence. A linker sequence from 1-6 nucleotides in length was also included between the PBS and the tag sequence. The linkers contained randomized nucleotide sequences, with any sequence having complementarity or identity to PBS, editing template, spacer, and tag sequence and sequences that would form additional secondary structure removed from the linker pool. The PEgRNA library was assembled, and prime editing efficiency was examined as described in Example 1.

The editing results in comparison to control PEgRNAs without the tag sequence for each target are shown in FIGS. 6 and 7. In each diagram, the horizontal solid line indicates editing efficiency with the control PEgRNA (horizontal dashed lines above and below the solid line indicates error margin of editing efficiency with the control PEgRNA). The vertical dashed line indicates the position of the junction between the editing template and the PBS. On the X axis, position 0 corresponds to the 5′ most position of the editing template. The “Comp-tag Start Position” on the X axis indicates the position of the tag sequence when hybridized to the editing template and/or RTT sequence via complementarity, where the “start position” of the tag sequence complementarity corresponding to the 5′ most position on region of the extension arm that is complementary to the tag sequence. For example, a start position being position 5 indicates that the extension arm has a region that is complementary to the tag sequence, and the 5′ most (the “first”) nucleotide in such region is at position 5, with position 0 being the 5′ most nucleotide of the editing template. For each start position, two PEgRNAs having the same tag sequence and the different linker sequence were tested, and the results are indicted by the two data points at each start position. For ATP7B H1069Q locus, tag sequence of 4, 6, and 8 nucleotides in length were tested (FIG. 6; 4Mer tag refers to a length of the tag sequence being 4 nucleotides). 8 nucleotide long tag sequences were tested for the other loci.

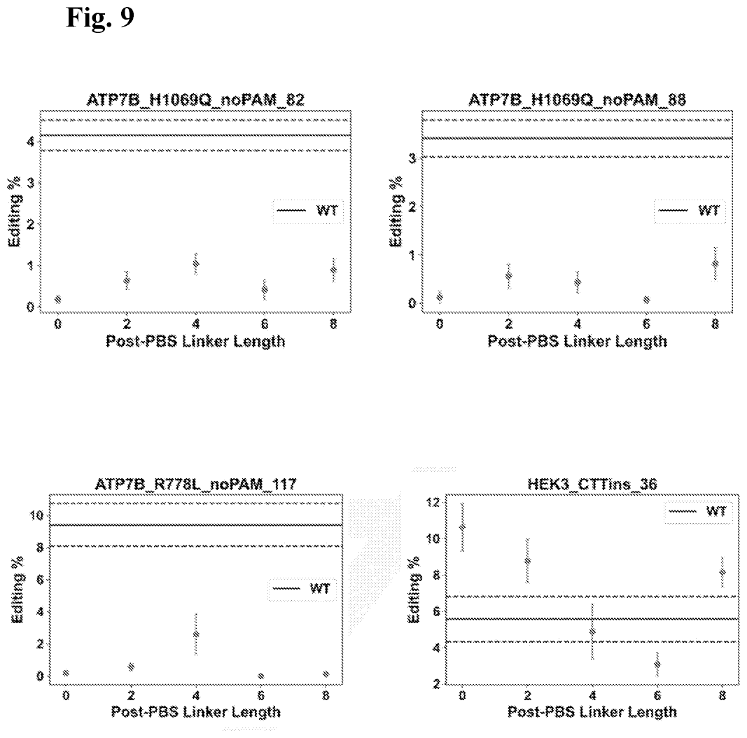

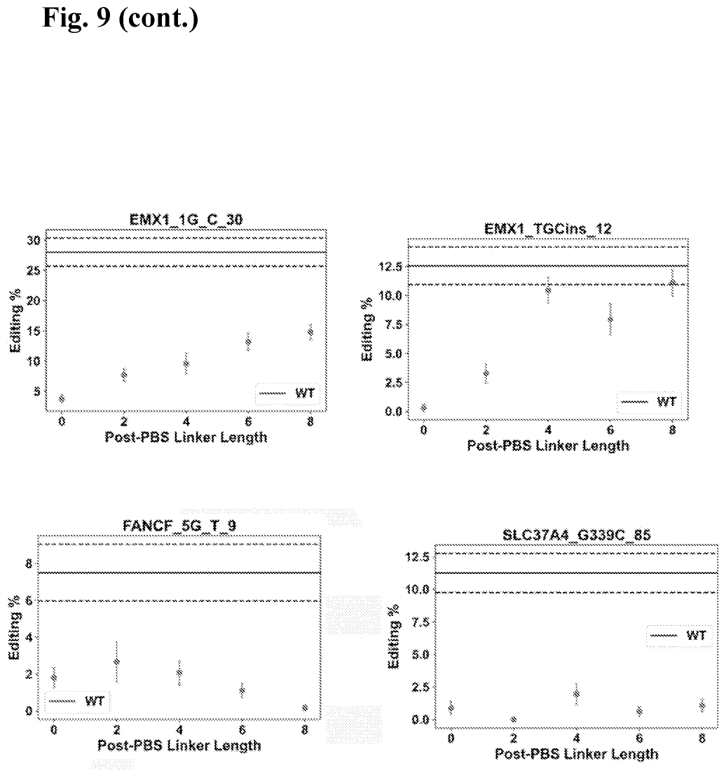

To examine editing efficiency of legRNAs, PEgRNAs were designed for prime editing seven target loci in five human genes (ATP7B, EMX1, SLC37A4, HEK3, and FANCF) as indicated in FIG. 9. Each PEgRNA contains, from 5′ to 3′: the spacer, the first half of the gRNA core, the editing template, the PBS, and the second half o the gRNA core. A linker is also included in between the PBS and the second half of the gRNA core. Sequences of the first half and second half of the gRNA core are provided below;

First half of gRNA core:
(SEQ ID NO: 6376)
GTTTAAGAGCTAGAAATAGCAAGTTTAAATAAGGCTAGTC
CGTTATCAGCGTGA
Second half of gRNA core:
(SEQ ID NO: 6377)
AAACGCGGCACCGAGTCGGTGC

PEgRNA library was assembled, and prime editing efficiency was examined as described in Example 1. The editing results in comparison to control PEgRNAs that has a configuration of 5′-spacer-gRNA core-editing template-PBS-3′ are shown in FIG. 9, where the horizontal solid line indicates editing efficiency with the control PEgRNA and horizontal dashed lines above and below the solid line indicates error margin of editing efficiency with the control PEgRNA.

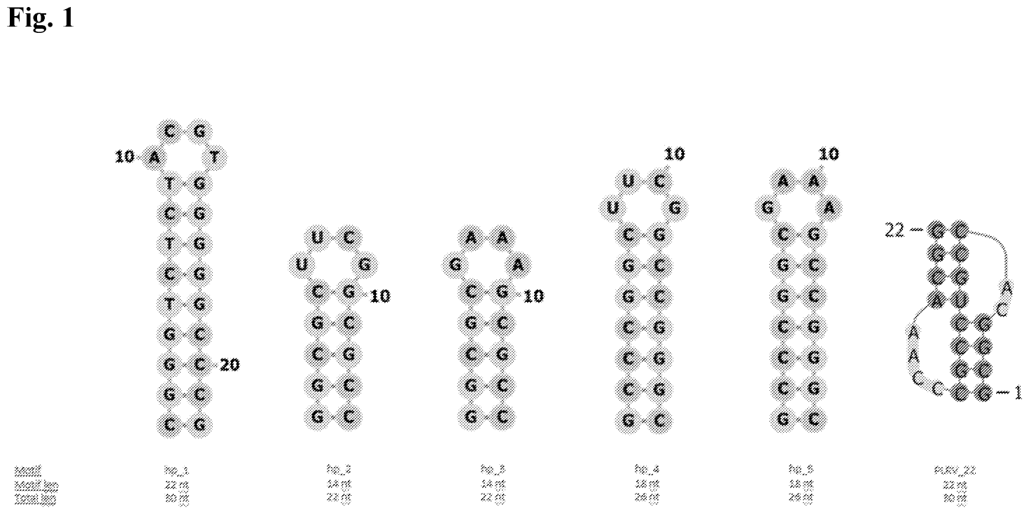

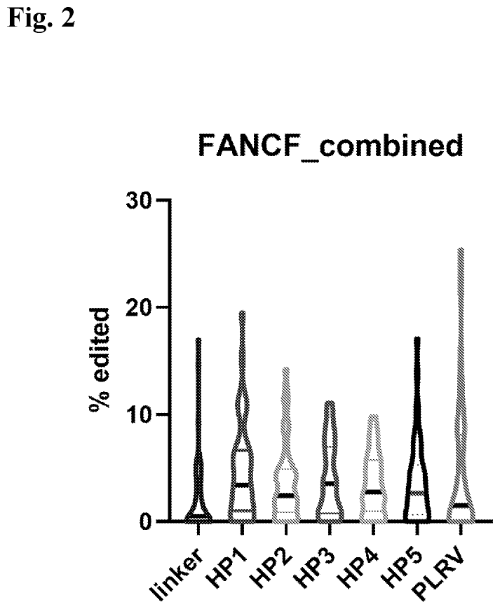

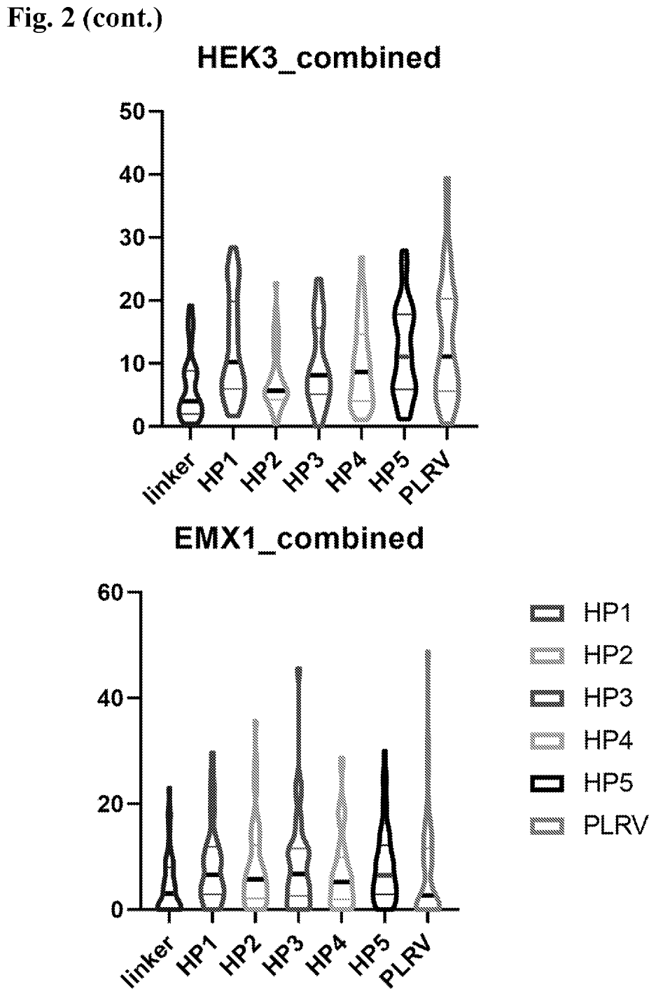

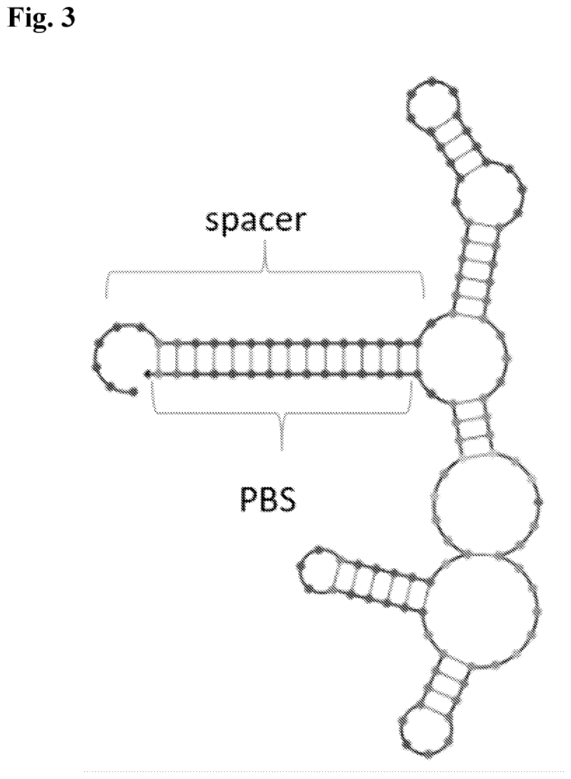

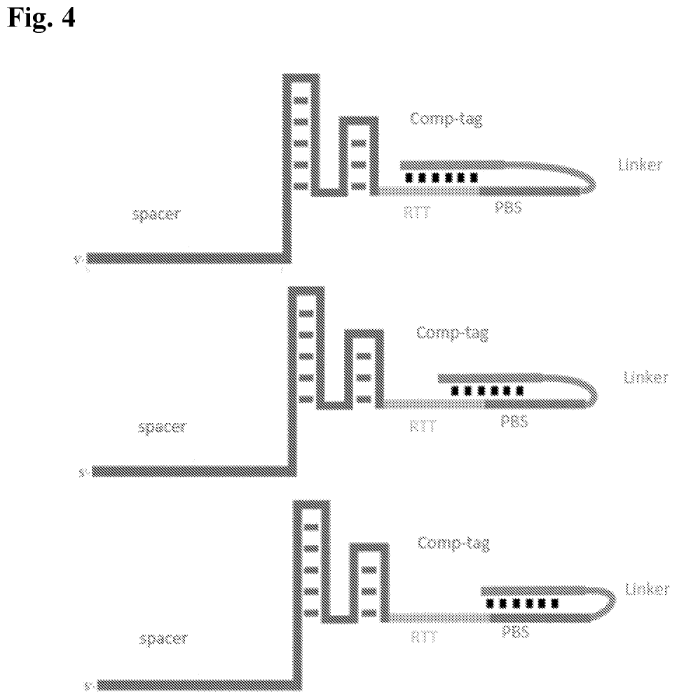

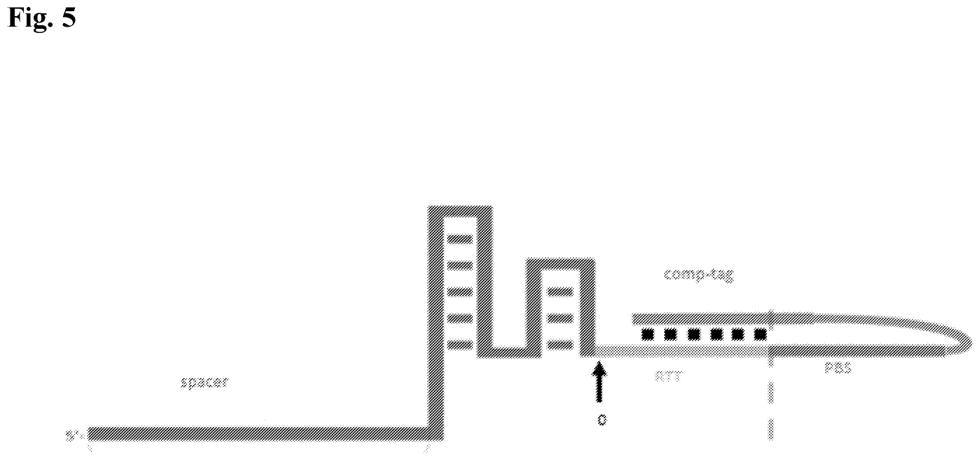

To examine editing efficiency of 3′ nucleic acid moieties, PEgRNAs were designed for prime editing three target loci human genes EMX1, HEK3, and FANCF. For each PEgRNA, a nucleotide moiety as shown in FIG. 1 was added to the 3′ end of the PBS. An 8 nucleotide linker was also included between the 3′ moiety and the PBS (linker sequence: AACAUUGA), such that each PEgRNA contains, from 5′ to 3′: the spacer, the gRNA core, the editing template, the PBS, the linker, and the 3′ nucleic acid moiety. Sequences of the 3′ nucleic acid moieties are provided in Table 3. For each 3′ nucleic acid moiety at a particular target locus, 48 unique PEgRNAs with the same 3′ nucleic acid moiety and each having a unique combination selected from 1 spacer, 3 nucleotide edit in the editing template, 4 PBS lengths, and 4 editing template lengths. PEgRNA library was assembled, and prime editing efficiency was examined as described in Example 1. For each nucleic acid moiety, editing efficiency using the 48 PEgRNAs having the nucleic acid moiety was plotted and the results in comparison to control PEgRNAs that has the 8 nucleotide linker only and not the 3′ moiety are shown in FIG. 2. In each violin plot in FIG. 2, “linker” refers to editing with the control PEgRNA. HP1, HP2, HP3, HP4, HP5, and PLRV each refers to hp_1 (SEQ ID NO:1), hp_2 (SEQ ID NO: 7), hp_3 (SEQ ID NO: 3), hp_4 (SEQ ID NO: 6), hp_5 (SEQ ID NO: 5), and PLRV_22 (SEQ ID NO:4), respectively, in the structure as shown in FIG. 1 and the sequences in Table 3.

### Example 3—Prime Editing of Human Gene Targets with Tag-Modified PEgRNA Sequences

In this example, the effect of various tag parameters on prime editing efficiency is further examined. PEgRNAs with various spacer, PBS, and RTT combinations targeting a locus in one of seven human genes (APT7B, NCF1, HEK3, EMX1, FANCF, RHO, and SLC37A4) were tested with and without tag sequences. For each PEgRNA, a tag sequence of various length and complementarity position with the editing template and/or PBS was added at the 3′ end of the PBS, such that the PEgRNA contains, from 5′ to 3′: the spacer, the gRNA core, the editing template, the PBS, and the tag sequence. The tag sequences used varied in length between 4 and 24 nucleotides. Linker sequences were included between the PBS and the tag sequence. The linkers contained randomized nucleotide sequences 4, 5, or 6 nucleotides in length, with any sequences that would form additional secondary structure or that had complementarity to the PBS, editing template, spacer, or tag sequence removed from the linker pool. The PEgRNA libraries were assembled, and prime editing efficiency was examined as described in Example 1.

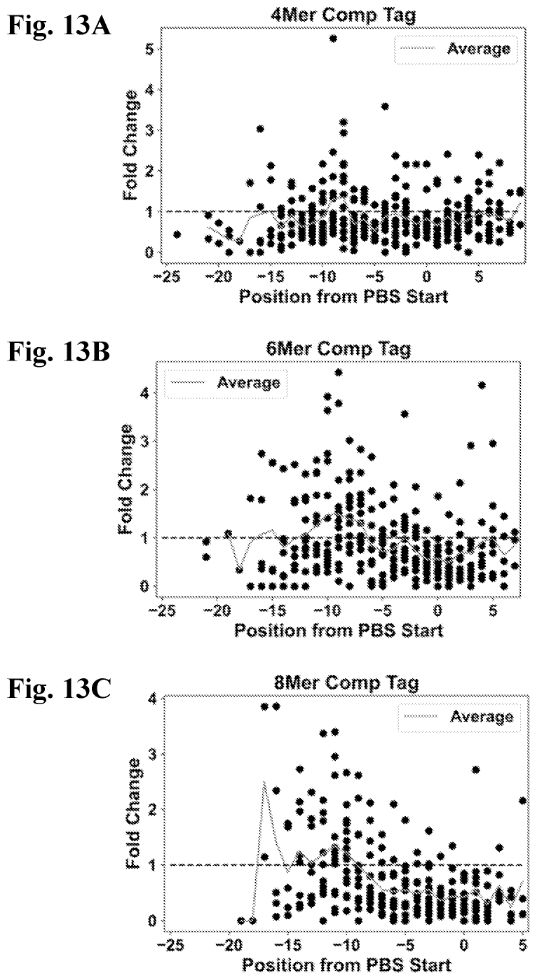

The effect of binding position on editing efficiency is examined in FIG. 13. In the graphs of FIG. 13, the x-axis shows the binding position of the 3′-most nucleotide of the tag relative to the RTT/PBS boundary, with position 0 representing the first nucleotide in the PBS. For a 4 mer tag (i.e., a tag 4 nucleotides in length), a binding position of −4 or less (i.e., −5, −6, −7, etc.) binds completely within the RTT, while a binding position of −3 or more binds at least partially within the PBS. The y-axis in FIG. 13 is the fold change in editing percentage of the tagged version of the PEgRNA verses the untagged version. Each dot shows the fold change for a single pair of PEgRNA (tagged vs. untagged). The average fold change for all PEgRNA of the indicated length having a tag binding at the same position is shown in the solid line on the graph. FIG. 13A shows the results for 4mer tags, FIG. 13B shows the results for 6 mer tags, and FIG. 13C shows the results for 8 mer tags. In each graph, there was a bump in the average fold change line in the region where the tags bind completely within the edit template.

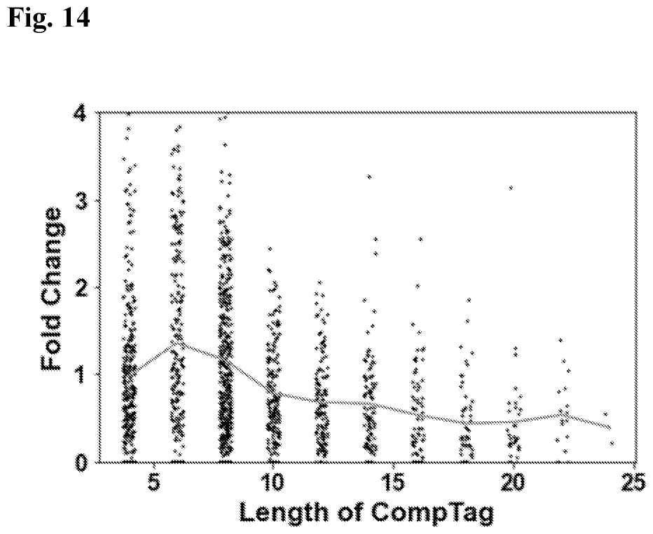

A second analysis was performed to examine the effect of tag length on prime editing performance. Based on the previous analyses, only those tags that bind completely within the edit template were included. The results are shown in FIG. 14 with the corresponding data in Table 18 below. In FIG. 14, the x-axis is the length of the tag (labeled Length of CompTag) and the y-axis is the fold change in percent editing of the tagged version of a PEgRNA verses the untagged version of the same PEgRNA. Each dot shows the fold change for a single pair of PEgRNA (tagged vs. untagged). The average fold change for all PEgRNA of the indicated length having a tag binding at the same position is shown in the solid line.

TABLE 18
The effect of tag length on prime editing efficiency.

| | | Percent w/ | Percent w/ | Percent w/ |
|---|---|---|---|---|
| | | 1.01 Fold | 1.5 Fold | 2 Fold |
| Tag Length | Total Tags | Improvement | Improvement | Improvement |
| (nt) | Tested | or Greater | or Greater | or Greater |
| 4 | 327 | 39.14% | 20.18% | 11.93% |
| 6 | 245 | 51.84% | 38.37% | 28.57% |
| 8 | 432 | 46.76% | 30.09% | 17.36% |
| 10 | 187 | 31.02% | 12.30% | 2.67% |
| 12 | 139 | 22.30% | 7.19% | 0.72% |
| 14 | 102 | 18.63% | 5.88% | 2.94% |
| 16 | 74 | 18.92% | 4.05% | 2.70% |
| 18 | 54 | 11.11% | 3.70% | 0.00% |
| 20 | 35 | 8.57% | 2.86% | 2.86% |
| 22 | 17 | 17.65% | 0.00% | 0.00% |
| 24 | 3 | 0.00% | 0.00% | 0.00% |

### Example 4. Prime Editing of Human Gene Targets with PEgRNAs Having gRNA Core Modifications

The effect of gRNA core modifications on prime editing efficiency was examined. PEgRNAs were designed for targeting and editing one locus in each of three human genes, referred to as target 1, target 2, and target 3, respectively. Each PEgRNA contains, from 5′ to 3′: a spacer, a gRNA core, and an extension arm that includes an editing template and a PBS. For each of targets 1, 2, and 3, all examined PEgRNAs have the same spacer sequence and the same extension arm sequence, and each contained a different gRNA core sequence: one control PEgRNA contains the canonical SpCas9 gRNA core according to SEQ ID NO: 16, and 492 examined PEgRNAs each contains a gRNA core that harbors one or more nucleotide modifications relative to SEQ ID NO: 16. Sequences of the gRNA cores are provided in Table 2.

The PEgRNAs were examined for editing of targets 1, 2, and 3 and prime editing efficiency in lentiviral transduced HEK293T cells as described in Example 1. The effect of gRNA core modifications on prime editing efficiency is shown in Table 16. Table 16 contains eight columns. From left to right, column 1 provides a brief name of the gRNA core, and column 2 provides the SEQ ID NO of each gRNA core. Columns 3-5 provide editing efficiency at target sites 1-3, respectively: for each target site, the editing efficiency of each modified PEgRNA was normalized to the editing efficiency of the control PEgRNA: Fold change=Editing Efficiency (modified gRNA core)/Editing Efficiency control, wherein the control PEgRNA has the canonical SpCas9 gRNA core according to SEQ ID NO: 16. For each PEgRNA, two replicates were tested, and the average fold change is reported. Column 6 reports the average fold change of columns 3-5. Column 7 provides nucleotide insertion location, for example, extension in the upper stem of the tetraloop (e.g., replacing nucleotides 11-12 of SEQ ID NO: 16 with 3, 4, or 5 contiguous nucleotides and replacing nucleotides 16-17 with the reverse complement of the 3, 4, or 5 contiguous nucleotide; complementary insertions between nucleotides 12 and 13 and between nucleotides 16 and 17 compared to SEQ ID NO: 16), or extension in stem loop 2 (e.g., replacing nucleotides 49-52 of SEQ ID NO: 16 with 7 or more contiguous nucleotides and replacing nucleotides 57-60 with the reverse complement of the 7 or more contiguous nucleotides; or complementary insertions between nucleotides 52 and 53 and between nucleotides 56-57 compared to SEQ ID NO: 16). Column 8 provides the length of the extension in basepairs; for example, a 2 basepair extension in stem loop 2 means complementary insertions of 2 nucleotides on each side of stem loop 2. Except for the control PEgRNA sequences and SEQ ID NO: 4351, other tested extended gRNA core sequences also included a “M4” modification (a U to A substitution at nucleotide 5 and an A to U substitution at nucleotide 26 relative to SEQ ID NO: 16).

PEgRNAs and corresponding editing efficiency in Table 16 are provided in ascending order from top to bottom. For both PEgRNAs having an extended tetraloop and PEgRNAs having an extended stem loop 2, improved average editing efficiency over the canonical SpCas9 PEgRNA was observed. Compared to PEgRNAs having the canonical SpCas9 gRNA core sequence, 491 of the 492 tested PEgRNAs showed increased or comparable editing efficiency for at least one target site, and 487 tested PEgRNAs showed increased average editing efficiency.

TABLE 16
Prime editing efficiency with PEgRNAs having modified gRNA cores.

| | gRNA | | | | | | |
|---|---|---|---|---|---|---|---|
| gRNA | core | target 1 | target 2 | target 3 | | | Extension |
| core | SEQ ID | Ave. fold | Ave. fold | Ave. fold | Ave. fold | Extended | base pair |
| name | NO. | change | change | change | change | feature | length |
| L2_e8_20 | 4254 | 0.32 | 0.19 | 0.08 | 0.20 | Stem loop 2 | 8 |
| L2_e6_94 | 4153 | 1.08 | 0.31 | 0.16 | 0.52 | Stem loop 2 | 6 |
| L2_e8_5 | 4239 | 0.90 | 0.94 | 0.38 | 0.74 | Stem loop 2 | 8 |
| L2_e7_12 | 4171 | 1.49 | 0.71 | 0.52 | 0.91 | Stem loop 2 | 7 |
| L2_e7_22 | 4181 | 1.46 | 0.89 | 0.49 | 0.95 | Stem loop 2 | 7 |
| canonical | 4350 or 16 | 1.00 | 1.00 | 1.00 | 1.00 | none | 0 |
| SpCas9 | | | | | | | |
| L2_e7_29 | 4188 | 1.42 | 1.08 | 0.53 | 1.01 | Stem loop 2 | 7 |
| L2_e8_22 | 4256 | 1.58 | 1.10 | 0.49 | 1.06 | Stem loop 2 | 8 |
| L2_e8_9 | 4243 | 1.59 | 0.92 | 0.70 | 1.07 | Stem loop 2 | 8 |
| L2_e6_85 | 4144 | 1.06 | 1.33 | 0.81 | 1.07 | Stem loop 2 | 6 |
| L2_e7_46 | 4205 | 1.82 | 0.66 | 0.77 | 1.08 | Stem loop 2 | 7 |
| L2_e7_58 | 4217 | 2.13 | 0.75 | 0.44 | 1.11 | Stem loop 2 | 7 |
| L2_e7_23 | 4182 | 1.51 | 1.30 | 0.51 | 1.11 | Stem loop 2 | 7 |
| L2_e8_11 | 4245 | 1.82 | 1.20 | 0.40 | 1.14 | Stem loop 2 | 8 |
| L2_e7_60 | 4219 | 2.00 | 0.94 | 0.55 | 1.16 | Stem loop 2 | 7 |
| L2_e6_6 | 4065 | 1.53 | 1.25 | 0.71 | 1.17 | Stem loop 2 | 6 |
| L2_e5_24 | 3983 | 1.81 | 1.20 | 0.51 | 1.17 | Stem loop 2 | 5 |
| L2_e7_10 | 4169 | 1.80 | 0.93 | 0.80 | 1.17 | Stem loop 2 | 7 |
| T_e2_12 | 4303 | 1.41 | 1.33 | 0.78 | 1.17 | Tetraloop | 2 |
| L2_e4_19 | 3903 | 1.25 | 1.71 | 0.61 | 1.19 | Stem loop 2 | 4 |
| L2_e6_95 | 4154 | 1.81 | 1.23 | 0.54 | 1.19 | Stem loop 2 | 6 |
| L2_e7_56 | 4215 | 1.87 | 1.07 | 0.66 | 1.20 | Stem loop 2 | 7 |
| L2_e8_23 | 4257 | 1.90 | 1.09 | 0.64 | 1.21 | Stem loop 2 | 8 |
| L2_e7_1 | 4160 | 1.82 | 1.35 | 0.51 | 1.23 | Stem loop 2 | 7 |
| L2_e6_76 | 4135 | 1.62 | 1.47 | 0.59 | 1.23 | Stem loop 2 | 6 |
| L2_e6_56 | 4115 | 2.07 | 1.02 | 0.59 | 1.23 | Stem loop 2 | 6 |
| L2_e6_38 | 4097 | 1.71 | 1.36 | 0.64 | 1.24 | Stem loop 2 | 6 |
| L2_e8_14 | 4248 | 2.15 | 0.71 | 0.86 | 1.24 | Stem loop 2 | 8 |
| L2_e7_32 | 4191 | 1.66 | 1.60 | 0.46 | 1.24 | Stem loop 2 | 7 |
| L2_e7_48 | 4207 | 2.01 | 1.05 | 0.66 | 1.24 | Stem loop 2 | 7 |
| L2_e5_26 | 3985 | 1.55 | 1.69 | 0.53 | 1.26 | Stem loop 2 | 5 |
| L2_e7_30 | 4189 | 2.36 | 0.78 | 0.64 | 1.26 | Stem loop 2 | 7 |
| L2_e7_37 | 4196 | 1.90 | 1.30 | 0.58 | 1.26 | Stem loop 2 | 7 |
| L2_e7_9 | 4168 | 1.91 | 1.22 | 0.68 | 1.27 | Stem loop 2 | 7 |
| L2_e7_16 | 4175 | 1.88 | 1.21 | 0.74 | 1.27 | Stem loop 2 | 7 |
| L2_e4_16 | 3900 | 1.67 | 1.37 | 0.79 | 1.28 | Stem loop 2 | 4 |
| L2_e6_81 | 4140 | 1.99 | 1.26 | 0.59 | 1.28 | Stem loop 2 | 6 |
| T_e2_23 | 4314 | 2.37 | 0.77 | 0.70 | 1.28 | Tetraloop | 2 |
| L2_e7_6 | 4165 | 2.12 | 1.28 | 0.44 | 1.28 | Stem loop 2 | 7 |
| L2_e6_8 | 4067 | 2.01 | 1.09 | 0.75 | 1.28 | Stem loop 2 | 6 |
| L2_e7_67 | 4226 | 1.54 | 1.55 | 0.76 | 1.28 | Stem loop 2 | 7 |
| L2_e7_24 | 4183 | 1.88 | 1.22 | 0.79 | 1.30 | Stem loop 2 | 7 |
| L2_e6_13 | 4072 | 1.73 | 1.44 | 0.73 | 1.30 | Stem loop 2 | 6 |
| L2_e5_44 | 4003 | 1.91 | 1.18 | 0.81 | 1.30 | Stem loop 2 | 5 |
| L2_e6_66 | 4125 | 1.60 | 1.70 | 0.61 | 1.30 | Stem loop 2 | 6 |
| L2_e7_64 | 4223 | 1.80 | 1.44 | 0.67 | 1.30 | Stem loop 2 | 7 |
| L2_e4_29 | 3913 | 1.54 | 1.56 | 0.82 | 1.30 | Stem loop 2 | 4 |
| L2_e7_68 | 4227 | 1.96 | 1.20 | 0.76 | 1.31 | Stem loop 2 | 7 |
| L2_e8_24 | 4258 | 1.90 | 1.05 | 0.98 | 1.31 | Stem loop 2 | 8 |
| L2_e6_4 | 4063 | 2.03 | 1.25 | 0.67 | 1.32 | Stem loop 2 | 6 |
| L2_e5_6 | 3965 | 2.16 | 1.26 | 0.54 | 1.32 | Stem loop 2 | 5 |
| L2_e7_69 | 4228 | 2.12 | 1.23 | 0.61 | 1.32 | Stem loop 2 | 7 |
| L2_e7_45 | 4204 | 1.36 | 1.84 | 0.76 | 1.32 | Stem loop 2 | 7 |
| L2_e6_55 | 4114 | 1.91 | 1.31 | 0.76 | 1.33 | Stem loop 2 | 6 |
| L2_e7_65 | 4224 | 1.57 | 1.56 | 0.85 | 1.33 | Stem loop 2 | 7 |
| L2_e8_15 | 4249 | 1.61 | 1.60 | 0.78 | 1.33 | Stem loop 2 | 8 |
| L2_e6_99 | 4158 | 2.37 | 0.88 | 0.74 | 1.33 | Stem loop 2 | 6 |
| L2_e5_3 | 3962 | 2.19 | 1.35 | 0.46 | 1.34 | Stem loop 2 | 5 |
| L2_e6_12 | 4071 | 1.65 | 1.49 | 0.87 | 1.34 | Stem loop 2 | 6 |
| L2_e5_66 | 4025 | 1.99 | 1.33 | 0.70 | 1.34 | Stem loop 2 | 5 |
| L2_e6_41 | 4100 | 2.06 | 1.22 | 0.76 | 1.35 | Stem loop 2 | 6 |
| L2_e6_78 | 4137 | 1.74 | 1.61 | 0.69 | 1.35 | Stem loop 2 | 6 |
| L2_e4_61 | 3945 | 1.33 | 1.72 | 1.00 | 1.35 | Stem loop 2 | 4 |
| L2_e6_63 | 4122 | 1.60 | 1.58 | 0.89 | 1.35 | Stem loop 2 | 6 |
| L2_e7_74 | 4233 | 2.19 | 1.15 | 0.72 | 1.35 | Stem loop 2 | 7 |
| L2_e7_51 | 4210 | 2.67 | 0.98 | 0.41 | 1.35 | Stem loop 2 | 7 |
| L2_e6_88 | 4147 | 1.85 | 1.63 | 0.59 | 1.35 | Stem loop 2 | 6 |
| L2_e4_22 | 3906 | 1.36 | 1.86 | 0.85 | 1.36 | Stem loop 2 | 4 |
| L2_e6_62 | 4121 | 1.60 | 1.80 | 0.67 | 1.36 | Stem loop 2 | 6 |
| L2_e7_72 | 4231 | 1.92 | 1.52 | 0.62 | 1.36 | Stem loop 2 | 7 |
| L2_e5_52 | 4011 | 1.68 | 1.65 | 0.75 | 1.36 | Stem loop 2 | 5 |
| L2_e6_21 | 4080 | 2.21 | 1.17 | 0.70 | 1.36 | Stem loop 2 | 6 |
| L2_e7_70 | 4229 | 2.12 | 1.25 | 0.71 | 1.36 | Stem loop 2 | 7 |
| L2_e8_16 | 4250 | 2.22 | 1.47 | 0.40 | 1.36 | Stem loop 2 | 8 |
| L2_e7_35 | 4194 | 1.75 | 1.76 | 0.59 | 1.36 | Stem loop 2 | 7 |
| L2_e7_53 | 4212 | 2.09 | 1.30 | 0.70 | 1.37 | Stem loop 2 | 7 |
| L2_e5_19 | 3978 | 2.04 | 1.16 | 0.90 | 1.37 | Stem loop 2 | 5 |
| L2_e5_93 | 4052 | 2.16 | 1.29 | 0.66 | 1.37 | Stem loop 2 | 5 |
| L2_e6_9 | 4068 | 2.08 | 1.26 | 0.77 | 1.37 | Stem loop 2 | 6 |
| L2_e5_50 | 4009 | 1.84 | 1.46 | 0.80 | 1.37 | Stem loop 2 | 5 |
| L2_e6_40 | 4099 | 2.34 | 1.22 | 0.55 | 1.37 | Stem loop 2 | 6 |
| L2_e7_2 | 4161 | 1.80 | 1.68 | 0.62 | 1.37 | Stem loop 2 | 7 |
| L2_e7_47 | 4206 | 2.01 | 1.36 | 0.74 | 1.37 | Stem loop 2 | 7 |
| L2_e6_24 | 4083 | 2.08 | 1.43 | 0.60 | 1.37 | Stem loop 2 | 6 |
| L2_e6_75 | 4134 | 1.65 | 1.66 | 0.81 | 1.37 | Stem loop 2 | 6 |
| L2_e7_61 | 4220 | 2.12 | 1.30 | 0.69 | 1.37 | Stem loop 2 | 7 |
| L2_e7_7 | 4166 | 1.60 | 1.81 | 0.72 | 1.38 | Stem loop 2 | 7 |
| L2_e5_21 | 3980 | 2.43 | 1.00 | 0.70 | 1.38 | Stem loop 2 | 5 |
| L2_e6_25 | 4084 | 1.98 | 1.46 | 0.71 | 1.38 | Stem loop 2 | 6 |
| L2_e6_27 | 4086 | 2.02 | 1.38 | 0.75 | 1.38 | Stem loop 2 | 6 |
| L2_e6_54 | 4113 | 2.04 | 1.47 | 0.64 | 1.38 | Stem loop 2 | 6 |
| L2_e4_6 | 3890 | 1.69 | 1.56 | 0.90 | 1.38 | Stem loop 2 | 4 |
| L2_e4_63 | 3947 | 2.08 | 1.39 | 0.67 | 1.38 | Stem loop 2 | 4 |
| L2_e6_59 | 4118 | 1.90 | 1.41 | 0.84 | 1.38 | Stem loop 2 | 6 |
| L2_e7_13 | 4172 | 2.34 | 1.34 | 0.48 | 1.39 | Stem loop 2 | 7 |
| L2_e7_15 | 4174 | 2.17 | 1.13 | 0.87 | 1.39 | Stem loop 2 | 7 |
| L2_e7_59 | 4218 | 1.77 | 1.73 | 0.67 | 1.39 | Stem loop 2 | 7 |
| L2_e5_45 | 4004 | 1.64 | 1.64 | 0.89 | 1.39 | Stem loop 2 | 5 |
| L2_e7_66 | 4225 | 1.68 | 1.63 | 0.87 | 1.39 | Stem loop 2 | 7 |
| L2_e7_39 | 4198 | 2.34 | 1.20 | 0.63 | 1.39 | Stem loop 2 | 7 |
| L2_e8_1 | 4235 | 1.94 | 1.62 | 0.61 | 1.39 | Stem loop 2 | 8 |
| L2_e7_36 | 4195 | 2.02 | 1.40 | 0.77 | 1.40 | Stem loop 2 | 7 |
| L2_e5_65 | 4024 | 2.19 | 1.25 | 0.75 | 1.40 | Stem loop 2 | 5 |
| L2_e5_49 | 4008 | 2.17 | 1.28 | 0.74 | 1.40 | Stem loop 2 | 5 |
| L2_e6_19 | 4078 | 1.65 | 1.78 | 0.77 | 1.40 | Stem loop 2 | 6 |
| L2_e7_19 | 4178 | 2.08 | 1.24 | 0.90 | 1.41 | Stem loop 2 | 7 |
| L2_e7_8 | 4167 | 2.00 | 1.48 | 0.74 | 1.41 | Stem loop 2 | 7 |
| L2_e7_41 | 4200 | 2.04 | 1.42 | 0.77 | 1.41 | Stem loop 2 | 7 |
| L2_e8_8 | 4242 | 2.30 | 1.45 | 0.48 | 1.41 | Stem loop 2 | 8 |
| L2_e6_15 | 4074 | 2.14 | 1.46 | 0.64 | 1.41 | Stem loop 2 | 6 |
| L2_e7_44 | 4203 | 2.60 | 0.84 | 0.79 | 1.41 | Stem loop 2 | 7 |
| L2_e6_5 | 4064 | 2.26 | 1.27 | 0.71 | 1.41 | Stem loop 2 | 6 |
| L2_e6_69 | 4128 | 2.11 | 1.47 | 0.67 | 1.42 | Stem loop 2 | 6 |
| L2_e6_2 | 4061 | 1.79 | 1.72 | 0.74 | 1.42 | Stem loop 2 | 6 |
| L2_e5_86 | 4045 | 1.64 | 1.90 | 0.70 | 1.42 | Stem loop 2 | 5 |
| L2_e6_97 | 4156 | 2.36 | 1.37 | 0.53 | 1.42 | Stem loop 2 | 6 |
| L2_e4_3 | 3887 | 1.99 | 1.45 | 0.83 | 1.42 | Stem loop 2 | 4 |
| L2_e3_25 | 3884 | 2.05 | 1.20 | 1.01 | 1.42 | Stem loop 2 | 3 |
| L2_e3_12 | 3871 | 2.18 | 1.26 | 0.83 | 1.42 | Stem loop 2 | 3 |
| L2_e6_49 | 4108 | 2.28 | 1.38 | 0.62 | 1.43 | Stem loop 2 | 6 |
| L2_e5_55 | 4014 | 1.95 | 1.15 | 1.18 | 1.43 | Stem loop 2 | 5 |
| L2_e5_29 | 3988 | 1.87 | 1.47 | 0.94 | 1.43 | Stem loop 2 | 5 |
| L2_e5_25 | 3984 | 1.75 | 1.59 | 0.95 | 1.43 | Stem loop 2 | 5 |
| L2_e6_65 | 4124 | 2.74 | 0.71 | 0.83 | 1.43 | Stem loop 2 | 6 |
| L2_e7_52 | 4211 | 1.97 | 1.75 | 0.57 | 1.43 | Stem loop 2 | 7 |
| L2_e5_99 | 4058 | 2.21 | 1.36 | 0.73 | 1.43 | Stem loop 2 | 5 |
| L2_e5_80 | 4039 | 2.19 | 1.28 | 0.84 | 1.44 | Stem loop 2 | 5 |
| L2_e7_42 | 4201 | 2.09 | 1.54 | 0.68 | 1.44 | Stem loop 2 | 7 |
| L2_e7_17 | 4176 | 2.06 | 1.52 | 0.73 | 1.44 | Stem loop 2 | 7 |
| L2_e6_39 | 4098 | 2.22 | 1.41 | 0.70 | 1.44 | Stem loop 2 | 6 |
| L2_e7_27 | 4186 | 1.74 | 1.69 | 0.91 | 1.45 | Stem loop 2 | 7 |
| L2_e7_62 | 4221 | 2.14 | 1.38 | 0.83 | 1.45 | Stem loop 2 | 7 |
| L2_e5_68 | 4027 | 2.06 | 1.31 | 0.98 | 1.45 | Stem loop 2 | 5 |
| L2_e6_98 | 4157 | 1.85 | 1.78 | 0.73 | 1.45 | Stem loop 2 | 6 |
| L2_e5_96 | 4055 | 2.66 | 1.06 | 0.64 | 1.45 | Stem loop 2 | 5 |
| L2_e6_73 | 4132 | 2.61 | 1.20 | 0.55 | 1.45 | Stem loop 2 | 6 |
| L2_e5_46 | 4005 | 2.41 | 1.16 | 0.81 | 1.46 | Stem loop 2 | 5 |
| L2_e6_29 | 4088 | 2.01 | 1.73 | 0.63 | 1.46 | Stem loop 2 | 6 |
| L2_e5_41 | 4000 | 1.98 | 1.59 | 0.81 | 1.46 | Stem loop 2 | 5 |
| L2_e6_30 | 4089 | 1.79 | 1.90 | 0.70 | 1.46 | Stem loop 2 | 6 |
| L2_e7_33 | 4192 | 1.77 | 1.71 | 0.91 | 1.46 | Stem loop 2 | 7 |
| L2_e5_79 | 4038 | 2.36 | 1.41 | 0.64 | 1.47 | Stem loop 2 | 5 |
| L2_e7_11 | 4170 | 2.26 | 1.56 | 0.61 | 1.48 | Stem loop 2 | 7 |
| L2_e8_21 | 4255 | 1.96 | 1.63 | 0.84 | 1.48 | Stem loop 2 | 8 |
| L2_e5_81 | 4040 | 2.12 | 1.60 | 0.72 | 1.48 | Stem loop 2 | 5 |
| L2_e5_9 | 3968 | 1.80 | 1.76 | 0.88 | 1.48 | Stem loop 2 | 5 |
| L2_e5_28 | 3987 | 1.80 | 1.72 | 0.94 | 1.48 | Stem loop 2 | 5 |
| L2_e6_1 | 4060 | 2.27 | 1.54 | 0.66 | 1.49 | Stem loop 2 | 6 |
| L2_e5_13 | 3972 | 1.81 | 1.68 | 0.99 | 1.49 | Stem loop 2 | 5 |
| L2_e3_19 | 3878 | 2.00 | 1.48 | 1.00 | 1.49 | Stem loop 2 | 3 |
| L2_e7_5 | 4164 | 2.54 | 1.31 | 0.63 | 1.49 | Stem loop 2 | 7 |
| L2_e8_17 | 4251 | 2.43 | 1.33 | 0.72 | 1.49 | Stem loop 2 | 8 |
| L2_e4_70 | 3954 | 1.96 | 1.55 | 0.98 | 1.50 | Stem loop 2 | 4 |
| L2_e7_3 | 4162 | 2.29 | 1.34 | 0.88 | 1.50 | Stem loop 2 | 7 |
| L2_e7_14 | 4173 | 2.25 | 1.53 | 0.74 | 1.50 | Stem loop 2 | 7 |
| L2_e5_47 | 4006 | 2.25 | 1.43 | 0.83 | 1.50 | Stem loop 2 | 5 |
| L2_e6_74 | 4133 | 1.99 | 1.87 | 0.66 | 1.51 | Stem loop 2 | 6 |
| L2_e6_28 | 4087 | 2.39 | 1.33 | 0.80 | 1.51 | Stem loop 2 | 6 |
| L2_e7_34 | 4193 | 2.17 | 1.79 | 0.57 | 1.5 | Stem loop 2 | 7 |
| L2_e4_71 | 3955 | 1.97 | 1.6 | 0.95 | 1.5 | Stem loop 2 | 4 |
| L2_e7_43 | 4202 | 2.45 | 1.50 | 0.58 | 1.51 | Stem loop 2 | 7 |
| L2_e7_25 | 4184 | 2.36 | 1.25 | 0.91 | 1.51 | Stem loop 2 | 7 |
| L2_e6_92 | 4151 | 2.04 | 1.69 | 0.80 | 1.51 | Stem loop 2 | 6 |
| L2_e7_4 | 4163 | 2.10 | 1.64 | 0.79 | 1.51 | Stem loop 2 | 7 |
| L2_e4_56 | 3940 | 1.78 | 1.91 | 0.84 | 1.51 | Stem loop 2 | 4 |
| L2_e4_17 | 3901 | 2.10 | 1.66 | 0.77 | 1.5 | Stem loop 2 | 4 |
| L2_e6_47 | 4106 | 2.31 | 1.43 | 0.80 | 1.51 | Stem loop 2 | 6 |
| L2_e6_18 | 4077 | 2.24 | 1.66 | 0.65 | 1.51 | Stem loop 2 | 6 |
| L2_e4_47 | 3931 | 2.17 | 1.39 | 0.99 | 1.51 | Stem loop 2 | 4 |
| L2_e8_13 | 4247 | 2.31 | 1.36 | 0.88 | 1.52 | Stem loop 2 | 8 |
| L2_e4_64 | 3948 | 1.75 | 1.80 | 1.00 | 1.52 | Stem loop 2 | 4 |
| L2_e5_91 | 4050 | 1.79 | 1.95 | 0.82 | 1.52 | Stem loop 2 | 5 |
| L2_e4_52 | 3936 | 2.38 | 1.50 | 0.67 | 1.52 | Stem loop 2 | 4 |
| L2_e6_33 | 4092 | 2.72 | 1.06 | 0.78 | 1.52 | Stem loop 2 | 6 |
| L2_e5_74 | 4033 | 2.11 | 1.71 | 0.75 | 1.52 | Stem loop 2 | 5 |
| L2_e5_94 | 4053 | 1.97 | 1.80 | 0.79 | 1.52 | Stem loop 2 | 5 |
| L2_e6_90 | 4149 | 2.60 | 1.19 | 0.77 | 1.52 | Stem loop 2 | 6 |
| L2_e7_26 | 4185 | 2.18 | 1.68 | 0.70 | 1.52 | Stem loop 2 | 7 |
| T_e1b_12 | 4279 | 2.44 | 1.19 | 0.93 | 1.52 | Tetraloop | 1 |
| L2_e6_11 | 4070 | 2.44 | 1.37 | 0.77 | 1.53 | Stem loop 2 | 6 |
| L2_e5_51 | 4010 | 2.15 | 1.76 | 0.67 | 1.53 | Stem loop 2 | 5 |
| L2_e4_57 | 3941 | 2.19 | 1.57 | 0.82 | 1.53 | Stem loop 2 | 4 |
| L2_e5_42 | 4001 | 1.93 | 1.86 | 0.79 | 1.53 | Stem loop 2 | 5 |
| L2_e7_57 | 4216 | 2.31 | 1.6 | 0.67 | 1.53 | Stem loop 2 | 7 |
| L2_e4_58 | 3942 | 2.10 | 1.38 | 1.12 | 1.53 | Stem loop 2 | 4 |
| L2_e6_3 | 4062 | 2.74 | 1.22 | 0.64 | 1.53 | Stem loop 2 | 6 |
| L2_e6_20 | 4079 | 2.27 | 1.55 | 0.77 | 1.53 | Stem loop 2 | 6 |
| L2_e6_61 | 4120 | 2.20 | 1.57 | 0.83 | 1.54 | Stem loop 2 | 6 |
| L2_e6_43 | 4102 | 2.07 | 1.69 | 0.85 | 1.54 | Stem loop 2 | 6 |
| L2_e8_19 | 4253 | 2.11 | 1.92 | 0.58 | 1.54 | Stem loop 2 | 8 |
| L2_e6_68 | 4127 | 2.71 | 1.07 | 0.83 | 1.54 | Stem loop 2 | 6 |
| L2_e6_26 | 4085 | 2.33 | 1.40 | 0.89 | 1.54 | Stem loop 2 | 6 |
| L2_e8_25 | 4259 | 1.82 | 2.15 | 0.65 | 1.54 | Stem loop 2 | 8 |
| L2_e5_92 | 4051 | 2.43 | 1.34 | 0.86 | 1.54 | Stem loop 2 | 5 |
| L2_e5_82 | 4041 | 1.98 | 1.95 | 0.71 | 1.55 | Stem loop 2 | 5 |
| L2_e6_70 | 4129 | 2.20 | 1.65 | 0.80 | 1.55 | Stem loop 2 | 6 |
| L2_e7_38 | 4197 | 2.33 | 1.65 | 0.67 | 1.55 | Stem loop 2 | 7 |
| L2_e5_54 | 4013 | 2.03 | 1.70 | 0.92 | 1.55 | Stem loop 2 | 5 |
| L2_e7_75 | 4234 | 2.38 | 1.49 | 0.79 | 1.55 | Stem loop 2 | 7 |
| L2_e5_75 | 4034 | 2.09 | 1.76 | 0.81 | 1.55 | Stem loop 2 | 5 |
| L2_e4_9 | 3893 | 2.16 | 1.52 | 0.99 | 1.56 | Stem loop 2 | 4 |
| L2_e5_33 | 3992 | 2.18 | 1.49 | 1.01 | 1.56 | Stem loop 2 | 5 |
| L2_e3_9 | 3868 | 1.92 | 1.84 | 0.92 | 1.56 | Stem loop 2 | 3 |
| L2_e6_37 | 4096 | 2.74 | 1.19 | 0.75 | 1.56 | Stem loop 2 | 6 |
| L2_e7_20 | 4179 | 2.03 | 1.91 | 0.74 | 1.56 | Stem loop 2 | 7 |
| L2_e4_12 | 3896 | 2.23 | 1.64 | 0.83 | 1.57 | Stem loop 2 | 4 |
| L2_e6_83 | 4142 | 2.24 | 1.51 | 0.94 | 1.57 | Stem loop 2 | 6 |
| L2_e4_30 | 3914 | 2.63 | 1.41 | 0.66 | 1.57 | Stem loop 2 | 4 |
| L2_e6_51 | 4110 | 2.52 | 1.56 | 0.63 | 1.57 | Stem loop 2 | 6 |
| L2_e4_45 | 3929 | 1.98 | 1.76 | 0.97 | 1.57 | Stem loop 2 | 4 |
| L2_e4_11 | 3895 | 2.62 | 1.31 | 0.78 | 1.57 | Stem loop 2 | 4 |
| L2_e6_93 | 4152 | 2.33 | 1.59 | 0.79 | 1.57 | Stem loop 2 | 6 |
| L2_e6_31 | 4090 | 2.71 | 1.20 | 0.80 | 1.57 | Stem loop 2 | 6 |
| L2_e4_14 | 3898 | 2.04 | 1.62 | 1.05 | 1.57 | Stem loop 2 | 4 |
| L2_e6_17 | 4076 | 2.12 | 1.65 | 0.94 | 1.57 | Stem loop 2 | 6 |
| L2_e6_16 | 4075 | 2.07 | 1.77 | 0.88 | 1.57 | Stem loop 2 | 6 |
| L2_e7_71 | 4230 | 2.21 | 1.87 | 0.64 | 1.57 | Stem loop 2 | 7 |
| L2_e4_42 | 3926 | 2.50 | 1.35 | 0.88 | 1.57 | Stem loop 2 | 4 |
| L2_e6_48 | 4107 | 2.39 | 1.21 | 1.13 | 1.58 | Stem loop 2 | 6 |
| L2_e6_79 | 4138 | 2.27 | 1.55 | 0.90 | 1.58 | Stem loop 2 | 6 |
| L2_e5_84 | 4043 | 2.63 | 1.49 | 0.61 | 1.58 | Stem loop 2 | 5 |
| L2_e3_16 | 3875 | 2.23 | 1.64 | 0.87 | 1.58 | Stem loop 2 | 3 |
| L2_e4_20 | 3904 | 2.49 | 1.16 | 1.08 | 1.58 | Stem loop 2 | 4 |
| L2_e5_38 | 3997 | 2.62 | 1.28 | 0.85 | 1.58 | Stem loop 2 | 5 |
| L2_e4_10 | 3894 | 1.95 | 1.94 | 0.86 | 1.58 | Stem loop 2 | 4 |
| L2_e6_23 | 4082 | 2.28 | 1.54 | 0.93 | 1.58 | Stem loop 2 | 6 |
| dnr6 | 4351 | 2.44 | 1.02 | 1.30 | 1.59 | Tetraloop | 1 |
| L2_e3_23 | 3882 | 2.35 | 1.58 | 0.84 | 1.59 | Stem loop 2 | 3 |
| L2_e6_67 | 4126 | 2.35 | 1.65 | 0.77 | 1.59 | Stem loop 2 | 6 |
| L2_e8_2 | 4236 | 1.84 | 1.79 | 1.14 | 1.59 | Stem loop 2 | 8 |
| L2_e5_1 | 3960 | 2.26 | 1.52 | 0.99 | 1.59 | Stem loop 2 | 5 |
| L2_e5_72 | 4031 | 2.50 | 1.58 | 0.69 | 1.59 | Stem loop 2 | 5 |
| L2_e6_82 | 4141 | 2.13 | 1.93 | 0.72 | 1.59 | Stem loop 2 | 6 |
| L2_e6_89 | 4148 | 2.80 | 0.94 | 1.05 | 1.59 | Stem loop 2 | 6 |
| L2_e5_85 | 4044 | 2.38 | 1.66 | 0.75 | 1.59 | Stem loop 2 | 5 |
| L2_e4_18 | 3902 | 2.46 | 1.42 | 0.91 | 1.60 | Stem loop 2 | 4 |
| L2_e8_3 | 4237 | 2.22 | 1.59 | 0.98 | 1.60 | Stem loop 2 | 8 |
| L2_e7_18 | 4177 | 2.08 | 1.82 | 0.90 | 1.60 | Stem loop 2 | 7 |
| L2_e6_80 | 4139 | 2.31 | 1.74 | 0.76 | 1.60 | Stem loop 2 | 6 |
| L2_e4_74 | 3958 | 2.49 | 1.52 | 0.80 | 1.60 | Stem loop 2 | 4 |
| L2_e5_67 | 4026 | 1.96 | 1.92 | 0.94 | 1.61 | Stem loop 2 | 5 |
| L2_e4_60 | 3944 | 2.37 | 1.61 | 0.85 | 1.61 | Stem loop 2 | 4 |
| L2_e5_35 | 3994 | 2.25 | 1.62 | 0.96 | 1.61 | Stem loop 2 | 5 |
| L2_e5_87 | 4046 | 2.03 | 2.08 | 0.71 | 1.61 | Stem loop 2 | 5 |
| L2_e5_69 | 4028 | 2.92 | 1.12 | 0.80 | 1.61 | Stem loop 2 | 5 |
| L2_e6_14 | 4073 | 2.30 | 1.80 | 0.74 | 1.61 | Stem loop 2 | 6 |
| L2_e6_50 | 4109 | 2.12 | 1.69 | 1.04 | 1.62 | Stem loop 2 | 6 |
| L2_e4_72 | 3956 | 2.46 | 1.56 | 0.83 | 1.62 | Stem loop 2 | 4 |
| L2_e5_77 | 4036 | 2.54 | 1.59 | 0.73 | 1.62 | Stem loop 2 | 5 |
| L2_e5_53 | 4012 | 2.32 | 1.75 | 0.79 | 1.62 | Stem loop 2 | 5 |
| L2_e6_87 | 4146 | 2.15 | 1.85 | 0.86 | 1.62 | Stem loop 2 | 6 |
| L2_e4_48 | 3932 | 1.86 | 2.13 | 0.87 | 1.62 | Stem loop 2 | 4 |
| L2_e7_21 | 4180 | 2.31 | 1.79 | 0.78 | 1.62 | Stem loop 2 | 7 |
| L2_e6_100 | 4159 | 2.33 | 1.79 | 0.75 | 1.62 | Stem loop 2 | 6 |
| L2_e4_15 | 3899 | 2.35 | 1.66 | 0.87 | 1.62 | Stem loop 2 | 4 |
| L2_e4_8 | 3892 | 2.42 | 1.86 | 0.60 | 1.63 | Stem loop 2 | 4 |
| L2_e3_20 | 3879 | 2.29 | 1.53 | 1.05 | 1.63 | Stem loop 2 | 3 |
| L2_e8_10 | 4244 | 2.43 | 1.47 | 0.98 | 1.63 | Stem loop 2 | 8 |
| L2_e4_75 | 3959 | 2.27 | 1.83 | 0.79 | 1.63 | Stem loop 2 | 4 |
| L2_e8_6 | 4240 | 2.38 | 1.93 | 0.58 | 1.63 | Stem loop 2 | 8 |
| L2_e6_45 | 4104 | 2.62 | 1.52 | 0.77 | 1.64 | Stem loop 2 | 6 |
| L2_e4_37 | 3921 | 2.40 | 1.69 | 0.82 | 1.64 | Stem loop 2 | 4 |
| L2_e6_58 | 4117 | 2.16 | 2.10 | 0.65 | 1.64 | Stem loop 2 | 6 |
| L2_e8_12 | 4246 | 2.18 | 2.04 | 0.70 | 1.64 | Stem loop 2 | 8 |
| L2_e3_14 | 3873 | 2.43 | 1.54 | 0.95 | 1.64 | Stem loop 2 | 3 |
| L2_e5_37 | 3996 | 2.42 | 1.75 | 0.76 | 1.64 | Stem loop 2 | 5 |
| L2_e6_35 | 4094 | 2.90 | 1.32 | 0.71 | 1.64 | Stem loop 2 | 6 |
| L2_e5_48 | 4007 | 2.92 | 1.29 | 0.73 | 1.64 | Stem loop 2 | 5 |
| L2_e5_31 | 3990 | 2.71 | 1.38 | 0.86 | 1.65 | Stem loop 2 | 5 |
| T_e3_13 | 4336 | 3.15 | 0.78 | 1.02 | 1.65 | Tetraloop | 3 |
| L2_e5_90 | 4049 | 2.64 | 1.62 | 0.70 | 1.65 | Stem loop 2 | 5 |
| L2_e7_73 | 4232 | 2.87 | 1.56 | 0.54 | 1.66 | Stem loop 2 | 7 |
| L2_e5_61 | 4020 | 2.68 | 1.26 | 1.03 | 1.66 | Stem loop 2 | 5 |
| L2_e5_59 | 4018 | 2.89 | 1.34 | 0.74 | 1.66 | Stem loop 2 | 5 |
| L2_e5_20 | 3979 | 2.26 | 1.81 | 0.90 | 1.66 | Stem loop 2 | 5 |
| L2_e4_27 | 3911 | 2.48 | 1.88 | 0.61 | 1.66 | Stem loop 2 | 4 |
| L2_e3_11 | 3870 | 2.12 | 1.99 | 0.89 | 1.66 | Stem loop 2 | 3 |
| L2_e6_32 | 4091 | 2.59 | 1.87 | 0.54 | 1.67 | Stem loop 2 | 6 |
| L2_e7_31 | 4190 | 2.43 | 1.69 | 0.89 | 1.67 | Stem loop 2 | 7 |
| L2_e7_63 | 4222 | 2.36 | 2.04 | 0.61 | 1.67 | Stem loop 2 | 7 |
| L2_e4_46 | 3930 | 2.64 | 1.40 | 0.97 | 1.67 | Stem loop 2 | 4 |
| L2_e5_57 | 4016 | 2.39 | 1.67 | 0.97 | 1.67 | Stem loop 2 | 5 |
| L2_e3_18 | 3877 | 2.52 | 1.52 | 0.98 | 1.68 | Stem loop 2 | 3 |
| L2_e5_88 | 4047 | 2.44 | 1.68 | 0.92 | 1.68 | Stem loop 2 | 5 |
| L2_e4_51 | 3935 | 2.74 | 1.49 | 0.82 | 1.68 | Stem loop 2 | 4 |
| L2_e4_69 | 3953 | 2.00 | 2.16 | 0.88 | 1.68 | Stem loop 2 | 4 |
| L2_e4_43 | 3927 | 2.16 | 2.07 | 0.82 | 1.68 | Stem loop 2 | 4 |
| L2_e4_2 | 3886 | 2.59 | 1.75 | 0.70 | 1.68 | Stem loop 2 | 4 |
| L2_e5_43 | 4002 | 2.77 | 1.68 | 0.60 | 1.69 | Stem loop 2 | 5 |
| L2_e8_4 | 4238 | 2.37 | 1.58 | 1.10 | 1.69 | Stem loop 2 | 8 |
| L2_e4_49 | 3933 | 2.42 | 1.74 | 0.91 | 1.69 | Stem loop 2 | 4 |
| L2_e3_24 | 3883 | 2.53 | 1.29 | 1.26 | 1.69 | Stem loop 2 | 3 |
| L2_e5_16 | 3975 | 2.94 | 1.56 | 0.58 | 1.69 | Stem loop 2 | 5 |
| L2_e7_28 | 4187 | 2.41 | 1.88 | 0.79 | 1.70 | Stem loop 2 | 7 |
| L2_e6_44 | 4103 | 2.78 | 1.52 | 0.78 | 1.70 | Stem loop 2 | 6 |
| L2_e7_55 | 4214 | 2.93 | 1.44 | 0.71 | 1.70 | Stem loop 2 | 7 |
| L2_e4_25 | 3909 | 2.25 | 1.86 | 0.98 | 1.70 | Stem loop 2 | 4 |
| L2_e6_46 | 4105 | 2.72 | 1.52 | 0.85 | 1.70 | Stem loop 2 | 6 |
| L2_e5_34 | 3993 | 2.38 | 1.88 | 0.83 | 1.70 | Stem loop 2 | 5 |
| L2_e5_62 | 4021 | 2.29 | 1.98 | 0.83 | 1.70 | Stem loop 2 | 5 |
| L2_e4_35 | 3919 | 1.74 | 2.40 | 0.97 | 1.70 | Stem loop 2 | 4 |
| L2_e4_39 | 3923 | 2.49 | 1.66 | 0.96 | 1.70 | Stem loop 2 | 4 |
| M4_GC | 4358 | 2.99 | 1.32 | 0.80 | 1.71 | none | 0 |
| L2_e7_54 | 4213 | 2.41 | 1.86 | 0.85 | 1.71 | Stem loop 2 | 7 |
| L2_e4_33 | 3917 | 2.73 | 1.62 | 0.78 | 1.71 | Stem loop 2 | 4 |
| L2_e4_41 | 3925 | 3.04 | 1.31 | 0.78 | 1.71 | Stem loop 2 | 4 |
| L2_e5_4 | 3963 | 2.95 | 1.40 | 0.78 | 1.71 | Stem loop 2 | 5 |
| L2_e8_18 | 4252 | 2.92 | 1.18 | 1.02 | 1.71 | Stem loop 2 | 8 |
| L2_e7_40 | 4199 | 2.71 | 1.62 | 0.81 | 1.71 | Stem loop 2 | 7 |
| L2_e3_5 | 3864 | 2.88 | 1.28 | 0.98 | 1.71 | Stem loop 2 | 3 |
| L2_e3_2 | 3861 | 2.70 | 1.85 | 0.60 | 1.72 | Stem loop 2 | 3 |
| L2_e3_15 | 3874 | 2.66 | 2.10 | 0.40 | 1.72 | Stem loop 2 | 3 |
| L2_e3_13 | 3872 | 2.17 | 2.32 | 0.67 | 1.72 | Stem loop 2 | 3 |
| L2_e5_17 | 3976 | 2.65 | 1.79 | 0.73 | 1.72 | Stem loop 2 | 5 |
| L2_e5_89 | 4048 | 2.87 | 1.64 | 0.65 | 1.72 | Stem loop 2 | 5 |
| L2_e8_7 | 4241 | 2.86 | 1.69 | 0.62 | 1.73 | Stem loop 2 | 8 |
| L2_e4_7 | 3891 | 2.37 | 1.92 | 0.89 | 1.73 | Stem loop 2 | 4 |
| T_e3_14 | 4337 | 3.22 | 1.13 | 0.84 | 1.73 | Tetraloop | 3 |
| T_e3_3 | 4326 | 3.39 | 0.91 | 0.90 | 1.73 | Tetraloop | 3 |
| L2_e4_68 | 3952 | 2.55 | 1.80 | 0.85 | 1.73 | Stem loop 2 | 4 |
| L2_e5_98 | 4057 | 2.25 | 2.04 | 0.92 | 1.74 | Stem loop 2 | 5 |
| L2_e4_26 | 3910 | 2.10 | 2.10 | 1.01 | 1.74 | Stem loop 2 | 4 |
| L2_e6_72 | 4131 | 2.95 | 1.59 | 0.68 | 1.74 | Stem loop 2 | 6 |
| L2_e4_40 | 3924 | 2.06 | 2.14 | 1.03 | 1.74 | Stem loop 2 | 4 |
| L2_e5_18 | 3977 | 2.95 | 1.36 | 0.92 | 1.74 | Stem loop 2 | 5 |
| L2_e6_53 | 4112 | 2.64 | 1.84 | 0.75 | 1.74 | Stem loop 2 | 6 |
| L2_e6_7 | 4066 | 1.88 | 2.40 | 0.95 | 1.75 | Stem loop 2 | 6 |
| L2_e5_71 | 4030 | 2.88 | 1.56 | 0.81 | 1.75 | Stem loop 2 | 5 |
| L2_e5_39 | 3998 | 2.61 | 1.86 | 0.79 | 1.75 | Stem loop 2 | 5 |
| L2_e6_36 | 4095 | 2.85 | 1.63 | 0.78 | 1.75 | Stem loop 2 | 6 |
| L2_e6_96 | 4155 | 2.66 | 1.71 | 0.89 | 1.75 | Stem loop 2 | 6 |
| L2_e6_22 | 4081 | 2.67 | 1.80 | 0.80 | 1.75 | Stem loop 2 | 6 |
| M4_CG | 4359 | 2.73 | 1.43 | 1.10 | 1.75 | none | 0 |
| L2_e4_13 | 3897 | 2.68 | 1.75 | 0.85 | 1.76 | Stem loop 2 | 4 |
| L2_e5_100 | 4059 | 2.21 | 2.20 | 0.87 | 1.76 | Stem loop 2 | 5 |
| T_e3_21 | 4344 | 3.36 | 0.96 | 0.94 | 1.76 | Tetraloop | 3 |
| L2_e5_97 | 4056 | 3.03 | 1.30 | 0.94 | 1.76 | Stem loop 2 | 5 |
| L2_e5_5 | 3964 | 2.87 | 1.51 | 0.89 | 1.76 | Stem loop 2 | 5 |
| L2_e6_91 | 4150 | 1.87 | 2.42 | 1.00 | 1.76 | Stem loop 2 | 6 |
| L2_e5_36 | 3995 | 2.54 | 1.50 | 1.24 | 1.76 | Stem loop 2 | 5 |
| L2_e6_57 | 4116 | 2.55 | 1.86 | 0.88 | 1.76 | Stem loop 2 | 6 |
| L2_e5_14 | 3973 | 2.99 | 1.61 | 0.70 | 1.77 | Stem loop 2 | 5 |
| L2_e3_21 | 3880 | 3.01 | 1.37 | 0.92 | 1.77 | Stem loop 2 | 3 |
| L2_e5_8 | 3967 | 2.37 | 2.03 | 0.91 | 1.77 | Stem loop 2 | 5 |
| L2_e6_10 | 4069 | 2.34 | 2.04 | 0.93 | 1.77 | Stem loop 2 | 6 |
| L2_e4_28 | 3912 | 3.14 | 1.40 | 0.78 | 1.78 | Stem loop 2 | 4 |
| T_e2_9 | 4300 | 3.33 | 1.11 | 0.90 | 1.78 | Tetraloop | 2 |
| L2_e4_50 | 3934 | 2.80 | 1.64 | 0.91 | 1.78 | Stem loop 2 | 4 |
| L2_e4_34 | 3918 | 2.63 | 2.12 | 0.60 | 1.79 | Stem loop 2 | 4 |
| L2_e6_77 | 4136 | 2.16 | 2.54 | 0.66 | 1.79 | Stem loop 2 | 6 |
| L2_e6_52 | 4111 | 2.48 | 1.99 | 0.89 | 1.79 | Stem loop 2 | 6 |
| L2_e4_44 | 3928 | 3.21 | 1.40 | 0.76 | 1.79 | Stem loop 2 | 4 |
| L2_e4_54 | 3938 | 3.32 | 1.27 | 0.79 | 1.79 | Stem loop 2 | 4 |
| L2_e5_27 | 3986 | 2.56 | 1.94 | 0.88 | 1.79 | Stem loop 2 | 5 |
| L2_e5_95 | 4054 | 2.57 | 1.99 | 0.83 | 1.80 | Stem loop 2 | 5 |
| L2_e4_67 | 3951 | 2.22 | 2.48 | 0.70 | 1.80 | Stem loop 2 | 4 |
| L2_e5_15 | 3974 | 2.54 | 1.90 | 0.96 | 1.80 | Stem loop 2 | 5 |
| L2_e5_7 | 3966 | 3.04 | 1.56 | 0.81 | 1.80 | Stem loop 2 | 5 |
| T_e1b_14 | 4281 | 3.51 | 1.04 | 0.86 | 1.80 | Tetraloop | 1 |
| L2_e5_22 | 3981 | 2.64 | 1.99 | 0.80 | 1.81 | Stem loop 2 | 5 |
| L2_e7_50 | 4209 | 2.90 | 1.60 | 0.93 | 1.81 | Stem loop 2 | 7 |
| L2_e7_49 | 4208 | 2.43 | 2.17 | 0.84 | 1.81 | Stem loop 2 | 7 |
| T_e1b_15 | 4282 | 3.23 | 1.54 | 0.67 | 1.81 | Tetraloop | 1 |
| L2_e5_60 | 4019 | 2.61 | 1.58 | 1.27 | 1.82 | Stem loop 2 | 5 |
| T_e2_7 | 4298 | 3.36 | 1.14 | 0.97 | 1.83 | Tetraloop | 2 |
| L2_e3_17 | 3876 | 2.84 | 1.80 | 0.84 | 1.83 | Stem loop 2 | 3 |
| L2_e4_55 | 3939 | 2.50 | 2.22 | 0.76 | 1.83 | Stem loop 2 | 4 |
| L2_e4_38 | 3922 | 2.15 | 2.46 | 0.87 | 1.83 | Stem loop 2 | 4 |
| L2_e5_30 | 3989 | 2.64 | 2.10 | 0.75 | 1.83 | Stem loop 2 | 5 |
| T_e1a_1 | 4260 | 3.52 | 1.32 | 0.67 | 1.83 | Tetraloop | 1 |
| L2_e5_2 | 3961 | 2.95 | 1.97 | 0.58 | 1.83 | Stem loop 2 | 5 |
| L2_e6_42 | 4101 | 2.96 | 1.71 | 0.86 | 1.84 | Stem loop 2 | 6 |
| L2_e6_34 | 4093 | 2.46 | 2.08 | 0.99 | 1.84 | Stem loop 2 | 6 |
| L2_e4_62 | 3946 | 2.83 | 1.85 | 0.85 | 1.84 | Stem loop 2 | 4 |
| L2_e4_53 | 3937 | 2.67 | 1.90 | 0.95 | 1.84 | Stem loop 2 | 4 |
| L2_e3_4 | 3863 | 2.55 | 2.18 | 0.80 | 1.84 | Stem loop 2 | 3 |
| L2_e3_7 | 3866 | 2.47 | 2.18 | 0.89 | 1.85 | Stem loop 2 | 3 |
| L2_e6_84 | 4143 | 3.11 | 1.86 | 0.57 | 1.85 | Stem loop 2 | 6 |
| L2_e4_36 | 3920 | 3.35 | 1.32 | 0.88 | 1.85 | Stem loop 2 | 4 |
| L2_e5_63 | 4022 | 2.29 | 2.28 | 0.99 | 1.85 | Stem loop 2 | 5 |
| T_e3_8 | 4331 | 3.06 | 1.44 | 1.06 | 1.85 | Tetraloop | 3 |
| flip_U46A | 4357 | 3.23 | 1.24 | 1.09 | 1.85 | none | 0 |
| T_e1b_6 | 4273 | 3.88 | 0.75 | 0.94 | 1.86 | Tetraloop | 1 |
| L2_e5_58 | 4017 | 2.77 | 1.87 | 0.93 | 1.86 | Stem loop 2 | 5 |
| L2_e4_4 | 3888 | 2.49 | 1.92 | 1.16 | 1.86 | Stem loop 2 | 4 |
| L2_e6_71 | 4130 | 2.50 | 2.08 | 1.00 | 1.86 | Stem loop 2 | 6 |
| L2_e3_8 | 3867 | 2.75 | 1.87 | 0.96 | 1.86 | Stem loop 2 | 3 |
| L2_e5_83 | 4042 | 2.73 | 2.03 | 0.83 | 1.86 | Stem loop 2 | 5 |
| L2_e6_86 | 4145 | 2.63 | 2.03 | 0.93 | 1.86 | Stem loop 2 | 6 |
| L2_e5_56 | 4015 | 2.88 | 1.89 | 0.82 | 1.86 | Stem loop 2 | 5 |
| T_e2_19 | 4310 | 3.55 | 1.13 | 0.92 | 1.86 | Tetraloop | 2 |
| L2_e5_12 | 3971 | 2.94 | 1.73 | 0.92 | 1.87 | Stem loop 2 | 5 |
| T_e3_17 | 4340 | 3.27 | 1.27 | 1.06 | 1.87 | Tetraloop | 3 |
| L2_e5_78 | 4037 | 3.11 | 1.82 | 0.67 | 1.87 | Stem loop 2 | 5 |
| L2_e6_60 | 4119 | 3.08 | 1.57 | 0.96 | 1.87 | Stem loop 2 | 6 |
| T_e3_12 | 4335 | 3.46 | 1.16 | 1.00 | 1.87 | Tetraloop | 3 |
| T_e2_17 | 4308 | 3.14 | 1.44 | 1.04 | 1.87 | Tetraloop | 2 |
| L2_e5_10 | 3969 | 2.81 | 1.82 | 0.98 | 1.87 | Stem loop 2 | 5 |
| T_e1a_8 | 4267 | 3.60 | 1.00 | 1.02 | 1.87 | Tetraloop | 1 |
| L2_e4_59 | 3943 | 2.75 | 2.04 | 0.83 | 1.87 | Stem loop 2 | 4 |
| L2_e4_23 | 3907 | 2.71 | 2.06 | 0.87 | 1.88 | Stem loop 2 | 4 |
| L2_e4_73 | 3957 | 2.81 | 1.76 | 1.08 | 1.89 | Stem loop 2 | 4 |
| L2_e4_1 | 3885 | 3.08 | 1.63 | 0.99 | 1.90 | Stem loop 2 | 4 |
| L2_e6_64 | 4123 | 3.04 | 1.67 | 0.99 | 1.90 | Stem loop 2 | 6 |
| L2_e4_32 | 3916 | 3.01 | 2.03 | 0.70 | 1.91 | Stem loop 2 | 4 |
| T_e2_16 | 4307 | 2.52 | 2.11 | 1.11 | 1.92 | Tetraloop | 2 |
| T_e3_4 | 4327 | 3.37 | 1.54 | 0.85 | 1.92 | Tetraloop | 3 |
| L2_e4_66 | 3950 | 2.82 | 2.17 | 0.77 | 1.92 | Stem loop 2 | 4 |
| L2_e4_5 | 3889 | 3.02 | 1.84 | 0.92 | 1.93 | Stem loop 2 | 4 |
| T_e3_2 | 4325 | 3.22 | 1.56 | 1.01 | 1.93 | Tetraloop | 3 |
| L2_e4_65 | 3949 | 2.84 | 1.92 | 1.02 | 1.93 | Stem loop 2 | 4 |
| L2_e4_21 | 3905 | 2.81 | 1.84 | 1.15 | 1.94 | Stem loop 2 | 4 |
| L2_e5_73 | 4032 | 3.23 | 1.89 | 0.70 | 1.94 | Stem loop 2 | 5 |
| T_e3_24 | 4347 | 3.31 | 1.54 | 0.97 | 1.94 | Tetraloop | 3 |
| L2_e5_64 | 4023 | 3.24 | 1.80 | 0.78 | 1.94 | Stem loop 2 | 5 |
| L2_e3_10 | 3869 | 3.44 | 1.47 | 0.91 | 1.94 | Stem loop 2 | 3 |
| L2_e3_22 | 3881 | 2.91 | 1.89 | 1.04 | 1.94 | Stem loop 2 | 3 |
| L2_e5_70 | 4029 | 2.61 | 2.39 | 0.84 | 1.95 | Stem loop 2 | 5 |
| L2_e5_11 | 3970 | 3.03 | 1.87 | 0.99 | 1.96 | Stem loop 2 | 5 |
| T_e2_27 | 4318 | 3.06 | 1.63 | 1.20 | 1.96 | Tetraloop | 2 |
| L2_e5_40 | 3999 | 2.53 | 2.31 | 1.09 | 1.98 | Stem loop 2 | 5 |
| M4 | 4353 | 3.32 | 1.47 | 1.14 | 1.98 | none | 0 |
| L2_e4_24 | 3908 | 3.14 | 1.93 | 0.87 | 1.98 | Stem loop 2 | 4 |
| L2_e5_76 | 4035 | 3.28 | 1.85 | 0.82 | 1.98 | Stem loop 2 | 5 |
| T_e3_22 | 4345 | 3.52 | 1.39 | 1.05 | 1.99 | Tetraloop | 3 |
| L2_e4_31 | 3915 | 3.22 | 1.78 | 0.97 | 1.99 | Stem loop 2 | 4 |
| T_e3_9 | 4332 | 3.73 | 1.37 | 0.87 | 1.99 | Tetraloop | 3 |
| T_e1b_20 | 4287 | 3.38 | 1.67 | 0.93 | 1.99 | Tetraloop | 1 |
| L2_e5_32 | 3991 | 3.19 | 1.95 | 0.85 | 1.99 | Stem loop 2 | 5 |
| L2_e3_3 | 3862 | 3.44 | 1.64 | 0.91 | 2.00 | Stem loop 2 | 3 |
| L2_e3_1 | 3860 | 2.79 | 2.18 | 1.02 | 2.00 | Stem loop 2 | 3 |
| T_e2_1 | 4292 | 3.97 | 1.21 | 0.81 | 2.00 | Tetraloop | 2 |
| T_e3_25 | 4348 | 3.59 | 1.73 | 0.72 | 2.01 | Tetraloop | 3 |
| T_e1b_9 | 4276 | 3.71 | 1.06 | 1.28 | 2.02 | Tetraloop | 1 |
| T_e2_18 | 4309 | 4.30 | 0.79 | 0.98 | 2.02 | Tetraloop | 2 |
| T_e2_10 | 4301 | 3.63 | 1.63 | 0.83 | 2.03 | Tetraloop | 2 |
| T_e1b_16 | 4283 | 3.40 | 1.81 | 0.94 | 2.05 | Tetraloop | 1 |
| T_e3_5 | 4328 | 3.46 | 1.77 | 0.93 | 2.05 | Tetraloop | 3 |
| T_e2_11 | 4302 | 3.98 | 1.17 | 1.05 | 2.07 | Tetraloop | 2 |
| T_e1b_11 | 4278 | 3.42 | 1.71 | 1.08 | 2.07 | Tetraloop | 1 |
| T_e2_5 | 4296 | 3.81 | 1.59 | 0.83 | 2.08 | Tetraloop | 2 |
| T_e1a_3 | 4262 | 3.62 | 1.57 | 1.04 | 2.08 | Tetraloop | 1 |
| T_e2_13 | 4304 | 3.91 | 1.51 | 0.84 | 2.08 | Tetraloop | 2 |
| T_e3_1 | 4324 | 4.06 | 1.24 | 0.96 | 2.09 | Tetraloop | 3 |
| T_e2_20 | 4311 | 3.73 | 1.56 | 0.97 | 2.09 | Tetraloop | 2 |
| T_e2_4 | 4295 | 4.07 | 1.36 | 0.82 | 2.09 | Tetraloop | 2 |
| T_e2_29 | 4320 | 4.15 | 1.14 | 0.97 | 2.09 | Tetraloop | 2 |
| T_e1b_24 | 4291 | 4.43 | 0.71 | 1.13 | 2.09 | Tetraloop | 1 |
| T_e1a_4 | 4263 | 4.00 | 1.26 | 1.01 | 2.09 | Tetraloop | 1 |
| T_e3_19 | 4342 | 4.32 | 1.24 | 0.71 | 2.09 | Tetraloop | 3 |
| T_e3_6 | 4329 | 3.48 | 1.73 | 1.08 | 2.10 | Tetraloop | 3 |
| T_e1a_7 | 4266 | 4.04 | 1.49 | 0.77 | 2.10 | Tetraloop | 1 |
| T_e1b_2 | 4269 | 3.61 | 1.66 | 1.06 | 2.11 | Tetraloop | 1 |
| T_e2_32 | 4323 | 4.01 | 1.44 | 0.89 | 2.11 | Tetraloop | 2 |
| T_e2_25 | 4316 | 4.03 | 1.43 | 0.89 | 2.12 | Tetraloop | 2 |
| sl2_flip | 4355 | 3.01 | 2.21 | 1.17 | 2.13 | Stem loop 2 | 0 |
| T_e1b_18 | 4285 | 4.39 | 1.22 | 0.80 | 2.14 | Tetraloop | 1 |
| T_e1b_7 | 4274 | 4.20 | 1.30 | 0.92 | 2.14 | Tetraloop | 1 |
| T_e2_24 | 4315 | 3.71 | 1.92 | 0.81 | 2.15 | Tetraloop | 2 |
| T_e1b_3 | 4270 | 4.13 | 1.34 | 0.98 | 2.15 | Tetraloop | 1 |
| T_e3_20 | 4343 | 4.12 | 1.47 | 0.87 | 2.15 | Tetraloop | 3 |
| T_e1a_2 | 4261 | 4.33 | 1.07 | 1.09 | 2.16 | Tetraloop | 1 |
| T_e3_18 | 4341 | 4.06 | 1.45 | 0.98 | 2.16 | Tetraloop | 3 |
| T_e3_16 | 4339 | 3.83 | 1.62 | 1.12 | 2.19 | Tetraloop | 3 |
| T_e2_15 | 4306 | 4.06 | 1.75 | 0.78 | 2.19 | Tetraloop | 2 |
| T_e3_11 | 4334 | 4.13 | 1.89 | 0.59 | 2.2 | Tetraloop | 3 |
| T_e1b_8 | 4275 | 4.33 | 1.54 | 0.76 | 2.2 | Tetraloop | 1 |
| T_e1a_6 | 4265 | 4.65 | 1.12 | 0.87 | 2.2 | Tetraloop | 1 |
| T_e3_10 | 4333 | 4.09 | 1.64 | 0.92 | 2.22 | Tetraloop | 3 |
| T_e2_22 | 4313 | 4.31 | 1.39 | 0.96 | 2.22 | Tetraloop | 2 |
| T_e1b_22 | 4289 | 3.81 | 1.92 | 0.93 | 2.22 | Tetraloop | 1 |
| T_e1b_21 | 4288 | 3.79 | 2.30 | 0.58 | 2.22 | Tetraloop | 1 |
| T_e2_2 | 4293 | 3.94 | 1.99 | 0.75 | 2.23 | Tetraloop | 2 |
| dnr6_flip | 4352 | 4.34 | 1.42 | 0.93 | 2.23 | Tetraloop | 1 |
| L2_e5_23 | 3982 | 2.82 | 2.86 | 1.04 | 2.24 | Stem loop 2 | 5 |
| L2_e3_6 | 3865 | 2.70 | 3.14 | 0.94 | 2.26 | Stem loop 2 | 3 |
| T_e2_6 | 4297 | 4.32 | 1.31 | 1.17 | 2.27 | Tetraloop | 2 |
| T_e2_14 | 4305 | 4.13 | 1.46 | 1.21 | 2.27 | Tetraloop | 2 |
| T_e1b_5 | 4272 | 4.21 | 1.53 | 1.08 | 2.28 | Tetraloop | 1 |
| T_e2_30 | 4321 | 4.49 | 1.59 | 0.77 | 2.28 | Tetraloop | 2 |
| T_e3_15 | 4338 | 4.00 | 1.91 | 0.93 | 2.28 | Tetraloop | 3 |
| T_e1b_1 | 4268 | 3.44 | 2.33 | 1.08 | 2.28 | Tetraloop | 1 |
| T_e1b_10 | 4277 | 4.26 | 1.74 | 0.88 | 2.29 | Tetraloop | 1 |
| T_e3_26 | 4349 | 4.28 | 1.73 | 0.89 | 2.30 | Tetraloop | 3 |
| T_e2_8 | 4299 | 4.28 | 1.57 | 1.04 | 2.30 | Tetraloop | 2 |
| T_e1b_17 | 4284 | 4.00 | 1.92 | 1.00 | 2.31 | Tetraloop | 1 |
| F + E | 4354 | 4.32 | 2.05 | 0.57 | 2.31 | Tetraloop | 5 |
| T_e2_3 | 4294 | 4.28 | 1.81 | 0.92 | 2.34 | Tetraloop | 2 |
| T_e2_28 | 4319 | 4.42 | 1.54 | 1.14 | 2.37 | Tetraloop | 2 |
| T_e2_31 | 4322 | 4.55 | 1.61 | 0.94 | 2.37 | Tetraloop | 2 |
| T_e1b_19 | 4286 | 4.13 | 1.74 | 1.25 | 2.37 | Tetraloop | 1 |
| T_e1b_23 | 4290 | 4.59 | 1.52 | 1.09 | 2.40 | Tetraloop | 1 |
| T_e3_23 | 4346 | 4.26 | 1.73 | 1.31 | 2.43 | Tetraloop | 3 |
| T_e1b_4 | 4271 | 4.62 | 1.63 | 1.16 | 2.47 | Tetraloop | 1 |
| T_e1a_5 | 4264 | 4.34 | 2.24 | 0.85 | 2.48 | Tetraloop | 1 |
| T_e2_26 | 4317 | 4.80 | 1.65 | 1.08 | 2.51 | Tetraloop | 2 |
| T_e3_7 | 4330 | 4.70 | 1.83 | 1.04 | 2.52 | Tetraloop | 3 |
| T_e2_21 | 4312 | 4.35 | 1.87 | 1.36 | 2.53 | Tetraloop | 2 |
| FE_sl2 | 4356 | 4.55 | 2.44 | 0.98 | 2.66 | Tetraloop | 5 |
| T_e1b_13 | 4280 | 5.20 | 2.29 | 0.99 | 2.83 | Tetraloop | 1 |

Table 16. Prime Editing Efficiency with PEgRNAs Having Modified gRNA Cores

Editing efficiency with PEgRNAs having a gRNA core sequence according to SEQ ID NO: 4452, which includes the “M4” substitution, a “s12_flip” modification (a A to G substitution at nucleotide 49, U to G substitution at nucleotide 51, A to C substitution at nucleotide 58, and U to C substitution at nucleotide 60 compared to SEQ ID NO: 16), and an extension in the tetraloop (replacement of nucleotides 11-12 with GGG and replacement of nucleotides 17-18 with UCC) was also tested. PEgRNA with various spacer, PBS, and editing template sequences were designed to target one of multiple loci in a human gene (referred to as target 4). Each PEgRNA contains, from 5′ to 3′, the spacer, a gRNA core, and an extension arm that includes an editing template and a PBS, where the gRNA core sequence is according to either SEQ ID NO: 4452 or the canonical SpCas9 gRNA core according to SEQ ID NO: 16. For each PEgRNA having the canonical SpCas9 gRNA core, a PEgRNA that contains the same spacer, the same extension arm, and a gRNA core according to SEQ ID NO: 4452 was designed; the two PEgRNAs are identical except for the gRNA core sequences. A total 381 PEgRNAs having the canonical SpCas9 gRNA core and 381 corresponding PEgRNAs having the gRNA core of SEQ ID NO: 4452 were assembled as described, and editing efficiency was examined in Example 1. Compared to PEgRNAs having the canonical SpCas9 gRNA core sequence, a 1.59 fold improvement of average editing efficiency was observed with PEgRNAs having the gRNA core sequence according to SEQ ID NO: 4452.

### INCORPORATION BY REFERENCE

All publications and patent applications mentioned herein are hereby incorporated by reference in their entirety as if each individual publication or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments of the methods and compositions provided herein. Such equivalents are intended to be encompassed by the following claims.

LENGTHY TABLES
The patent application contains a lengthy table section. A copy of the table is available in electronic form from the USPTO web site (https://seqdata.uspto.gov/?pageRequest=docDetail&DocID=US20250297246A1). An electronic copy of the table will also be available from the USPTO upon request and payment of the fee set forth in 37 CFR 1.19(b)(3).

1.-24. (canceled)

25. A prime editing guide RNA (PEgRNA) comprising:
(a) a spacer that comprises a region of complementarity to a search target sequence in target strand of a double stranded target DNA;
(b) an extension arm comprising:
(i) an editing template that comprises an intended edit compared to the double stranded target DNA, and
(ii) a primer binding site (PBS) that comprises a region of complementarity to a region upstream of a nick site in a non-target strand of the double stranded target DNA; and
(c) a guide RNA (gRNA) core comprising at least 80% identity to SEQ ID NO: 16 and containing one or more modifications relative to SEQ ID NO: 16, the one or more modifications comprising:
i. a first insertion between nucleotides 12 and 13 and a second insertion between nucleotides 16 and 17, wherein the first insertion is the reverse complement of the second insertion;
ii. a first insertion between nucleotides 52 and 53 and a second insertion between nucleotides 56 and 57, wherein the first insertion is the reverse complement of the second insertion;
iii. complementary substitutions of nucleotides 2 and 29, 3 and 28, 4 and 27, 51 and 58, or combinations thereof;
iv. replacement of nucleotides 11-12 with a replacement sequence 1 and replacement of nucleotides 17-18 with a replacement sequence 2, wherein the replacement sequences 1 is at least 3 nucleotides in length and wherein the replacement sequence 2 is the reverse compliment of replacement sequence 1;
v. a T to G or T to C substitution at nucleotide 5 and a complementary substitution at nucleotide 26; or
vi. any combination thereof.

26. The PEgRNA of claim 25, wherein the gRNA core comprises:
the first insertion between nucleotides 12 and 13, and the second insertion between nucleotides 16 and 17, wherein the first insertion is 1 to 6 nucleotides in length or 1 to 3 nucleotides in length.

27. (canceled)

28. The PEgRNA of claim 26, wherein the first insertion comprises the sequence UGCUG.

29-30. (canceled)

31. The PEgRNA of claim 26, wherein the first insertion comprises a sequence selected from the group consisting of C, CC, CA, CG, A, AC, AA, AG, CCC, CCAC, CCAAC, and CCACAC.

32. The PEgRNA of claim 25, wherein the gRNA core comprises the first insertion between nucleotides 52 and 53 and the second insertion between nucleotides 56 and 57, wherein the first insertion is 1 to 8 nucleotides in length.

33. (canceled)

34. The PEgRNA of claim 25, wherein the gRNA core comprises the complementary substitutions of nucleotides 2 and 29, 3 and 28, 4 and 27, 11 and 18, 12 and 17, 51 and 58, or combinations thereof.

35. The PEgRNA of claim 32, wherein the gRNA core comprises:
(i) a U to A substitution at nucleotide 2;
(ii) a U to A substitution at nucleotide 3; or
(iii) a U to A substitution at nucleotide 4.

36.-37. (canceled)

38. The PEgRNA of claim 34, wherein the gRNA core comprises a U to G substitution at nucleotide 51 and an A to C substitution at nucleotide 58.

39. The PEgRNA of claim 25, wherein the gRNA core comprises the replacement of nucleotides 11-12 with the replacement sequence 1 and the replacement of nucleotides 17-18 with the replacement sequence 2, wherein the replacement sequence 1 is 3 to 5 nucleotides in length, and wherein the replacement sequence 1 comprises a sequence selected from the group consisting of CAGC, CCGC, GGAC, UGC, UCC, GAGGC, AGC, GGC, CGCA, GCACA, GGUC, and GGG.

40.-41. (canceled)

42. The PEgRNA of claim 25, wherein the gRNA core further comprises a U to A substitution at nucleotide 5 and an A to U substitution at nucleotide 26.

43. The PEgRNA of claim 25, wherein the gRNA core comprises complementary substitutions at nucleotides 52 and 57.

44. The PEgRNA of claim 43, wherein the gRNA core comprises:
(i) a U to G substitution at nucleotide 52 and an A to C substitution at nucleotide 57; or
(ii) a U to C substitution at nucleotide 52 and an A to G substitution at nucleotide 57.

45. (canceled)

46. The PEgRNA of claim 25, wherein the gRNA core comprises complementary substitutions at nucleotides 49 and 60.

47. The PEgRNA of claim 46, wherein the gRNA core comprises an A to G substitution at nucleotide 49 and a U to C substitution at nucleotide 60.

48.-49. (canceled)

50. A prime editing guide RNA (PEgRNA) comprising:
(a) a spacer that comprises a region of complementarity to a search target sequence in target strand of a double stranded target DNA;
(b) an extension arm comprising:
(i) an editing template that comprises an intended edit compared to the double stranded target DNA, and
(ii) a primer binding site (PBS) that comprises a region of complementarity to a region upstream of a nick site in a non-target strand of the double stranded target DNA; and
(c) a guide RNA (gRNA) core comprising a sequence selected from the group consisting of SEQ ID NOs: 17-61, 3860-4253, 4255-4349, 4351-4359, and 4452.

51. The PEgRNA of claim 50, wherein the gRNA core comprises:
(i) a sequence selected from the group consisting of SEQ ID NOs: 4352, 3860, 3862, 3865, 3908, 3915, 3982, 3991, 4035, 4261, 4262, 4263, 4264, 4265, 4266, 4268, 4277, 4278, 4280, 4283, 4284, 4285, 4286, 4269, 4287, 4288, 4289, 4290, 4291, 4270, 4271, 4272, 4274, 4275, 4276, 4292, 4301, 4302, 4304, 4305, 4306, 4309, 4293, 4311, 4312, 4313, 4315, 4316, 4317, 4319, 4320, 4294, 4321, 4322, 4323, 4295, 4296, 4297, 4299, 4324, 4333, 4334, 4338, 4339, 4341, 4342, 4343, 4345, 4346, 4348, 4349, 4328, 4329, 4330, and 4332;
(ii) a sequence selected from the group consisting of SEQ ID NOs: 4294, 4319, 4322, 4286, 4290, 4346, 4271, 4264, 4317, 4330, 4312, 4356, 4280, and 4452; or
(iii) SEQ ID NO: 4354.

52.-100. (canceled)

101. A prime editing system comprising:
(a) the PEgRNA of claim 25 or one or more polynucleotides encoding the PEgRNA; and
(b) a prime editor comprising a Cas9 protein and a reverse transcriptase or one or more polynucleotides encoding the prime editor, wherein the Cas9 protein comprises a mutation in an HNH domain.

102.-111. (canceled)

112. The prime editing system of claim 101, comprising one or more AAV vectors that comprises the one or more polynucleotides encoding the PEgRNA and the one or more polynucleotides encoding the prime editor, wherein the one or more polynucleotides encoding the prime editor comprise (a) a first sequence encoding an N-terminal portion of the Cas protein and an intein-N and (b) a second sequence encoding an intein-C, a C-terminal portion of the Cas protein and the DNA polymerase.

113.-115. (canceled)

116. A lipid nanoparticle (LNP) or ribonucleoprotein (RNP) comprising the prime editing system of claim 101.

117. A method for editing a double stranded target DNA, the method comprising contacting the target DNA with the PEgRNA of claim 25 and a prime editor comprising a Cas9 nickase and a reverse transcriptase.

118.-120. (canceled)
